SEQUENCE LISTING (227 sequences)

SEQ ID NO: 1 - DNA, 321 bp, Artificial Sequence
  Description of Artificial Sequence: Synthetic polynucleotide
acgtgcgatgatgcgtagctagtagtgatgatgcgtagctagtagtacgtgcgatgatgcgtagctagtagtgatgatgcgtagctagtagtacgtgcgatgatgcgtagctagtagtgatgatgcgtagctagtagtacgtgcgatgatgcgtagctagtagtgatgatgcgtagctagtagtacgtgcttggtaccatccgggccggccgcttaagcgacgcctataaaaaataggttgcatgctaggcctagcgctgccagtccatcttcgctagcctgtgctgcgtcagtccagcgctgcgctgcgtaacggccgcc

SEQ ID NO: 2 - DNA, 304 bp, Artificial Sequence
  Description of Artificial Sequence: Synthetic polynucleotide
ctgcacgtagatgatgcgtagctagtagtctgcacgtagatgatgcgtagctagtagtctgcacgtagatgatgcgtagctagtagtctgcacgtagatgatgcgtagctagtagtctgcacgtagatgatgcgtagctagtagtctgcacgtagtagtcgtatgctgatgcgcagttagcgtagctgaggtaccgtcgacgatatcggatccaggtctatataagcagagctcgtttagtgaaccgtcagatcgcctagatacgccatccacgctgttttgacctccatagaagatcgccacc

SEQ ID NO: 3 - DNA, 153 bp, Artificial Sequence
  Description of Artificial Sequence: Synthetic polynucleotide
ctgcacgtactgcacgtactgcacgtactgcacgtatgggtaccgtcgacgatatcggatccaggtctatataagcagagctcgtttagtgaaccgtcagatcgcctagatacgccatccacgctgttttgacctccatagaagatcgccacc

SEQ ID NO: 4 - DNA, 189 bp, Artificial Sequence
  Description of Artificial Sequence: Synthetic polynucleotide
accttgagtacgtgcgtctctgcacgtatggcgattaagaccttgagtacgtgcgtctctgcacgtatggcgattaagaccttgagtacgtgcgtctctgcacgtatggcgattaagaccttgagtacgtgcgtctctgcacgtatggcgattaatccatatgctctagagggtatataatgggggcca

SEQ ID NO: 5 - DNA, 255 bp, Artificial Sequence
  Description of Artificial Sequence: Synthetic polynucleotide
accttgagtacgtgcgtctctgcacgtatggcgattaagaccttgagtacgtgcgtctctgcacgtatggcgattaagaccttgagtacgtgcgtctctgcacgtatggcgattaagaccttgagtacgtgcgtctctgcacgtatggcgattaatccatatgcaggtctatataagcagagctcgtttagtgaaccgtcagatcgcctagatacgccatccacgctgttttgacctccatagaagatcgccacc

SEQ ID NO: 6 - DNA, 371 bp, Artificial Sequence
  Description of Artificial Sequence: Synthetic polynucleotide
tgacgtcacgattaccattgacgtcacgattaccattgacgtcacgattaccattgacgtcacgattaccattgacgtcagcgattaagatgactcagcgattaagatgactcagcgattaagatgactcagcgattaagatgactcactagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaa

SEQ ID NO: 7 - DNA, 256 bp, Artificial Sequence
  Description of Artificial Sequence: Synthetic polynucleotide
tgacgtcacgattaccattgacgtcacgattaccattgacgtcacgattaccattgacgtcacgattaccattgacgtcagcgattaagatgactcagcgattaagatgactcagcgattaagatgactcagcgattaagatgactcagcgattaatccatatgcaggtctatataagcagagctcgtttagtgaaccgtcagatcgcctagatacgccatccacgctgttttgacctccatagaagatcgccacc

SEQ ID NO: 8 - DNA, 318 bp, Artificial Sequence
  Description of Artificial Sequence: Synthetic polynucleotide
tgagtcagatgatgcgtagctagtagttgagtcagatgatgcgtagctagtagttgagtcagatgatgcgtagctagtagttgagtcagatgatgcgtagctagtagttgagtcagatgatgcgtagctagtagttgagtcagatgatgcgtagctagtagttgagtcagatgatgcgtagctagtagttgagtcagtagtcgtatgctgatgcgcagttagcgtagctgaggtaccgtcgacgatatcggatccttcgcatattaaggtgacgcgtgtggcctcgaacaccgagcgaccctgcagcgacccgcttaa

SEQ ID NO: 9 - DNA, 331 bp, Artificial Sequence
  Description of Artificial Sequence: Synthetic polynucleotide
tgagtcagatgatgcgtagctagtagttgagtcagatgatgcgtagctagtagttgagtcagatgatgcgtagctagtagttgagtcagatgatgcgtagctagtagttgagtcagatgatgcgtagctagtagttgagtcagatgatgcgtagctagtagttgagtcagatgatgcgtagctagtagttgagtcagtagtcgtatgctgatgcgcagttagcgtagctgaggtaccgtcgacgatatcggatccgggcatataaaacaggggcaaggcacagactcatagcagagcaatcaccaccaagcctggaataactgcagccacc

SEQ ID NO: 10 - DNA, 511 bp, Artificial Sequence
  Description of Artificial Sequence: Synthetic polynucleotide
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 11 - DNA, 1184 bp, Artificial Sequence
  Description of Artificial Sequence: Synthetic polynucleotide
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 12 - DNA, 492 bp, Artificial Sequence
  Description of Artificial Sequence: Synthetic polynucleotide
ccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccggg

SEQ ID NO: 13 - DNA, 1701 bp, Artificial Sequence
  Description of Artificial Sequence: Synthetic polynucleotide
actagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaa

SEQ ID NO: 14 - DNA, 1056 bp, Artificial Sequence
  Description of Artificial Sequence: Synthetic polynucleotide
atggtgcccaagaagaagaggaaagtctccaacctgctgactgtgcaccaaaacctgcctgccctccctgtggatgccacctctgatgaagtcaggaagaacctgatggacatgttcagggacaggcaggccttctctgaacacacctggaagatgctcctgtctgtgtgcagatcctgggctgcctggtgcaagctgaacaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacattgtgatgaactacatcagaaacctggactctgagactggggccatggtgaggctgctcgaggatggggactga

SEQ ID NO: 15 - DNA, 1299 bp, Artificial Sequence
  Description of Artificial Sequence: Synthetic polynucleotide
atggctcctaagaagaagaggaaggtgatgagccagttcgacatcctgtgcaagaccccccccaaggtgctggtgcggcagttcgtggagagattcgagaggcccagcggcgagaagatcgccagctgtgccgccgagctgacctacctgtgctggatgatcacccacaacggcaccgccatcaagagggccaccttcatgagctacaacaccatcatcagcaacagcctgagcttcgacatcgtgaacaagagcctgcagttcaagtacaagacccagaaggccaccatcctggaggccagcctgaagaagctgatccccgcctgggagttcaccatcatcccttacaacggccagaagcaccagagcgacatcaccgacatcgtgtccagcctgcagctgcagttcgagagcagcgaggaggccgacaagggcaacagccacagcaagaagatgctgaaggccctgctgtccgagggcgagagcatctgggagatcaccgagaagatcctgaacagcttcgagtacaccagcaggttcaccaagaccaagaccctgtaccagttcctgttcctggccacattcatcaactgcggcaggttcagcgacatcaagaacgtggaccccaagagcttcaagctggtgcagaacaagtacctgggcgtgatcattcagtgcctggtgaccgagaccaagacaagcgtgtccaggcacatctactttttcagcgccagaggcaggatcgaccccctggtgtacctggacgagttcctgaggaacagcgagcccgtgctgaagagagtgaacaggaccggcaacagcagcagcaacaagcaggagtaccagctgctgaaggacaacctggtgcgcagctacaacaaggccctgaagaagaacgccccctaccccatcttcgctatcaagaacggccctaagagccacatcggcaggcacctgatgaccagctttctgagcatgaagggcctgaccgagctgacaaacgtggtgggcaactggagcgacaagagggcctccgccgtggccaggaccacctacacccaccagatcaccgccatccccgaccactacttcgccctggtgtccaggtactacgcctacgaccccatcagcaaggagatgatcgccctgaaggacgagaccaaccccatcgaggagtggcagcacatcgagcagctgaagggcagcgccgagggcagcatcagataccccgcctggaacggcatcatcagccaggaggtgctggactacctgagcagctacatcaacaggcggatctga

SEQ ID NO: 16 - DNA, 1050 bp, Artificial Sequence
  Description of Artificial Sequence: Synthetic polynucleotide
atgcctaagaagaagaggaaggtttctgagctgattattagtggttcatctggtggattcctgcgaaacatcggcaaagagtatcaggaggccgctgaaaacttcatgaggtttatgaatgaccagggggcgtacgctcctaacactttgagggatttgaggttggtctttcatagctgggccagatggtgccatgctcggcagcttgcatggtttccaattagtcctgaaatggcacgcgaatactttcttcagttgcacgatgcagacctggcctccactaccatcgacaagcactatgctatgcttaatatgcttctgtcccactgcggactgccacccttgtccgacgacaagtcagtgagtcttgccatgagaagaattagaagagaagccgcaaccgaaaagggtgagaggacaggacaggcaatccccctgcgctgggacgacctgaagctgctggatgtgctgctcagcaggagcgagcggctggtcgacctgcgcaacagggctttcctgttcgtagcctataacaccctcatgagaatgtctgaaatatcacgcatcagggttggggacttggatcagacaggagacacagtgaccctgcacatcagtcacactaagacaatcaccacagctgcgggccttgacaaagtgctctcccggcgaaccacagcagtgctcaatgactggctggacgtcagtgggcttagagaacatccagacgctgtgctcttcccacctatacaccggtcaaacaaagcccgcattactaccacgcccctgaccgcccctgccatggagaagattttcagtgatgcctgggtgctgctgaacaaacgggacgccacccccaataaagggaggtataggacctggaccggccattccgccagggtgggtgccgcaatagacatggccgagaaacaggtgtctatggtcgagattatgcaggaagggacatggaagaagcctgaaacactgatgcggtatctcagaaggggcggagtgtccgtgggagccaattctcgactgatggatagctaa

SEQ ID NO: 17 - DNA, 1137 bp, Artificial Sequence
  Description of Artificial Sequence: Synthetic polynucleotide
atgcccaagaaaaagcggaaggtgtccctgctgaccaccaacaaccacagcgtggccctgagctacggcgagcctcctagcaccctgaacgacagcctgaaggacagctaccagcggagcaccgatgagctgcaggccctgctgtctaagcctctggcccagctgaccgacgccgacaagctgcggatcagagagatcacccaggccaagctgaagcacttcctggacaacggccaccggaccagaagggccaacacttggagagccctgatgagcagatgggccaagttcgagagctggtgcctgaccaacaatctgacccccctgcctgccacccctgaggtggtggccacattcatcgagtactaccaggccagcagctacaccaccctgagccagtatgcctgggccatcaacagctttcacgtggaatgcggcctgctgagccccgtgtctagcaagaccgtgcaggacaagcagaacgagatcagaatcgtgaagctggaatctggcggcctggcccaggaacaggccaccccttttagactgcaccatctgcagatgctgatcgagagctatggcgagagcgagcggctgctggacaagagaaacctggctctgctgaatatcgcctacgagagcctgctgcgcgagtccgagctgctgagaatcaaagtgggccacctgaagtccaccttcgagggcgactacgtgctgagcgtgccctacaccaagaccaacgacagcggcgaagaggaagtcgtgaacatcacccccctgggcttcaagctgatccagcggtacatccagggcgctggcctgacaaaagaggactacctgttccagcccatcggccggtccaacaaggtgtccgtgcaggccaaacccatgagcacccggaccgtggacagagtgttcctgtgggcctttgagagcctgggcatcgacagacacagcgcttggagcggccacagcgccagaattggagccgctcaggatctgctggccgctggctattctatcgcccagatccaggaaaacggccgctggaagtcccccatgatggtgctgagatacggcaaggacatcaaggccaaagaaagcgccatggccaagatgctggccgagcggagatga

SEQ ID NO: 18 - DNA, 1164 bp, Artificial Sequence
  Description of Artificial Sequence: Synthetic polynucleotide
atgcccaagaaaaagcggaaagtgatcgagaaccagctgagcctgctgggcgacttttctggcgtgcggcccgacgatgtgaaaaccgccattcaggccgcccagaaaaagggcatcaacgtggccgagaacgagcagttcaaggccgccttcgagcatctgctgaacgagttcaagaagcgggaagagagatacagccccaacaccctgcggcggctggaaagcgcctggacctgcttcgtggattggtgcctggccaaccacagacacagcctgcctgccacccccgataccgtggaagccttcttcatcgagcgggccgaggaactgcaccggaacaccctgagcgtgtacagatgggccatcagccgggtgcacagagtggccggatgccctgatccctgcctggacatctacgtggaagatcggctgaaggccattgcccggaagaaagtgcgggaaggcgaggccgtgaagcaggccagccctttcaacgagcagcatctgctgaagctgaccagcctgtggtacagaagcgacaagctgctgctgcggcggaacctggctctgctggctgtggcctacgagagcatgctgagagccagcgagctggccaacatccgggtgtccgatatggaactggccggcgacggaaccgccatcctgaccatccctatcaccaagaccaaccactccggcgagcccgatacctgcatcctgtcccaggatgtggtgtccctgctgatggactacaccgaggccggcaagctggatatgagcagcgacggcttcctgttcgtgggcgtgtccaagcacaacacctgtatcaagcccaagaaggacaagcagaccggcgaggtgctgcacaagcccatcaccaccaagacagtggaaggcgtgttctacagcgcctgggagacactggacctgggcagacagggcgtgaagcctttcacagcccacagcgccagagtgggagccgctcaggacctgctgaagaagggctacaataccctgcagatccagcagtccggccggtggtctagcggagccatggtggccagatacggcagagccatcctggctagggatggcgctatggcccacagcagagtgaaaaccagatccgcccccatgcagtggggcaaggacgagaaggactga

SEQ ID NO: 19 - DNA, 1107 bp, Artificial Sequence
  Description of Artificial Sequence: Synthetic polynucleotide
atgcccaagaaaaagcggaaagtgaccgacctgaccccattcccccccctggaacacctggaacccgacgagtttgccgacctcgtgcggaaggccatcaagagggatcctcaggctggcgcccaccctgccatccagtctgccatcagccacttccaggacgagttcgtgcggagacagggcgaatggcagcctgccacactgcagagactgagaaacgcctggaatgtgtttgtgcggtggtgcacccaccagggcattccagctctgcctgccagacaccaggacgtggaaagatacctgatcgagcggcggaacgagctgcaccggaacaccctgaaagtgcacctgtgggccatcggcaagacccacgtgatcagcggcctgcccaatccctgcgcccacagatacgtgaaagcccagatggcccagatcacacaccagaaagtgcgcgagagagagcggatcgaacaggcccctgccttcagagagtccgacctggacagactgaccgagctgtggagcgccaccagaagcgtgacccagcagcgggacctgatgatcgtgtccctggcctacgagacactgctgcggaagaacaatctggaacagatgaaagtgggcgacatcgagttctgccaggacggctctgccctgatcaccatccccttcagcaagaccaaccacagcggcagggatgacgtgcggtggatctctccccaggtggccaatcaggtgcacgcctacctgcagctgcccaacatcgacgccgacccccagtgcttcctgctgcagagagtgaagagaagcggcaaggccctgaaccccgagagccacaataccctgaacggccaccaccccgtgtccgagaagctgatctcccgggtgttcgagcgggcttggagagccctgaatcacgagacaggccccagatacaccggccacagcgctagagtgggagccgctcaggatctgctgcaggaaggctacagcaccctgcaagtgatgcaggcaggcggctggtccagcgagaagatggtgctgagatacggccggcatctgcacgcccacacatctgccatggctcagaaacggcggcagcggtga

SEQ ID NO: 20 - DNA, 1728 bp, Artificial Sequence
  Description of Artificial Sequence: Synthetic polynucleotide
atgcccaagaaaaagcggaaggtgtccagctacatggacctggtggacgacgagcccgccaccctgtaccacaagttcgtggaatgcctgaaggccggcgagaacttctgcggcgataagctgagcggcatcatcaccatggccattctgaaggccatcaaggccctgaccgaagtgaagaaaaccaccttcaacaagtacaagaccaccatcaagcagggcctgcagtacgacgtgggcagcagcaccatcagcttcgtgtaccacctgaaggactgcgacgagctgagcagaggcctgagcgacgccttcgagccctacaagttcaagatcaagagcaacaaagaggccaccagcttcaagaccctgttcaggggccctagcttcggcagccagaagaactggcggaagaaagaggtggaccgcgaggtggacaacctgttccacagcaccgagacagacgagagcatcttcaagttcatcctgaacaccctggacagcatcgaaacccagaccaacaccgaccggcagaaaaccgtgctgacctttatcctgctgatgaccttcttcaactgctgccggaacaacgacctgatgaacgtggaccccagcaccttcaagatcgtgaagaacaagtttgtgggctacctgctgcaggctgaagtgaagcagaccaagaccagaaagagccggaatatcttcttcttccccatccgggaaaaccgcttcgacctgttcctggccctgcacgacttcttcagaacctgccagcccacccccaagagcagactgagcgatcaggtgtccgagcagaagtggcagctgttccgggacagcatggtcatcgactacaaccggttctttcggaagttccccgccagccccatcttcgccattaagcacggccccaagtcccacctgggccggcatctgatgaacagctttctgcacaagaacgagctggacagctgggccaacagcctgggcaattggagcagctcccagaaccagagagagagcggcgccagactgggctacacacacggcggaagagatctgccccagcccctgtttggcttcctggccggatactgcgtgcggaacgaagagggccacatcgtgggcctgggcctggaaaaggacatcaacgatctgttcgacggcatcatggaccccctgaacgagaaagaggacaccgagatctgcgagagctacggcgagtgggccaagattgtgtccaaggacgtgctgatcttcctgaagagataccacagcaagaacgcctgtcggagataccagaacagcaccctgtatgcccggaccttcctgaaaaccgagagcgtgaccctgagcggctccaagggcagcgaggaaccttctagccctgtgcggatccccatcctgagcatgggaaaggccagcccctccgagggaagaaagctgagagccagcgagcacgccaacgacgacaacgagatcgagaagatcgacagcgacagcagccagagcgaagagatccctatcgagatgagcgactccgaggacgagacaaccgccagcaacatcagcggcatctacctggacatgagcaaggccaactccaacgtggtgtacagcccccctagccagacaggcagagctgctggcgccggaagaaaaagaggcgtgggaggcagacggaccgtggaaagcaagcggagaagagtgctggcccccatcaaccggtga

SEQ ID NO: 21 - DNA, 1365 bp, Artificial Sequence
  Description of Artificial Sequence: Synthetic polynucleotide
atgcccaagaaaaagcggaaggtgtccaccttcgccgaggccgcccatctgacacctcaccagtgcgccaacgagatcaatgagatcctggaaagcgacaccttcaacatcaacgccaaagagatccggaacaagctggcctccctgttcagcatcctgaccatgcagagcctgagcatccgcagagagatgaagatcaacacctaccggtcctacaagagcgccatcggcaagagcctgtccttcgacaaggacgacaagatcatcaagttcaccgtgcggctgagaaagaccgagagcctgcagaaggacatcgagagcgccctgcccagctacaaggtggtggtgtccccattcaagaaccaggaagtgtccctgttcgaccgctacgaggaaacccacaaatacgacgccagcatggtgggactgcagttcaccaacatcctgagcaaagagaaggatatctggaagatcgtgtcccggatcgcctgcttcttcgaccagagctgcgtgaccaccaccaagcgggccgagtacagactgctgctgctgggcgctgtgggcaactgctgcagatacagcgacctgaagaacctggacccccggaccttcgagatctacaacaacagcttcctgggccccatcgtgcgggccaccgtgacagagacaaagagccggaccgagagatacgtgaacttctaccccgtgaacggcgactgcgacctgctgatctccctgtacgactacctgagagtgtgcagccccatcgagaaaaccgtgtccagcaaccggcccaccaaccagacccaccagtttctgcctgagagcctggccagaaccttcagccggttcctgacccagcacgtggacgagcccgtgttcaagatctggaacggccccaagagccacttcggcagacacctgatggccacctttctgagcagaagcgagaagggcaaatacgtgtcctccctgggcaattgggctggcgaccgggaaatccagtctgccgtggccagaagccactacagccacggctctgtgaccgtggacgaccgggtgttcgccttcatcagcggcttctacaaagaggcccccctgggcagcgagatctatgtgctgaaggaccccagcaacaagcccctgagcagagaggaactgctggaagaggaaggcaacagcctgggctccccacctctgagccctccaagctctcctagactggtggcccagagcttcagcgcccacccaagcctgcagctgttcgagcagtggcacggcatcatcagcgacgaggtgctgcagtttatcgccgagtaccggcggaagcacgagctgagaagccagagaaccgtggtggcctga

SEQ ID NO: 22 - DNA, 1446 bp, Artificial Sequence
  Description of Artificial Sequence: Synthetic polynucleotide
atgcccaagaaaaagcggaaggtgtccgagttcagcgagctcgtgcggatcctgcccctggatcaggtggccgagatcaagagaatcctgagcagaggcgaccccatccccctgcagagactggcctctctgctgaccatggtcatcctgaccgtgaacatgagcaagaagagaaagagcagccccatcaagctgagcaccttcaccaagtaccggcggaacgtggccaagagcctgtactacgacatgagcagcaagaccgtgttcttcgagtaccacctgaagaacacccaggacctgcaggaaggcctggaacaggccattgccccctacaacttcgtcgtgaaagtgcacaagaagcccatcgactggcagaaacagctgagcagcgtgcacgagcggaaggccggccacagatccatcctgtccaacaacgtgggcgccgagatctccaagctggccgagacaaaggacagcacctggtccttcatcgagcggaccatggacctgatcgaggccagaaccagacagcccaccaccagagtggcctaccggttcctgctgcagctgaccttcatgaactgctgccgggccaacgatctgaagaacgccgaccccagcaccttccagatcattgccgatccccacctgggccggatcctgagagccttcgtgcccgagactaagacctctatcgagcggtttatctacttcttcccatgcaagggccgctgcgaccctctgctggccctggattcttacctgctgtgggtgggacccgtgcccaagacccagaccaccgatgaggaaacccagtacgactaccagctgctgcaggacaccctgctgatctcttacgaccggtttatcgccaaagagagcaaagagaacatcttcaagatccccaacggccccaaggcccatctgggcagacatctgatggccagctacctgggcaacaacagcctgaagtccgaggccaccctgtacggcaattggagcgtggaaagacaggaaggcgtgtccaaaatggccgacagccggtacatgcacaccgtgaagaagtcccccccctcctacctgttcgcctttctgagcggctactacaagaagtccaaccagggcgagtacgtgctggccgaaaccctgtacaaccccctggactacgataagaccctgcccatcaccaccaacgagaagctgatctgcagacgctacggcaagaacgccaaagtgatccccaaggatgccctgctgtacctgtacacctacgcccagcagaagcggaagcagctggctgaccccaacgagcagaaccggctgttcagcagcgagagccctgcccacccatttctgacccctcagagcacaggcagcagcacccctctgacatggaccgcccctaagacactgagcaccggcctgatgacccctggcgaggaatga

SEQ ID NO: 23 - DNA, 1494 bp, Artificial Sequence
  Description of Artificial Sequence: Synthetic polynucleotide
atgcccaagaaaaagcggaaggtgcagctgaccaaggacaccgagatcagcaccatcaaccggcagatgagcgacttcagcgagctgagccagatcctgcccctgcaccagatctccaagatcaaggacatcctggaaaacgagaaccccctgcccaaagagaagctggcctcccacctgaccatgatcatcctgatggccaacctggccagccagaaacggaaggacgtgcccgtgaagcggagcaccttcctgaagtaccagcggagcatcagcaagaccctgcagtacgacagcagcaccaagaccgtgtccttcgagtaccacctgaaggaccccagcaagctgatcaagggcctggaagatgtggtgtccccctacagattcgtcgtgggcgtgcacgagaagcccgacgacgtgatgtctcacctgagcgccgtgcacatgcggaaagaggccggcagaaagcgggacctgggcaacaagatcaacgacgagatcacaaagatcgccgagacacaggaaaccatctggggcttcgtgggcaagaccatggacctgatcgaggccagaaccacccggcctacaacaaaggccgcctacaacctgctgctgcaggccaccttcatgaactgctgcagagccgacgacctgaagaacaccgacatcaagaccttcgaagtgatccccgacaagcacctgggccggatgctgagagccttcgtgcccgagacaaagaccggaaccagattcgtgtacttcttcccatgcaagggcagatgcgaccccctgctggccctggattcttacctgcagtggaccgaccccatccccaagaccagaacaaccgacgaggacgccagatacgactaccagctgctgcggaacagcctgctgggcagctacgacggcttcatctccaagcagagcgacgagagcatcttcaagatccccaacggccccaaggcccacctgggcagacatgtgacagccagctacctgagcaacaacgagatggacaaagaggccaccctgtacggcaattggagcgccgctagagaagagggcgtgtccagagtggccaaggcccggtacatgcacaccatcgagaagtcccccccctcctacctgttcgccttcctgagcggcttctacaacatcaccgccgagagggcctgcgagctggtggaccccaatagcaacccctgcgagcaggacaagaacatccccatgatcagcgacatcgagacactgatggctcgctacggcaagaacgccgagatcatccctatggacgtgctggtgttcctgagcagctacgcccggttcaagaacaacgagggcaaagagtacaagctgcaggctcggagcagcagaggcgtgcccgacttccccgataatggcagaaccgccctgtacaacgccctgacagccgcccacgtgaagaggcggaagatcagcattgtcgtgggccggtccatcgacaccagctga

SEQ ID NO: 24 - DNA, 1839 bp, Artificial Sequence
  Description of Artificial Sequence: Synthetic polynucleotide
atgcctaagaaaaagcggaaagtggatacctacgccggagcctacgacagacagagccgggagagagagaacagcagcgccgccagccccgccacccagagaagcgccaacgaggataaggccgccgatctgcagagagaggtggagagggacggcggcagattcagatttgtgggccacttcagcgaggcccctggcaccagcgccttcggcaccgccgagagacccgagttcgagagaatcctgaacgagtgtagggccggcaggctgaacatgatcatcgtgtacgacgtgtcccggttcagcaggctgaaggtgatggacgccatccctatcgtgtccgagctgctggccctgggcgtgaccatcgtgtccacccaggaaggcgtctttagacagggcaacgtgatggacctgatccacctgatcatgaggctggacgccagccacaaggagagcagcctgaagagcgccaagatcctggacaccaagaacctgcagagggagctgggcggctatgtgggcggcaaggccccctacggcttcgagctggtgtccgagaccaaggagatcacccggaacggcaggatggtgaacgtggtgatcaacaagctggcccacagcaccacccccctgaccggccccttcgagtttgagcccgacgtgatcaggtggtggtggcgggagatcaagacccacaagcacctgcctttcaagcccggcagccaggccgccatccaccccggcagcatcaccggcctgtgtaagagaatggacgccgacgccgtgcccaccagaggcgagaccatcggcaagaaaaccgccagcagcgcctgggaccccgccaccgtgatgagaatcctgagggaccctaggatcgccggcttcgccgccgaggtgatctacaagaagaagcccgacggcacccccaccaccaagatcgagggctacagaatccagagagaccccatcaccctgagacctgtggagctggactgtggccctatcatcgagcctgccgagtggtacgagctgcaggcctggctggacggcagaggcagaggcaagggcctgagcagaggccaggccatcctgagcgccatggacaagctgtactgtgagtgtggcgccgtgatgaccagcaagagaggcgaggagagcatcaaggacagctaccggtgccggagaagaaaggtggtggaccccagcgcccctggccagcacgagggcacctgtaatgtgagcatggccgccctggacaagttcgtggccgagcggatcttcaacaagatccggcacgccgagggcgacgaggagaccctggccctgctgtgggaggccgccagaagattcggcaagctgaccgaggcccccgagaagagcggcgagagggccaacctggtggccgagagagccgacgccctgaacgccctggaggagctgtacgaggacagagccgccggagcctatgacggccctgtgggcaggaagcacttcagaaagcagcaggccgccctgaccctgagacagcagggcgccgaggaaagactggccgagctggaggccgccgaggcccctaagctgcccctggatcagtggttccccgaggatgccgacgccgaccccaccggccccaagtcctggtggggcagagccagcgtggacgacaagagggtgttcgtgggcctgttcgtggataagatcgtggtgaccaagagcaccaccggcaggggccagggcacccccatcgagaagagagccagcatcacctgggccaagcctcccaccgacgacgacgaggatgacgcccaggacggcaccgaggacgtggccgcctga

SEQ ID NO: 25 - DNA, 1530 bp, Artificial Sequence
  Description of Artificial Sequence: Synthetic polynucleotide
atggatcctaagaaaaagcgaaaagtgatgcgagccctggtggtcattcgcctgagcagagtcacagacgctactacaagccctgagcggcagctggagtcctgtcagcagctgtgcgcacagcgaggatgggatgtggtcggagtggcagaggatctggacgtgagcggggctgtcgatccattcgaccgaaagcggagacccaacctggcacgatggctggctttcgaggaacagccctttgatgtgatcgtcgcctacagagtggacaggctgacacgctcaattcgacatctgcagcagctggtgcattgggccgaggatcacaagaaactggtggtcagcgcaactgaagcccacttcgacaccacaactccttttgccgctgtggtcatcgcactgatgggcaccgtggcccagatggagctggaagctatcaaggagcgaaaccggagcgcagcccatttcaatattcgggccgggaaatacagaggcagcctgcccccttggggctatctgcctacccgggtggatggggagtggagactggtgccagaccccgtccagagagagaggattctggaagtgtaccacagagtggtggacaaccacgaaccactgcatctggtggcccacgatctgaataggcgcggagtcctgtctccaaaggactattttgctcagctgcagggaagggagccacagggacgagaatggagtgctaccgcactgaagcggtctatgatcagtgaggctatgctgggctatgcaactctgaatgggaaaaccgtgagagacgatgacggagcaccactggtgcgggctgagcctattctgacaagagagcagctggaagctctgagggcagaactggtgaaaaccagtagggccaagcctgctgtgtcaacaccaagcctgctgctgcgagtgctgttctgcgcagtctgtggcgagccagcatacaaatttgccggcgggggaaggaagcatccccgctatcgatgccggagcatggggttccctaagcactgtggaaacggcactgtggctatggccgaatgggacgccttttgtgaggaacaggtgctggatctgctgggggacgcagagcgcctggaaaaagtgtgggtcgctggaagcgattccgctgtggagctggcagaagtcaatgccgagctggtggacctgacctccctgatcggatctcctgcatacagggcaggctccccacagcgagaagctctggatgcacgaattgctgcactggcagctcgacaggaggaactggaggggctggaagccagaccctctggatgggagtggcgagaaacaggccagcggtttggggattggtggagggagcaggacacagcagccaagaacacttggctgagatccatgaatgtcaggctgactttcgacgtgcgaggaggactgacccgaacaatcgattttggcgacctgcaggagtatgaacagcatctgcgcctgggaagtgtggtcgagcgactgcacaccggcatgtcataa

SEQ ID NO: 26 - DNA, 34 bp, Artificial Sequence
  Description of Artificial Sequence: Synthetic oligonucleotide
ataacttcgtatagcatacattatacgaagttat

SEQ ID NO: 27 - DNA, 34 bp, Artificial Sequence
  Description of Artificial Sequence: Synthetic oligonucleotide
ataacttcgtataggataccttatacgaagttat

SEQ ID NO: 28 - DNA, 34 bp, Artificial Sequence
  Description of Artificial Sequence: Synthetic oligonucleotide
ataacttcgtatagtataccttatacgaagttat

SEQ ID NO: 29 - DNA, 34 bp, Artificial Sequence
  Description of Artificial Sequence: Synthetic oligonucleotide
gaagttcctattctctagaaagtataggaacttc

SEQ ID NO: 30 - DNA, 34 bp, Artificial Sequence
  Description of Artificial Sequence: Synthetic oligonucleotide
gaagttcctattcttcaaatagtataggaacttc

SEQ ID NO: 31 - DNA, 34 bp, Artificial Sequence
  Description of Artificial Sequence: Synthetic oligonucleotide
gaagttcctattctatcagaagtataggaacttc

SEQ ID NO: 32 - DNA, 32 bp, Artificial Sequence
  Description of Artificial Sequence: Synthetic oligonucleotide
taactttaaataattggcattatttaaagtta

SEQ ID NO: 33 - DNA, 34 bp, Artificial Sequence
  Description of Artificial Sequence: Synthetic oligonucleotide
tcaatttctgagaactgtcattctcggaaattga

SEQ ID NO: 34 - DNA, 34 bp, Artificial Sequence
  Description of Artificial Sequence: Synthetic oligonucleotide
tcaatttctgagaagtgtctttctcggaaattga

SEQ ID NO: 35 - DNA, 34 bp, Artificial Sequence
  Description of Artificial Sequence: Synthetic oligonucleotide
ctcgtgtccgataactgtaattatcggacatgat

SEQ ID NO: 36 - DNA, 34 bp, Artificial Sequence
  Description of Artificial Sequence: Synthetic oligonucleotide
ctcgtgtccgataactgtaattatcggacacgag

SEQ ID NO: 37 - DNA, 34 bp, Artificial Sequence
  Description of Artificial Sequence: Synthetic oligonucleotide
ctcgtgtccgataagtgtatttatcggacatgat

SEQ ID NO: 38 - DNA, 34 bp, Artificial Sequence
  Description of Artificial Sequence: Synthetic oligonucleotide
aataggtctgagaacgcccattctcagacgtatt

SEQ ID NO: 39 - DNA, 33 bp, Artificial Sequence
  Description of Artificial Sequence: Synthetic oligonucleotide
ggttgcttaagaataagtaattcttaagcaacc

SEQ ID NO: 40 - DNA, 61 bp, Artificial Sequence
  Description of Artificial Sequence: Synthetic oligonucleotide
aaacgatatcagacatttgtctgataatgcttcattatcagacaaatgtctgatatcgttt

SEQ ID NO: 41 - DNA, 39 bp, Artificial Sequence
  Description of Artificial Sequence: Synthetic oligonucleotide
gagtttcattaaggaataactaattccctaatgaaactc

SEQ ID NO: 42 - DNA, 31 bp, Artificial Sequence
  Description of Artificial Sequence: Synthetic oligonucleotide
ttgatgaaagaataacgtattctttcatcaa

SEQ ID NO: 43 - DNA, 45 bp, Artificial Sequence
  Description of Artificial Sequence: Synthetic oligonucleotide
tgcgggtgccagggcgtgcccttgggctccccgggcgcgtactcc

SEQ ID NO: 44 - DNA, 42 bp, Artificial Sequence
  Description of Artificial Sequence: Synthetic oligonucleotide
gtgccccaactggggtaacctttgagttctctcagttggggg

SEQ ID NO: 45 - DNA, 50 bp, Artificial Sequence
  Description of Artificial Sequence: Synthetic oligonucleotide
tcggccggcttgtcgacgacggcggtctccgtcgtcaggatcatccgggc

SEQ ID NO: 46 - DNA, 53 bp, Artificial Sequence
  Description of Artificial Sequence: Synthetic oligonucleotide
tcgtggtttgtctggtcaaccaccgcggtctcagtggtgtacggtacaaaccc

SEQ ID NO: 47 - DNA, 1308 bp, Artificial Sequence
  Description of Artificial Sequence: Synthetic polynucleotide
atgctggagcccggcgagaagccctacaagtgccccgagtgcggcaagagcttcagcagccccgccgacctgaccaggcaccagaggacccacaccggcgagaagccctacaagtgccccgagtgcggcaagagcttcagcgacaagaaggacctgaccaggcaccagaggacccacaccggcgagaagccctacaagtgccccgagtgcggcaagagcttcagcaggaacgacgccctgaccgagcaccagaggacccacaccggcgagaagccctacaagtgccccgagtgcggcaagagcttcagcgagaggagccacctgagggagcaccagaggacccacaccggcgagaagccctacaagtgccccgagtgcggcaagagcttcagcgagaggagccacctgagggagcaccagaggacccacaccggcgagaagccctacaagtgccccgagtgcggcaagagcttcagcgagaggagccacctgagggagcaccagaggacccacaccggcgagaagccctacaagtgccccgagtgcggcaagagcttcagcgactgcagggacctggccaggcaccagaggacccacaccggcgagaagccctacaagtgccccgagtgcggcaagagcttcagccacaggaccaccctgaccaaccaccagaggacccacaccggcgagaagccctacaagtgccccgagtgcggcaagagcttcagcacccacctggacctgatcaggcaccagaggacccacaccggcgagaagccctacaagtgccccgagtgcggcaagagcttcagcgactgcagggacctggccaggcaccagaggacccacaccggcgagaagccctacaagtgccccgagtgcggcaagagcttcagcaggagcgacaagctggtgaggcaccagaggacccacaccggcgagaagccctacaagtgccccgagtgcggcaagagcttcagcaggagggacgagctgaacgtgcaccagaggacccacaccggcgagaagccctacaagtgccccgagtgcggcaagagcttcagccagaagagcagcctgatcgcccaccagaggacccacaccggcaagaagaccagcccgaaaaagaaacgcaaagttgggcgcgccgacgcgctggacgatttcgatctcgacatgctgggttctgatgccctcgatgactttgacctggatatgttgggaagcgacgcattggatgactttgatctggacatgctcggctccgatgctctggacgatttcgatctcgatatgttaattaactaa

SEQ ID NO: 48 - DNA, 69 bp, Artificial Sequence
  Description of Artificial Sequence: Synthetic oligonucleotide
tctagagggtatataatgggggccactagtgccagcagcagcctgaccacatctcatcctccagccacc

SEQ ID NO: 49 - DNA, 1935 bp, Artificial Sequence
  Description of Artificial Sequence: Synthetic polynucleotide
tctagagggtatataatgggggccactagtgccagcagcagcctgaccacatctcatcctccagccaccatgccggggttttacgagattgtgattaaggtccccagcgaccttgacgagcatctgcccggcatttctgacagctttgtgaactgggtggccgagaaggagtgggagttgccgccagattctgacttggatctgaatctgattgagcaggcacccctgaccgtggccgagaagctgcagcgcgactttctgacggagtggcgccgtgtgagtaaggccccggaggcccttttctttgtgcaatttgagaagggagagagctacttccacttacacgtgctcgtggaaaccaccggggtgaaatccttagttttgggacgtttcctgagtcagattcgcgaaaaactgattcagagaatttaccgcgggatcgagccgactttgccaaactggttcgcggtcacaaagaccagaaacggcgccggaggcgggaacaaggtggtggacgagtgctacatccccaattacttgctccccaaaacccagcctgagctccagtgggcgtggactaatatagaacagtatttaagcgcctgtttgaatctcacggagcgtaaacggttggtggcgcagcatctgacgcacgtgtcgcagacgcaggagcagaacaaagagaatcagaatcccaattctgacgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 50 - RNA, 14 nt, Artificial Sequence
  Description of Artificial Sequence: Synthetic oligonucleotide
  modified_base (2)..(2): a, c, u, g, unknown or other
  modified_base (5)..(5): a, c, u, g, unknown or other
  modified_base (7)..(7): a, c, u, g, unknown or other
  modified_base (9)..(9): a, c, u, g, unknown or other
  modified_base (12)..(12): a, c, u, g, unknown or other
  See specification as filed for detailed description of substitutions and preferred embodiments
cngcngncngcngs

SEQ ID NO: 51 - RNA, 7 nt, Artificial Sequence
  Description of Artificial Sequence: Synthetic oligonucleotide
  modified_base (2)..(2): a, c, u, g, unknown or other
  modified_base (4)..(4): a, c, u, g, unknown or other
  modified_base (6)..(6): a, c, u, g, unknown or other
  See specification as filed for detailed description of substitutions and preferred embodiments
cngncng

SEQ ID NO: 52 - RNA, 20 nt, Artificial Sequence
  Description of Artificial Sequence: Synthetic oligonucleotide
cacucagacuacgugcaccu

SEQ ID NO: 53 - RNA, 20 nt, Artificial Sequence
  Description of Artificial Sequence: Synthetic oligonucleotide
cacucagacuacgugcaccu

SEQ ID NO: 54 - RNA, 20 nt, Artificial Sequence
  Description of Artificial Sequence: Synthetic oligonucleotide
cacucagacuacgugcuccu

SEQ ID NO: 55 - RNA, 20 nt, Artificial Sequence
  Description of Artificial Sequence: Synthetic oligonucleotide
cacucagacuacgugccccu

SEQ ID NO: 56 - RNA, 20 nt, Artificial Sequence
  Description of Artificial Sequence: Synthetic oligonucleotide
cacucagacuacgugcgccu

SEQ ID NO: 57 - RNA, 20 nt, Artificial Sequence
  Description of Artificial Sequence: Synthetic oligonucleotide
cacucagacuaugugcaccu

SEQ ID NO: 58 - RNA, 20 nt, Artificial Sequence
  Description of Artificial Sequence: Synthetic oligonucleotide
acgggcaacuuuacacgacg

SEQ ID NO: 59 - RNA, 33 nt, Artificial Sequence
  Description of Artificial Sequence: Synthetic oligonucleotide
  modified_base (2)..(2): a, c, u, g, unknown or other
  modified_base (5)..(5): a, c, u, g, unknown or other
  modified_base (28)..(28): a, c, u, g, unknown or other
  modified_base (31)..(31): a, c, u, g, unknown or other
cngcngcacucagacuacgugcaccucngcngc

SEQ ID NO: 60 - RNA, 33 nt, Artificial Sequence
  Description of Artificial Sequence: Synthetic oligonucleotide
cagcagcacucagacuacgugcaccucugcugc

SEQ ID NO: 61 - RNA, 32 nt, Artificial Sequence
  Description of Artificial Sequence: Synthetic oligonucleotide
  modified_base (2)..(2): a, c, u, g, unknown or other
  modified_base (31)..(31): a, c, u, g, unknown or other
cngcagcacucagacuacgugcaccucugcng

SEQ ID NO: 62 - RNA, 32 nt, Artificial Sequence
  Description of Artificial Sequence: Synthetic oligonucleotide
  modified_base (2)..(2): a, c, u, g, unknown or other
  modified_base (31)..(31): a, c, u, g, unknown or other
cngcagcacucagacuacgugcuccucugcng

SEQ ID NO: 63 - RNA, 32 nt, Artificial Sequence
  Description of Artificial Sequence: Synthetic oligonucleotide
  modified_base (2)..(2): a, c, u, g, unknown or other
  modified_base (31)..(31): a, c, u, g, unknown or other
cngcagcacucagacuacgugccccucugcng

SEQ ID NO: 64 - RNA, 32 nt, Artificial Sequence
  Description of Artificial Sequence: Synthetic oligonucleotide
  modified_base (2)..(2): a, c, u, g, unknown or other
  modified_base (31)..(31): a, c, u, g, unknown or other
cngcagcacucagacuacgugcgccucugcng

SEQ ID NO: 65 - RNA, 32 nt, Artificial Sequence
  Description of Artificial Sequence: Synthetic oligonucleotide
  modified_base (2)..(2): a, c, u, g, unknown or other
  modified_base (31)..(31): a, c, u, g, unknown or other
cngcagcacucagacuaugugcaccucugcng

SEQ ID NO: 66 - RNA, 33 nt, Artificial Sequence; identical to SEQ ID NO: 60
  Description of Artificial Sequence: Synthetic oligonucleotide

SEQ ID NO: 67 - RNA, 33 nt, Artificial Sequence
  Description of Artificial Sequence: Synthetic oligonucleotide
cagcagcacucagacuacgugcuccucugcugc

SEQ ID NO: 68 - RNA, 33 nt, Artificial Sequence
  Description of Artificial Sequence: Synthetic oligonucleotide
cagcagcacucagacuacgugccccucugcugc

SEQ ID NO: 69 - RNA, 33 nt, Artificial Sequence
  Description of Artificial Sequence: Synthetic oligonucleotide
cagcagcacucagacuacgugcgccucugcugc

SEQ ID NO: 70 - RNA, 33 nt, Artificial Sequence
  Description of Artificial Sequence: Synthetic oligonucleotide
cagcagcacucagacuaugugcaccucugcugc

SEQ ID NO: 71 - RNA, 35 nt, Artificial Sequence
  Description of Artificial Sequence: Synthetic oligonucleotide
cagcugcagcacucagacuacgugcaccucugcag

SEQ ID NO: 72 - RNA, 36 nt, Artificial Sequence
  Description of Artificial Sequence: Synthetic oligonucleotide
cagcugcagcacucagacuacgugcaccucugcugg

SEQ ID NO: 73 - RNA, 23 nt, Artificial Sequence
  Description of Artificial Sequence: Synthetic oligonucleotide
cagcacucagacuacgugcaccu

SEQ ID NO: 74 - RNA, 32 nt, Artificial Sequence
  Description of Artificial Sequence: Synthetic oligonucleotide
  modified_base (2)..(2): a, c, u, g, unknown or other
  modified_base (31)..(31): a, c, u, g, unknown or other
cngcagacgggcaacuuuacacgacgcugcng

SEQ ID NO: 75 - DNA, 5 bp, Artificial Sequence
  Description of Artificial Sequence: Synthetic oligonucleotide
  modified_base (1)..(1): a, c, t, g, unknown or other
ncgtg

SEQ ID NO: 76 - DNA, 5 bp, Artificial Sequence
  Description of Artificial Sequence: Synthetic oligonucleotide
rcgtg

SEQ ID NO: 77 - DNA, 5 bp, Artificial Sequence
  Description of Artificial Sequence: Synthetic oligonucleotide
cacgy

SEQ ID NO: 78 - DNA, 6 bp, Artificial Sequence
  Description of Artificial Sequence: Synthetic oligonucleotide
acgtgc

SEQ ID NO: 79 - DNA, 6 bp, Artificial Sequence
  Description of Artificial Sequence: Synthetic oligonucleotide
gcacgt

SEQ ID NO: 80 - DNA, 9 bp, Artificial Sequence
  Description of Artificial Sequence: Synthetic oligonucleotide
ctgcacgta

SEQ ID NO: 81 - DNA, 9 bp, Artificial Sequence
  Description of Artificial Sequence: Synthetic oligonucleotide
tacgtgcag

SEQ ID NO: 82 - DNA, 30 bp, Artificial Sequence
  Description of Artificial Sequence: Synthetic oligonucleotide
accttgagtacgtgcgtctctgcacgtatg

SEQ ID NO: 83 - DNA, 30 bp, Artificial Sequence
  Description of Artificial Sequence: Synthetic oligonucleotide
catacgtgcagagacgcacgtactcaaggt

SEQ ID NO: 84 - DNA, 33 bp, Artificial Sequence
  Description of Artificial Sequence: Synthetic oligonucleotide
  modified_base (1)..(10): a, c, t, g, unknown or other
  misc_feature (1)..(10): This region may encompass 8-10 nucleotides
  modified_base (16)..(21): a, c, t, g, unknown or other
  misc_feature (16)..(21): This region may encompass 4-6 nucleotides
  modified_base (31)..(33): a, c, t, g, unknown or other
  misc_feature (31)..(33): This region may encompass 1-3 nucleotides
  See specification as filed for detailed description of substitutions and preferred embodiments
nnnnnnnnnnacgtgnnnnnnctgcacgtannn

SEQ ID NO: 85 - DNA, 30 bp, Artificial Sequence
  Description of Artificial Sequence: Synthetic oligonucleotide
  modified_base (1)..(9): a, c, t, g, unknown or other
  modified_base (15)..(19): a, c, t, g, unknown or other
  modified_base (29)..(30): a, c, t, g, unknown or other
nnnnnnnnnacgtgnnnnnctgcacgtann

SEQ ID NO: 86 - DNA, 6 bp, Artificial Sequence
  Description of Artificial Sequence: Synthetic oligonucleotide
acgtgc

SEQ ID NO: 87 - DNA, 6 bp, Artificial Sequence
  Description of Artificial Sequence: Synthetic oligonucleotide
acgtgc

SEQ ID NO: 88 - DNA, 6 bp, Artificial Sequence
  Description of Artificial Sequence: Synthetic oligonucleotide
acgtgc

SEQ ID NO: 89 - DNA, 40 bp, Artificial Sequence
  Description of Artificial Sequence: Synthetic oligonucleotide
gatgatgcgtagctagtagtgatgatgcgtagctagtagt

SEQ ID NO: 90 - DNA, 190 bp, Artificial Sequence
  Description of Artificial Sequence: Synthetic polynucleotide
acgtgcgatgatgcgtagctagtagtgatgatgcgtagctagtagtacgtgcgatgatgcgtagctagtagtgatgatgcgtagctagtagtacgtgcgatgatgcgtagctagtagtgatgatgcgtagctagtagtacgtgcgatgatgcgtagctagtagtgatgatgcgtagctagtagtacgtgc

SEQ ID NO: 91 - DNA, 9 bp, Artificial Sequence
  Description of Artificial Sequence: Synthetic oligonucleotide
ctgcacgta

SEQ ID NO: 92 - DNA, 9 bp, Artificial Sequence
  Description of Artificial Sequence: Synthetic oligonucleotide
ctgcacgta

SEQ ID NO: 93 - DNA, 9 bp, Artificial Sequence
  Description of Artificial Sequence: Synthetic oligonucleotide
ctgcacgta

SEQ ID NO: 94 - DNA, 20 bp, Artificial Sequence
  Description of Artificial Sequence: Synthetic oligonucleotide
gatgatgcgtagctagtagt

SEQ ID NO: 95 - DNA, 154 bp, Artificial Sequence
  Description of Artificial Sequence: Synthetic polynucleotide
ctgcacgtagatgatgcgtagctagtagtctgcacgtagatgatgcgtagctagtagtctgcacgtagatgatgcgtagctagtagtctgcacgtagatgatgcgtagctagtagtctgcacgtagatgatgcgtagctagtagtctgcacgta

SEQ ID NO: 96 - DNA, 36 bp, Artificial Sequence
  Description of Artificial Sequence: Synthetic oligonucleotide
ctgcacgtactgcacgtactgcacgtactgcacgta

SEQ ID NO: 97 - DNA, 30 bp, Artificial Sequence; identical to SEQ ID NO: 82
  Description of Artificial Sequence: Synthetic oligonucleotide

SEQ ID NO: 98 - DNA, 30 bp, Artificial Sequence; identical to SEQ ID NO: 82
  Description of Artificial Sequence: Synthetic oligonucleotide

SEQ ID NO: 99 - DNA, 30 bp, Artificial Sequence; identical to SEQ ID NO: 82
  Description of Artificial Sequence: Synthetic oligonucleotide

SEQ ID NO: 100 - DNA, 30 bp, Artificial Sequence; identical to SEQ ID NO: 82
  Description of Artificial Sequence: Synthetic oligonucleotide

SEQ ID NO: 101 - DNA, 30 bp, Artificial Sequence; identical to SEQ ID NO: 82
  Description of Artificial Sequence: Synthetic oligonucleotide

SEQ ID NO: 102 - DNA, 9 bp, Artificial Sequence
  Description of Artificial Sequence: Synthetic oligonucleotide
gcgattaag

SEQ ID NO: 103 - DNA, 147 bp, Artificial Sequence
  Description of Artificial Sequence: Synthetic polynucleotide
accttgagtacgtgcgtctctgcacgtatggcgattaagaccttgagtacgtgcgtctctgcacgtatggcgattaagaccttgagtacgtgcgtctctgcacgtatggcgattaagaccttgagtacgtgcgtctctgcacgtatg

SEQ ID NO: 104 - DNA, 91 bp, Artificial Sequence
  Description of Artificial Sequence: Synthetic oligonucleotide
aggtctatataagcagagctcgtttagtgaaccgtcagatcgcctagatacgccatccacgctgttttgacctccatagaagatcgccacc

SEQ ID NO: 105 - DNA, 25 bp, Artificial Sequence
  Description of Artificial Sequence: Synthetic oligonucleotide
tctagagggtatataatgggggcca

SEQ ID NO: 106 - DNA, 89 bp, Artificial Sequence
  Description of Artificial Sequence: Synthetic oligonucleotide
gcgattaatccatatgctctagagggtatataatgggggccactagtctactaccagaaagcttggtaccgagctcggatccagccacc

SEQ ID NO: 107 - DNA, 63 bp, Artificial Sequence
  Description of Artificial Sequence: Synthetic oligonucleotide
ttcgcatattaaggtgacgcgtgtggcctcgaacaccgagcgaccctgcagcgacccgcttaa

SEQ ID NO: 108 - DNA, 202 bp, Artificial Sequence
  Description of Artificial Sequence: Synthetic polynucleotide
gcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagctt

SEQ ID NO: 109 - DNA, 131 bp, Artificial Sequence
  Description of Artificial Sequence: Synthetic polynucleotide
ttggtaccatccgggccggccgcttaagcgacgcctataaaaaataggttgcatgctaggcctagcgctgccagtccatcttcgctagcctgtgctgcgtcagtccagcgctgcgctgcgtaacggccgcc

SEQ ID NO: 110 - DNA, 158 bp, Artificial Sequence
  Description of Artificial Sequence: Synthetic polynucleotide
tgacgtcacgatttgacgtcaaccattgacgtcacgatttgacgtcaaccattgacgtcacgatttgacgtcacgatttgacgtcaaccattgactcacgatttgactcaaccattgactcacgatttgactcaaccattgactcacgatttgactca

SEQ ID NO: 111 - DNA, 60 bp, Artificial Sequence
  Description of Artificial Sequence: Synthetic oligonucleotide
tataaaaggcagagctcgtttagtgaaccgaagcttggactaaagcggacttgtctcgag

SEQ ID NO: 112 - DNA, 8 bp, Artificial Sequence
  Description of Artificial Sequence: Synthetic oligonucleotide
tgacgtca

SEQ ID NO: 113 - DNA, 7 bp, Artificial Sequence
  Description of Artificial Sequence: Synthetic oligonucleotide
tgastca

SEQ ID NO: 114 - DNA, 7 bp, Artificial Sequence
  Description of Artificial Sequence: Synthetic oligonucleotide
tgagtca

SEQ ID NO: 115 - DNA, 8 bp, Artificial Sequence
  Description of Artificial Sequence: Synthetic oligonucleotide
tgactcag

SEQ ID NO: 116 - DNA, 7 bp, Artificial Sequence
  Description of Artificial Sequence: Synthetic oligonucleotide
tgactca

SEQ ID NO: 117 - DNA, 6 bp, Artificial Sequence
  Description of Artificial Sequence: Synthetic oligonucleotide
tgacgt

SEQ ID NO: 118 - DNA, 7 bp, Artificial Sequence
  Description of Artificial Sequence: Synthetic oligonucleotide
tgacgtg

SEQ ID NO: 119 - DNA, 9 bp, Artificial Sequence
  Description of Artificial Sequence: Synthetic oligonucleotide
tgacgtgct

SEQ ID NO: 120 - DNA, 8 bp, Artificial Sequence
  Description of Artificial Sequence: Synthetic oligonucleotide
tgacgtca

SEQ ID NO: 121 - DNA, 8 bp, Artificial Sequence
  Description of Artificial Sequence: Synthetic oligonucleotide
tgacgtca

SEQ ID NO: 122 - DNA, 7 bp, Artificial Sequence
  Description of Artificial Sequence: Synthetic oligonucleotide
tgastca

SEQ ID NO: 123 - DNA, 6 bp, Artificial Sequence
  Description of Artificial Sequence: Synthetic oligonucleotide
tgacgt

SEQ ID NO: 124 - DNA, 8 bp, Artificial Sequence
  Description of Artificial Sequence: Synthetic oligonucleotide
tgacgtca

SEQ ID NO: 125 - DNA, 8 bp, Artificial Sequence
  Description of Artificial Sequence: Synthetic oligonucleotide
tgacgtca

SEQ ID NO: 126 - DNA, 7 bp, Artificial Sequence
  Description of Artificial Sequence: Synthetic oligonucleotide
tgagtca

SEQ ID NO: 127 - DNA, 9 bp, Artificial Sequence
  Description of Artificial Sequence: Synthetic oligonucleotide
tgacgtgct

SEQ ID NO: 128 - DNA, 8 bp, Artificial Sequence
  Description of Artificial Sequence: Synthetic oligonucleotide
tgacgtca

SEQ ID NO: 129 - DNA, 8 bp, Artificial Sequence
  Description of Artificial Sequence: Synthetic oligonucleotide
tgacgtca

SEQ ID NO: 130 - DNA, 8 bp, Artificial Sequence
  Description of Artificial Sequence: Synthetic oligonucleotide
tgacgtca

SEQ ID NO: 131 - DNA, 132 bp, Artificial Sequence
  Description of Artificial Sequence: Synthetic polynucleotide
tgacgtcacgattaccattgacgtcacgattaccattgacgtcacgattaccattgacgtcacgattaccattgacgtcagcgattaagatgactcagcgattaagatgactcagcgattaagatgactcag

SEQ ID NO: 132 - DNA, 149 bp, Artificial Sequence
  Description of Artificial Sequence: Synthetic polynucleotide
tgacgtcacgattaccattgacgtcacgattaccattgacgtcacgattaccattgacgtcacgattaccattgacgtcagcgattaagatgactcagcgattaagatgactcagcgattaagatgactcagcgattaagatgactcag

SEQ ID NO: 133 - DNA, 196 bp, Artificial Sequence
  Description of Artificial Sequence: Synthetic polynucleotide
tgagtcagatgatgcgtagctagtagttgagtcagatgatgcgtagctagtagttgagtcagatgatgcgtagctagtagttgagtcagatgatgcgtagctagtagttgagtcagatgatgcgtagctagtagttgagtcagatgatgcgtagctagtagttgagtcagatgatgcgtagctagtagttgagtca

SEQ ID NO: 134 - DNA, 262 bp, Artificial Sequence
  Description of Artificial Sequence: Synthetic polynucleotide
tgacgtgctgatgatgcgtagctagtagttgacgtgctgatgatgcgtagctagtagttgacgtgctgatgatgcgtagctagtagttgagtcagatgatgcgtagctagtagttgagtcagatgatgcgtagctagtagttgagtcagatgatgcgtagctagtagttgagtcagatgatgcgtagctagtagtctgcacgtagatgatgcgtagctagtagtctgcacgtagatgatgcgtagctagtagtctgcacgta

SEQ ID NO: 135 - DNA, 148 bp, Artificial Sequence
  Description of Artificial Sequence: Synthetic polynucleotide
tgacgtcacgattaccattgacgtcacgattaccattgacgtcacgattaccattgacgtcacgattaccattgacgtcagcgattaagatgactcagcgattaagatgactcagcgattaagatgactcagcgattaagatgactca

SEQ ID NO: 136 - DNA, 216 bp, Artificial Sequence
  Description of Artificial Sequence: Synthetic polynucleotide
aataaaatatctttattttcattacatctgtgtgttggttttttgtgtgccattgacgtcacgatttgacgtcaaccattgacgtcacgatttgacgtcaaccattgacgtcacgatttgacgtcacgatttgacgtcaaccattgactcacgatttgactcaaccattgactcacgatttgactcaaccattgactcacgatttgactcacgatt

SEQ ID NO: 137 - DNA, 89 bp, Artificial Sequence; identical to SEQ ID NO: 106
  Description of Artificial Sequence: Synthetic oligonucleotide

SEQ ID NO: 138 - DNA, 8 bp, Artificial Sequence
  Description of Artificial Sequence: Synthetic oligonucleotide
tgacgtca

SEQ ID NO: 139 - DNA, 8 bp, Artificial Sequence
  Description of Artificial Sequence: Synthetic oligonucleotide
tgacgtca

SEQ ID NO: 140 - DNA, 7 bp, Artificial Sequence
  Description of Artificial Sequence: Synthetic oligonucleotide
tgastca

SEQ ID NO: 141 - DNA, 6 bp, Artificial Sequence
  Description of Artificial Sequence: Synthetic oligonucleotide
tgacgt

SEQ ID NO: 142 - DNA, 8 bp, Artificial Sequence
  Description of Artificial Sequence: Synthetic oligonucleotide
tgacgtca

SEQ ID NO: 143 - DNA, 256 bp, Artificial Sequence
  Description of Artificial Sequence: Synthetic polynucleotide
ccattgacgtcacgatttgacgtcaaccattgacgtcacgatttgacgtcaaccattgacgtcacgatttgacgtcacgatttgacgtcaaccattgactcacgatttgactcaaccattgactcacgatttgactcaaccattgactcacgatttgactcacgattgcgattaatccatatgctctagagggtatataatgggggccactagtctactaccagaaagcttggtaccgagctcggatccagccacc

SEQ ID NO: 144 - DNA, 218 bp, Artificial Sequence
  Description of Artificial Sequence: Synthetic polynucleotide
ccattgacgtcacgatttgacgtcaaccattgacgtcacgatttgacgtcaaccattgacgtcacgatttgacgtcacgatttgacgtcaaccattgactcacgatttgactcaaccattgactcacgatttgactcaaccattgactcacgatttgactcacgatttataaaaggcagagctcgtttagtgaaccgaagcttggactaaagcggact

SEQ ID NO: 145 - DNA, 346 bp, Artificial Sequence
  Description of Artificial Sequence: Synthetic polynucleotide
tgagtcagatgatgcgtagctagtagttgagtcagatgatgcgtagctagtagttgagtcagatgatgcgtagctagtagttgagtcagatgatgcgtagctagtagttgagtcagatgatgcgtagctagtagttgagtcagatgatgcgtagctagtagttgagtcagatgatgcgtagctagtagttgagtcagtagtcgtatgctgatgcgcagttagcgtagctgaggtaccgtcgacgatatcggatccaggtctatataagcagagctcgtttagtgaaccgtcagatcgcctagatacgccatccacgctgttttgacctccatagaagatcgccacc

SEQ ID NO: 146 - DNA, 414 bp, Artificial Sequence
  Description of Artificial Sequence: Synthetic polynucleotide
tgacgtgctgatgatgcgtagctagtagttgacgtgctgatgatgcgtagctagtagttgacgtgctgatgatgcgtagctagtagttgagtcagatgatgcgtagctagtagttgagtcagatgatgcgtagctagtagttgagtcagatgatgcgtagctagtagttgagtcagatgatgcgtagctagtagtctgcacgtagatgatgcgtagctagtagtctgcacgtagatgatgcgtagctagtagtctgcacgtagatgatgcgtagctagtagtgcagttagcgtagctgaggtaccgtcgacgatatcggatccaggtctatataagcagagctcgtttagtgaaccgtcagatcgcctagatacgccatccacgctgttttgacctccatagaagatcgccacc

SEQ ID NO: 147 - DNA, 237 bp, Artificial Sequence
  Description of Artificial Sequence: Synthetic polynucleotide
tgacgtcacgattaccattgacgtcacgattaccattgacgtcacgattaccattgacgtcacgattaccattgacgtcagcgattaagatgactcagcgattaagatgactcagcgattaagatgactcagcgattaagatgactcagcgattaatccatatgctctagagggtatataatgggggccactagtctactaccagaaagcttggtaccgagctcggatccagccacc

SEQ ID NO: 148 - skipped

SEQ ID NO: 149 - DNA, 118 bp, Artificial Sequence
  Description of Artificial Sequence: Synthetic polynucleotide
acgcaatgccgccgggggagcgaacaaagttgttgacgagtgctacatccccaattacttgctccccaaaacccagcctgagctccaatggggatggacaaacatagaacagtacctg

SEQ ID NO: 150 - DNA, 1866 bp, Artificial Sequence
  Description of Artificial Sequence: Synthetic polynucleotide
ctgccggggttttacgagattgtgattaaggtccccagcgaccttgacgagcatctgcccggcatttctgacagctttgtgaactgggtggccgagaaggagtgggagttgccgccagattctgacttggatctgaatctgattgagcaggcacccctgaccgtggccgagaagctgcagcgcgactttctgacggagtggcgccgtgtgagtaaggccccggaggcccttttctttgtgcaatttgagaagggagagagctacttccacttacacgtgctcgtggaaaccaccggggtgaaatccttagttttgggacgtttcctgagtcagattcgcgaaaaactgattcagagaatttaccgcgggatcgagccgactttgccaaactggttcgcggtcacaaagaccagaaacggcgccggaggcgggaacaaggtggtggacgagtgctacatccccaattacttgctccccaaaacccagcctgagctccagtgggcgtggactaatatagaacagtatttaagcgcctgtttgaatctcacggagcgtaaacggttggtggcgcagcatctgacgcacgtgtcgcagacgcaggagcagaacaaagagaatcagaatcccaattctgacgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 151 - protein, 621 aa, Artificial Sequence
  Description of Artificial Sequence: Synthetic polypeptide
MPGFYEIVIKVPSDLDEHLPGISDSFVNWVAEKEWELPPDSDLDLNLIEQAPLTVAEKLQRDFLTEWRRVSKAPEALFFVQFERGESYFHLHVLVETTGVKSLVLGRFLSQIREKLIQRIYRGIEPTLPNWFAVTKTRNGAGGGNKVVDECYIPNYLLPKTQPELQWAWTNIEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 152 - DNA, 1866 bp, Artificial Sequence
  Description of Artificial Sequence: Synthetic polynucleotide
ctgccggggttttacgagattgtgattaaggtccccagcgaccttgacgagcatctgcccggcatttctgacagctttgtgaactgggtggccgagaaggagtgggagttgccgccagattctgacttggatctgaatctgattgagcaggcacccctgaccgtggccgagaagctgcagcgcgactttctgacggagtggcgccgtgtgagtaaggccccggaggcccttttctttgtgcaatttgagagaggagagagctacttccacttacacgtgctcgtggaaaccaccggggtgaaatccttagttttgggacgtttcctgagtcagattcgcgaaaaactgattcagagaatttaccgcgggatcgagccgactttgccaaactggttcgcggtcacaaagacacgcaatgccgccgggggagcgaacaaagttgttgacgagtgctacatccccaattacttgctccccaaaacccagcctgagctccaatggggatggacaaacatagaacagtacctgagcgcctgtttgaatctcacggagcgtaaacggttggtggcgcagcatctgacgcacgtgtcgcagacgcaggagcagaacaaagagaatcagaatcccaattctgacgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 153 - protein, 621 aa, Artificial Sequence
  Description of Artificial Sequence: Synthetic polypeptide
LPGFYEIVIKVPSDLDEHLPGISDSFVNWVAEKEWELPPDSDLDLNLIEQAPLTVAEKLQRDFLTEWRRVSKAPEALFFVQFERGESYFHLHVLVETTGVKSLVLGRFLSQIREKLIQRIYRGIEPTLPNWFAVTKTRNAAGGANKVVDECYIPNYLLPKTQPELQWGWTNIEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 154 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 155 - protein, 984 aa, Campylobacter jejuni
MARILAFDIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK

SEQ ID NO: 156 - protein, 1307 aa, Acidaminococcus sp
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 157 - DNA, 20 bp, Artificial Sequence
  Description of Artificial Sequence: Synthetic primer
tgtgtctgaacctgagcctg

SEQ ID NO: 158 - DNA, 27 bp, Artificial Sequence
  Description of Artificial Sequence: Synthetic probe
agcccgagccagaaccggagcctgcaa

SEQ ID NO: 159 - DNA, 20 bp, Artificial Sequence
  Description of Artificial Sequence: Synthetic primer
atagcaggcgccattttagg

SEQ ID NO: 160 - DNA, 19 bp, Artificial Sequence
  Description of Artificial Sequence: Synthetic primer
gtgtgcatctgcaacgccg

SEQ ID NO: 161 - DNA, 23 bp, Artificial Sequence
  Description of Artificial Sequence: Synthetic primer
gactcgtggtacatgcagttctc

SEQ ID NO: 162 - DNA, 20 bp, Artificial Sequence
  Description of Artificial Sequence: Synthetic probe
acatcaccgtgagcgtggag

SEQ ID NO: 163 - DNA, 39 bp, Artificial Sequence
  Description of Artificial Sequence: Synthetic primer
gcccttttctttgtgcaatttgagaggggagagagctac

SEQ ID NO: 164 - DNA, 33 bp, Artificial Sequence
  Description of Artificial Sequence: Synthetic primer
tctcccctctcaaattgcacaaagaaaagggcc

SEQ ID NO: 165 - skipped

SEQ ID NO: 166 - DNA, 20 bp, Artificial Sequence
  Description of Artificial Sequence: Synthetic primer
ctttggtagctgccttccca

SEQ ID NO: 167 - DNA, 20 bp, Artificial Sequence
  Description of Artificial Sequence: Synthetic primer
agggatcgcctacctccttt

SEQ ID NO: 168 - DNA, 20 bp, Artificial Sequence
  Description of Artificial Sequence: Synthetic primer
gccgaggacttgcatttctg

SEQ ID NO: 169 - DNA, 20 bp, Artificial Sequence
  Description of Artificial Sequence: Synthetic primer
ctgacagctttgtgaactgg

SEQ ID NO: 170 - DNA, 15 bp, Artificial Sequence
  Description of Artificial Sequence: Synthetic primer
cagttgcgcagccat

SEQ ID NO: 171 - DNA, 20 bp, Artificial Sequence
  Description of Artificial Sequence: Synthetic primer
agtgttacattcgggcagca

SEQ ID NO: 172 - DNA, 18 bp, Artificial Sequence
  Description of Artificial Sequence: Synthetic primer
tcggccaaagccattctc

SEQ ID NO: 173 - DNA, 17 bp, Artificial Sequence
  Description of Artificial Sequence: Synthetic primer
gaatctggcggcaactc

SEQ ID NO: 174 - DNA, 22 bp, Artificial Sequence
  Description of Artificial Sequence: Synthetic primer
caattacagattacgagtcagg

SEQ ID NO: 175 - DNA, 19 bp, Artificial Sequence
  Description of Artificial Sequence: Synthetic probe
tagtggcatcagaaggtga

SEQ ID NO: 176 - DNA, 20 bp, Artificial Sequence
  Description of Artificial Sequence: Synthetic probe
taccatactggaggatcatc

SEQ ID NO: 177 - DNA, 22 bp, Artificial Sequence
  Description of Artificial Sequence: Synthetic probe
tccacgcgcaccttgcttcctc

SEQ ID NO: 178 - DNA, 18 bp, Artificial Sequence
  Description of Artificial Sequence: Synthetic probe
gtggccgagaaggaatgg

SEQ ID NO: 179 - DNA, 22 bp, Artificial Sequence
  Description of Artificial Sequence: Synthetic probe
ttcgaccaccttcagtgcggca

SEQ ID NO: 180 - DNA, 21 bp, Artificial Sequence
  Description of Artificial Sequence: Synthetic primer
ggaacccctagtgatggagtt

SEQ ID NO: 181 - DNA, 16 bp, Artificial Sequence
  Description of Artificial Sequence: Synthetic primer
cggcctcagtgagcga

SEQ ID NO: 182 - DNA, 21 bp, Artificial Sequence
  Description of Artificial Sequence: Synthetic probe
cactccctctctgcgcgctcg

SEQ ID NO: 183 - DNA, 69 bp, Artificial Sequence; identical to SEQ ID NO: 48
  Description of Artificial Sequence: Synthetic oligonucleotide

SEQ ID NO: 184 - DNA, 10 bp, Artificial Sequence
  Description of Artificial Sequence: Synthetic oligonucleotide
ccagaaacgg

SEQ ID NO: 185 - DNA, 9 bp, Artificial Sequence
  Description of Artificial Sequence: Synthetic oligonucleotide
cacgcaatg

SEQ ID NO: 186 - DNA, 7 bp, Artificial Sequence
  Description of Artificial Sequence: Synthetic oligonucleotide
gaggcgg

SEQ ID NO: 187 - DNA, 6 bp, Artificial Sequence
  Description of Artificial Sequence: Synthetic oligonucleotide
ggggag

SEQ ID NO: 188 - DNA, 7 bp, Artificial Sequence
  Description of Artificial Sequence: Synthetic oligonucleotide
ggtggtg

SEQ ID NO: 189 - DNA, 7 bp, Artificial Sequence
  Description of Artificial Sequence: Synthetic oligonucleotide
agttgtt

SEQ ID NO: 190 - DNA, 10 bp, Artificial Sequence
  Description of Artificial Sequence: Synthetic oligonucleotide
agtgggcgtg

SEQ ID NO: 191 - DNA, 10 bp, Artificial Sequence
  Description of Artificial Sequence: Synthetic oligonucleotide
aatggggatg

SEQ ID NO: 192 - DNA, 6 bp, Artificial Sequence
  Description of Artificial Sequence: Synthetic oligonucleotide
taatat

SEQ ID NO: 193 - DNA, 6 bp, Artificial Sequence
  Description of Artificial Sequence: Synthetic oligonucleotide
tattta

SEQ ID NO: 194 - DNA, 6 bp, Artificial Sequence
  Description of Artificial Sequence: Synthetic oligonucleotide
tacctg

SEQ ID NO: 195 - DNA, 5465 bp, Artificial Sequence
  Description of Artificial Sequence: Synthetic polynucleotide
acgtgcgatgagctccccgggttaattaacatatgactagtgaattcattgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggcggccgcgacctgcaggcgcagaactggtaggtatggaagatccctcgagatccattgtgctggcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgcttgacactttatcactgataaacataatatgtccaccaacttatcagtgataaagaatccgcgccagcacaatggatctcgaggtcgagggatctctagaggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgacctcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctgatggctctttgcggcacccatcgttcgtaatgttccgtggcaccgaggacaaccctcaagagaaaatgtaatcacactggctcaccttcgggtgggcctttctgcgtttataaggagacactttatgtttaagaaggttggtaaattccttgcggctttggcagccaagctagatccggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctagcttggggccaccgctcagagcaccttccaccatggccacctcagcaagttcccacttgaacaaaaacatcaagcaaatgtacttgtgcctgccccagggtgagaaagtccaagccatgtatatctgggttgatggtactggagaaggactgcgctgcaaaacccgcaccctggactgtgagcccaagtgtgtagaagagttacctgagtggaattttgatggctctagtacctttcagtctgagggctccaacagtgacatgtatctcagccctgttgccatgtttcgggaccccttccgcagagatcccaacaagctggtgttctgtgaagttttcaagtacaaccggaagcctgcagagaccaatttaaggcactcgtgtaaacggataatggacatggtgagcaaccagcacccctggtttggaatggaacaggagtatactctgatgggaacagatgggcacccttttggttggccttccaatggctttcctgggccccaaggtccgtattactgtggtgtgggcgcagacaaagcctatggcagggatatcgtggaggctcactaccgcgcctgcttgtatgctggggtcaagattacaggaacaaatgctgaggtcatgcctgcccagtgggagttccaaataggaccctgtgaaggaatccgcatgggagatcatctctgggtggcccgtttcatcttgcatcgagtatgtgaagactttggggtaatagcaacctttgaccccaagcccattcctgggaactggaatggtgcaggctgccataccaactttagcaccaaggccatgcgggaggagaatggtctgaagcacatcgaggaggccatcgagaaactaagcaagcggcaccggtaccacattcgagcctacgatcccaaggggggcctggacaatgcccgtcgtctgactgggttccacgaaacgtccaacatcaacgacttttctgctggtgtcgccaatcgcagtgccagcatccgcattccccggactgtcggccaggagaagaaaggttactttgaagaccgccgcccctctgccaattgtgacccctttgcagtgacagaagccatcgtccgcacatgccttctcaatgagactggcgacgagcccttccaatacaaaaactaattagactttgagtgatcttgagcctttcctagttcatcccaccccgccccagagagatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctctagcttcgtgtcaaggacggtgagg

SEQ ID NO: 196 - DNA, 4199 bp, Artificial Sequence
  Description of Artificial Sequence: Synthetic polynucleotide
aataaaatatctttattttcattacatctgtgtgttggttttttgtgtgctgcacgtactgcacgtactgcacgtactgcacgtatgggtaccgtcgacgatatcggatccaggtctatataagcagagctcgtttagtgaaccgtcagatcgcctagatacgccatccacgctgttttgacctccatagaagatcgccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaatgaaagcttggtctctacgagtaatagacgcccagttgaattccttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaa

SEQ ID NO: 197 - DNA, 2358 bp, Artificial Sequence
  Description of Artificial Sequence: Synthetic polynucleotide
aataaaatatctttattttcattacatctgtgtgttggttttttgtgtgccattgacgtcacgatttgacgtcaaccattgacgtcacgatttgacgtcaaccattgacgtcacgatttgacgtcacgatttgacgtcaaccattgactcacgatttgactcaaccattgactcacgatttgactcaaccattgactcacgatttgactcacgattgcgattaatccatatgctctagagggtatataatgggggccactagtctactaccagaaagcttggtaccgagctcggatccagccaccctcgagatgccggggttttacgagattgtgattaaggtccccagcgaccttgacgagcatctgcccggcatttctgacagctttgtgaactgggtggccgagaaggagtgggagttgccgccagattctgacttggatctgaatctgattgagcaggcacccctgaccgtggccgagaagctgcagcgcgactttctgacggagtggcgccgtgtgagtaaggccccggaggcccttttctttgtgcaatttgagaagggagagagctacttccacttacacgtgctcgtggaaaccaccggggtgaaatccttagttttgggacgtttcctgagtcagattcgcgaaaaactgattcagagaatttaccgcgggatcgagccgactttgccaaactggttcgcggtcacaaagaccagaaacggcgccggaggcgggaacaaggtggtggacgagtgctacatccccaattacttgctccccaaaacccagcctgagctccagtgggcgtggactaatatagaacagtatttaagcgcctgtttgaatctcacggagcgtaaacggttggtggcgcagcatctgacgcacgtgtcgcagacgcaggagcagaacaaagagaatcagaatcccaattctgacgcgccggtgatcagatcaaaaacttcagccaggtacggagagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataatgaaagcttggtctctacgagtaatagacgcccagttgaattccttcgagcagacatgataagatacattgatgagtttggacaaaccacaactagaatgcagtgaaaaaaatgctttatttgtgaaatttgtgatgctattgctttatttgtaaccattataagctgcaataaacaagtt

SEQ ID NO: 198 - DNA, 263 bp, Artificial Sequence
  Description of Artificial Sequence: Synthetic polynucleotide
aataaaatatctttattttcattacatctgtgtgttggttttttgtgtgccattgacgtcacgatttgacgtcaaccattgacgtcacgatttgacgtcaaccattgacgtcacgatttgacgtcacgatttgacgtcaaccattgactcacgatttgactcaaccattgactcacgatttgactcaaccattgactcacgatttgactcacgattggggggctataaaagggggtgggggcgttcgtcctcactctgccacc

SEQ ID NO: 199 - DNA, 425 bp, Artificial Sequence
  Description of Artificial Sequence: Synthetic polynucleotide
aataaaatatctttattttcattacatctgtgtgttggttttttgtgtgccattgacgtcacgatttgacgtcaaccattgacgtcacgatttgacgtcaaccattgacgtcacgatttgacgtcacgatttgacgtcaaccattgactcacgatttgactcaaccattgactcacgatttgactcaaccattgactcacgatttgactcacgatttgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttgccacc

SEQ ID NO: 200 - DNA, 285 bp, Artificial Sequence
  Description of Artificial Sequence: Synthetic polynucleotide
aataaaatatctttattttcattacatctgtgtgttggttttttgtgtgccattgacgtcacgatttgacgtcaaccattgacgtcacgatttgacgtcaaccattgacgtcacgatttgacgtcacgatttgacgtcaaccattgactcacgatttgactcaaccattgactcacgatttgactcaaccattgactcacgatttgactcacgattttcgcatattaaggtgacgcgtgtggcctcgaacaccgagcgaccctgcagcgacccgcttaagccacc

SEQ ID NO: 201 - DNA, 281 bp, Artificial Sequence
  Description of Artificial Sequence: Synthetic polynucleotide
aataaaatatctttattttcattacatctgtgtgttggttttttgtgtgccattgacgtcacgatttgacgtcaaccattgacgtcacgatttgacgtcaaccattgacgtcacgatttgacgtcacgatttgacgtcaaccattgactcacgatttgactcaaccattgactcacgatttgactcaaccattgactcacgatttgactcacgattgtaggcgtgtacggtgggaggtctatataagcagagctcgtttagtgaaccgtcagatcgccacc

SEQ ID NO: 202 - DNA, 298 bp, Artificial Sequence
  Description of Artificial Sequence: Synthetic polynucleotide
aataaaatatctttattttcattacatctgtgtgttggttttttgtgtgccattgacgtcacgatttgacgtcaaccattgacgtcacgatttgacgtcaaccattgacgtcacgatttgacgtcacgatttgacgtcaaccattgactcacgatttgactcaaccattgactcacgatttgactcaaccattgactcacgatttgactcacgattcaacaaaatgtcgtaacaagggcggtaggcgtgtacggtgggaggtctatataagcagagctcgtttagtgaaccggccacc

SEQ ID NO: 203 - DNA, 4030 bp, Artificial Sequence
  Description of Artificial Sequence: Synthetic polynucleotide
atggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaagtgtaatgaaagcttggtctctacgagtaatagacgcccagttgaattccttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtaacaacaacaattgcattcattttatgtttcaggttcagggggaggtgtgggaggttttttaaagcaagtaaaacctctacaaatgtggtaaaatcgataaggatccgtctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 204 - DNA, 3412 bp, Artificial Sequence
  Description of Artificial Sequence: Synthetic polynucleotide
cgatagatctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattgcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctatc

SEQ ID NO: 205 - DNA, 410 bp, Artificial Sequence
  Description of Artificial Sequence: Synthetic polynucleotide
ccattgacgtcacgattaccattgacgtcacgattaccattgacgtcacgattaccattgacgtcacgattaccattgacgtcagcgattaagatgactcagcgattaagatgactcagcgattaagatgactcagcgattaagatgactcactagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttggcattccggtactgttggtaaagccacc

SEQ ID NO: 206 - DNA, 260 bp, Artificial Sequence
  Description of Artificial Sequence: Synthetic polynucleotide
ccattgacgtcacgattaccattgacgtcacgattaccattgacgtcacgattaccattgacgtcacgattaccattgacgtcagcgattaagatgactcagcgattaagatgactcagcgattaagatgactcagcgattaagatgactcagcgattaatccatatgcaggtctatataagcagagctcgtttagtgaaccgtcagatcgcctagatacgccatccacgctgttttgacctccatagaagatcgccacc

SEQ ID NO: 207 - DNA, 197 bp, Artificial Sequence
  Description of Artificial Sequence: Synthetic polynucleotide
tgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaa

SEQ ID NO: 208 - DNA, 76 bp, Artificial Sequence
  Description of Artificial Sequence: Synthetic oligonucleotide
gggcatataaaacaggggcaaggcacagactcatagcagagcaatcaccaccaagcctggaataactgcagccacc

SEQ ID NO: 209 - DNA, 276 bp, Artificial Sequence
  Description of Artificial Sequence: Synthetic polynucleotide
aataaaatatctttattttcattacatctgtgtgttggttttttgtgtgccattgacgtcacgatttgacgtcaaccattgacgtcacgatttgacgtcaaccattgacgtcacgatttgacgtcacgatttgacgtcaaccattgactcacgatttgactcaaccattgactcacgatttgactcaaccattgactcacgatttgactcacgatttataaaaggcagagctcgtttagtgaaccgaagcttggactaaagcggacttgtctcgag

SEQ ID NO: 210 - DNA, 345 bp, Artificial Sequence
  Description of Artificial Sequence: Synthetic polynucleotide
ccattgacgtcacgatttgacgtcaaccattgacgtcacgatttgacgtcaaccattgacgtcacgatttgacgtcacgatttgacgtcaaccattgactcacgatttgactcaaccattgactcacgatttgactcaaccattgactcacgatttgactcacgattctgacaaattcagtataaaagcttggggctggggccgagcactggggactttgagggtggccaggccagcgtaggaggccagcgtaggatcctgctgggagcggggaactgagggaagcgacgccgagaaagcaggcgtaccacggagggagagaaaagctccggaagcccagcagcg

SEQ ID NO: 211 - DNA, 400 bp, Artificial Sequence
  Description of Artificial Sequence: Synthetic polynucleotide
aataaaatatctttattttcattacatctgtgtgttggttttttgtgtgccattgacgtcacgatttgacgtcaaccattgacgtcacgatttgacgtcaaccattgacgtcacgatttgacgtcacgatttgacgtcaaccattgactcacgatttgactcaaccattgactcacgatttgactcaaccattgactcacgatttgactcacgattctgacaaattcagtataaaagcttggggctggggccgagcactggggactttgagggtggccaggccagcgtaggaggccagcgtaggatcctgctgggagcggggaactgagggaagcgacgccgagaaagcaggcgtaccacggagggagagaaaagctccggaagcccagcagcggccacc

SEQ ID NO: 212 - DNA, 20 bp, Artificial Sequence
  Description of Artificial Sequence: Synthetic primer
gtatcctaacgcccagaccg

SEQ ID NO: 213 - DNA, 6 bp, Artificial Sequence
  Description of Artificial Sequence: Synthetic oligonucleotide
aaacat

SEQ ID NO: 214 - DNA, 29 bp, Artificial Sequence
  Description of Artificial Sequence: Synthetic oligonucleotide
  modified_base (1)..(10): a, c, t, g, unknown or other
  misc_feature (1)..(10): This sequence may encompass 3-10 nucleotides
  modified_base (12)..(12): a, c, t, g, unknown or other
  modified_base (15)..(15): a, c, t, g, unknown or other
  modified_base (17)..(19): a, c, t, g, unknown or other
  misc_feature (17)..(19): This sequence may encompass 0-3 nucleotides
  modified_base (20)..(29): a, c, t, g, unknown or other
  misc_feature (20)..(29): This sequence may encompass 3-10 nucleotides
  See specification as filed for detailed description of substitutions and preferred embodiments
nnnnnnnnnncngcngnnnnnnnnnnnnn

SEQ ID NO: 215 - DNA, 30 bp, Artificial Sequence
  Description of Artificial Sequence: Synthetic oligonucleotide
  modified_base (1)..(10): a, c, t, g, unknown or other
  misc_feature (1)..(10): This sequence may encompass 3-10 nucleotides
  modified_base (12)..(12): a, c, t, g, unknown or other
  modified_base (15)..(15): a, c, t, g, unknown or other
  modified_base (18)..(20): a, c, t, g, unknown or other
  misc_feature (18)..(20): This sequence may encompass 0-3 nucleotides
  modified_base (21)..(30): a, c, t, g, unknown or other
  misc_feature (21)..(30): This sequence may encompass 3-10 nucleotides
  See specification as filed for detailed description of substitutions and preferred embodiments
nnnnnnnnnncngcngsnnnnnnnnnnnnn

SEQ ID NO: 216 - DNA, 5 bp, Artificial Sequence
  Description of Artificial Sequence: Synthetic oligonucleotide
rcgtg

SEQ ID NO: 217 - DNA, 420 bp, Artificial Sequence
  Description of Artificial Sequence: Synthetic polynucleotide
ataaaatatctttattttcattacatctgtgtgttggttttttgtgtgaataaaatatctttattttcattacatctgtgtgttggttttttgtgtgaataaaatatctttattttcattacatctgtgtgttggttttttgtgtgaggtggggatggggcagactcatcccattcttgaccaagcccttgttctgctcccttctctccagataacttcgtatagtacacattatacgaagttatgcctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacct

SEQ ID NO: 218 - DNA, 630 bp, Artificial Sequence
  Description of Artificial Sequence: Synthetic polynucleotide
cgtcgtagactgcgtgcacagcgtctgcgtcctcgtcttgtttctcttagtcttagggttaagactacgcggccactagtctagtttttgaagtcggtccatgtacctctattgaagcatatcatgtgtaatatgcttcaatacactcaccgaaaccaagggccgactccacctcacccgactcctgatctgactcgggagcctgtaccttattttatagaaataaaagtaatgtagacacacaaccaaaaaacacacttattttatagaaataaaagtaatgtagacacacaaccaaaaaacacacttattttatagaaataaaagtaatgtagacacacaaccaaaaaacacactccacccctaccccgtctgagtagggtaagaactggttcgggaacaagacgagggaagagaggtctattgaagcatatcatgtgtaatatgcttcaatacggaccagcccaccgagcacctgttcccctaatggagcctcttcgtcacctaggtcctcctggtccggagtatgtagaggaagttacgccggaggttgagcgccagggtttagttccgacggaacctgttacgccctttctaatactcggactgattttggcgggggctgatgga

SEQ ID NO: 219 - protein, 62 aa, Artificial Sequence
  Description of Artificial Sequence: Synthetic polypeptide
QHLTHVSQTQEQNKENQNPNSDAPVIRSKTSARYMEITSYSTHYTKLCEWLWFPAEVEWAED

SEQ ID NO: 220 - protein, 7 aa, Artificial Sequence
  Description of Artificial Sequence: Synthetic peptide
TEPSDME

SEQ ID NO: 221 - protein, 14 aa, Artificial Sequence
  Description of Artificial Sequence: Synthetic peptide
NIFIFITSVCWFFV

SEQ ID NO: 222 - protein, 124 aa, Artificial Sequence
  Description of Artificial Sequence: Synthetic polypeptide
IKYLYFHYICVLVFCVNKISLFSLHLCVGFLCEVGMGQTHPILDQALVLLPSLQITSYSTHYTKLCLVGWLVDKGITSEKQWIQEDQASYISFNAASNSRSQIKAALDNAGKIMSLTKTAPDYL

SEQ ID NO: 223 - protein, 28 aa, Artificial Sequence
  Description of Artificial Sequence: Synthetic peptide
MEITSYSTHYTKLCEWLWFPAEVEWAED

SEQ ID NO: 224 - DNA, 320 bp, Artificial Sequence
  Description of Artificial Sequence: Synthetic polynucleotide
tgcgccggtgatcagatcaaaaacttcagccaggtacatggagataacttcgtatagtacacattatacgaagttatgcctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggt

SEQ ID NO: 225 - DNA, 320 bp, Artificial Sequence
  Description of Artificial Sequence: Synthetic polynucleotide
acgcggccactagtctagtttttgaagtcggtccatgtacctctattgaagcatatcatgtgtaatatgcttcaatacggaccagcccaccgagcacctgttcccctaatggagcctcttcgtcacctaggtcctcctggtccggagtatgtagaggaagttacgccggaggttgagcgccagggtttagttccgacggaacctgttacgccctttctaatactcggactgattttggcgggggctgatggaccacccggtcgtcgggcacctcctgtaaaggtcgttagcctaaatattttaaaaccttgatttgccca

SEQ ID NO: 226 - protein, 106 aa, Artificial Sequence
  Description of Artificial Sequence: Synthetic polypeptide
APVIRSKTSARYMEITSYSTHYTKLCLVGWLVDKGITSEKQWIQEDQASYISFNAASNSRSQIKAALDNAGKIMSLTKTAPDYLVGQQPVEDISSNRIYKILELNG

SEQ ID NO: 227 - protein, 94 aa, Artificial Sequence
  Description of Artificial Sequence: Synthetic polypeptide
MEITSYSTHYTKLCLVGWLVDKGITSEKQWIQEDQASYISFNAASNSRSQIKAALDNAGKIMSLTKTAPDYLVGQQPVEDISSNRIYKILELNG

(19) United States
(12) Patent Application Publication
(10) Pub. No.: US 2023/0407326 A1
(43) Pub. Date: Dec. 21, 2023

(54) INDUCIBLE PROMOTER FOR VIRAL VECTOR PRODUCTION

(71) Applicant: ASKLEPIOS BIOPHARMACEUTICAL, INC., Research Triangle Park, NC (US)

(72) Inventors: Graham Whyteside, Midlothian (GB); Michael L. Roberts, Midlothian (GB); Victoria Fiona Torrance, Midlothian (GB)

(73) Assignee: ASKLEPIOS BIOPHARMACEUTICAL, INC., Research Triangle Park, NC (US)

(21) Appl. No.: 17/907,229

(22) PCT Filed: Mar. 26, 2021
(86) PCT No.: PCT/US2021/024350

§ 371 Date: Sep. 23, 2022

Related U.S. Application Data
(60) Provisional application No. 63/000,155, filed on Mar. 26, 2020.
(60) Provisional application No. 63/010,330, filed on Apr. 15, 2020.
(60) Provisional application No. 63/148,905, filed on Feb. 12, 2021.

(51) Int. Cl. C12N 15/86

(52) U.S. Cl. CPC C12N 15/86; C12N 2750/14143; C12N 2750/14152; C12N 2830/002

(57) ABSTRACT

Aspects described herein relate to stable cell line for recombinant viral vector (e.g., recombinant adeno associated viral vector) production comprising at least one inducible promoter operatively linked to a heterologous gene that encodes a toxic protein. Methods for making stable cell lines, and methods for viral vector production are further provided herein.

### CROSS REFERENCE TO RELATED APPLICATIONS

This application is a 35 U.S.C. § 371 National Phase Entry Application of international patent application No. PCT/US2021/024350 filed Mar. 26, 2021, which designated the U.S., which claims benefit under 35 U.S.C. § 119(e) of U.S. Provisional Application Nos 63/000,155 filed Mar. 26, 2020; 63/010,330 filed Apr. 15, 2020; and 63/148,905 filed Feb. 12, 2021, the contents of which are incorporated herein by reference in their entireties.

### SEQUENCE LISTING

The instant application contains a Sequence Listing which has been submitted electronically in ASCII format and is hereby incorporated by reference in its entirety. Said ASCII copy, created on Oct. 12, 2022, is named 046192-097100WOPT_SL.txt and is 173,128 bytes in size.

### FIELD OF THE INVENTION

The present invention relates to cell lines for rapid and scalable production of viral vectors, for example, adeno-associated virus (AAV).

### BACKGROUND OF THE INVENTION

Recombinant viral vectors, for example recombinant adeno-associated viral vectors, lentiviral vectors and adeno vectors, all carrying a heterologous DNA (transgene) are used to deliver genes to cells, where the gene may be expressed to permit, e.g., production of recombinant proteins in vitro or in vivo, vaccination, or treatment of disease states or genetic defects. One may treat a disease state or genetic defects by, e.g., a viral gene, such as replication providing an effective level of normal gene products, increased levels of gene products thereby correcting a mal-functioning gene, or by blocking endogenous production of a gene, whose expression is deleterious to the cell or organism.

Methods for delivering an exogenous gene to a mammalian cell include the use of mammalian viral vectors, such as those that are derived from retroviruses (e.g., lentiviruses), adenoviruses, herpes viruses, vaccinia viruses, polio viruses, adeno-associated viruses, hybrid viruses and the like. A limiting factor in the gene therapy field is identifying an efficient, scalable method for producing such viral vectors in a large quantity.

Adeno-associated virus (AAV) systems have many advantages that can be exploited for delivery of transgenes, and are thus an ideal viral vector for a gene therapeutic. With respect to AAV production, viral protein, replication (rep) has long been known to be required for replication and excision of the AAV genome, however, it is unclear how much Rep protein is required for effective rAAV production. Rep proteins have been shown to be toxic to cell lines, resulting in difficulties with producing stable cell lines expressing Rep. It has further been suggested that attenuation of Rep78/68 production during viral propagation results in higher levels of production of rAAV. In contrast, other art explicitly states that high expression of Rep proteins—a result of replacing the native rep p5 promoter with a stronger promoter—results in high level expression of rAAV. When considering the art, it appears that varying levels of Rep may be required given a particular viral producer cell for the production of a rAAV vector. Similarly, capsid proteins can be toxic to cells. Thus, is important to obtain fine temporal and/or spatial control of Rep, and/or other toxic genes required for viral production, in order to optimize production of viral vectors.

### SUMMARY OF INVENTION

One aspect of the invention described herein provides a stable cell line for recombinant viral vector production, comprising at least one inducible promoter operatively linked to a heterologous gene that encodes a toxic protein.

In one embodiment of any aspect provided herein, the regulatable promoter is an inducible or repressible promoter. In one embodiment of any aspect provided herein, the regulatable promoter is an inducible promoter.

In one embodiment of any aspect provided herein, the toxic protein is a viral protein. Exemplary viral proteins include replication (rep), capsid (cap), envelope (env), and polymerase (pol).

In one embodiment of any aspect provided herein, the toxic protein is associated with nucleic acid transcription.

In one embodiment of any aspect provided herein, the toxic protein is associated with capsid or envelope production.

In one embodiment of any aspect provided herein, the inducible promoter is selected from the group selected from a forskolin inducible promoter, a hypoxia inducible promoter, a tetracycline inducible promoter, an alcohol inducible promoter, a steroid inducible promoter, an RU486 inducible promoter, an ecdysone inducible promoter, a rapamycin inducible promoter, a metallothionein inducible promoter, a hormone inducible promoter and a metal inducible promoter.

In one embodiment of any aspect provided herein, the cell comprises at least two inducible promoters, and wherein the at least two inducible promoters are induced by different compositions, and the at least two inducible promoters are operatively linked to distinct heterologous genes that encode different toxic proteins.

In one embodiment of any aspect provided herein, the cell comprises a first inducible promoter that is operatively linked to a repressible element that can stop protein expression.

In one embodiment of any aspect provided herein, the first inducible promoter that further encodes a protein that represses expression of the first inducible promoter.

In one embodiment of any aspect provided herein, the cell comprises a first inducible promoter that further encodes a protein that induces expression of a second inducible promoter.

In one embodiment of any aspect provided herein, the cell is a eukaryotic cell or a prokaryotic cell.

In one embodiment of any aspect provided herein, the cell is selected from the cell types listed in Table 2. In one embodiment of any aspect provided herein, the cell is derived from a cell type selected from the cell types listed in Table 2.

In one embodiment of any aspect provided herein, contacting the cell with an inducer results in expression of the at least one toxic protein.

In one embodiment of any aspect provided herein, the cell is for use in production of a viral particles selected from the group consisting of: an adenovirus vector, a lentivirus vector, a retrovirus vector, a herpesvirus vector, an alphavirus vector, a poxvirus vector, a baculovirus vector, and a chimeric virus vector.

One aspect of the invention described herein is a stable cell line for recombinant AAV vector production, comprising at least one inducible promoter, wherein the inducible promoter is operatively linked to a heterologous rep gene that encodes a rep protein.

In one embodiment of any aspect provided herein, the inducible promoter is further operatively linked to a heterologous cap gene that encodes a cap protein.

In one embodiment of any aspect provided herein, the stable cell further comprises a second inducible promoter operatively linked to a heterologous cap gene that encodes a cap protein, wherein the second inducible promoter is induced by a compound different from the first inducible promoter.

One aspect of the invention described herein is a stable cell line for recombinant AAV vector production, comprising at least one inducible promoter, wherein the inducible promoter is operatively linked to a heterologous cap gene that encodes a cap protein.

One aspect of the invention described herein is a method of producing any of the stable cell lines provided herein, the method comprising: (a) transforming a population of cells with at least one nucleic acid cassette containing an inducible promoter operatively linked to a heterologous gene that encodes a toxic protein; (b) culturing the population of cells of (a) under conditions and for a time sufficient to permit expression of the nucleic acid cassette; (c) selecting for a cell that stably expresses the nucleic acid cassette; and (d) growing the cell of (c) to produce the cell line.

One aspect of the invention described herein is a method of producing adeno associate virus (AAV) particles, comprising; (a) providing any of the stable cell lines for rAAV production in an AAV expression system; (b) culturing the cells under conditions in which the at least one toxic protein is expressed; (c) culturing the cells under conditions in which AAV particles are produced; and (d) optionally isolating the AAV particles.

In one embodiment of any aspect provided herein, the cells are cultured in suspension.

In one embodiment of any aspect provided herein, the cells are cultured in animal component-free conditions.

In one embodiment of any aspect provided herein, step (c) comprises isolating the AAV particles from the cells.

In one embodiment of any aspect provided herein, step (c) comprises isolating the AAV particles from medium in which the cells are cultured.

In one embodiment of any aspect provided herein, the cells are cultured in shaker flasks.

In one embodiment of any aspect provided herein, the cells are cultured in bioreactors.

In one embodiment of any aspect provided herein, step (c) occurs after the toxic protein is expressed.

In one embodiment of any aspect provided herein, if the stable cell comprises at least two inducible promoters, the at least two inducible promoters are induced at substantially the same time.

In one embodiment of any aspect provided herein, if the stable cell comprises at least two inducible promoters, the at least two inducible promoters are induced at different time and/or for different durations.

In one embodiment of any aspect provided herein, the method is capable of producing all serotypes, chimeras, and hybrids of AAV. Exemplary AAVs include AAV1, AAV2, AAV3, AAV4, AAV5, AAV6, AAV7, AAV8, AAV9, AAV 10, AAV 11, AAV12, and AAV13 or a chimeric AAV that is composed of AAV1-13 2.5, 218, 9.45 and other chimeric or hybrid capsids.

In one embodiment of any aspect provided herein, the AAV particle comprises a rational haploid capsid.

In one embodiment of any aspect provided herein, the AAV expression system comprises at least one of a recombinant AAV plasmid, a plasmid expressing Rep, a plasmid expressing Cap, and an adenovirus helper plasmid.

In one embodiment of any aspect provided herein, the recombinant AAV plasmid encodes a transgene. In one embodiment of any aspect provided herein, the transgene is a therapeutic transgene.

In one embodiment of any aspect provided herein, the method provides at least about 4×10⁴ vector genome-containing particles per cell prior to purification. In one embodiment of any aspect provided herein, the method provides at least about 1×10⁵ vector genome-containing particles per cell prior to purification. In one embodiment of any aspect provided herein, the method provides at least about 1×10¹² purified vector genome-containing particles per liter of cell culture. In one embodiment of any aspect provided herein, the method provides at least about 1×10¹³ purified vector genome-containing particles per liter of cell culture.

One aspect of the invention described herein is a method of producing viral particles, comprising; (a) providing and of the stable cell line described herein in a viral expression system; (b) culturing the cells under conditions in which at least one toxic protein is expressed, wherein the at least one toxic protein is operatively linked to at least one inducible promoter; (c) culturing the cells under conditions in which viral particles are produced; and (d) optionally isolating the viral particles.

In one embodiment of any aspect provided herein, the viral particles are selected from the group consisting of: an adenovirus vector, a lentivirus vector, a retrovirus vector, a herpesvirus vector, an alphavirus vector, a poxvirus vector, a baculovirus vector, and a chimeric virus vector.

One aspect of the invention described herein provides a cell line for recombinant viral vector production, comprising transient expression of at least one inducible promoter, wherein the inducible promoter is operatively linked to a heterologous gene that encodes a toxic protein.

One aspect of the invention described herein provides a stable cell line for recombinant viral vector production, comprising at least one inducible promoter operatively linked to a heterologous gene that encodes a toxic protein, wherein the at least one inducible promoter is selected from the group consisting of: a forskolin inducible promoter, a hypoxia inducible promoter, a tetracycline inducible promoter, an alcohol inducible promoter, a steroid inducible promoter, an RU486 inducible promoter, an ecdysone inducible promoter, a rapamycin inducible promoter, a metallothionein inducible promoter, a hormone inducible promoter and a metal inducible promoter.

One aspect of the invention described herein provides a stable cell line for recombinant viral vector production, comprising at least one forskolin inducible promoter operatively linked to at least one heterologous gene that encodes a toxic protein.

One aspect of the invention described herein provides a stable cell line for recombinant viral vector production, comprising at least one hypoxia inducible promoter operatively linked to at least one heterologous gene that encodes a toxic protein.

One aspect of the invention described herein provides a stable cell line for recombinant viral vector production, comprising at least one forskolin inducible promoter operatively linked to at least one heterologous gene that encodes a toxic protein, and at least one inducible promoter operatively linked to at least one heterologous gene that encodes a toxic protein, wherein the at least one inducible promoter is selected from the group consisting of: a forskolin inducible promoter, a hypoxia inducible promoter, a tetracycline inducible promoter, an alcohol inducible promoter, a steroid inducible promoter, an RU486 inducible promoter, an ecdysone inducible promoter, a rapamycin inducible promoter, a metallothionein inducible promoter, a hormone inducible promoter and a metal inducible promoter.

One aspect of the invention described herein provides a stable cell line for recombinant viral vector production, comprising at least one hypoxia inducible promoter operatively linked to at least one heterologous gene that encodes a toxic protein, and at least one inducible promoter operatively linked to at least one heterologous gene that encodes a toxic protein, wherein the at least one inducible promoter is selected from the group consisting of: a forskolin inducible promoter, a hypoxia inducible promoter, a tetracycline inducible promoter, an alcohol inducible promoter, a steroid inducible promoter, an RU486 inducible promoter, an ecdysone inducible promoter, a rapamycin inducible promoter, a metallothionein inducible promoter, a hormone inducible promoter and a metal inducible promoter.

One aspect of the invention described herein provides a stable cell line for recombinant viral vector production, comprising at least one forskolin inducible promoter operatively linked to at least one heterologous gene that encodes a toxic protein, and at least one hypoxia inducible promoter operatively linked to at least one heterologous gene that encodes a toxic protein.

One aspect of the invention described herein provides a stable cell line for recombinant viral vector production, comprising at least one inducible promoter having a sequence of SEQ ID NO: 1 or SEQ ID NO: 3 operatively linked to at least one heterologous gene that encodes a toxic protein. Variants of these and other promoters suitable for use in the present invention are described below.

One aspect of the invention described herein provides a stable cell line for recombinant viral vector production, comprising at least one inducible promoter having a sequence of any one of SEQ ID NO: 6 to SEQ ID NO: 9 operatively linked to at least one heterologous gene that encodes a toxic protein. Variants of these and other promoters suitable for use in the present invention are described below.

One aspect of the invention described herein provides a stable cell line for recombinant viral vector production, comprising at least one inducible promoter having a sequence of SEQ ID NO: 1 or SEQ ID NO: 3 operatively linked to at least one heterologous gene that encodes a toxic protein, and at least one inducible promoter operatively linked to at least one heterologous gene that encodes a toxic protein, wherein the at least one inducible promoter is selected from the group consisting of: a forskolin inducible promoter, a hypoxia inducible promoter, a tetracycline inducible promoter, an alcohol inducible promoter, a steroid inducible promoter, an RU486 inducible promoter, an ecdysone inducible promoter, a rapamycin inducible promoter, a metallothionein inducible promoter, a hormone inducible promoter and a metal inducible promoter.

One aspect of the invention described herein provides a stable cell line for recombinant viral vector production, comprising at least one inducible promoter having a sequence of any one of SEQ ID NO: 6 to SEQ ID NO: 9 operatively linked to at least one heterologous gene that encodes a toxic protein, and at least one inducible promoter operatively linked to at least one heterologous gene that encodes a toxic protein, wherein the at least one inducible promoter is selected from the group consisting of: a forskolin inducible promoter, a hypoxia inducible promoter, a tetracycline inducible promoter, an alcohol inducible promoter, a steroid inducible promoter, an RU486 inducible promoter, an ecdysone inducible promoter, a rapamycin inducible promoter, a metallothionein inducible promoter, a hormone inducible promoter and a metal inducible promoter.

One aspect of the invention described herein provides a stable cell line for recombinant viral vector production, comprising at least one inducible promoter having a sequence of SEQ ID NO: 1 or SEQ ID NO: 3 operatively linked to at least one heterologous gene that encodes a toxic protein, and at least one inducible promoter having a sequence of any one of SEQ ID NO: 6 to SEQ ID NO: 9 operatively linked to at least one heterologous gene that encodes a toxic protein.

One aspect of the invention described herein provides a stable cell line for rAAV production, comprising at least one inducible promoter operatively linked to a heterologous gene that encodes a toxic protein, wherein the at least one inducible promoter is selected from the group consisting of: a forskolin inducible promoter, a hypoxia inducible promoter, a tetracycline inducible promoter, an alcohol inducible promoter, a steroid inducible promoter, an RU486 inducible promoter, an ecdysone inducible promoter, a rapamycin inducible promoter, a metallothionein inducible promoter, a hormone inducible promoter and a metal inducible promoter.

One aspect of the invention described herein provides a stable cell line for rAAV production, comprising at least one forskolin inducible promoter operatively linked to at least one heterologous gene that encodes a toxic protein.

One aspect of the invention described herein provides a stable cell line for rAAV production, comprising at least one hypoxia inducible promoter operatively linked to at least one heterologous gene that encodes a toxic protein.

One aspect of the invention described herein provides a stable cell line for rAAV production, comprising at least one forskolin inducible promoter operatively linked to at least one heterologous gene that encodes a toxic protein, and at least one inducible promoter operatively linked to at least one heterologous gene that encodes a toxic protein, wherein the at least one inducible promoter is selected from the group consisting of: a forskolin inducible promoter, a hypoxia inducible promoter, a tetracycline inducible promoter, an alcohol inducible promoter, a steroid inducible promoter, an RU486 inducible promoter, an ecdysone inducible promoter, a rapamycin inducible promoter, a metallothionein inducible promoter, a hormone inducible promoter and a metal inducible promoter.

One aspect of the invention described herein provides a stable cell line for rAAV production, comprising at least one hypoxia inducible promoter operatively linked to at least one heterologous gene that encodes a toxic protein, and at least one inducible promoter operatively linked to at least one heterologous gene that encodes a toxic protein, wherein the at least one inducible promoter is selected from the group consisting of: a forskolin inducible promoter, a hypoxia inducible promoter, a tetracycline inducible promoter, an alcohol inducible promoter, a steroid inducible promoter, an RU486 inducible promoter, an ecdysone inducible promoter, a rapamycin inducible promoter, a metallothionein inducible promoter, a hormone inducible promoter and a metal inducible promoter.

One aspect of the invention described herein provides a stable cell line for rAAV production, comprising at least one forskolin inducible promoter operatively linked to at least one heterologous gene that encodes a toxic protein, and at least one hypoxia inducible promoter operatively linked to at least one heterologous gene that encodes a toxic protein.

One aspect of the invention described herein provides a stable cell line for rAAV production, comprising at least one inducible promoter having a sequence of SEQ ID NO: 1 or SEQ ID NO: 3 operatively linked to at least one heterologous gene that encodes a toxic protein.

One aspect of the invention described herein provides a stable cell line for rAAV production, comprising at least one inducible promoter having a sequence of any one of SEQ ID NO: 6 to SEQ ID NO: 9 operatively linked to at least one heterologous gene that encodes a toxic protein.

One aspect of the invention described herein provides a stable cell line for rAAV production, comprising at least one inducible promoter having a sequence of SEQ ID NO: 1 or SEQ ID NO: 3 operatively linked to at least one heterologous gene that encodes a toxic protein, and at least one inducible promoter operatively linked to at least one heterologous gene that encodes a toxic protein, wherein the at least one inducible promoter is selected from the group consisting of: a forskolin inducible promoter, a hypoxia inducible promoter, a tetracycline inducible promoter, an alcohol inducible promoter, a steroid inducible promoter, an RU486 inducible promoter, an ecdysone inducible promoter, a rapamycin inducible promoter, a metallothionein inducible promoter, a hormone inducible promoter and a metal inducible promoter.

One aspect of the invention described herein provides a stable cell line for rAAV production, comprising at least one inducible promoter having a sequence of any one of SEQ ID NO: 6 to SEQ ID NO: 9 operatively linked to at least one heterologous gene that encodes a toxic protein, and at least one inducible promoter operatively linked to at least one heterologous gene that encodes a toxic protein, wherein the at least one inducible promoter is selected from the group consisting of: a forskolin inducible promoter, a hypoxia inducible promoter, a tetracycline inducible promoter, an alcohol inducible promoter, a steroid inducible promoter, an RU486 inducible promoter, an ecdysone inducible promoter, a rapamycin inducible promoter, a metallothionein inducible promoter, a hormone inducible promoter and a metal inducible promoter.

One aspect of the invention described herein provides a stable cell line for rAAV production, comprising at least one inducible promoter having a sequence of SEQ ID NO: 1 or SEQ ID NO: 3 operatively linked to at least one heterologous gene that encodes a toxic protein, and at least one inducible promoter having a sequence of any one of SEQ ID NO: 6 to SEQ ID NO: 9 operatively linked to at least one heterologous gene that encodes a toxic protein.

In one embodiment of any aspect provided herein, the stable cell line further comprises at least one repressible element operatively linked to at least one heterologous gene that encodes a toxic protein.

In one embodiment of any aspect provided herein, the stable cell line has at least two inducible promoters, and the at least two inducible promoters are the same.

In one embodiment of any aspect provided herein, the stable cell line has at least two inducible promoters, and the at least two inducible promoters are different.

Certain aspects of the technology described herein generally relates to nucleic acid constructs, methods, and systems for sequential and/or temporal regulation of gene expression of one or more viral proteins. Such nucleic acid constructs described herein are suitable for viral vector expression systems, e.g., AAV expression systems, and for the generations of a stable cell line for viral vector expression systems and AAV vector expression systems.

One aspect described herein provides a nucleic acid construct comprising a nucleic acid sequence comprising at least one of a nucleic acid sequence encoding a viral, e.g., E4, protein, a nucleic acid sequence encoding a second viral, e.g., E2A, protein, and a nucleic acid sequence encoding a viral RNA, e.g., VA RNA, wherein each nucleic acid sequence encoding any one of E4, E2A, VA is operatively linked to a first regulatable promoter; and a nucleic acid sequence encoding a Rep protein, wherein the nucleic acid encoding a Rep protein is under the control of a second regulatable promoter or regulatable transcriptional activator, wherein the first and the second regulatable promoters are different types of regulatable promoters. In one embodiment, the regulatable promoters are inducible promoters e.g., hypoxia and/or forskolin inducible promoters.

Exemplary transcriptional activators include homeodomain transcriptional activator, zinc-finger transcriptional activator, winged-helix (Forkhead) transcriptional activator, leucine-zipper transcriptional activator, and helix-loop-helix transcriptional activator. In one embodiment, the transcriptional activator is a zinc-finger transcriptional activator (ZF-TA).

In one embodiment, a first and a second Rep protein are encoded by the nucleic acid sequence, or by one or more nucleic acid sequences. In one embodiment, the first Rep protein is large Rep, e.g., Rep78, and the second Rep protein is small Rep, e.g., Rep 52. In one embodiment, the nucleic acid encoding the first and second Rep proteins is under the control of a second regulatable promoter or regulatable transcriptional activator. In one embodiment, each nucleic acid encoding the first and second Rep proteins is under the control of a regulatable transcriptional activator. In one embodiment, each nucleic acid encoding the first and second Rep proteins is under the control of the same regulatable transcriptional activator. In one embodiment, the transcriptional activator is a zinc-finger transcriptional activator (ZF-TA).

In one embodiment, the nucleic acid sequence encoding the Rep protein comprises a modified start codon. In one embodiment, the nucleic acid sequence encoding the Rep78 protein comprises a modified start codon. In one embodiment, the nucleic acid sequence encoding the Rep78 protein comprises a modified start codon selected from: ACC, AUC, CUG, and AGG. In such embodiments, the nucleic acid sequence encoding the Rep52 protein comprises a typical start codon.

In such embodiments, the nucleic acid sequence encoding the Rep52 protein comprises and ATG start codon.

In one embodiment of any aspect, the Rep protein is a modified Rep protein. In one embodiment of any aspect, the Rep protein is a modified Rep78 protein.

In one embodiment of any aspect, the modified Rep protein has a lysine to arginine mutation at amino acid 84. In one embodiment of any aspect, the modified Rep78 protein has a lysine to arginine mutation at amino acid 84.

In one embodiment of any aspect, the nucleic acid encoding the Rep protein further comprises a nucleic acid encoding a ribozyme at its 3′ end.

In one embodiment of any aspect, the second regulatable promoter operatively linked to the nucleic acid encoding the Rep protein is an inducible promoter or comprises a binding site for a regulatable transcriptional activator. In one embodiment of any aspect, the first regulatable promoter operatively linked to the nucleic acid encoding any one of E4, E2A, VA is an inducible promoter.

In one embodiment of any aspect, the inducible promoter is selected from the group consisting of a forskolin inducible promoter, a hypoxia inducible promoter, a tetracycline inducible promoter, an alcohol inducible promoter, a steroid inducible promoter, an RU486 inducible promoter, an ecdysone inducible promoter, a rapamycin inducible promoter, a metallothionein inducible promoter, a hormone inducible promoter and a metal inducible promoter. In one embodiment, the inducible promoter is a forskolin inducible promoter or a hypoxia inducible promoter.

In one embodiment of any aspect, the inducible promoter lacks a minimal promoter.

In one embodiment of any aspect, the inducible promoter further comprises a TATA box sequence, or a p5 replication sequence, or both a TATA box sequence and p5 replication sequence. In one embodiment of any aspect, the inducible promoter comprises a TATA box sequence, or a p5 replication sequence, or both a TATA box sequence and p5 replication sequence, instead of a minimal promoter. In such embodiments, the inducible promoter may be the second regulatable promoter.

Another aspect described herein provides a nucleic acid construct comprising a nucleic acid encoding a promoter, and a TATA box and/or p5, wherein the promoter does not comprise a minimal promoter.

Another aspect described herein provides a nucleic acid construct comprising a nucleic acid sequence encoding a regulatable transcriptional activator operatively linked to an inducible promoter, wherein the inducible promoter is a E4-responsive promoter, or a E2-responsive promoter or other helper-gene responsive promoter. In one embodiment, the helper gene responsive promoter comprises a sequence bound by a transcriptional activator, and wherein the expression or activity of the transcriptional activator is inducible by expression of one or more helper genes. In some embodiments, the regulatable transcriptional activator is a zinc-finger transcriptional activator.

Another aspect described herein provides a nucleic acid construct comprising a toxic protein operatively linked to a promoter comprising a target site for binding of a regulatable transcriptional activator, e.g., a zinc-finger transcriptional activator (ZF-TA). In one embodiment, the toxic protein is a Rep protein. In one embodiment, the helper gene responsive promoter comprises a sequence bound by a transcriptional activator, and wherein the expression or activity of the transcriptional activator is inducible by expression of one or more helper genes.

Another aspect described herein provides a nucleic acid construct comprising a gene encoding a Rep protein operatively linked to a promoter comprising a target site for binding of a regulatable transcriptional activator, e.g., a zinc-finger transcriptional activator (ZF-TA), and a promoter.

In one embodiment of any aspect, the regulatable transcriptional activator, e.g. zinc-finger transcriptional activator (ZF-TA), is expressed from the nucleic acid construct encoding a regulatable transcriptional activator, e.g., zinc finger (ZF) transcriptional activator operatively linked to an inducible promoter, wherein the inducible promoter is a helper gene responsive promoter, e.g., an E4-responsive promoter, or a E2-responsive promoter or other helper-gene responsive promoter.

In one embodiment of any aspect, the nucleic acid construct further comprises at least one of: a nucleic acid sequence encoding at least one helper protein, wherein each nucleic acid construct is operatively linked to a regulatable promoter; a nucleic acid encoding a toxic protein wherein the toxic protein under the control of a second regulatable promoter or a regulatable transcriptional activator; a nucleic acid sequence encoding a E4 protein, a nucleic acid sequence encoding a E2A protein, and a nucleic acid sequence encoding a VA RNA, wherein each nucleic acid sequence encoding any one of E4, E2A, and VA RNA is operatively linked to a regulatable promoter; or a nucleic acid encoding a Rep protein, wherein the Rep protein is under the control of a second regulatable promoter or a regulatable transcriptional activator. In one embodiment, the regulatable transcriptional activator is a zinc-finger transcriptional activator.

Another aspect described herein provides a nucleic acid construct comprising a nucleic acid sequence encoding a tetracycline-responsive transactivator protein operatively linked to a promoter, e.g., a constitutive promoter; a nucleic acid sequence comprising at least one of: a nucleic acid sequence encoding a E4 protein, a nucleic acid sequence encoding a E2A protein, and a nucleic acid sequence encoding a VA RNA, wherein each nucleic acid sequences encoding any one of E4, E2A, VA RNA is operatively linked to a regulatable promoter; and/or a nucleic acid sequence encoding a regulatable transcriptional activator operatively linked to an inducible promoter, wherein the inducible promoter is a E4-responsive promoter, or a E2-responsive promoter; and a nucleic acid construct comprising a Rep protein operatively linked to a promoter comprising a target site for binding of the regulatable transcriptional activator. In one embodiment, the regulatable transcriptional activator is a zinc-finger transcriptional activator.

Another aspect described herein provides a nucleic acid construct comprising a nucleic acid sequence encoding a Cap protein and a recombinase recognition sequence (RRS) located 3′ of the nucleic acid sequence encoding the Cap protein. Another aspect described herein provides a nucleic acid construct comprising a nucleic acid sequence encoding a Cap protein and a recombinase recognition sequence (RRS) located 5′ of the nucleic acid sequence encoding the Cap protein.

In one embodiment of any aspect, the nucleic acid sequence encoding a Cap protein is operatively linked to a constitutive promoter.

In one embodiment of any aspect, the nucleic acid sequence encoding a Cap protein is operatively linked a regulatable promoter. In one embodiment of any aspect, the regulatable promoter is an inducible promoter.

In one embodiment of any aspect, the RRS is a Flippase-responsive RRSs.

In one embodiment of any aspect, the nucleic acid construct further comprises a nucleic acid encoding a recombinase protein operatively linked to an inducible promoter.

Another aspect provided herein is a nucleic acid construct comprising a first nucleic acid construct comprising a promoter, a stop nucleic acid sequence flanked by a first pair of recombinase recognition sequences (RRS), and nucleic acid sequence encoding a Rep protein, wherein the promoter is operatively linked to the nucleic acid encoding the Rep protein, and a second nucleic acid construct comprising, in a 5′ to 3′ direction: a promoter, a stop nucleic acid sequence flanked by a second pair of recombinase recognition sequences (RRSs), and nucleic acid sequence encoding one or more of E2A, E4, and VA RNA, wherein the promoter is operatively linked to the nucleic acid encoding the one or more of E2A, E4, and VA RNA.

Another aspect provided herein is a nucleic acid construct comprising a first nucleic acid construct comprising in a 5′ to 3′ direction: a promoter, a stop nucleic acid sequence flanked by a first pair of recombinase recognition sequences (RRS), and nucleic acid sequence encoding a Rep protein, wherein the promoter is operatively linked to the nucleic acid encoding the Rep protein, and a second nucleic acid construct comprising, in a 5′ to 3′ direction: a promoter, a stop nucleic acid sequence flanked by a second pair of recombinase recognition sequences (RRSs), and nucleic acid sequence encoding one or more of E2A, E4, and VA RNA, wherein the promoter is operatively linked to the nucleic acid encoding the one or more of E2A, E4, and VA RNA.

In one embodiment of any aspect, the nucleic acid construct further comprises a nucleic acid encoding one or more selection markers flanked between a third pair of recombinase recognition sequences (RRSs), wherein the pair of RRS are in the same orientation with respect to each other, and wherein the nucleic acid encoding the one or more selection markers is operatively linked to one or more promoters described herein.

In one embodiment of any aspect, the first pair of RRS and the second pair of RRS are in the same orientation with respect to each other.

In one embodiment of any aspect, the first pair of RRS and the second pair of RRS are in the inverse orientation with respect to each other.

In one embodiment of any aspect, the first pair of RRS, second pair of RRS, and third pair of RRS are each responsive to different tyrosine recombinase or serine integrase enzymes.

In one embodiment of any aspect, the first pair of RRS and second pair of RRS, are responsive to the same tyrosine recombinase or serine integrase enzyme.

In one embodiment of any aspect, the first pair of RRS, or second pair of RRS, or both are Cre-responsive RRS.

In one embodiment of any aspect, the third pair of RRSs are Flipase-responsive RRS.

In one embodiment of any aspect, the cell further comprises a construct comprising a nucleic acid encoding a recombinase protein operatively linked to an inducible promoter. In one embodiment of any aspect, the cell further comprises a nucleic acid encoding a Flipase recombinase protein operatively linked to an inducible promoter.

In one embodiment of any aspect, the cell further comprises a nucleic acid encoding a Cre recombinase protein operatively linked to an inducible promoter.

Another aspect described herein provides a cell comprising any of the nucleic acid constructs described herein.

Another aspect described herein provides a cell comprising at least one of any of the nucleic acid constructs described herein. In one embodiment of any aspect, the cell comprises at least two of any of the nucleic acid constructs described herein. In one embodiment of any aspect, the cell comprises at least three of any of the nucleic acid constructs described herein.

In one embodiment of any aspect, the cell further comprises a construct comprising a nucleic acid sequence encoding a tetracycline-responsive transactivator protein operatively linked to a promoter (e.g., a constitutive promoter).

In one embodiment of any aspect, the cell further comprises a construct comprising a nucleic acid sequence encoding a marker protein. In one embodiment of any aspect, the nucleic sequence encoding a marker protein is flanked by recombinase recognition sequences (RRSs) in the same orientation with respect to each other.

In embodiment of any aspect, the cell further comprises a synthetic gene regulation system, wherein the synthetic gene regulation system comprises a targeted DNA binding protein or a nucleic acid sequence encoding a targeted DNA binding protein operably linked to a promoter; and a nucleic acid sequence encoding a gene of interest operably linked to a target promoter, wherein the targeted DNA binding protein is capable of binding to a target sequence, wherein the target sequence is located within the target promoter and/or the nucleic acid sequence encoding the gene of interest to thereby moderate or prevent expression of the gene of interest.

In one embodiment of any aspect, expression of the construct is a stable expression.

In one embodiment of any aspect, expression of the construct is a transient expression.

In one embodiment of any aspect, the cell comprises at least two nucleic acid constructs, and the expression of the at least two nucleic acid constructs is a stable expression.

In one embodiment of any aspect, the cell comprises at least two nucleic acid constructs, and the expression of the at least two nucleic acid constructs is a transient expression.

In one embodiment of any aspect, wherein the cell comprises at least two nucleic acid constructs, and the expression of at least one nucleic acid construct is a stable expression.

Another aspect described herein provides a stable cell comprising stable expression of at least one nucleic acid construct described herein.

Another aspect described herein provides a cell comprising transient expression of at least one nucleic acid construct described herein.

Another aspect described herein provides a method of producing viral particles, comprising providing any cell lines described herein, any stable cell line described herein, or any transient cell line described herein in a viral expression system; culturing the cells for a time sufficient and under conditions in which the at least one nucleic acid under the control of an regulatable promoter is expressed; culturing the cells under conditions in which viral particles are produced; and optionally isolating the viral particles.

Another aspect described herein provides a method of producing viral particles, comprising; (a) providing the cell line expressing a nucleic acid sequence comprising at least one of: a nucleic acid sequence encoding a E4 protein, a nucleic acid sequence encoding a E2A protein, and a nucleic acid sequence encoding a VA protein, wherein each nucleic acid sequences encoding any one of E4, E2A, and VA RNA is operatively linked to a first regulatable promoter; and a nucleic acid sequence encoding a Rep protein, wherein the nucleic acid encoding a Rep protein is under the control of a second regulatable promoter, wherein the first and the second regulatable promoters are different; (b) culturing the cells for a time sufficient and under conditions in which at least the nucleic acid sequence encoding a E4 protein, the nucleic acid sequence encoding a E2A protein, or the nucleic acid sequence encoding a VA RNA is expressed first in the viral vector production protocol; (c) culturing the cells for a time sufficient and under conditions in which the nucleic acid sequence encoding a Rep protein is expressed second in the viral vector production protocol; (d) culturing the cells under conditions in which viral particles are produced; and (e) optionally isolating the viral particles. In one embodiment, the nucleic acid sequence encoding a E4 protein, the nucleic acid sequence encoding a E2A protein, or the nucleic acid sequence encoding a VA RNA is expressed between hours 3-4 of the viral vector production protocol. In one embodiment, the nucleic acid sequence encoding a Rep protein is expressed between hours 6-8 of the viral vector production protocol.

In one embodiment of any aspect, culturing in step (b) is culturing with an inducer of the first regulatable promoter. In one embodiment of any aspect, culturing in step (c) is culturing with an inducer of the second regulatable promoter. In one embodiment of any aspect, the or each inducer may act directly or indirectly to induce expression of a given nucleic acid.

Another aspect described herein provides a method of producing viral particles, comprising; (a) providing the cell line expressing at least one of a nucleic acid construct comprising a nucleic acid sequence encoding a regulatable transcriptional activator operatively linked to an inducible promoter, wherein the inducible promoter is a helper gene responsive promoter, e.g., an E4-responsive promoter, or a E2-responsive promoter or other helper-gene responsive promoter; nucleic acid construct comprising a toxic protein operatively linked to a promoter comprising a target site for binding of a regulatable transcriptional activator; nucleic acid construct comprising a Rep protein operatively linked to a promoter comprising a target site for binding of a regulatable transcriptional activator; a nucleic acid sequence encoding at least one helper protein, wherein each nucleic acid construct is operatively linked to a regulatable promoter; a nucleic acid encoding the toxic protein is under the control of a second regulatable promoter or a regulatable transcriptional activator; a nucleic acid sequence encoding a E4 protein, a nucleic acid sequence encoding a E2A protein, and a nucleic acid sequence encoding a VA RNA, wherein each nucleic acid sequences encoding any one of E4, E2A, and VA RNA is operatively linked to a regulatable promoter; or a nucleic acid encoding the Rep protein is under the control of a second regulatable promoter or a regulatable transcriptional activator; (b) culturing the cells for a time sufficient and under conditions in which at least the nucleic acid sequence encoding the E4 protein, the nucleic acid sequence encoding the E2A protein, or the nucleic acid sequence encoding the VA protein is expressed first in the viral vector production protocol; (c) culturing the cells for a time sufficient and under conditions in which the nucleic acid sequence encoding a toxic protein or Rep protein is expressed second in the viral vector production protocol; (d) culturing the cells under conditions in which viral particles are produced; and (e) optionally isolating the viral particles. In one embodiment, the nucleic acid sequence encoding a E4 protein, the nucleic acid sequence encoding a E2A protein, or the nucleic acid sequence encoding a VA RNA is expressed between hours 3-4 of the viral vector production protocol. In one embodiment, the nucleic acid sequence encoding a Rep protein is expressed between hours 6-8 of the viral vector production protocol. In one embodiment, the regulatable transcriptional activator is a zinc-finger transcriptional activator (ZF-TA). In one embodiment the expression or activity of the transcriptional activator is inducible by expression of one or more helper genes.

In one embodiment of any aspect, culturing in step (b) is culturing with an inducer of the regulatable promoter operatively linked to a nucleic acid encoding at least E4, E2A, or VA RNA. In one embodiment of any aspect, culturing in step (c) is culturing with the transcriptional activator (e.g., ZF-TA) or an inducer of a second regulatable promoter. In one embodiment of any aspect, expression of the transcriptional activator (e.g., ZA-TA) is induced by expression of E4 or E2 or VA RNA. In one embodiment of any aspect, expression of the transcriptional activator (e.g., ZA-TA) is induced by expression of E4 or E2 or VA RNA directly or indirectly.

Another aspect described herein provides a method of producing viral particles, comprising (a) providing the cell line expressing a nucleic acid sequence encoding a tetracycline-responsive transactivator protein operatively linked to a promoter (e.g., a constitutive promoter); a nucleic acid sequence comprising at least one of: a nucleic acid sequence encoding a E4 protein, a nucleic acid sequence encoding a E2A protein, and a nucleic acid sequence encoding a VA RNA, wherein each nucleic acid sequences encoding any one of E4, E2A, and VA RNA is operatively linked to a regulatable promoter; a nucleic acid sequence encoding a regulatable transcriptional activator operatively linked to an inducible promoter, wherein the inducible promoter is a E4-responsive promoter, or a E2-responsive promoter or a VA RNA-responsive promoter; and a nucleic acid construct comprising a Rep protein operatively linked to a promoter comprising a target site for binding of a regulatable transcriptional activator (b) culturing the cells for a time sufficient and under conditions in which at least the nucleic acid sequence encoding the E4 protein, the nucleic acid sequence encoding the E2A protein, or the nucleic acid sequence encoding the VA RNA is expressed first in viral vector production protocol; (c) culturing the cells for a time sufficient and under conditions in which the nucleic acid sequence encoding a toxic protein or Rep protein is expressed second in viral vector production protocol; (d) culturing the cells under conditions in which viral particles are produced; and (e) optionally isolating the viral particles. In one embodiment, the nucleic acid sequence encoding a E4 protein, the nucleic acid sequence encoding a E2A protein, or the nucleic acid sequence encoding a VA RNA is expressed between hours 3-4 of the viral vector production protocol. In one embodiment, the nucleic acid sequence encoding a Rep protein is expressed between hours 6-8 of the viral vector production protocol. In one embodiment, the regulatable transcriptional activator is a zinc-finger transcriptional activator. In one embodiment the expression or activity of the transcriptional activator is inducible by expression of one or more helper genes.

In one embodiment of any aspect, culturing in step (b) is culturing with an inducer of the regulatable promoter operatively linked to at least E4, E2A, or VA RNA. In one embodiment of any aspect, culturing in step (c) is culturing with the transcriptional activator (e.g., ZF-TA). In one embodiment of any aspect, expression of the transcriptional activator (ZF-TA) is induced by expression of E4 or E2 or VA RNA. In one embodiment of any aspect, expression of the zinc-finger transcriptional activator (ZF-TA) is induced by expression of E4 or E2 or VA directly or indirectly.

Another aspect described herein provides a method of producing viral particles, comprising (a) providing the cell line expressing a nucleic acid construct comprising a nucleic acid sequence encoding a Cap protein and a recombinase recognition sequence (RRS) located 3′ of the nucleic acid sequence encoding the Cap protein; (b) culturing the cells for a time sufficient and under conditions in which at least the nucleic acid sequence encoding the Cap protein is highly expressed for the first 24 hours of the viral vector production protocol, and is moderately expressed for the remaining 48 hours of the viral vector production protocol; (c) culturing the cells for a time sufficient and under conditions in which viral particles are produced; and (d) optionally isolating the viral particles.

In one embodiment of any aspect, culturing in step (b) is culturing with an inducer of the regulatable promoter operatively linked to the Cap protein.

Another aspect described herein provides a method of producing viral particles, comprising (a) providing the cell expressing a first nucleic acid construct comprising in a 5′ to 3′ direction: a promoter, a stop nucleic acid sequence flanked by a first pair of recombinase recognition sequences (RRS), and nucleic acid sequence encoding a Rep protein, wherein the promoter is operatively linked to the nucleic acid encoding the Rep protein, and a second nucleic acid construct comprising, in a 5′ to 3′ direction: a promoter, a stop nucleic acid sequence flanked by a second pair of recombinase recognition sequences (RRSs), and nucleic acid sequence encoding one or more of E2A, E4, and VA RNA, wherein the promoter is operatively linked to the nucleic acid encoding the one or more of E2A, E4, and VA RNA; (b) culturing the cells for a time sufficient and under conditions in which at least the nucleic acid sequence encoding the E4 protein, the nucleic acid sequence encoding the E2A protein, or the nucleic acid sequence encoding the VA RNA is expressed first in viral vector production protocol, e.g., at hour 10 of production; (c) culturing the cells for a time sufficient and under conditions in which the nucleic acid sequence encoding a toxic protein or Rep protein is expressed second in viral vector production protocol, e.g., at hour 12 of production; (d) culturing the cells under conditions in which viral particles are produced; and (e) optionally isolating the viral particles. In one embodiment, the nucleic acid sequence encoding a E4 protein, the nucleic acid sequence encoding a E2A protein, or the nucleic acid sequence encoding a VA RNA is expressed between hours 3-4 of the viral vector production protocol. In one embodiment, the nucleic acid sequence encoding a Rep protein is expressed between hours 6-8 of the viral vector production protocol.

In one embodiment of any aspect, culturing in step (b) is culturing with a recombinase specific for the first pair of recombinase recognition sequences (RRSs). In one embodiment of any aspect, culturing in step (c) is culturing with a recombinase specific for the second pair of recombinase recognition sequences (RRSs).

Yet another aspect described herein provides a method of producing a stable cell line for producing viral particles, containing a packaging sequence (sometimes referred to as an AAV genome).

### BRIEF DESCRIPTION OF THE DRAWINGS

FIG. 1A shows a schematic diagram of hypoxia-inducible gene expression. Transcription factor HIF1A (HIF1α) is degraded under normal oxygen conditions, but under hypoxic conditions, it is stabilized, dimerizes with HIF1B (HIF1β) to form HIF1 and is translocated to the nucleus. In the nucleus, the HIF1 complex can bind to the hypoxia response element and initiate expression of the gene of interest.

FIG. 1B shows a schematic diagram of the structural organisation of HIF1α and HIF1β. Both HIF1α and HIF1β have a bHLH domain for DNA binding. HIF1β has a Per-ARNT-Sim (PAS) domain for central heterodimerisation and HIF1α's C terminal domain (TAD N/TAD C) recruits transcriptional coregulatory proteins. When HIF1α and HIF1β dimerise, they translocate to the nucleus and turn on expression of hypoxia-regulated genes after binding to a hypoxia-responsive element.

FIG. 2 shows a schematic diagram of promoters RTV-015 and Synp-HYP-001. RTV-015 promoter comprises of five HRE1 and a synthetic minimal promoter MPl. These elements are spaced apart with spacers (not shown). Synp-HYP-001 comprises of four HRE2 and a CMV minimal promoter. The HRE2 elements are not spaced apart with spacers but there is a spacer between the last HRE2 element and the CMV minimal promoter (not shown).

FIG. 3 shows a time course of luciferase expression from the RTV-015, SYNP-HYP-001, and CMV-IE constructs in transiently transduced HEK293-F cells under hypoxia. Cells were placed in hypoxia at 0 hours and then luciferase activity monitored. Luciferase expression from the CMV minimal promoter, which was used as a control, does not change but the rest of the constructs show increase in luciferase activity with time.

FIG. 4 shows measurement of luciferase expression from the RTV-015 and CMV-IE constructs in transiently transduced HEK293-T in normoxic conditions and after 24 hours in hypoxia. The luciferase expression from the CMV-IE promoter is the same in normoxia and hypoxia. RTV-015 constructs show almost no luciferase activity in normoxia but is induced after 24 hours in hypoxia.

FIG. 5 shows measurement of luciferase expression from the RTV-015 and CMV-IE constructs in transiently transduced CHO_GS suspension cell line in normoxic conditions and after 24 hours in hypoxia. The luciferase expression from the CMV-IE is the same in normoxia and hypoxia. Similar to the results shown in FIG. 4, RTV-015 showed almost no luciferase activity in normoxia but is induced after 24 hours in hypoxia.

FIG. 6 is an illustration of the mechanism of action of forskolin and other adenylyl cyclase activators.

FIG. 7 shows the activity of the promoters after transient transfection into the suspension cell line HEK293-F. The cells were induced (at time 0 h) with 20 μM forskolin and luciferase expression was measured at 0 h, 3 h, 5 h and 24 h. All constructs showed increase in activity (to a varying degree) while the activity of CMV-IE remained constant.

FIGS. 8A-8C show brightfield microscopy pictures of C2C12 cells at passage 11. FIG. 8A) shows the cells prior to transformation (day 2). FIG. 8B) shows the C2C12 cells 24 hours after transfection (day 3). FIG. 8C) shows the differentiated C2C12 cells after 5.5 days into differentiation medium (day 7.5). Scale bar is 50 μm.

FIG. 9 presents exemplary data showing differences in E1A gene expression between Hek293 cells and Pro10. Results are an average of three (3) experiments and the error bars are standard deviation. Pro10 cells express E1A RNA at ˜½ of the level of the parental Hek293 cells.

FIG. 10 presents exemplary data showing viable cell density at point of transfection and point of harvest.

FIG. 11 presents exemplary data showing viability at transfection and at point of harvest.

FIG. 12 presents exemplary data showing E1A RNA levels at point of harvest.

FIG. 13 presents exemplary data showing vg/cell of rAAV produced from Pro10 at different concentrations of E1.

FIG. 14 presents exemplary data showing viable cell density for each of the indicated conditions tested.

FIG. 15 presents exemplary data showing cell viability of the cells for each of the indicated conditions tested. Mutated Rep has no significant effect on cell viability or growth during production.

FIG. 16 presents exemplary data showing final viral titer from virus production.

FIG. 17 presents exemplary data showing viral titer from wtAAV and rAAV productions. wtAAV produces up to 2 log more vg/ml than rAAV.

FIG. 18 presents exemplary data showing levels of the E2A gene expression during wt and rAAV production.

FIG. 19 presents exemplary data showing levels of the E4 gene expression during wt and rAAV production

FIG. 20 presents exemplary data showing levels of Rep78, 52 and Cap2 expression during wtAAV production

FIG. 21 presents exemplary data showing levels of Rep78, 52 and Cap2 expression during rAAV production

FIG. 22 presents exemplary schematic showing mechanism of action of leaky scanning.

FIG. 23 presents exemplary data showing comparison of vg/ml for wt vs mut-Rep. Removal of the 9 potential ATG's that could be used as a translation initiation site from Rep78 had no effect its ability to make rAAV

FIG. 24 presents exemplary data showing western blot of Rep78 and Rep52 protein expression from rA AV production nos using vanous start codons. % numbers are published translation initiation rates compared to ATG,

FIG. 25 presents exemplary data showing vg/nl of rAAV produced using alernate start codons to control the ratio of Rep78/52. Only the ACG mutation makes a significant improvement on viral titer.

FIG. 26 presents exemplary data showing expression of the Cap2 gene induced via the adenovirus helper genes.

FIG. 27 presents exemplary data showing viral titer from the C7 and C8 cells induced with adenovirus helper functions plus E1. Control is standard triple transfection of non-inducible cassettes. P=passage number

FIG. 28 presents exemplary data showing effect of the adenovirus helper functions on the forskolin promoter controlling Cap2 expression in stable cell line.

FIG. 29 presents exemplary data showing effect of forskolin on new promoter designs.

FIG. 30 presents exemplary data showing effect of the adenovirus helper functions on the activity of FORN-pJB42.

FIG. 31 presents exemplary data showing configuration of the Rep LoxP constructs. FIG. 31 discloses SEQ ID NOS 218-220, 223, 217, 221-222, 224-227, respectively, in order of appearance.

FIG. 32 presents exemplary data showing diagram of the Rep constructs. Effect of Cre and or NHK477 treatment on the induction of Rep expression. There is no expression of either Rep protein in the untreated samples for both promoters.

FIG. 33 presents exemplary data showing AAV production using cre-recombinase control of Rep expression.

FIG. 34 presents exemplary data showing diagram of expected outcomes and results of the experiment. Mean of n=3

FIG. 35 presents exemplary data showing concept of cascaded gene expression for rAAV production in Pro10 cells.

FIG. 36 presents exemplary data showing exemplification of ZF-TF design.

FIG. 37 presents exemplary data showing comparison of standard Rep2-Cap8 transfection vs the new novel plasmid configuration.

### DETAILED DESCRIPTION OF THE INVENTION

In general, the invention described herein provides stable cell lines for recombinant viral vector production having expression of at least one toxic protein under control of at least one regulatable promoter, e.g., an inducible promoter, methods for producing such cells, a promoter linked to another sequence providing a regulatory control, such as a Zinc finger, and methods for manufacturing a recombinant viral vector using the same. The described stable cell lines provide temporal and/spatial control of at least one (e.g., at least one, at least two, at least three) toxic proteins during viral vector production. A toxic protein, for example, expressed from a viral gene, such as replication (rep), capsid (cap), helper gene products, polymerase (pol), reverse polymerase, or envelope (env), is one that is detrimental to a cell when expressed. These viral genes, however, are essential to the manufacture of recombinant viral vectors. Current methods to mitigate the negative effects of expression of toxic proteins include using cells which transiently express the toxic protein during production, however, these methods can adversely affect the yield of production. Our stable cells described herein provide a stable cell line that allows for expression of a toxic protein primarily at the time in which it is needed, thus limiting the negative effects of its expression. Our methods described herein, which utilize these stable cell lines, result in a higher yield of recombinant viral vector production as compared to current methods.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used herein, the terms “nucleotide sequence”, “nucleic acid sequence”, “RNA sequence,” and “DNA sequence,” are used interchangeably herein and refer to a sequence of a nucleic acid, e.g., a circular nucleic acid that is to be introduced into a target cell and encodes a gene product or polypeptide. The nucleic acid sequence may comprise at least a sequence that encodes a toxic polypeptide (i.e., protein). A heterologous nucleic acid sequence refers to a nucleic acid sequence that is not “naturally occurring,” that is, a sequence not normally expressed in the cell.

As used herein, the phrase “promoter” refers to a region of DNA that generally is located upstream of a nucleic acid sequence to be transcribed that is needed for transcription to occur. Promoters permit the proper activation or repression of transcription of sequence under their control. A promoter typically contains specific sequences that are recognized and bound by transcription factors, e.g. enhancer sequences. Transcription factors bind to the promoter DNA sequences and result in the recruitment of RNA polymerase, an enzyme that synthesizes RNA from the coding region of the gene. A great many promoters are known in the art.

Regulatable promoters include both inducible promoters and repressible promoters. As used herein, an “inducible promoter” refers to a promoter that is characterized by initiating or enhancing transcriptional activity when in the presence of, influenced by, or contacted by an inducer or inducing agent or when or suitable inducing conditions are applied (e.g. hypoxia). An “inducer” or “inducing agent,” as defined herein, can be endogenous, or a normally exogenous compound or protein that is administered in such a way as to be active in inducing transcriptional activity from the inducible promoter. The term “inducer” as used herein can also relate to applying suitable conditions such that transcriptional activity from the inducible promoter is induced (e.g. hypoxia). In some embodiments, the inducer or inducing agent, i.e., a chemical, a compound, a protein, or suitable conditions, can itself be the result of transcription or expression of a nucleic acid sequence (i.e., an inducer can be an inducer protein expressed by another component or module), which itself can be under the control of, e.g., an inducible or repressible promoter. In some embodiments, an inducible promoter is induced in the absence of certain agents, such as a repressor. Examples of inducible promoters include but are not limited to, forskolin-inducible, hypoxia-inducible, temperature-inducible, tetracycline-inducible (Tet-ON), pH-inducible, osmolarity-inducible, metallothionine-inducible, hormone- (e.g. ecdysone-) inducible, carbon source-inducible, alcohol (e.g. ethanol)-inducible, amino acid-inducible, mifepristone (RU-486)-inducible, cumate-inducible, 4-hydroxytamoxifen (OHT)-inducible, gas-inducible, riboswitch-, ribozyme- and aptazyme-inducible, rapamycin-inducible, chemically-induced proximity-inducible, Rheoswitch® promoters, CRISPR-Inducible and inducible promoters from mammalian viruses (e.g., the adenovirus late promoter; and the mouse mammary tumor virus long terminal repeat (MMTV-LTR)) and other steroid-responsive promoters, rapamycin responsive promoters and the like.

An inducible promoter can be said to drive expression of the nucleic acid sequence that it regulates, e.g., a heterologous gene that encodes a toxic protein. The phrases “operably linked,” “operatively linked,” and “under control,” indicate that a promoter is in a correct functional location and/or orientation in relation to a nucleic acid sequence it regulates to control transcriptional initiation and/or expression of that sequence.

As used herein, a “repressible promoter” refers to a promoter that is characterized by stopping or preventing transcriptional activity such that the activity is down-regulated when in the presence of, influenced by, or contacted by a repressor or repressing agent. An “repressor” or “repressing agent,” as defined herein, can be endogenous, or a normally exogenous compound or protein that is administered in such a way as to be active in stopping transcriptional activity from the repressible promoter. In some embodiments, the repressor or repressing agent, i.e., a chemical, a compound or a protein, can itself be the result of transcription or expression of a nucleic acid sequence (i.e., a repressor can be a repressor protein expressed by another component or module), which itself can be under the control of, e.g., an inducible or repressible promoter. In some embodiments, a repressible promoter is repressed in the absence of certain agents, such as an inducer. Examples of repressible promoters include but are not limited to tetracycline-off (Tet-OFF), glucose, copper ion, L-Methionine, low-phosphate, ADH1, Ga180, and MET25.

In some embodiments a promoter can be both repressible and inducible, e.g. through the use of different repressing or inducing agents (e.g. an agonist and agonist of a relevant transcription regulator or pathway) or by varying conditions which repress or induce expression. Thus, for example, a promoter can be induced when an inducer is administered and repressed when a repressor is administered.

As used herein, “introducing” refers broadly to placing the synthetic nucleic acid, expression vector, or plasmid into a viral expression system (e.g., a cell or viral vector) such that it is present in, e.g., a cell, or viral vector expression system. Less broadly, introducing refers to any appropriate means of placing the synthetic nucleic acid, expression vector, or plasmid in a viral expression system described herein. Introducing can be by such means that the synthetic nucleic acid, expression vector, or plasmid is appropriately transported into the interior of the cell or viral expression system such that, e.g., the synthetic nucleic acid, expression vector, or plasmid is produced by the host cell machinery. Such introducing may involve, for example transformation, transfection, electroporation, or lipofection.

The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, artificial chromosome, virus, virion, etc.

As used herein, “expression vector” refers to a nucleic acid that includes an open reading frame (ORF) and, when introduced to a cell, contains all of the nucleic acid components necessary to allow mRNA expression of said open reading frame. “Expression vectors” of the invention also include elements necessary for replication and propagation of the vector in a host cell. In particular, as used herein, “expression vector” refers to a vector that directs expression of a heterologous nucleic acid described herein. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing.

As used herein, the term “viral vector” refers to a nucleic acid vector construct that includes at least one element of viral origin and has the capacity to be packaged into a viral vector particle. The viral vector can contain a nucleic acid encoding a polypeptide as described herein in place of non-essential viral genes. The vector and/or particle may be utilized for the purpose of transferring synthetic nucleic acids described herein into cells either in vitro or in vivo. Numerous forms of viral vectors are known in the art.

As used herein the term “cap protein” refers to a capsid protein, suitably an AAV capsid protein, e.g. one or more of the VP capsid proteins of AAV. For example, AAV viral particles typically comprise the capsid proteins VP1, VP2 and VP3. The capsid proteins can be naturally occurring or modified, as is well known in the art.

As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, snake AAV, avian AAV, bovine AAV, canine AAV, equine AAV, ovine AAV, goat AAV, shrimp AAV, and any other AAV now known or later discovered. See, e.g., FIELDS et al. VIROLOGY, volume 2, chapter 69 (4th ed., Lippincott-Raven Publishers). A number of relatively new AAV serotypes and clades have been identified (See, e.g., Gao et al., J. Virol. 78:6381 (2004); Moris et al., Virol. 33:375 (2004); and Table 1). A “rAAV vector genome” or “rAAV genome” is an AAV genome (i.e., vDNA) that comprises one or more heterologous nucleic acid sequences. rAAV vectors generally require only the 145 base ITR in cis to generate virus. All other viral sequences are dispensable and may be supplied in trans (Muzyczka, Curr. Topics Microbiol. Immunol. 158:97 (1992)). Typically, the rAAV vector genome will only retain the one or more ITR sequence so as to maximize the size of the transgene that can be efficiently packaged by the vector. The structural and non-structural protein coding sequences may be provided in trans (e.g., from a vector, such as a plasmid, or by stably integrating the sequences into a packaging cell). In embodiments of the invention the rAAV vector genome comprises at least one ITR sequence (e.g., AAV ITR sequence), optionally two ITRs (e.g., two AAV ITRs), which typically will be at the 5′ and 3′ ends of the vector genome and flank the heterologous nucleic acid, but need not be contiguous thereto. The ITRs can be the same or different from each other.

TABLE 1

| | GenBank | | | | |
|---|---|---|---|---|---|
| | Accession | | GenBank | | GenBank |
| | Number | | Access. No. | | Access. No. |
| Complete | | Hu T88 | AY695375 | Hu42 | AY530605 |
| Genomes | | | | | |
| Adeno-associated | NC_002077, | Hu T71 | AY695374 | 1Iu67 | AY530627 |
| virus 1 | AF063497 | | | | |
| Adeno-associated | NC_001401 | Hu T70 | AY695373 | Hu40 | AY530603 |
| virus 2 | | | | | |
| Adeno-associated | NC 001729 | Hu T40 | AY695372 | Hu41 | AY530604 |
| virus 3 | | | | | |
| Adeno-associated | NC 001863 | Hu T32 | AY695371 | Hu37 | AY530600 |
| virus 3B | | | | | |
| Adeno-associated | NC 001829 | Hu T17 | AY695370 | Rh40 | AY530559 |
| virus 4 | | | | | |
| Adeno-associated | Y18065, | Hu LG15 | AY695377 | Rh2 | AY243007 |
| virus 5 | AF085716 | | | | |
| Adeno-associated | NC_001862 | Clade C | | Bbl | AY243023 |
| virus 6 | | | | | |
| Avian AAV | AY186198, | Hu9 | AY530629 | Bb2 | AY243022 |
| ATCC VR-865 | AY629583, | | | | |
| | NQ004828 | | | | |
| Avian AAV strain | NC_006263, | Hull) | AY530576 | Rh10 | AY243015 |
| DA-1 | AY629583 | | | | |
| Bovine AAV | NC_005889, | Hull | AY530577 | Hu17 | AY530582 |
| | AY388617 | | | | |
| Clade A | | Hu53 | AY530615 | Hu6 | AY530621 |
| AAV1 | NC_002077, | Hu55 | AY530617 | Rh25 | AY530557 |
| | AF063497 | | | | |
| AAV6 | NC 001862 | Hu54 | AY530616 | Pit | AY530554 |
| Hu. 48 | AY530611 | Hu7 | AY530628 | Pil | AY530553 |
| Hu 43 | AY530606 | Hu18 | AY530583 | Pi3 | AY530555 |
| Hu 44 | AY530607 | Hul5 | AY530580 | R1157 | AY530569 |
| Hu 46 | AY530609 | Hul6 | AY530581 | Rh50 | AY530563 |
| Clade B | | Hu25 | AY530591 | Rh49 | AY530562 |
| Hu. 19 | AY530584 | Hu60 | AY530622 | Hu39 | AY530601 |
| Hu. 20 | AY530586 | Ch5 | AY243021 | Rh58 | AY530570 |
| Hu 23 | AY530589 | Hu3 | AY530595 | Rh61 | AY530572 |
| Hu22 | AY530588 | Hul | AY530575 | Rh52 | AY530565 |
| Hu24 | AY530590 | Hu4 | AY530602 | R1153 | AY530566 |
| Hu21 | AY530587 | Hut | AY530585 | R1151 | AY530564 |
| Hu27 | AY530592 | Hu61 | AY530623 | Rh64 | AY530574 |
| Hu28 | AY530593 | Clade D | | Rh43 | AY530560 |
| Hu 29 | AY530594 | Rh62 | AY530573 | AAV8 | AF513852 |
| Hu63 | AY530624 | Rh48 | AY530561 | Rh8 | AY242997 |
| Hu64 | AY530625 | R1154 | AY530567 | Rhl | AY530556 |
| IIu13 | AY530578 | Rh55 | AY530568 | Clade F | |
| Hu56 | AY530618 | Cy2 | AY243020 | Hul4 | AY530579 |
| | | | | (AAV9) | |
| 11u57 | AY530619 | AAV7 | AF513851 | Hu31 | AY530596 |
| Hu49 | AY530612 | Rh35 | AY243000 | Hu32 | AY530597 |
| Hu58 | AY530620 | Rh37 | AY242998 | Clonal | |
| | | | | Isolate | |
| Hu34 | AY530598 | Rh36 | AY242999 | AAV5 | Y18065, |
| | | | | | AF085716 |
| Hu35 | AY530599 | Cy6 | AY243016 | AAV 3 | NC_00172 9 |
| AAV2 | NC_001401 | Cy4 | AY243018 | AAV 3B | NC_00186 3 |
| Hu45 | AY530608 | Cy3 | AY243019 | AAV4 | NC_00182 9 |
| Hu47 | AY530610 | Cy5 | AY243017 | Rh34 | AY243001 |
| Hu51 | AY530613 | Rh13 | AY243013 | Rh33 | AY243002 |
| 11u52 | AY530614 | Clade E | | R1132 | AY243003 |
| Hu T41 | AY695378 | Rh38 | AY530558 | | |
| Hu S17 | AY695376 | Hu66 | AY530626 | | |

The genomic sequences of various serotypes of AAV, as well as the sequences of the native ITRs, Rep proteins, and capsid subunits are known in the art. Such sequences may be found in the literature or in public databases such as GenBank. See, e.g., GenBank Accession Numbers NC_002077, NC_001401, NC_001729, NC_001863, NC_001829, NC_001862, NC_000883, NC_001701, NC_001510, NC_006152, NC_006261, AF063497, U89790, AF043303, AF028705, AF028704, 102275, 101901, 102275, X01457, AF288061, AH009962, AY028226, AY028223, AY631966, AX753250, EU285562, NC_001358, NC_001540, AF513851, AF513852 and AY530579; the disclosures of which are incorporated by reference herein for teaching AAV nucleic acid and amino acid sequences. See also. e.g., Bantel-Schaal et al., J Virol. 73:939 (1999): Chiorini et al., J Virol. 71:6823 (1997); Chiorini et al., Ji Virol. 73:1309 (1999); Gao et al., Proc. Nat. Acad. Sci. USA 99:11854 (2002); Moris et al., Virology, 33:375 (2004); Mon et al., Virology, 330:375 (2004); Muramatsu et al., Virology, 221:208 (1996); Ruffing et al., J. Gen. Virol. 75:3385 (1994); Rutledge et al., J Virol. 72:309 (1998); Schmidt et al., Virol. 82:8911 (2008); Shade et al., J. Virol. 58:921 (1986); Srivastava et al., J Virol. 45:555 (1983); Xiao et al., J Virol. 73:3994 (1999); international patent publications WO 00/28061, WO 99/61601, WO 98/11244; and U.S. Pat. No. 6,156,303; the disclosures of which are incorporated by reference herein for teaching AAV nucleic acid and amino acid sequences. See also Table 1. An early description of the AAV1, AAV2 and AAV3 ITR sequences is provided by Xiao, X., (1996), “Characterization of Adeno-associated virus (AAV) DNA replication and integration,” Ph.D. Dissertation, University of Pittsburgh, Pittsburgh, PA (incorporated herein it its entirety).

As used herein, the term “expression” refers to the cellular processes involved in producing RNA and proteins, including where applicable, but not limited to, for example, transcription, transcript processing, translation and protein folding, post-translational modification and other types of processing.

The term “gene” means the nucleic acid sequence which is transcribed (DNA) to RNA in vitro or in vivo when operably linked to appropriate regulatory sequences. The gene may or may not include regions preceding and following the coding region, e.g., 5′ untranslated (5′UTR) or “leader” sequences and 3′ UTR or “trailer” sequences, as well as intervening sequences (introns) between individual coding segments (exons).

As used herein, the term “polypeptide” encompasses both peptides and proteins, unless indicated otherwise. A “polypeptide” or “protein” is a sequence of nucleotide bases, and may be RNA, DNA or DNA-RNA hybrid sequences (including both naturally occurring and non-naturally occurring nucleotide), and can be either single or double stranded DNA sequences.

The term “cell culture,” as used herein, refers to a proliferating mass of cells that may be in either an undifferentiated or differentiated state.

A “viral vector expression system” is a system of one or more polynucleotides that are sufficient, when introduced into a suitable host cell, to support production of viral vector. A viral vector expression system will typically include polynucleotides encoding the appropriate viral proteins, for example, an envelope and polymerase gene, e.g., for producing a lentivirus or adeno virus.

An “AAV expression system” is a system of one or more polynucleotides that are sufficient, when introduced into a suitable host cell, to support production of recombinant AAV (rAAV). An AAV expression system will typically include polynucleotides encoding AAV rep and cap, helper genes, and a rAAV genome.

AAV genomes have palindromic sequences at both their 5′ and 3′ ends. The palindromic nature of the sequences leads to the formation of a hairpin structure that is stabilized by the formation of hydrogen bonds between the complementary base pairs. This hairpin structure is believed to adopt a “Y” or a “T” shape. See, e.g., FIELDS et al. VIROLOGY, volume 2, chapters 69 & 70 (4th ed., Lippincott-Raven Publishers).

The term “terminal repeat” or “TR” includes any viral terminal repeat or synthetic sequence that forms a hairpin structure and functions as an inverted terminal repeat (i.e., mediates the desired functions such as replication, virus packaging, integration and/or provirus rescue, and the like). The ITR can be an AAV ITR or a non-AAV ITR. For example, a non-AAV ITR sequence such as those of other parvoviruses (e.g., canine parvovirus, bovine parvovirus, mouse parvovirus, porcine parvovirus, human parvovirus B-19) or the SV40 hairpin that serves as the origin of SV40 replication can be used as an ITR, which can further be modified by truncation, substitution, deletion, insertion and/or addition. Further, the ITR can be partially or completely synthetic, such as the “double-D sequence” as described in U.S. Pat. No. 5,478,745 to Samulski et al.

An “AAV inverted terminal repeat” or “AAV ITR” may be from any AAV, including but not limited to serotypes 1, 2, 3a, 3b, 4, 5, 6, 7, 8, 9, 10, 11, or 13, snake AAV, avian AAV, bovine AAV, canine AAV, equine AAV, ovine AAV, goat AAV, shrimp AAV, or any other AAV now known or later discovered (see, e.g., Table 1). An AAV ITR need not have the native terminal repeat sequence (e.g., a native AAV ITR sequence may be altered by insertion, deletion, truncation and/or missense mutations), as long as the terminal repeat mediates the desired functions, e.g., replication, virus packaging, integration, and/or provirus rescue, and the like.

As used herein, “a,” “an” or “the” can be singular or plural, depending on the context of such use. For example, “a cell” can mean a single cell or it can mean a multiplicity of cells.

Also as used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of a composition of this invention, dose, time, temperature, and the like, is meant to encompass variations of ±20%, ±10%, ±5%, ±1%, ±0.5%, or even ±0.1% of the specified amount.

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment. The term “consisting of” refers to compositions, methods, and respective components thereof as described herein, which are exclusive of any element not recited in that description of the embodiment.

### Nucleic Acid Constructs

One aspect provided herein is a nucleic acid construct comprising at least a nucleic acid sequence encoding at least one helper protein, wherein each nucleic acid construct is operatively linked to a regulatable promoter. Exemplary helper genes, for example for AAV viral particle particles, include, but are not limited to E1, E2A, E4, and VA. Further helper genes may include one or more of the following elements known in the Pro10 cell line. Helper genes used for the production of other viral particles, for example, lentiviral particles or adeno viral particles can further be used in the nucleic acid constructs described herein. In one embodiment, the nucleic acid construct encoding at least one helper protein, comprises a nucleic acid sequence encoding a tetracycline-responsive transactivator protein operatively linked to a constitutive promoter. In one embodiment, the nucleic acid construct encoding at least one helper protein further comprises a nucleic sequence encoding a marker protein. In one embodiment, the nucleic acid construct encoding at least one helper protein, comprises a nucleic acid sequence encoding a tetracycline-responsive transactivator protein operatively linked to a constitutive promoter and a nucleic sequence encoding a marker protein.

In one embodiment, the regulatable promoter is a repressible promoter, for example a tetracycline repressible promoter. In one embodiment, the repressible promoter is repressed by a tetracycline-responsive transactivator and repression is reversed by contact with tetracycline or doxycycline.

In one embodiment, expression of the at least one helper gene is responsive to the inducer of a regulatable promotor. In one embodiment, expression of the at least one helper gene is responsive to an inducer of a repressible promotor. In one embodiment, expression of the at least one helper gene is responsive to tetracycline or doxycycline.

Another aspect provided herein is a nucleic acid construct encoding a tetracycline-responsive transactivator protein operatively linked to a constitutive promoter.

Another aspect provided herein is a nucleic sequence encoding a marker protein.

Another aspect provided herein is a nucleic acid construct comprising at least a nucleic acid sequence encoding a toxic protein, wherein the nucleic acid encoding a toxic protein is operatively linked to a regulatable promoter. In one embodiment, the nucleic acid encoding a toxic protein further encodes a ribozyme enzyme at the 3′ of the nucleic acid encoding the toxic protein, e.g., a small self-cleaving ribozyme enzyme.

In one embodiment, the regulatable promoter is an inducible promoter or comprises a binding site for a regulatable transcriptional activator. In one embodiment, the inducible promoter comprises a TATA box sequence, or a p5 replication sequence, or both a TATA box sequence and p5 replication sequence. In one embodiment, the inducible promoter comprises a TATA box sequence, or a p5 replication sequence, or both a TATA box sequence and p5 replication sequence instead of a minimal promoter. In one embodiment, the regulatable promoter comprises a binding site for a Zinc-Finger transcriptional activator (ZF-TA).

Another aspect provided herein is a nucleic acid construct comprising a nucleic acid sequence encoding at least one helper protein, wherein the at least one helper gene is operatively linked to a first regulatable promoter; and a nucleic acid sequence encoding a toxic protein, wherein the nucleic acid encoding the toxic protein is under the control of a second regulatable promoter or a transcriptional activator.

Another aspect provided herein is a nucleic acid construct comprising a nucleic acid sequence comprising at least one of a nucleic acid sequence encoding a helper gene, e.g., a nucleic acid sequence encoding a E4 protein, a nucleic acid sequence encoding a E2A protein, and a nucleic acid sequence encoding a VA RNA, wherein each nucleic acid sequences encoding any one of E4, E2A, and VA RNA is operatively linked to a regulatable promoter. In certain embodiments, any helper protein, for example for producing a lentiviral particle, an adeno viral particle, can be used. For example, a nucleic acid encoding Gag, Pol, etc.

Another aspect provided herein is a nucleic acid construct encoding a Rep protein, wherein the nucleic acid encoding a Rep protein is under the control of a regulatable promoter or a zinc-finger transcriptional activator (ZF-TA).

Another aspect provided herein is a nucleic acid construct comprising a nucleic acid sequence comprising at least one of: a nucleic acid sequence encoding a E4 protein, a nucleic acid sequence encoding a E2A protein, and a nucleic acid sequence encoding a VA protein, wherein each nucleic acid sequences encoding any one of E4, E2A, and VA RNA is operatively linked to a first regulatable promoter; and a nucleic acid sequence encoding a Rep protein, wherein the nucleic acid encoding a Rep protein is under the control of a second regulatable promoter or transcriptional activator, wherein the first and the second regulatable promoters are different kinds of regulatable promoter, e.g., the first promoter can be a hypoxia promoter, and the second promoter another type. In one embodiment the regulatable promoter is an inducible promoter, or an inducible promoter that comprises a TATA box sequence, or a p5 replication sequence, or both a TATA box sequence and p5 replication sequence. In one embodiment the regulatable promoter is an inducible promoter that lacks a minimal promoter but comprises a TATA box sequence, and/or a p5 replication sequence.

Exemplary transcriptional activators include homeodomain transcriptional activator, zinc-finger transcriptional activator, winged-helix (Forkhead) transcriptional activator, leucine-zipper transcriptional activator, and helix-loop-helix transcriptional activator. In one embodiment, the transcriptional activator is a zinc-finger transcriptional activator (ZF-TA).

Another aspect provided herein is a nucleic acid construct comprising a nucleic acid sequence encoding a zinc finger (ZF) transcriptional activator operatively linked to an inducible promoter, wherein the inducible promoter is a E4-responsive promoter, or a E2-responsive promoter or other helper-gene responsive promoter. In various embodiments, this construct is used to induce expression of the zinc-finger transcriptional activator (ZF-TA) of other nucleic acid constructs described herein

Another aspect provided herein is a nucleic acid construct comprising a nucleic acid sequence encoding a zinc finger (ZF) transcriptional activator operatively linked to a constitutive promoter.

Another aspect provided herein is a nucleic acid construct comprising a toxic protein operatively linked to a promoter comprising a target site for binding of a zinc finger transcriptional activator (ZF-TA).

Another aspect provided herein is a nucleic acid construct comprising a Rep protein operatively linked to a promoter comprising a target site for binding of a zinc-finger transcriptional activator (ZF-TA).

In various embodiments, any of the nucleic acid constructs described herein further comprises at least one of a nucleic acid sequence encoding at least one helper protein, wherein each nucleic acid construct is operatively linked to a regulatable promoter; a nucleic acid encoding the toxic protein is under the control of a second regulatable promoter or a zinc-finger transcriptional activator; a nucleic acid sequence encoding a E4 protein, a nucleic acid sequence encoding a E2A protein, and a nucleic acid sequence encoding a VA protein, wherein each nucleic acid sequences encoding any one of E4, E2A, and VA RNA is operatively linked to a regulatable promoter; or a nucleic acid encoding the Rep protein is under the control of a second regulatable promoter or a zinc-finger transcriptional activator.

Another aspect provided herein is a nucleic acid construct comprising a nucleic acid sequence encoding a tetracycline-responsive transactivator protein operatively linked to a constitutive promoter; a nucleic acid sequence comprising at least one of: a nucleic acid sequence encoding a E4 protein, a nucleic acid sequence encoding a E2A protein, and a nucleic acid sequence encoding a VA protein, wherein each nucleic acid sequences encoding any one of E4, E2A, and VA RNA, or any combination thereof, is operatively linked to a regulatable promoter; and a nucleic acid sequence encoding a zinc finger (ZF) transcriptional activator operatively linked to an inducible promoter, wherein the inducible promoter is a E4-responsive promoter, or a E2-responsive promoter; and a nucleic acid construct comprising a Rep protein operatively linked to a promoter comprising a target site for binding of a zinc-finger transcriptional activator (ZF-TA).

Another aspect provided herein is a nucleic acid construct comprising a nucleic acid sequence encoding a toxic protein and a recombinase recognition sequence (RRS) located 3′ of the nucleic acid sequence encoding the toxic protein. By way of an example only, a toxic protein or polypeptide that when expressed by the cell, results in decreased viability of the cell that it is expressed in, or decreased protein production or protein synthesis. Exemplary toxic proteins include, but are not limited to Cap, Rep, or proteins encoded by helper genes. In one embodiment, the toxic protein is operatively linked to a constitutive promoter. In another embodiment, the toxic protein is operatively linked to a regulatable promoter, e.g., an inducible promoter. In one embodiment, the nucleic acid construct comprises a nucleic acid encoding a recombinase protein operatively linked to an inducible promoter.

Another aspect provided herein is a nucleic acid construct comprising a nucleic acid sequence encoding a Cap protein and a recombinase recognition sequence (RRS) located 3′ of the nucleic acid sequence encoding the Cap protein. In one embodiment, the Cap protein is operatively linked to a constitutive promoter. In another embodiment, the Cap protein is operatively linked to a regulatable promoter, e.g., an inducible promoter. In one embodiment, the nucleic acid construct comprises a nucleic acid encoding a recombinase protein operatively linked to an inducible promoter.

In one embodiment, the RRS is a Flippase-responsive RRSs.

Another aspect provided herein is a nucleic acid construct comprising a nucleic acid construct comprising a nucleic acid encoding a recombinase protein operatively linked to an inducible promoter.

Another aspect provided herein is a nucleic acid construct comprising a first nucleic acid construct comprising in a 5′ to 3′ direction: a promoter, a stop nucleic acid sequence flanked by a first pair of recombinase recognition sequences (RRS), and nucleic acid sequence encoding a Rep protein, wherein the promoter is operatively linked to the nucleic acid encoding the Rep protein, and a second nucleic acid construct comprising, in a 5′ to 3′ direction: a promoter, a stop nucleic acid sequence flanked by a second pair of recombinase recognition sequences (RRSs), and nucleic acid sequence encoding one or more of E2A, E4, and VA RNA, wherein the promoter is operatively linked to the nucleic acid encoding the one or more of E2A, E4, and VA RNA.

In one embodiment, the RRS flanking the nucleic acid encoding Rep and the RRS flanking a helper gene (e.g., E2A, E4, or VA) are the same such that they are controlled by the same inducer. In one embodiment, the RRS flanking the nucleic acid encoding Rep and the RRS flanking a helper gene (e.g., E2A, E4, or VA) are different. Different flanking RRSs allows for temporal control of the Rep protein and the helper gene.

In one embodiment, the nucleic acid construct further comprises a nucleic acid encoding one or more selection markers flanked between a third pair of recombinase recognition sequences (RRSs), wherein the pair of RRS are in the same orientation with respect to each other, and wherein the nucleic acid encoding the one or more selection markers is operatively linked to one or more promoters from another construct provided herein.

When a nucleic acid sequence (e.g., a target gene) is flanked by a pair of recombinase recognition sequences (RRS) in the same orientation, the nucleic acid sequence can be excised upon the recognition of the RRS by the proper recombinase. Alternatively, when a nucleic acid sequence is flanked by a pair of recombinase recognition sequences (RRS) in an inverse orientation with respect to each other, the nucleic acid sequence can be inverted upon the recognition of the RRS by the proper recombinase. In some embodiments, the inversion and/or excision reactions will place the nucleic acid sequence (e.g., a target gene) in a new location and/or orientation, and depending on the elements outside of the RRS site, to make the nucleic acid operatively linked to a promoter, thereby driving expression of a gene encoding by the nucleic acid sequence.

Depending on the location of the recombinase recognition sequences, the excision reaction by a recombinase can lead to activation or inhibition of gene expression. For example, to activate gene expression, one can put a transcription termination sequence, flanked on both sides by recombination sites, upstream of a target gene of interest. In the presence of the recombinase, the transcription termination sequence is removed or excised, thus allowing gene expression to occur. In contrast, to inhibit gene expression, the recombination sites can be engineered to flank the gene of interest. In such embodiment, the gene is expressed without the recombinase. Once the recombinase is induced, the gene will be excised and thus the gene expression is completely and irreversibly inhibited. The determinant factor that governs the on/off state of the gene resides in the presence of the recombinase and the location of the RRS. The expression of the recombinase can be regulated by a signal inducible promoter, thus restricting the expression of the recombinase in a certain subset of cells that are undergoing that particular signal. For instance, by controlling the expression of Cre recombinase with neuron specific promoters, one can turn genes on or off in neurons only while leaving the same gene unperturbed in other tissues. These enzymes have been engineered to be very active in a wide range of organisms, including bacteria, mammals, insects, plants and fish.

In one embodiment, the first pair of RRS, second pair of RRS, and third pair of RRS are each responsive to different tyrosine recombinase or serine integrase enzymes. In one embodiment, the first pair of RRS and second pair of RRS, are responsive to the same tyrosine recombinase or serine integrase enzyme. In one embodiment, the first pair of RRS, or second pair of RRS, or both are Cre-responsive RRS. In one embodiment, the third pair of RRSs are Flipase-responsive RRS.

In various embodiments, any nucleic construct described herein further comprises a nucleic acid sequence encoding a tetracycline-responsive transactivator protein operatively linked to a constitutive promoter.

In various embodiments, any nucleic construct described herein further comprises a nucleic sequence encoding a marker protein. Exemplary marker proteins include fluorescent proteins, such as green fluorescent protein (GFP), red fluorescent protein (RFP), or luciferase; molecular tags, such as a Myc tag, a Flag tag, or a His tag; and molecular barcodes.

In various embodiments, any nucleic construct described herein further comprises a nucleic acid sequence encoding a tetracycline-responsive transactivator protein operatively linked to a constitutive promoter and a nucleic sequence encoding a marker protein.

In one embodiment, the nucleic sequence encoding a marker protein is removed from the construct. In one embodiment, the nucleic sequence encoding a marker protein is flanked by recombinase recognitions sequences (RRSs) in the same orientation with respect to each other, e.g., to facilitate its removal from the construct.

In certain embodiments, the transactivator is a CRISPR transactivator or a CAS transactivator. As used herein, “CRISPR transactivator” or a “CAS transactivator” refer to transcriptional activators protein domains or whole proteins linked, e.g., to dCas9 or sgRNAs that assist in the recruitment of co-factors or RNA Polymerase for transcription of the gene(s) targeted by the system. Transcriptional activators have, e.g., at least a DNA binding domain and a domain for activation of transcription. The activation domain can recruit general transcription factors or RNA polymerase to the gene sequence, or function by facilitating transcription by stalled RNA polymerases, and in eukaryotes can act to move nucleosomes on the DNA or modify histones to increase gene expression. These activators can be introduced into the system through attachment to dCas9 or to the sgRNA. CRISPR transactivator or a CAS transactivator are known in the art and can be readily identified by one skilled in the art.

Recombinase Recognitions Sequences (RRSs)

A “recombinase,” as used herein, is a site-specific enzyme that recognizes short DNA sequence(s), referred to as recombinase recognition sequences or RRS herein, which are typically between about 30 base pairs (bp) and 40 bp, and that mediates the recombination between these recombinase recognition sequences, which results in the excision, integration, inversion, or exchange of DNA fragments between the recombinase recognition sequences.

Exemplary recombinases include, but are not limited to, Cre, Flp, Dre, SCre, VCre, Vika, B2, B3, KD, ΦC31, Bxb1, λ, HK022, HP1, γδ, ParA, Tn3, Gin, R4, TP901-1, TG1, PhiRvl, PhiBT1, SprA, XisF, TnpX, R, A118, spoIVCA, PhiMR11, SCCmec, TndX, XerC, XerD, XisA, Hin, Cin, mrpA, beta, PhiFC1, Fre, Clp, sTre, FimE, and HbiF.

Exemplary recombinase recognitions sequences (RRSs) include, but are not limited to, loxP, loxN, lox 511, lox 5171, lox 2272, M2, M3, M7, M11, lox71, lox66, FRT, rox, SloxM1, VloxP, vox, B3RT, KDRT, F3, F14, attB/P, F5, F13, Vlox2272, Slox2272, SloxP, RSRT, and B2RT.

Recombinases can be classified into two distinct families: serine recombinases (e.g., resolvases and invertases) and tyrosine recombinases (e.g., integrases), based on distinct biochemical properties. Serine recombinases and tyrosine recombinases are further divided into bidirectional recombinases and unidirectional recombinases. Examples of bidirectional serine recombinases include, without limitation, β-six, CinH, ParA and γδ; and examples of unidirectional serine recombinases include, without limitation, Bxb1, φC31 (phiC31), TP901, TGI, φBTI, R4, cpRVl, cpFCl, MRU, A118, U153 and gp29. Examples of bidirectional tyrosine recombinases include, without limitation, Cre, FLP, and R; and unidirectional tyrosine recombinases include, without limitation, Lambda, HK1O1, HK022 and pSAM2. The serine and tyrosine recombinase names stem from the conserved nucleophilic amino acid residue that the recombinase uses to attack the DNA and which becomes covalently linked to the DNA during strand exchange. Recombinases have been used for numerous standard biological applications, including the creation of gene knockouts and the solving of sorting problems.

In some embodiments, the recombinases for use in the present invention are orthogonal recombinases. When a first recombinase is orthogonal to the second recombinase, it means that the second recombinase does not recognize the RRS specific for the first recombinase, neither does the first recombinase recognize the RRS specific for the second recombinase.

The outcome of recombination depends, in part, on the location and orientation of two short repeated DNA sequences (e.g., RRS) that are to be recombined, typically less than 30 bp long. The site-specific recombinases bind to these repeated sequences, which are specific to each recombinase, and are herein referred to as “recombinase recognition sequences” or “recombinase recognition sites.” Thus, as used herein, a recombinase is “specific for” a recombinase recognition site when the recombinase can mediate inversion or excision between the repeat DNA sequences. As used herein, a recombinase may also be said to recognize its “cognate recombinase recognition sites,” which flank an intervening genetic element (e.g., promoter, terminator, or target gene). A genetic element is said to be “flanked” by recombinase recognition sites when the element is located between and immediately adjacent to two repeated DNA sequences. In some embodiments, the recombinase recognition sites do not overlap each other. However, in other embodiments, recombinase recognition sites do overlap each other, such as described herein below, which permits greatly increased combinatorial complexity.

Inversion recombination happens between two short, inverted, repeated DNA sequences. Without wishing to be bound by theory, a DNA loop formation, assisted by DNA bending proteins, brings the two repeat sequences together, at which point DNA cleavage and ligation occur. This reaction is ATP independent and requires supercoiled DNA. The end result of such an inversion recombination event is that the DNA located between the repeated site inverts (i.e., the DNA between the two RRS reverses orientation) such that what was the coding strand is now the non-coding strand and vice versa. In such reactions, the DNA is conserved with no net gain or no loss of DNA.

Conversely, excision (integration) recombination occurs between two short, repeated DNA sequences that are oriented in the same direction. In this case, the intervening DNA is excised/removed. For example, an AND gate can be assembled by placing a terminator between each of two different sets of recombinase sites oriented for excision, flanked by a promoter and an output such as a GFP-encoding sequence. In this example, both terminators must be excised by input-dependent action of the recombinase(s) to permit readthrough from the promoter to the GFP-encoding sequence. Thus two inputs are needed to excise both terminators to generate output.

Recombinases can also be classified as irreversible or reversible. As used herein, an “irreversible recombinase” refers to a recombinase that can catalyze recombination between two complementary recombination sites, but cannot catalyze recombination between the hybrid sites that are formed by this recombination without the assistance of an additional factor. Thus, an “irreversible recognition site” refers to a recombinase recognition site that can serve as the first of two DNA recognition sequences for an irreversible recombinase and that is modified to a hybrid recognition site following recombination at that site. A “complementary irreversible recognition site” refers to a recombinase recognition site that can serve as the second of two DNA recognition sequences for an irreversible recombinase and that is modified to a hybrid recombination site following homologous recombination at that site. For example, attB and attP, described below, are the irreversible recombination sites for Bxbl and phiC31 recombinases—attB is the complementary irreversible recombination site of attP, and vice versa. Recently, it was shown that the attB/attP sites can be mutated to create orthogonal B/P pairs that only interact with each other but not the other mutants [72]. This allows a single recombinase to control the excision or integration or inversion of multiple orthogonal B/P pairs.

The phiC31 (OC31) integrase, for example, catalyzes only the attB×attP reaction in the absence of an additional factor not found in eukaryotic cells. The recombinase cannot mediate recombination between the attL and attR hybrid recombination sites that are formed upon recombination between attB and attP. Because recombinases such as the phiC31 integrase cannot alone catalyze the reverse reaction, the phiC31 attB×attP recombination is stable.

Irreversible recombinases, and nucleic acids that encode the irreversible recombinases, are described in the art and can be obtained using routine methods. Examples of irreversible recombinases include, without limitation, phiC31 (ϕC31) recombinase, coliphage P4 recombinase, coliphage lambda integrase, Listeria A118 phage recombinase, and actinophage R4 Sre recombinase, HK101, HK022, pSAM2, Bxbl, TP901, TGI, φBTI, cpRVl, cpFCl, MRU, U153 and gp29. Conversely, a “reversible recombinase” refers to a recombinase that can catalyze recombination between two complementary recombinase recognition sites and, without the assistance of an additional factor, can catalyze recombination between the sites that are formed by the initial recombination event, thereby reversing it. The product-sites generated by recombination are themselves substrates for subsequent recombination. Examples of reversible recombinase systems include, without limitation, the Cre-lox and the Flp-frt systems, R, β-six, CinH, ParA and γδ.

The recombinases provided herein are not meant to be exclusive examples of recombinases that can be used in embodiments of the invention. Other examples of recombinases that are useful in the invention described herein are known to those of skill in the art, and any new recombinase that is discovered or generated is expected to be able to be used in the different embodiments of the invention.

In some embodiments, the recombinase is serine recombinase. Thus, in some embodiments, the recombinase is considered to be irreversible. For some serine recombinases, an initial recombination event can be reversed when a recombinase directionality factor (RDF) is present. RDFs are a diverse group of proteins involved in controlling the directionality of integrase-mediated site-specific recombination reactions. Typically, RDFs are small DNA-binding proteins acting as accessory factors to influence the choice of substrates that are recombined by their cognate recombinase. See Lewis and Hatfull, Nucleic Acids Res. 2001 Jun. 1; 29(11): 2205-2216. For example, when the recombination sites, attB and attP are placed in the antiparallel orientation, the presence of recombinases will stably invert the DNA sequence between the two sites and generate an attL and attR site (“BP reaction”). This inversion remains stable unless a RDF is also expressed along with bxb1 or phiC, which will invert the sequence between attL and attR and regenerate attB and attP site (“LR reaction”). Examples of RDF include, but are not limited to, gp47 for bxb1, gp3 for phiC31, gp3 for PhiBT1, ORF7 for TP901-1, gp25 for TG1, and gp3 for PhiRv1.

In some embodiments, the recombinase is a tyrosine recombinase. Thus, in some embodiments, the recombinase is considered to be reversible.

In some embodiments, all the recombinases for an AAV expression system described herein can be of the same type (e.g., serine or tyrosine). In some embodiments, tyrosine recombinases and serine recombinases can be used together in the same nucleic acid construct described herein.

In some embodiments, the recombinase comprises the sequence of Bxbl recombinase, and the corresponding recombinase recognition sequences are Bxbl attB and Bxbl attP.

In some embodiments, the recombinase comprises the sequence of phiC31 (ϕC31) recombinase and the corresponding recombinase recognition sequences comprise phiC31attB and phiC31 attP.

A recombinase can recognize multiple pairs of RRS. In some embodiments, the recombinase comprises the sequence of Cre and the corresponding recombinase recognition sequences comprise loxP. In some embodiments, the recombinase comprises the sequence of Cre and the corresponding recombinase recognition sequences comprise lox2272. In some embodiments, the recombinase comprises the sequence of Cre and the corresponding recombinase recognition sequences comprise loxN.

In some embodiments, the recombinase comprises the sequence of Dre and the corresponding recombinase recognition sequences comprise rox.

In some embodiments, the recombinase comprises the sequence of VCre and the corresponding recombinase recognition sequences comprise VloxP.

In some embodiments, the recombinase comprises the sequence of VCre and the corresponding recombinase recognition sequences comprise VloxP.

In some embodiments, the recombinase comprises the sequence of Flp and the corresponding recombinase recognition sequences comprise FRT.

In some embodiments, the recombinase comprises the sequence of SCre and the corresponding recombinase recognition sequences comprise SloxM1.

In some embodiments, the recombinase comprises the sequence of Vika and the corresponding recombinase recognition sequences comprise vox.

In some embodiments, the recombinase comprises the sequence of B3 and the corresponding recombinase recognition sequences comprise B3RT.

In some embodiments, the recombinase comprises the sequence of KD and the corresponding recombinase recognition sequences comprise KDRT.

The sequences for some recombinases are shown below:

hPGK (SEQ ID NO: 10):
ggggttggggttgcgccttttccaaggcagccctgggtttgcgcagggacgcggctgctctgggcgtggttccggg
aaacgcagcggcgccgaccctgggtctcgcacattcttcacgtccgttcgcagcgtcacccggatcttcgccgcta
cccttgtgggccccccggcgacgcttcctgctccgcccctaagtcgggaaggttccttgcggttcgcggcgtgccg
gacgtgacaaacggaagccgcacgtctcactagtaccctcgcagacggacagcgccagggagcaatggcagcgcgc
cgaccgcgatgggctgtggccaatagcggctgctcagcagggcgcgccgagagcagcggccgggaaggggcggtgc
gggaggcggggtgtggggcggtagtgtgggccctgttcctgcccgcgcggtgttccgcattctgcaagcctccgga
gcgcacgtcggcagtcggctccctcgttgaccgaatcaccgacctctctccccag
EF1alpha (SEQ ID NO: 11):
cgtgaggctccggtgcccgtcagtgggcagagcgcacatcgcccacagtccccgagaagttggggggaggggtcgg
caattgaaccggtgcctagagaaggtggcgcggggtaaactgggaaagtgatgtcgtgtactggctccgccttttt
cccgaggggggggagaaccgtatataagtgcagtagtcgccgtgaacgttctttttcgcaacgggtttgccgccag
aacacaggtaagtgccgtgtgtggttcccgcgggcctggcctctttacgggttatggcccttgcgtgccttgaatt
acttccacctggctgcagtacgtgattcttgatcccgagcttcgggttggaagtgggtgggagagttcgaggcctt
gcgcttaaggagccccttcgcctcgtgcttgagttgaggcctggcctgggcgctggggccgccgcgtgcgaatctg
gtggcaccttcgcgcctgtctcgctgctttcgataagtctctagccatttaaaatttttgatgacctgctgcgacg
ctttttttctggcaagatagtcttgtaaatgcgggccaagatctgcacactggtatttcggtttttggggccgcgg
gcggcgacggggcccgtgcgtcccagcgcacatgttcggcgaggcggggcctgcgagcgcggccaccgagaatcgg
acgggggtagtctcaagctggccggcctgctctggtgcctggcctcgcgccgccgtgtatcgccccgccctgggcg
gcaaggctggcccggtcggcaccagttgcgtgagcggaaagatggccgcttcccggccctgctgcagggagctcaa
aatggaggacgcggcgctcgggagagcgggcgggtgagtcacccacacaaaggaaaagggcctttccgtcctcagc
cgtcgcttcatgtgactccacggagtaccgggcgccgtccaggcacctcgattagttctcgagcttttggagtacg
tcgtctttaggttggggggaggggttttatgcgatggagtttccccacactgagtgggtggagactgaagttaggc
cagcttggcacttgatgtaattctccttggaatttgccctttttgagtttggatcttggttcattctcaagcctca
gacagtggttcaaagtttttttcttccatttcaggtgtcgtga
SFFV (SEQ ID NO: 12):
ccgataaaataaaagattttatttagtctccagaaaaaggggggaatgaaagaccccacctgtaggtttggcaagc
tagctgcagtaacgccattttgcaaggcatggaaaaataccaaaccaagaatagagaagttcagatcaagggcggg
tacatgaaaatagctaacgttgggccaaacaggatatctgcggtgagcagtttcggccccggcccggggccaagaa
cagatggtcaccgcagtttcggccccggcccgaggccaagaacagatggtccccagatatggcccaaccctcagca
gtttcttaagacccatcagatgtttccaggctcccccaaggacctgaaatgaccctgcgccttatttgaattaacc
aatcagcctgcttctcgcttctgttcgcgcgcttctgcttcccgagctctataaaagagctcacaacccctcactc
ggcgcgccagtcctccgacagactgagtcgcccggg
CAG (SEQ ID NO: 13):
ACTAGTTATTAATAGTAATCAATTACGGGGTCATTAGTTCATAGCCCATATATGG
AGTTCCGCGTTACATAACTTACGGTAAATGGCCCGCCTGGCTGACCGCCCAACGA
CCCCCGCCCATTGACGTCAATAATGACGTATGTTCCCATAGTAACGCCAATAGGG
ACTTTCCATTGACGTCAATGGGTGGACTATTTACGGTAAACTGCCCACTTGGCAG
TACATCAAGTGTATCATATGCCAAGTACGCCCCCTATTGACGTCAATGACGGTAA
ATGGCCCGCCTGGCATTATGCCCAGTACATGACCTTATGGGACTTTCCTACTTGGC
AGTACATCTACGTATTAGTCATCGCTATTACCATGGGTCGAGGTGAGCCCCACGT
TCTGCTTCACTCTCCCCATCTCCCCCCCCTCCCCACCCCCAATTTTGTATTTATTTA
TTTTTTAATTATTTTGTGCAGCGATGGGGGCGGGGGGGGGGGGGGCGCGCGCCAG
GCGGGGCGGGGCGGGGCGAGGGGCGGGGCGGGGCGAGGCGGAGAGGTGCGGCG
GCAGCCAATCAGAGCGGCGCGCTCCGAAAGTTTCCTTTTATGGCGAGGCGGCGGC
GGCGGCGGCCCTATAAAAAGCGAAGCGCGCGGCGGGCGGGAGTCGCTGCGTTGC
CTTCGCCCCGTGCCCCGCTCCGCGCCGCCTCGCGCCGCCCGCCCCGGCTCTGACT
GACCGCGTTACTCCCACAGGTGAGCGGGCGGGACGGCCCTTCTCCTCCGGGCTGT
AATTAGCGCTTGGTTTAATGACGGCTCGTTTCTTTTCTGTGGCTGCGTGAAAGCCT
TAAAGGGCTCCGGGAGGGCCCTTTGTGCGGGGGGGAGCGGCTCGGGGGGTGCGT
GCGTGTGTGTGTGCGTGGGGAGCGCCGCGTGCGGCCCGCGCTGCCCGGCGGCTGT
GAGCGCTGCGGGCGCGGCGCGGGGCTTTGTGCGCTCCGCGTGTGCGCGAGGGGA
GCGCGGCCGGGGGCGGTGCCCCGCGGTGCGGGGGGGCTGCGAGGGGAACAAAG
GCTGCGTGCGGGGTGTGTGCGTGGGGGGGTGAGCAGGGGGTGTGGGCGCGGCGG
TCGGGCTGTAACCCCCCCCTGCACCCCCCTCCCCGAGTTGCTGAGCACGGCCCGG
CTTCGGGTGCGGGGCTCCGTGCGGGGCGTGGCGCGGGGCTCGCCGTGCCGGGCG
GGGGGTGGCGGCAGGTGGGGGTGCCGGGCGGGGCGGGGCCGCCTCGGGCCGGG
GAGGGCTCGGGGGAGGGGCGCGGCGGCCCCGGAGCGCCGGCGGCTGTCGAGGC
GCGGCGAGCCGCAGCCATTGCCTTTTATGGTAATCGTGCGAGAGGGCGCAGGGA
CTTCCTTTGTCCCAAATCTGGCGGAGCCGAAATCTGGGAGGCGCCGCCGCACCCC
CTCTAGCGGGCGCGGGCGAAGCGGTGCGGCGCCGGCAGGAAGGAAATGGGCGG
GGAGGGCCTTCGTGCGTCGCCGCGCCGCCGTCCCCTTCTCCATCTCCAGCCTCGG
GGCTGCCGCAGGGGGACGGCTGCCTTCGGGGGGGACGGGGCAGGGCGGGGTTCG
GCTTCTGGCGTGTGACCGGCGGCTCTAGAGCCTCTGCTAACCATGTTCATGCCTTC
TTCTTTTTCCTACAGCTCCTGGGCAACGTGCTGGTTATTGTGCTGTCTCATCATTTT
GGCAAA
NLS-iCre (SEQ ID NO: 14):
atggtgcccaagaagaagaggaaagtctccaacctgctgactgtgcaccaaaacctgcctgccctccctgtggatg
ccacctctgatgaagtcaggaagaacctgatggacatgttcagggacaggcaggccttctctgaacacacctggaa
gatgctcctgtctgtgtgcagatcctgggctgcctggtgcaagctgaacaacaggaaatggttccctgctgaacct
gaggatgtgagggactacctcctgtacctgcaagccagaggcctggctgtgaagaccatccaacagcacctgggcc
agctcaacatgctgcacaggagatctggcctgcctcgcccttctgactccaatgctgtgtccctggtgatgaggag
aatcagaaaggagaatgtggatgctggggagagagccaagcaggccctggcctttgaacgcactgactttgaccaa
gtcagatccctgatggagaactctgacagatgccaggacatcaggaacctggccttcctgggcattgcctacaaca
ccctgctgcgcattgccgaaattgccagaatcagagtgaaggacatctcccgcaccgatggtgggagaatgctgat
ccacattggcaggaccaagaccctggtgtccacagctggtgtggagaaggccctgtccctgggggttaccaagctg
gtggagagatggatctctgtgtctggtgtggctgatgaccccaacaactacctgttctgccgggtcagaaagaatg
gtgtggctgccccttctgccacctcccaactgtccacccgggccctggaagggatctttgaggccacccaccgcct
gatctatggtgccaaggatgactctgggcagagatacctggcctggtctggccactctgccagagtgggtgctgcc
agggacatggccagggctggtgtgtccatccctgaaatcatgcaggctggtggctggaccaatgtgaacattgtga
tgaactacatcagaaacctggactctgagactggggccatggtgaggctgctcgaggatggggactga
NLS-FlpO (SEQ ID NO: 15):
atggctcctaagaagaagaggaaggtgatgagccagttcgacatcctgtgcaagaccccccccaaggtgctggtgc
ggcagttcgtggagagattcgagaggcccagcggcgagaagatcgccagctgtgccgccgagctgacctacctgtg
ctggatgatcacccacaacggcaccgccatcaagagggccaccttcatgagctacaacaccatcatcagcaacagc
ctgagcttcgacatcgtgaacaagagcctgcagttcaagtacaagacccagaaggccaccatcctggaggccagcc
tgaagaagctgatccccgcctgggagttcaccatcatcccttacaacggccagaagcaccagagcgacatcaccga
catcgtgtccagcctgcagctgcagttcgagagcagcgaggaggccgacaagggcaacagccacagcaagaagatg
ctgaaggccctgctgtccgagggcgagagcatctgggagatcaccgagaagatcctgaacagcttcgagtacacca
gcaggttcaccaagaccaagaccctgtaccagttcctgttcctggccacattcatcaactgcggcaggttcagcga
catcaagaacgtggaccccaagagcttcaagctggtgcagaacaagtacctgggcgtgatcattcagtgcctggtg
accgagaccaagacaagcgtgtccaggcacatctactttttcagcgccagaggcaggatcgaccccctggtgtacc
tggacgagttcctgaggaacagcgagcccgtgctgaagagagtgaacaggaccggcaacagcagcagcaacaagca
ggagtaccagctgctgaaggacaacctggtgcgcagctacaacaaggccctgaagaagaacgccccctaccccatc
ttcgctatcaagaacggccctaagagccacatcggcaggcacctgatgaccagctttctgagcatgaagggcctga
ccgagctgacaaacgtggtgggcaactggagcgacaagagggcctccgccgtggccaggaccacctacacccacca
gatcaccgccatccccgaccactacttcgccctggtgtccaggtactacgcctacgaccccatcagcaaggagatg
atcgccctgaaggacgagaccaaccccatcgaggagtggcagcacatcgagcagctgaagggcagcgccgagggca
gcatcagataccccgcctggaacggcatcatcagccaggaggtgctggactacctgagcagctacatcaacaggcg
gatctga
NLS-DreO (SEQ ID NO: 16):
ATGCCTAAGAAGAAGAGGAAGGTTTCTGAGCTGATTATTAGTGGTTCATCTGGTG
GATTCCTGCGAAACATCGGCAAAGAGTATCAGGAGGCCGCTGAAAACTTCATGA
GGTTTATGAATGACCAGGGGGCGTACGCTCCTAACACTTTGAGGGATTTGAGGTT
GGTCTTTCATAGCTGGGCCAGATGGTGCCATGCTCGGCAGCTTGCATGGTTTCCA
ATTAGTCCTGAAATGGCACGCGAATACTTTCTTCAGTTGCACGATGCAGACCTGG
CCTCCACTACCATCGACAAGCACTATGCTATGCTTAATATGCTTCTGTCCCACTGC
GGACTGCCACCCTTGTCCGACGACAAGTCAGTGAGTCTTGCCATGAGAAGAATTA
GAAGAGAAGCCGCAACCGAAAAGGGTGAGAGGACAGGACAGGCAATCCCCCTG
CGCTGGGACGACCTGAAGCTGCTGGATGTGCTGCTCAGCAGGAGCGAGCGGCTG
GTCGACCTGCGCAACAGGGCTTTCCTGTTCGTAGCCTATAACACCCTCATGAGAA
TGTCTGAAATATCACGCATCAGGGTTGGGGACTTGGATCAGACAGGAGACACAG
TGACCCTGCACATCAGTCACACTAAGACAATCACCACAGCTGCGGGCCTTGACAA
AGTGCTCTCCCGGCGAACCACAGCAGTGCTCAATGACTGGCTGGACGTCAGTGGG
CTTAGAGAACATCCAGACGCTGTGCTCTTCCCACCTATACACCGGTCAAACAAAG
CCCGCATTACTACCACGCCCCTGACCGCCCCTGCCATGGAGAAGATTTTCAGTGA
TGCCTGGGTGCTGCTGAACAAACGGGACGCCACCCCCAATAAAGGGAGGTATAG
GACCTGGACCGGCCATTCCGCCAGGGTGGGTGCCGCAATAGACATGGCCGAGAA
ACAGGTGTCTATGGTCGAGATTATGCAGGAAGGGACATGGAAGAAGCCTGAAAC
ACTGATGCGGTATCTCAGAAGGGGCGGAGTGTCCGTGGGAGCCAATTCTCGACTG
ATGGATAGCTAA
NLS-SCre (SEQ ID NO: 17):
ATGCCCAAGAAAAAGCGGAAGGTGTCCCTGCTGACCACCAACAACCACAGCGTG
GCCCTGAGCTACGGCGAGCCTCCTAGCACCCTGAACGACAGCCTGAAGGACAGC
TACCAGCGGAGCACCGATGAGCTGCAGGCCCTGCTGTCTAAGCCTCTGGCCCAGC
TGACCGACGCCGACAAGCTGCGGATCAGAGAGATCACCCAGGCCAAGCTGAAGC
ACTTCCTGGACAACGGCCACCGGACCAGAAGGGCCAACACTTGGAGAGCCCTGA
TGAGCAGATGGGCCAAGTTCGAGAGCTGGTGCCTGACCAACAATCTGACCCCCCT
GCCTGCCACCCCTGAGGTGGTGGCCACATTCATCGAGTACTACCAGGCCAGCAGC
TACACCACCCTGAGCCAGTATGCCTGGGCCATCAACAGCTTTCACGTGGAATGCG
GCCTGCTGAGCCCCGTGTCTAGCAAGACCGTGCAGGACAAGCAGAACGAGATCA
GAATCGTGAAGCTGGAATCTGGCGGCCTGGCCCAGGAACAGGCCACCCCTTTTAG
ACTGCACCATCTGCAGATGCTGATCGAGAGCTATGGCGAGAGCGAGCGGCTGCT
GGACAAGAGAAACCTGGCTCTGCTGAATATCGCCTACGAGAGCCTGCTGCGCGA
GTCCGAGCTGCTGAGAATCAAAGTGGGCCACCTGAAGTCCACCTTCGAGGGCGA
CTACGTGCTGAGCGTGCCCTACACCAAGACCAACGACAGCGGCGAAGAGGAAGT
CGTGAACATCACCCCCCTGGGCTTCAAGCTGATCCAGCGGTACATCCAGGGCGCT
GGCCTGACAAAAGAGGACTACCTGTTCCAGCCCATCGGCCGGTCCAACAAGGTG
TCCGTGCAGGCCAAACCCATGAGCACCCGGACCGTGGACAGAGTGTTCCTGTGG
GCCTTTGAGAGCCTGGGCATCGACAGACACAGCGCTTGGAGCGGCCACAGCGCC
AGAATTGGAGCCGCTCAGGATCTGCTGGCCGCTGGCTATTCTATCGCCCAGATCC
AGGAAAACGGCCGCTGGAAGTCCCCCATGATGGTGCTGAGATACGGCAAGGACA
TCAAGGCCAAAGAAAGCGCCATGGCCAAGATGCTGGCCGAGCGGAGATGA
NLS-VCre (SEQ ID NO: 18):
ATGCCCAAGAAAAAGCGGAAAGTGATCGAGAACCAGCTGAGCCTGCTGGGCGAC
TTTTCTGGCGTGCGGCCCGACGATGTGAAAACCGCCATTCAGGCCGCCCAGAAAA
AGGGCATCAACGTGGCCGAGAACGAGCAGTTCAAGGCCGCCTTCGAGCATCTGC
TGAACGAGTTCAAGAAGCGGGAAGAGAGATACAGCCCCAACACCCTGCGGCGGC
TGGAAAGCGCCTGGACCTGCTTCGTGGATTGGTGCCTGGCCAACCACAGACACAG
CCTGCCTGCCACCCCCGATACCGTGGAAGCCTTCTTCATCGAGCGGGCCGAGGAA
CTGCACCGGAACACCCTGAGCGTGTACAGATGGGCCATCAGCCGGGTGCACAGA
GTGGCCGGATGCCCTGATCCCTGCCTGGACATCTACGTGGAAGATCGGCTGAAGG
CCATTGCCCGGAAGAAAGTGCGGGAAGGCGAGGCCGTGAAGCAGGCCAGCCCTT
TCAACGAGCAGCATCTGCTGAAGCTGACCAGCCTGTGGTACAGAAGCGACAAGC
TGCTGCTGCGGCGGAACCTGGCTCTGCTGGCTGTGGCCTACGAGAGCATGCTGAG
AGCCAGCGAGCTGGCCAACATCCGGGTGTCCGATATGGAACTGGCCGGCGACGG
AACCGCCATCCTGACCATCCCTATCACCAAGACCAACCACTCCGGCGAGCCCGAT
ACCTGCATCCTGTCCCAGGATGTGGTGTCCCTGCTGATGGACTACACCGAGGCCG
GCAAGCTGGATATGAGCAGCGACGGCTTCCTGTTCGTGGGCGTGTCCAAGCACAA
CACCTGTATCAAGCCCAAGAAGGACAAGCAGACCGGCGAGGTGCTGCACAAGCC
CATCACCACCAAGACAGTGGAAGGCGTGTTCTACAGCGCCTGGGAGACACTGGA
CCTGGGCAGACAGGGCGTGAAGCCTTTCACAGCCCACAGCGCCAGAGTGGGAGC
CGCTCAGGACCTGCTGAAGAAGGGCTACAATACCCTGCAGATCCAGCAGTCCGG
CCGGTGGTCTAGCGGAGCCATGGTGGCCAGATACGGCAGAGCCATCCTGGCTAG
GGATGGCGCTATGGCCCACAGCAGAGTGAAAACCAGATCCGCCCCCATGCAGTG
GGGCAAGGACGAGAAGGACTGA
NLS-VikaO (SEQ ID NO: 19):
ATGCCCAAGAAAAAGCGGAAAGTGACCGACCTGACCCCATTCCCCCCCCTGGAA
CACCTGGAACCCGACGAGTTTGCCGACCTCGTGCGGAAGGCCATCAAGAGGGAT
CCTCAGGCTGGCGCCCACCCTGCCATCCAGTCTGCCATCAGCCACTTCCAGGACG
AGTTCGTGCGGAGACAGGGCGAATGGCAGCCTGCCACACTGCAGAGACTGAGAA
ACGCCTGGAATGTGTTTGTGCGGTGGTGCACCCACCAGGGCATTCCAGCTCTGCC
TGCCAGACACCAGGACGTGGAAAGATACCTGATCGAGCGGCGGAACGAGCTGCA
CCGGAACACCCTGAAAGTGCACCTGTGGGCCATCGGCAAGACCCACGTGATCAG
CGGCCTGCCCAATCCCTGCGCCCACAGATACGTGAAAGCCCAGATGGCCCAGATC
ACACACCAGAAAGTGCGCGAGAGAGAGCGGATCGAACAGGCCCCTGCCTTCAGA
GAGTCCGACCTGGACAGACTGACCGAGCTGTGGAGCGCCACCAGAAGCGTGACC
CAGCAGCGGGACCTGATGATCGTGTCCCTGGCCTACGAGACACTGCTGCGGAAG
AACAATCTGGAACAGATGAAAGTGGGCGACATCGAGTTCTGCCAGGACGGCTCT
GCCCTGATCACCATCCCCTTCAGCAAGACCAACCACAGCGGCAGGGATGACGTG
CGGTGGATCTCTCCCCAGGTGGCCAATCAGGTGCACGCCTACCTGCAGCTGCCCA
ACATCGACGCCGACCCCCAGTGCTTCCTGCTGCAGAGAGTGAAGAGAAGCGGCA
AGGCCCTGAACCCCGAGAGCCACAATACCCTGAACGGCCACCACCCCGTGTCCG
AGAAGCTGATCTCCCGGGTGTTCGAGCGGGCTTGGAGAGCCCTGAATCACGAGA
CAGGCCCCAGATACACCGGCCACAGCGCTAGAGTGGGAGCCGCTCAGGATCTGC
TGCAGGAAGGCTACAGCACCCTGCAAGTGATGCAGGCAGGCGGCTGGTCCAGCG
AGAAGATGGTGCTGAGATACGGCCGGCATCTGCACGCCCACACATCTGCCATGG
CTCAGAAACGGCGGCAGCGGTGA
NLS-B3 (SEQ ID NO: 20):
ATGCCCAAGAAAAAGCGGAAGGTGTCCAGCTACATGGACCTGGTGGACGACGAG
CCCGCCACCCTGTACCACAAGTTCGTGGAATGCCTGAAGGCCGGCGAGAACTTCT
GCGGCGATAAGCTGAGCGGCATCATCACCATGGCCATTCTGAAGGCCATCAAGG
CCCTGACCGAAGTGAAGAAAACCACCTTCAACAAGTACAAGACCACCATCAAGC
AGGGCCTGCAGTACGACGTGGGCAGCAGCACCATCAGCTTCGTGTACCACCTGA
AGGACTGCGACGAGCTGAGCAGAGGCCTGAGCGACGCCTTCGAGCCCTACAAGT
TCAAGATCAAGAGCAACAAAGAGGCCACCAGCTTCAAGACCCTGTTCAGGGGCC
CTAGCTTCGGCAGCCAGAAGAACTGGCGGAAGAAAGAGGTGGACCGCGAGGTGG
ACAACCTGTTCCACAGCACCGAGACAGACGAGAGCATCTTCAAGTTCATCCTGAA
CACCCTGGACAGCATCGAAACCCAGACCAACACCGACCGGCAGAAAACCGTGCT
GACCTTTATCCTGCTGATGACCTTCTTCAACTGCTGCCGGAACAACGACCTGATG
AACGTGGACCCCAGCACCTTCAAGATCGTGAAGAACAAGTTTGTGGGCTACCTGC
TGCAGGCTGAAGTGAAGCAGACCAAGACCAGAAAGAGCCGGAATATCTTCTTCT
TCCCCATCCGGGAAAACCGCTTCGACCTGTTCCTGGCCCTGCACGACTTCTTCAG
AACCTGCCAGCCCACCCCCAAGAGCAGACTGAGCGATCAGGTGTCCGAGCAGAA
GTGGCAGCTGTTCCGGGACAGCATGGTCATCGACTACAACCGGTTCTTTCGGAAG
TTCCCCGCCAGCCCCATCTTCGCCATTAAGCACGGCCCCAAGTCCCACCTGGGCC
GGCATCTGATGAACAGCTTTCTGCACAAGAACGAGCTGGACAGCTGGGCCAACA
GCCTGGGCAATTGGAGCAGCTCCCAGAACCAGAGAGAGAGCGGCGCCAGACTGG
GCTACACACACGGCGGAAGAGATCTGCCCCAGCCCCTGTTTGGCTTCCTGGCCGG
ATACTGCGTGCGGAACGAAGAGGGCCACATCGTGGGCCTGGGCCTGGAAAAGGA
CATCAACGATCTGTTCGACGGCATCATGGACCCCCTGAACGAGAAAGAGGACAC
CGAGATCTGCGAGAGCTACGGCGAGTGGGCCAAGATTGTGTCCAAGGACGTGCT
GATCTTCCTGAAGAGATACCACAGCAAGAACGCCTGTCGGAGATACCAGAACAG
CACCCTGTATGCCCGGACCTTCCTGAAAACCGAGAGCGTGACCCTGAGCGGCTCC
AAGGGCAGCGAGGAACCTTCTAGCCCTGTGCGGATCCCCATCCTGAGCATGGGA
AAGGCCAGCCCCTCCGAGGGAAGAAAGCTGAGAGCCAGCGAGCACGCCAACGA
CGACAACGAGATCGAGAAGATCGACAGCGACAGCAGCCAGAGCGAAGAGATCC
CTATCGAGATGAGCGACTCCGAGGACGAGACAACCGCCAGCAACATCAGCGGCA
TCTACCTGGACATGAGCAAGGCCAACTCCAACGTGGTGTACAGCCCCCCTAGCCA
GACAGGCAGAGCTGCTGGCGCCGGAAGAAAAAGAGGCGTGGGAGGCAGACGGA
CCGTGGAAAGCAAGCGGAGAAGAGTGCTGGCCCCCATCAACCGGTGA
NLS-KD (SEQ ID NO: 21):
ATGCCCAAGAAAAAGCGGAAGGTGTCCACCTTCGCCGAGGCCGCCCATCTGACA
CCTCACCAGTGCGCCAACGAGATCAATGAGATCCTGGAAAGCGACACCTTCAAC
ATCAACGCCAAAGAGATCCGGAACAAGCTGGCCTCCCTGTTCAGCATCCTGACCA
TGCAGAGCCTGAGCATCCGCAGAGAGATGAAGATCAACACCTACCGGTCCTACA
AGAGCGCCATCGGCAAGAGCCTGTCCTTCGACAAGGACGACAAGATCATCAAGT
TCACCGTGCGGCTGAGAAAGACCGAGAGCCTGCAGAAGGACATCGAGAGCGCCC
TGCCCAGCTACAAGGTGGTGGTGTCCCCATTCAAGAACCAGGAAGTGTCCCTGTT
CGACCGCTACGAGGAAACCCACAAATACGACGCCAGCATGGTGGGACTGCAGTT
CACCAACATCCTGAGCAAAGAGAAGGATATCTGGAAGATCGTGTCCCGGATCGC
CTGCTTCTTCGACCAGAGCTGCGTGACCACCACCAAGCGGGCCGAGTACAGACTG
CTGCTGCTGGGCGCTGTGGGCAACTGCTGCAGATACAGCGACCTGAAGAACCTG
GACCCCCGGACCTTCGAGATCTACAACAACAGCTTCCTGGGCCCCATCGTGCGGG
CCACCGTGACAGAGACAAAGAGCCGGACCGAGAGATACGTGAACTTCTACCCCG
TGAACGGCGACTGCGACCTGCTGATCTCCCTGTACGACTACCTGAGAGTGTGCAG
CCCCATCGAGAAAACCGTGTCCAGCAACCGGCCCACCAACCAGACCCACCAGTTT
CTGCCTGAGAGCCTGGCCAGAACCTTCAGCCGGTTCCTGACCCAGCACGTGGACG
AGCCCGTGTTCAAGATCTGGAACGGCCCCAAGAGCCACTTCGGCAGACACCTGAT
GGCCACCTTTCTGAGCAGAAGCGAGAAGGGCAAATACGTGTCCTCCCTGGGCAA
TTGGGCTGGCGACCGGGAAATCCAGTCTGCCGTGGCCAGAAGCCACTACAGCCA
CGGCTCTGTGACCGTGGACGACCGGGTGTTCGCCTTCATCAGCGGCTTCTACAAA
GAGGCCCCCCTGGGCAGCGAGATCTATGTGCTGAAGGACCCCAGCAACAAGCCC
CTGAGCAGAGAGGAACTGCTGGAAGAGGAAGGCAACAGCCTGGGCTCCCCACCT
CTGAGCCCTCCAAGCTCTCCTAGACTGGTGGCCCAGAGCTTCAGCGCCCACCCAA
GCCTGCAGCTGTTCGAGCAGTGGCACGGCATCATCAGCGACGAGGTGCTGCAGTT
TATCGCCGAGTACCGGCGGAAGCACGAGCTGAGAAGCCAGAGAACCGTGGTGGC
CTGA
NLS-B2 (SEQ ID NO: 22):
ATGCCCAAGAAAAAGCGGAAGGTGTCCGAGTTCAGCGAGCTCGTGCGGATCCTG
CCCCTGGATCAGGTGGCCGAGATCAAGAGAATCCTGAGCAGAGGCGACCCCATC
CCCCTGCAGAGACTGGCCTCTCTGCTGACCATGGTCATCCTGACCGTGAACATGA
GCAAGAAGAGAAAGAGCAGCCCCATCAAGCTGAGCACCTTCACCAAGTACCGGC
GGAACGTGGCCAAGAGCCTGTACTACGACATGAGCAGCAAGACCGTGTTCTTCG
AGTACCACCTGAAGAACACCCAGGACCTGCAGGAAGGCCTGGAACAGGCCATTG
CCCCCTACAACTTCGTCGTGAAAGTGCACAAGAAGCCCATCGACTGGCAGAAAC
AGCTGAGCAGCGTGCACGAGCGGAAGGCCGGCCACAGATCCATCCTGTCCAACA
ACGTGGGCGCCGAGATCTCCAAGCTGGCCGAGACAAAGGACAGCACCTGGTCCT
TCATCGAGCGGACCATGGACCTGATCGAGGCCAGAACCAGACAGCCCACCACCA
GAGTGGCCTACCGGTTCCTGCTGCAGCTGACCTTCATGAACTGCTGCCGGGCCAA
CGATCTGAAGAACGCCGACCCCAGCACCTTCCAGATCATTGCCGATCCCCACCTG
GGCCGGATCCTGAGAGCCTTCGTGCCCGAGACTAAGACCTCTATCGAGCGGTTTA
TCTACTTCTTCCCATGCAAGGGCCGCTGCGACCCTCTGCTGGCCCTGGATTCTTAC
CTGCTGTGGGTGGGACCCGTGCCCAAGACCCAGACCACCGATGAGGAAACCCAG
TACGACTACCAGCTGCTGCAGGACACCCTGCTGATCTCTTACGACCGGTTTATCG
CCAAAGAGAGCAAAGAGAACATCTTCAAGATCCCCAACGGCCCCAAGGCCCATC
TGGGCAGACATCTGATGGCCAGCTACCTGGGCAACAACAGCCTGAAGTCCGAGG
CCACCCTGTACGGCAATTGGAGCGTGGAAAGACAGGAAGGCGTGTCCAAAATGG
CCGACAGCCGGTACATGCACACCGTGAAGAAGTCCCCCCCCTCCTACCTGTTCGC
CTTTCTGAGCGGCTACTACAAGAAGTCCAACCAGGGCGAGTACGTGCTGGCCGA
AACCCTGTACAACCCCCTGGACTACGATAAGACCCTGCCCATCACCACCAACGAG
AAGCTGATCTGCAGACGCTACGGCAAGAACGCCAAAGTGATCCCCAAGGATGCC
CTGCTGTACCTGTACACCTACGCCCAGCAGAAGCGGAAGCAGCTGGCTGACCCCA
ACGAGCAGAACCGGCTGTTCAGCAGCGAGAGCCCTGCCCACCCATTTCTGACCCC
TCAGAGCACAGGCAGCAGCACCCCTCTGACATGGACCGCCCCTAAGACACTGAG
CACCGGCCTGATGACCCCTGGCGAGGAATGA
NLS-R (SEQ ID NO: 23):
ATGCCCAAGAAAAAGCGGAAGGTGCAGCTGACCAAGGACACCGAGATCAGCACC
ATCAACCGGCAGATGAGCGACTTCAGCGAGCTGAGCCAGATCCTGCCCCTGCACC
AGATCTCCAAGATCAAGGACATCCTGGAAAACGAGAACCCCCTGCCCAAAGAGA
AGCTGGCCTCCCACCTGACCATGATCATCCTGATGGCCAACCTGGCCAGCCAGAA
ACGGAAGGACGTGCCCGTGAAGCGGAGCACCTTCCTGAAGTACCAGCGGAGCAT
CAGCAAGACCCTGCAGTACGACAGCAGCACCAAGACCGTGTCCTTCGAGTACCA
CCTGAAGGACCCCAGCAAGCTGATCAAGGGCCTGGAAGATGTGGTGTCCCCCTA
CAGATTCGTCGTGGGCGTGCACGAGAAGCCCGACGACGTGATGTCTCACCTGAGC
GCCGTGCACATGCGGAAAGAGGCCGGCAGAAAGCGGGACCTGGGCAACAAGAT
CAACGACGAGATCACAAAGATCGCCGAGACACAGGAAACCATCTGGGGCTTCGT
GGGCAAGACCATGGACCTGATCGAGGCCAGAACCACCCGGCCTACAACAAAGGC
CGCCTACAACCTGCTGCTGCAGGCCACCTTCATGAACTGCTGCAGAGCCGACGAC
CTGAAGAACACCGACATCAAGACCTTCGAAGTGATCCCCGACAAGCACCTGGGC
CGGATGCTGAGAGCCTTCGTGCCCGAGACAAAGACCGGAACCAGATTCGTGTAC
TTCTTCCCATGCAAGGGCAGATGCGACCCCCTGCTGGCCCTGGATTCTTACCTGC
AGTGGACCGACCCCATCCCCAAGACCAGAACAACCGACGAGGACGCCAGATACG
ACTACCAGCTGCTGCGGAACAGCCTGCTGGGCAGCTACGACGGCTTCATCTCCAA
GCAGAGCGACGAGAGCATCTTCAAGATCCCCAACGGCCCCAAGGCCCACCTGGG
CAGACATGTGACAGCCAGCTACCTGAGCAACAACGAGATGGACAAAGAGGCCAC
CCTGTACGGCAATTGGAGCGCCGCTAGAGAAGAGGGCGTGTCCAGAGTGGCCAA
GGCCCGGTACATGCACACCATCGAGAAGTCCCCCCCCTCCTACCTGTTCGCCTTC
CTGAGCGGCTTCTACAACATCACCGCCGAGAGGGCCTGCGAGCTGGTGGACCCC
AATAGCAACCCCTGCGAGCAGGACAAGAACATCCCCATGATCAGCGACATCGAG
ACACTGATGGCTCGCTACGGCAAGAACGCCGAGATCATCCCTATGGACGTGCTGG
TGTTCCTGAGCAGCTACGCCCGGTTCAAGAACAACGAGGGCAAAGAGTACAAGC
TGCAGGCTCGGAGCAGCAGAGGCGTGCCCGACTTCCCCGATAATGGCAGAACCG
CCCTGTACAACGCCCTGACAGCCGCCCACGTGAAGAGGCGGAAGATCAGCATTG
TCGTGGGCCGGTCCATCGACACCAGCTGA
NLS-PhiC31 (SEQ ID NO: 24):
atgcctaagaaaaagcggaaagtggatacctacgccggagcctacgacagacagagccgggagagagagaacagca
gcgccgccagccccgccacccagagaagcgccaacgaggataaggccgccgatctgcagagagaggtggagaggga
cggcggcagattcagatttgtgggccacttcagcgaggcccctggcaccagcgccttcggcaccgccgagagaccc
gagttcgagagaatcctgaacgagtgtagggccggcaggctgaacatgatcatcgtgtacgacgtgtcccggttca
gcaggctgaaggtgatggacgccatccctatcgtgtccgagctgctggccctgggcgtgaccatcgtgtccaccca
ggaaggcgtctttagacagggcaacgtgatggacctgatccacctgatcatgaggctggacgccagccacaaggag
agcagcctgaagagcgccaagatcctggacaccaagaacctgcagagggagctgggcggctatgtgggcggcaagg
ccccctacggcttcgagctggtgtccgagaccaaggagatcacccggaacggcaggatggtgaacgtggtgatcaa
caagctggcccacagcaccacccccctgaccggccccttcgagtttgagcccgacgtgatcaggtggtggtggcgg
gagatcaagacccacaagcacctgcctttcaagcccggcagccaggccgccatccaccccggcagcatcaccggcc
tgtgtaagagaatggacgccgacgccgtgcccaccagaggcgagaccatcggcaagaaaaccgccagcagcgcctg
ggaccccgccaccgtgatgagaatcctgagggaccctaggatcgccggcttcgccgccgaggtgatctacaagaag
aagcccgacggcacccccaccaccaagatcgagggctacagaatccagagagaccccatcaccctgagacctgtgg
agctggactgtggccctatcatcgagcctgccgagtggtacgagctgcaggcctggctggacggcagaggcagagg
caagggcctgagcagaggccaggccatcctgagcgccatggacaagctgtactgtgagtgtggcgccgtgatgacc
agcaagagaggcgaggagagcatcaaggacagctaccggtgccggagaagaaaggtggtggaccccagcgcccctg
gccagcacgagggcacctgtaatgtgagcatggccgccctggacaagttcgtggccgagcggatcttcaacaagat
ccggcacgccgagggcgacgaggagaccctggccctgctgtgggaggccgccagaagattcggcaagctgaccgag
gcccccgagaagagcggcgagagggccaacctggtggccgagagagccgacgccctgaacgccctggaggagctgt
acgaggacagagccgccggagcctatgacggccctgtgggcaggaagcacttcagaaagcagcaggccgccctgac
cctgagacagcagggcgccgaggaaagactggccgagctggaggccgccgaggcccctaagctgcccctggatcag
tggttccccgaggatgccgacgccgaccccaccggccccaagtcctggtggggcagagccagcgtggacgacaaga
gggtgttcgtgggcctgttcgtggataagatcgtggtgaccaagagcaccaccggcaggggccagggcacccccat
cgagaagagagccagcatcacctgggccaagcctcccaccgacgacgacgaggatgacgcccaggacggcaccgag
gacgtggccgcctga
NLS-bxb1 (SEQ ID NO: 25):
ATGGATCCTAAGAAAAAGCGAAAAGTGATGCGAGCCCTGGTGGTCATTCGCCTG
AGCAGAGTCACAGACGCTACTACAAGCCCTGAGCGGCAGCTGGAGTCCTGTCAG
CAGCTGTGCGCACAGCGAGGATGGGATGTGGTCGGAGTGGCAGAGGATCTGGAC
GTGAGCGGGGCTGTCGATCCATTCGACCGAAAGCGGAGACCCAACCTGGCACGA
TGGCTGGCTTTCGAGGAACAGCCCTTTGATGTGATCGTCGCCTACAGAGTGGACA
GGCTGACACGCTCAATTCGACATCTGCAGCAGCTGGTGCATTGGGCCGAGGATCA
CAAGAAACTGGTGGTCAGCGCAACTGAAGCCCACTTCGACACCACAACTCCTTTT
GCCGCTGTGGTCATCGCACTGATGGGCACCGTGGCCCAGATGGAGCTGGAAGCT
ATCAAGGAGCGAAACCGGAGCGCAGCCCATTTCAATATTCGGGCCGGGAAATAC
AGAGGCAGCCTGCCCCCTTGGGGCTATCTGCCTACCCGGGTGGATGGGGAGTGG
AGACTGGTGCCAGACCCCGTCCAGAGAGAGAGGATTCTGGAAGTGTACCACAGA
GTGGTGGACAACCACGAACCACTGCATCTGGTGGCCCACGATCTGAATAGGCGC
GGAGTCCTGTCTCCAAAGGACTATTTTGCTCAGCTGCAGGGAAGGGAGCCACAG
GGACGAGAATGGAGTGCTACCGCACTGAAGCGGTCTATGATCAGTGAGGCTATG
CTGGGCTATGCAACTCTGAATGGGAAAACCGTGAGAGACGATGACGGAGCACCA
CTGGTGCGGGCTGAGCCTATTCTGACAAGAGAGCAGCTGGAAGCTCTGAGGGCA
GAACTGGTGAAAACCAGTAGGGCCAAGCCTGCTGTGTCAACACCAAGCCTGCTG
CTGCGAGTGCTGTTCTGCGCAGTCTGTGGCGAGCCAGCATACAAATTTGCCGGCG
GGGGAAGGAAGCATCCCCGCTATCGATGCCGGAGCATGGGGTTCCCTAAGCACT
GTGGAAACGGCACTGTGGCTATGGCCGAATGGGACGCCTTTTGTGAGGAACAGG
TGCTGGATCTGCTGGGGGACGCAGAGCGCCTGGAAAAAGTGTGGGTCGCTGGAA
GCGATTCCGCTGTGGAGCTGGCAGAAGTCAATGCCGAGCTGGTGGACCTGACCTC
CCTGATCGGATCTCCTGCATACAGGGCAGGCTCCCCACAGCGAGAAGCTCTGGAT
GCACGAATTGCTGCACTGGCAGCTCGACAGGAGGAACTGGAGGGGCTGGAAGCC
AGACCCTCTGGATGGGAGTGGCGAGAAACAGGCCAGCGGTTTGGGGATTGGTGG
AGGGAGCAGGACACAGCAGCCAAGAACACTTGGCTGAGATCCATGAATGTCAGG
CTGACTTTCGACGTGCGAGGAGGACTGACCCGAACAATCGATTTTGGCGACCTGC
AGGAGTATGAACAGCATCTGCGCCTGGGAAGTGTGGTCGAGCGACTGCACACCG
GCATGTCATAA

The sequences for some recombinase recognitions sequences (RRSs) are shown below:

loxP (SEQ ID NO: 26):
ATAACTTCGTATAgcatacatTATACGAAGTTAT
lox2272 (SEQ ID NO: 27):
ATAACTTCGTATAggatacctTATACGAAGTTAT
loxN (SEQ ID NO: 28):
ATAACTTCGTATAgtatacctTATACGAAGTTAT
FRT (SEQ ID NO: 29):
GAAGTTCCTATTCTCTAGAAAGTATAGGAACTTC
F3 (SEQ ID NO: 30):
GAAGTTCCTATTCttcaaataGTATAGGAACTTC
F14 (SEQ ID NO: 31):
GAAGTTCCTATTCtatcagaaGTATAGGAACTTC
Rox (SEQ ID NO: 32):
TAACTTTAAATAattggcatTATTTAAAGTTA
VloxP (SEQ ID NO: 33):
TCAATTTCTGAGAactgtcatTCTCGGAAATTGA
Vlox2272 (SEQ ID NO: 34):
TCAATTTCTGAGAagtgtcttTCTCGGAAATTGA
SloxP (SEQ ID NO: 35):
CTCGTGTCCGATAactgtaatTATCGGACATGAT
SloxM1 (SEQ ID NO: 36):
CTCGTGTCCGATAactgtaatTATCGGACACGAG
Slox2272 (SEQ ID NO: 37):
CTCGTGTCCGATAagtgtattTATCGGACATGAT
Vox (SEQ ID NO: 38):
AATAGGTCTGAGAacgcccatTCTCAGACGTATT
B3RT (SEQ ID NO: 39):
GGTTGCTTAAGAATAAGTAATTCTTAAGCAACC
KDRT (SEQ ID NO: 40):
AAACGATATCAGACATTTGTCTGATAATGCTTCATTATCAGACAAATGTC
TGATATCGTTT
B2RT (SEQ ID NO: 41):
GAGTTTCATTAAGGAATAACTAATTCCCTAATGAAACTC
RSRT (SEQ ID NO: 42):
TTGATGAAAGAATAACGTATTCTTTCATCAA
PhiC31 attB (SEQ ID NO: 43):
TGCGGGTGCCAGGGCGTGCCCTTGGGCTCCCCGGGCGCGTACTCC
PhiC31 attP (SEQ ID NO: 44):
GTGCCCCAACTGGGGTAACCTTTGAGTTCTCTCAGTTGGGGG
Bxb1 attB (SEQ ID NO: 45):
TCGGCCGGCTTGTCGACGACGGCGGTCTCCGTCGTCAGGATCATCCGGGC
Bxb1 attP (SEQ ID NO: 46):
TCGTGGTTTGTCTGGTCAACCACCGCGGTCTCAGTGGTGTACGGTACAAA
CCC

### Cell Lines

Provided herein are stable cell lines for recombinant viral vector production comprising at least one inducible promoter operatively linked to a heterologous gene that encodes a toxic protein.

One aspect of this application provides a stable cell line for recombinant viral vector production, for example, AAV vector production, comprising at least one inducible promoter, wherein the inducible promoter is operatively linked to a heterologous rep or pol gene that encodes a rep protein or polymerase protein, respectively. In one embodiment, the inducible promoter is further operatively linked to a heterologous cap or env gene that encodes a cap protein or env protein, respectively. Alternatively, in one embodiment, the stable cell line further comprises a second inducible promoter operatively linked to a heterologous cap or env gene that encodes a cap protein or env protein, respectively, wherein the second inducible promoter is induced by a different inducer from the first inducible promoter.

Another aspect of this application provides a stable cell line for recombinant AAV vector production comprising at least one inducible promoter, wherein the inducible promoter is operatively linked to a heterologous cap gene that encodes a cap protein.

Another aspect of this application provides a stable cell line for recombinant AAV vector production comprising at least one regulatable promoter, e.g., an inducible promoter, wherein the inducible promoter is operatively linked to a heterologous helper gene that encodes a helper gene product. Helper genes are commonly used in the production of AAV vectors. Exemplary helper genes conventionally used include in AAV production include E1 (E1A and E1B), E2A, E4 and VA RNA.

Another aspect of this application provides a stable cell line for recombinant viral vector production, comprising at least one inducible promoter operatively linked to a heterologous gene that encodes a toxic protein, wherein the at least one inducible promoter is selected from the group consisting of: a forskolin inducible promoter, a hypoxia inducible promoter, a tetracycline inducible promoter, an alcohol inducible promoter, a steroid inducible promoter, an RU486 inducible promoter, an ecdysone inducible promoter, a rapamycin inducible promoter, a metallothionein inducible promoter, a hormone inducible promoter and a metal inducible promoter.

Another aspect of this application provides a stable cell line for recombinant viral vector production, comprising at least one forskolin inducible promoter operatively linked to at least one heterologous gene that encodes a toxic protein.

Another aspect of this application provides a stable cell line for recombinant viral vector production, comprising at least one hypoxia inducible promoter operatively linked to at least one heterologous gene that encodes a toxic protein.

Another aspect of this application provides a stable cell line for recombinant viral vector production, comprising at least one forskolin inducible promoter operatively linked to at least one heterologous gene that encodes a toxic protein, and at least one inducible promoter operatively linked to at least one heterologous gene that encodes a toxic protein, wherein the at least one inducible promoter is selected from the group consisting of: a forskolin inducible promoter, a hypoxia inducible promoter, a tetracycline inducible promoter, an alcohol inducible promoter, a steroid inducible promoter, an RU486 inducible promoter, an ecdysone inducible promoter, a rapamycin inducible promoter, a metallothionein inducible promoter, a hormone inducible promoter and a metal inducible promoter.

Another aspect of this application provides a stable cell line for recombinant viral vector production, comprising at least one hypoxia inducible promoter operatively linked to at least one heterologous gene that encodes a toxic protein, and at least one inducible promoter operatively linked to at least one heterologous gene that encodes a toxic protein, wherein the at least one inducible promoter is selected from the group consisting of: a forskolin inducible promoter, a hypoxia inducible promoter, a tetracycline inducible promoter, an alcohol inducible promoter, a steroid inducible promoter, an RU486 inducible promoter, an ecdysone inducible promoter, a rapamycin inducible promoter, a metallothionein inducible promoter, a hormone inducible promoter and a metal inducible promoter.

Another aspect of this application provides a stable cell line for recombinant viral vector production, comprising at least one forskolin inducible promoter operatively linked to at least one heterologous gene that encodes a toxic protein, and at least one hypoxia inducible promoter operatively linked to at least one heterologous gene that encodes a toxic protein.

Another aspect of this application provides a stable cell line for recombinant viral vector production, comprising at least one inducible promoter having a sequence of SEQ ID NO: 1 or SEQ ID NO: 3 operatively linked to at least one heterologous gene that encodes a toxic protein.

Another aspect of this application provides a stable cell line for recombinant viral vector production, comprising at least one inducible promoter having a sequence of any one of SEQ ID NO: 6 to SEQ ID NO: 9 operatively linked to at least one heterologous gene that encodes a toxic protein.

Another aspect of this application provides a stable cell line for recombinant viral vector production, comprising at least one inducible promoter having a sequence of SEQ ID NO: 1 or SEQ ID NO: 3 operatively linked to at least one heterologous gene that encodes a toxic protein, and at least one inducible promoter operatively linked to at least one heterologous gene that encodes a toxic protein, wherein the at least one inducible promoter is selected from the group consisting of: a forskolin inducible promoter, a hypoxia inducible promoter, a tetracycline inducible promoter, an alcohol inducible promoter, a steroid inducible promoter, an RU486 inducible promoter, an ecdysone inducible promoter, a rapamycin inducible promoter, a metallothionein inducible promoter, a hormone inducible promoter and a metal inducible promoter.

Another aspect of this application provides a stable cell line for recombinant viral vector production, comprising at least one inducible promoter having a sequence of any one of SEQ ID NO: 6 to SEQ ID NO: 9 operatively linked to at least one heterologous gene that encodes a toxic protein, and at least one inducible promoter operatively linked to at least one heterologous gene that encodes a toxic protein, wherein the at least one inducible promoter is selected from the group consisting of: a forskolin inducible promoter, a hypoxia inducible promoter, a tetracycline inducible promoter, an alcohol inducible promoter, a steroid inducible promoter, an RU486 inducible promoter, an ecdysone inducible promoter, a rapamycin inducible promoter, a metallothionein inducible promoter, a hormone inducible promoter and a metal inducible promoter.

Another aspect of this application provides a stable cell line for recombinant viral vector production, comprising at least one inducible promoter having a sequence of SEQ ID NO: 1 or SEQ ID NO: 3 operatively linked to at least one heterologous gene that encodes a toxic protein, and at least one inducible promoter having a sequence of any one of SEQ ID NO: 6 to SEQ ID NO: 9 operatively linked to at least one heterologous gene that encodes a toxic protein.

Another aspect of this application provides a stable cell line for rAAV production, comprising at least one inducible promoter operatively linked to a heterologous gene that encodes a toxic protein, wherein the at least one inducible promoter is selected from the group consisting of: a forskolin inducible promoter, a hypoxia inducible promoter, a tetracycline inducible promoter, an alcohol inducible promoter, a steroid inducible promoter, an RU486 inducible promoter, an ecdysone inducible promoter, a rapamycin inducible promoter, a metallothionein inducible promoter, a hormone inducible promoter and a metal inducible promoter.

Another aspect of this application provides a stable cell line for rAAV production, comprising at least one forskolin inducible promoter operatively linked to at least one heterologous gene that encodes a toxic protein.

Another aspect of this application provides a stable cell line for rAAV production, comprising at least one hypoxia inducible promoter operatively linked to at least one heterologous gene that encodes a toxic protein.

Another aspect of this application provides a stable cell line for rAAV production, comprising at least one forskolin inducible promoter operatively linked to at least one heterologous gene that encodes a toxic protein, and at least one inducible promoter operatively linked to at least one heterologous gene that encodes a toxic protein, wherein the at least one inducible promoter is selected from the group consisting of: a forskolin inducible promoter, a hypoxia inducible promoter, a tetracycline inducible promoter, an alcohol inducible promoter, a steroid inducible promoter, an RU486 inducible promoter, an ecdysone inducible promoter, a rapamycin inducible promoter, a metallothionein inducible promoter, a hormone inducible promoter and a metal inducible promoter.

Another aspect of this application provides a stable cell line for rAAV production, comprising at least one hypoxia inducible promoter operatively linked to at least one heterologous gene that encodes a toxic protein, and at least one inducible promoter operatively linked to at least one heterologous gene that encodes a toxic protein, wherein the at least one inducible promoter is selected from the group consisting of: a forskolin inducible promoter, a hypoxia inducible promoter, a tetracycline inducible promoter, an alcohol inducible promoter, a steroid inducible promoter, an RU486 inducible promoter, an ecdysone inducible promoter, a rapamycin inducible promoter, a metallothionein inducible promoter, a hormone inducible promoter and a metal inducible promoter.

Another aspect of this application provides a stable cell line for rAAV production, comprising at least one forskolin inducible promoter operatively linked to at least one heterologous gene that encodes a toxic protein, and at least one hypoxia inducible promoter operatively linked to at least one heterologous gene that encodes a toxic protein.

Another aspect of this application provides a stable cell line for rAAV production, comprising at least one inducible promoter having a sequence of SEQ ID NO: 1 or SEQ ID NO: 3 operatively linked to at least one heterologous gene that encodes a toxic protein.

Another aspect of this application provides a stable cell line for rAAV production, comprising at least one inducible promoter having a sequence of any one of SEQ ID NO: 6 to SEQ ID NO: 9 operatively linked to at least one heterologous gene that encodes a toxic protein.

Another aspect of this application provides a stable cell line for rAAV production, comprising at least one inducible promoter having a sequence of SEQ ID NO: 1 or SEQ ID NO: 3 operatively linked to at least one heterologous gene that encodes a toxic protein, and at least one inducible promoter operatively linked to at least one heterologous gene that encodes a toxic protein, wherein the at least one inducible promoter is selected from the group consisting of: a forskolin inducible promoter, a hypoxia inducible promoter, a tetracycline inducible promoter, an alcohol inducible promoter, a steroid inducible promoter, an RU486 inducible promoter, an ecdysone inducible promoter, a rapamycin inducible promoter, a metallothionein inducible promoter, a hormone inducible promoter and a metal inducible promoter.

Another aspect of this application provides a stable cell line for rAAV production, comprising at least one inducible promoter having a sequence of any one of SEQ ID NO: 6 to SEQ ID NO: 9 operatively linked to at least one heterologous gene that encodes a toxic protein, and at least one inducible promoter operatively linked to at least one heterologous gene that encodes a toxic protein, wherein the at least one inducible promoter is selected from the group consisting of: a forskolin inducible promoter, a hypoxia inducible promoter, a tetracycline inducible promoter, an alcohol inducible promoter, a steroid inducible promoter, an RU486 inducible promoter, an ecdysone inducible promoter, a rapamycin inducible promoter, a metallothionein inducible promoter, a hormone inducible promoter and a metal inducible promoter.

A stable cell line for rAAV production, comprising at least one inducible promoter having a sequence of SEQ ID NO: 1 or SEQ ID NO: 3 operatively linked to at least one heterologous gene that encodes a toxic protein, and at least one inducible promoter having a sequence of any one of SEQ ID NO: 6 to SEQ ID NO: 9 operatively linked to at least one heterologous gene that encodes a toxic protein.

In one embodiment, the stable cell further comprises at least one repressible element operatively linked to at least one heterologous gene that encodes a toxic protein.

In one embodiment, if the stable cell line has at least two inducible promoters, and the at least two inducible promoters are the same, e.g., the stable cell comprises two forskolin promoters. In one embodiment, the stable cell line has at least two inducible promoters, and the at least two inducible promoters are different, e.g., the stable cell comprises a forskolin and a hypoxia promoter.

A stable cell line described herein can comprise at least 1, 2, 3, 4, 5, or more inducible promoters operatively linked to distinct toxic genes. Alternatively, an inducible promoter can be operatively linked to at least 2, 3, 4, 5, or more genes. For example, an inducible promoter can be operatively linked to at least 2 different toxic genes, for example, rep and cap, such that inducing expression (e.g. by contacting the cell with an inducer for the inducible promoter) will result in expression of the at least 2 different toxic genes. An inducible promoter can be operatively linked to at least 2 different helper genes, such that inducing expression (e.g. by contacting the cell with an inducer for the inducible promoter) will result in expression of the at least 2 different helper genes. An inducible promoter can be operatively linked to at least one toxic protein and at least one different helper gene, such that inducing expression (e.g. by contacting the cell with an inducer for the inducible promoter) will result in expression of the at least one toxic protein and at least one different helper gene.

In one embodiment, the cell comprises at least two separate inducible promoters, wherein the at least two inducible promoters are induced by different inducers (e.g. by hypoxia and forskolin), and the at least two inducible promoters are operatively linked to distinct heterologous genes that encode different toxic proteins. For example, the cell comprises a first inducible promoter operatively linked to a first toxic gene, and a second inducible promoter operatively linked to a second toxic gene.

In one embodiment, the cell comprises at least two inducible promoters, wherein the at least two inducible promoters are induced by different inducers, and the at least two inducible promoters are induced by the same inducer. For example, the cell comprises a first inducible promoter operatively linked to a first toxic gene, and a second inducible promoter operatively linked to a second toxic gene.

In one embodiment, the cell comprises at least two inducible promoters, wherein the at least two inducible promoters are induced by different inducers, and the at least two inducible promoters are each operatively linked to at least one distinct heterologous genes that encode at least one toxic protein. For example, the cell comprises a first inducible promoter operatively linked to a first and second toxic gene, and a second inducible promoter operatively linked to a third toxic gene.

In one embodiment the stable cell line comprises the at least one inducible promoter operatively linked to a heterologous gene that encodes a toxic protein incorporated in the genome. In another embodiment the stable cell line comprises the at least one inducible promoter operatively linked to a heterologous gene that encodes a toxic protein in a stable episomal form (e.g. as a stable plasmid).

In one embodiment, a cell line for recombinant viral vector production, e.g., AAV vector production, comprising at least one inducible promoter need not be a stable cell line. For example, a vector comprising the at least one inducible promoter may be transiently introduced (e.g. transiently transfected) into the cell. One aspect of this application provides a cell line for recombinant viral vector production comprising transient introduction of at least one inducible promoter, wherein the inducible promoter is operatively linked to a heterologous gene that encodes a toxic protein. Another aspect of this application provides a cell line for rAAV vector production comprising transient introduction of at least one inducible promoter, wherein the inducible promoter is operatively linked to a heterologous gene that encodes a toxic protein required for rAAV production (e.g., rep or cap). Transient expression can be achieved by, e.g., introducing to a cell a synthetic nucleic acid, expression vector, or plasmid for expressing at least one inducible promoter, wherein the inducible promoter is operatively linked to a heterologous gene that encodes a toxic protein. Such introducing may involve, for example, transformation, transfection, electroporation, or lipofection. One skilled in the art can determine if a cell has transient introduction of a synthetic nucleic acid, expression vector, or plasmid by, e.g., using PCR-based assays or western-blotting to assess mRNA or protein levels of the synthetic nucleic acid, expression vector, or plasmid, respectively.

Another aspect provided herein is a cell expressing a nucleic acid construct encoding a tetracycline-responsive transactivator protein operatively linked to a constitutive promoter.

Another aspect provided herein is a cell expressing a nucleic sequence encoding a marker protein.

Another aspect provided herein is a cell expressing a nucleic acid construct comprising at least a nucleic acid sequence encoding a toxic protein, wherein the nucleic acid encoding a toxic protein is operatively linked to a regulatable promoter.

Another aspect provided herein is a cell expressing a nucleic acid construct comprising a nucleic acid sequence encoding at least one helper protein, wherein the at least one helper gene is operatively linked to a first regulatable promoter; and a nucleic acid sequence encoding a toxic protein, wherein the nucleic acid encoding the toxic protein is under the control of a second regulatable promoter or a zinc-finger transcriptional activator (ZF-TA).

Another aspect provided herein is a cell expressing a nucleic acid construct comprising a nucleic acid sequence comprising at least one of a nucleic acid sequence encoding a E4 protein, a nucleic acid sequence encoding a E2A protein, and a nucleic acid sequence encoding a VA protein, wherein each nucleic acid sequences encoding any one of E4, E2A, and VA RNA is operatively linked to a regulatable promoter.

Another aspect provided herein is a cell expressing a nucleic acid construct encoding a Rep protein, wherein the nucleic acid encoding a Rep protein is under the control of a regulatable promoter or a zinc-finger transcriptional activator (ZF-TA).

Another aspect provided herein is a cell expressing a nucleic acid construct comprising a nucleic acid sequence comprising at least one of: a nucleic acid sequence encoding a E4 protein, a nucleic acid sequence encoding a E2A protein, and a nucleic acid sequence encoding a VA protein, wherein each nucleic acid sequences encoding any one of E4, E2A, and VA RNA is operatively linked to a first regulatable promoter; and a nucleic acid sequence encoding a Rep protein, wherein the nucleic acid encoding a Rep protein is under the control of a second regulatable promoter or transcriptional activator, wherein the first and the second regulatable promoters are different. In one embodiment, the transcriptional activator is a zinc-finger transcriptional activator (ZF-TA).

Another aspect provided herein is a cell expressing a nucleic acid construct comprising a nucleic acid sequence encoding a zinc finger (ZF) transcriptional activator operatively linked to an inducible promoter, wherein the inducible promoter is a E4-responsive promoter, or a E2-responsive promoter or other helper-gene responsive promoter. In various embodiments, the zinc-finger transcriptional activator (ZF-TA) is expressed from this construct.

Another aspect provided herein is a cell expressing a nucleic acid construct comprising a target site for binding of a zinc-finger transcriptional activator (ZF-TA).

Another aspect provided herein is a cell expressing a nucleic acid construct comprising a Rep protein operatively linked to a promoter comprising a target site for binding of a zinc-finger transcriptional activator (ZF-TA).

Another aspect provided herein is a cell expressing a nucleic acid construct comprising a nucleic acid sequence encoding a tetracycline-responsive transactivator protein operatively linked to a constitutive promoter; a nucleic acid sequence comprising at least one of: a nucleic acid sequence encoding a E4 protein, a nucleic acid sequence encoding a E2A protein, and a nucleic acid sequence encoding a VA protein, wherein each nucleic acid sequences encoding any one of E4, E2A, and VA RNA is operatively linked to a regulatable promoter; and a nucleic acid sequence encoding, e.g., a zinc finger (ZF) transcriptional activator operatively linked to an inducible promoter, wherein the inducible promoter is a E4-responsive promoter, or a E2-responsive promoter; and a nucleic acid construct comprising a Rep protein operatively linked to a promoter comprising a target site for binding of a zinc-finger transcriptional activator (ZF-TA).

Another aspect provided herein is a cell expressing a nucleic acid construct comprising a nucleic acid sequence encoding a toxic protein and a recombinase recognition sequence (RRS) located 3′ of the nucleic acid sequence encoding the toxic protein.

Another aspect provided herein is a cell expressing a nucleic acid construct comprising a nucleic acid sequence encoding a Cap protein and a recombinase recognition sequence (RRS) located 5″ and 3′ of the nucleic acid sequence encoding the Cap protein, such that the RRSs are flanking the nucleic acid sequence encoding the Cap protein.

Another aspect provided herein is a cell expressing a nucleic acid construct comprising a first nucleic acid construct comprising in a 5′ to 3′ direction: a promoter, a stop nucleic acid sequence flanked by a first pair of recombinase recognition sequences (RRS), and nucleic acid sequence encoding a Rep protein, wherein the promoter is operatively linked to the nucleic acid encoding the Rep protein, and a second nucleic acid construct comprising, in a 5′ to 3′ direction: a promoter, a stop nucleic acid sequence flanked by a second pair of recombinase recognition sequences (RRSs), and nucleic acid sequence encoding one or more of E2A, E4, and VA RNA, wherein the promoter is operatively linked to the nucleic acid encoding the one or more of E2A, E4, and VA RNA.

Expression of the construct can be stably expressed, i.e., integrated into the genome of the cell. One aspect herein provides a stable cell expressing at least one nucleic acid construct described herein. In one embodiment, the stable cell expresses at least 2, at least 3, at least 4, at least 5, at least 6, at least 7, at least 8, or at least 9 of the nucleic acid constructs described herein.

One aspect provided herein is a stable cell expressing a nucleic acid construct comprising at least a nucleic acid sequence encoding at least one helper protein, wherein each nucleic acid construct is operatively linked to a regulatable promoter.

Another aspect provided herein is a stable cell expressing a nucleic acid construct encoding a tetracycline-responsive transactivator protein operatively linked to a constitutive promoter.

Another aspect provided herein is a stable cell expressing a nucleic sequence encoding a marker protein.

Another aspect provided herein is a stable cell expressing a nucleic acid construct comprising at least a nucleic acid sequence encoding a toxic protein, wherein the nucleic acid encoding a toxic protein is operatively linked to a regulatable promoter.

Another aspect provided herein is a stable cell expressing a nucleic acid construct comprising a nucleic acid sequence encoding at least one helper protein, wherein the at least one helper gene is operatively linked to a first regulatable promoter; and a nucleic acid sequence encoding a toxic protein, wherein the nucleic acid encoding the toxic protein is under the control of a second regulatable promoter or a zinc-finger transcriptional activator (ZF-TA).

Another aspect provided herein is a stable cell expressing a nucleic acid construct comprising a nucleic acid sequence comprising at least one of a nucleic acid sequence encoding a E4 protein, a nucleic acid sequence encoding a E2A protein, and a nucleic acid sequence encoding a VA protein, wherein each nucleic acid sequences encoding any one of E4, E2A, and VA RNA is operatively linked to a regulatable promoter.

Another aspect provided herein is a stable cell expressing a nucleic acid construct encoding a Rep protein, wherein the nucleic acid encoding a Rep protein is under the control of a regulatable promoter or a zinc-finger transcriptional activator (ZF-TA).

Another aspect provided herein is a stable cell expressing a nucleic acid construct comprising a nucleic acid sequence comprising at least one of: a nucleic acid sequence encoding a E4 protein, a nucleic acid sequence encoding a E2A protein, and a nucleic acid sequence encoding a VA protein, wherein each nucleic acid sequences encoding any one of E4, E2A, and VA RNA is operatively linked to a first regulatable promoter; and a nucleic acid sequence encoding a Rep protein, wherein the nucleic acid encoding a Rep protein is under the control of a second regulatable promoter or transcriptional activator, wherein the first and the second regulatable promoters are different

Another aspect provided herein is a stable cell expressing a nucleic acid construct comprising a nucleic acid sequence encoding a zinc finger (ZF) transcriptional activator operatively linked to an inducible promoter, wherein the inducible promoter is a E4-responsive promoter, or a E2-responsive promoter or other helper-gene responsive promoter. In various embodiments, the zinc-finger transcriptional activator (ZF-TA) is expressed from this construct.

Another aspect provided herein is a stable cell expressing a nucleic acid construct comprising a toxic protein operatively linked to a promoter comprising a zinc-finger transcriptional activator (ZF-TA).

Another aspect provided herein is a stable cell expressing a nucleic acid construct comprising a Rep protein operatively linked to a promoter comprising a target site for binding of a transcriptional activator, e.g., a zinc-finger transcriptional activator (ZF-TA).

Another aspect provided herein is a stable cell expressing a nucleic acid construct comprising a nucleic acid sequence encoding a tetracycline-responsive transactivator protein operatively linked to a constitutive promoter; a nucleic acid sequence comprising at least one of: a nucleic acid sequence encoding a E4 protein, a nucleic acid sequence encoding a E2A protein, and a nucleic acid sequence encoding a VA protein, wherein each nucleic acid sequences encoding any one of E4, E2A, and VA RNA is operatively linked to a regulatable promoter; and a nucleic acid sequence encoding a zinc finger (ZF) transcriptional activator operatively linked to an inducible promoter, wherein the inducible promoter is a E4-responsive promoter, or a E2-responsive promoter; and a nucleic acid construct comprising a Rep protein operatively linked to a promoter comprising a target site for binding of a zinc-finger transcriptional activator (ZF-TA).

Another aspect provided herein is a stable cell expressing a nucleic acid construct comprising a nucleic acid sequence encoding a toxic protein and a recombinase recognition sequence (RRS) located 3′ of the nucleic acid sequence encoding the toxic protein.

Another aspect provided herein is a stable cell expressing a nucleic acid construct comprising a nucleic acid sequence encoding a Cap protein and a recombinase recognition sequence (RRS) located 3′ of the nucleic acid sequence encoding the Cap protein.

Another aspect provided herein is a stable cell expressing a nucleic acid construct comprising a first nucleic acid construct comprising in a 5′ to 3′ direction: a promoter, a stop nucleic acid sequence flanked by a first pair of recombinase recognition sequences (RRS), and nucleic acid sequence encoding a Rep protein, wherein the promoter is operatively linked to the nucleic acid encoding the Rep protein, and a second nucleic acid construct comprising, in a 5′ to 3′ direction: a promoter, a stop nucleic acid sequence flanked by a second pair of recombinase recognition sequences (RRSs), and nucleic acid sequence encoding one or more of E2A, E4, and VA RNA, wherein the promoter is operatively linked to the nucleic acid encoding the one or more of E2A, E4, and VA RNA.

Expression of the construct can be transiently expressed, i.e., not integrated into the genome of the cell. One aspect herein provides a cell having transient expression of at least one nucleic acid construct described herein. In one embodiment, the cell has transient expression of at least 2, at least 3, at least 4, at least 5, at least 6, at least 7, at least 8, or at least 9 of the nucleic acid constructs described herein.

One aspect provided herein is a cell having transient expression of a nucleic acid construct comprising at least a nucleic acid sequence encoding at least one helper protein, wherein each nucleic acid construct is operatively linked to a regulatable promoter.

Another aspect provided herein is a cell having transient expression of a nucleic acid construct encoding a tetracycline-responsive transactivator protein operatively linked to a constitutive promoter.

Another aspect provided herein is a cell having transient expression of a nucleic sequence encoding a marker protein.

Another aspect provided herein is a cell having transient expression of a nucleic acid construct comprising at least a nucleic acid sequence encoding a toxic protein, wherein the nucleic acid encoding a toxic protein is operatively linked to a regulatable promoter.

Another aspect provided herein is a cell having transient expression of a nucleic acid construct comprising a nucleic acid sequence encoding at least one helper protein, wherein the at least one helper gene is operatively linked to a first regulatable promoter; and a nucleic acid sequence encoding a toxic protein, wherein the nucleic acid encoding the toxic protein is under the control of a second regulatable promoter or a zinc-finger transcriptional activator (ZF-TA).

Another aspect provided herein is a cell having transient expression of a nucleic acid construct comprising a nucleic acid sequence comprising at least one of a nucleic acid sequence encoding a E4 protein, a nucleic acid sequence encoding a E2A protein, and a nucleic acid sequence encoding a VA protein, wherein each nucleic acid sequences encoding any one of E4, E2A, and VA RNA is operatively linked to a regulatable promoter.

Another aspect provided herein is a cell having transient expression of a nucleic acid construct encoding a Rep protein, wherein the nucleic acid encoding a Rep protein is under the control of a regulatable promoter or a zinc-finger transcriptional activator (ZF-TA).

Another aspect provided herein is a cell having transient expression of a nucleic acid construct comprising a nucleic acid sequence comprising at least one of: a nucleic acid sequence encoding a E4 protein, a nucleic acid sequence encoding a E2A protein, and a nucleic acid sequence encoding a VA protein, wherein each nucleic acid sequences encoding any one of E4, E2A, and VA RNA is operatively linked to a first regulatable promoter; and a nucleic acid sequence encoding a Rep protein, wherein the nucleic acid encoding a Rep protein is under the control of a second regulatable promoter or transcriptional activator, wherein the first and the second regulatable promoters are different.

Another aspect provided herein is a cell having transient expression of a nucleic acid construct comprising a nucleic acid sequence encoding a zinc finger (ZF) transcriptional activator operatively linked to an inducible promoter, wherein the inducible promoter is a E4-responsive promoter, or a E2-responsive promoter or other helper-gene responsive promoter. In various embodiments, the zinc-finger transcriptional activator (ZF-TA) is expressed from this construct.

Another aspect provided herein is a cell having transient expression of a nucleic acid construct comprising a toxic protein operatively linked to a promoter comprising a zinc-finger transcriptional activator (ZF-TA).

Another aspect provided herein is a cell having transient expression of a nucleic acid construct comprising a Rep protein operatively linked to a promoter comprising a target site for binding of a transcriptional activator, e.g., a zinc-finger transcriptional activator (ZF-TA).

Another aspect provided herein is a cell having transient expression of a nucleic acid construct comprising a nucleic acid sequence encoding a tetracycline-responsive transactivator protein operatively linked to a constitutive promoter; a nucleic acid sequence comprising at least one of: a nucleic acid sequence encoding a E4 protein, a nucleic acid sequence encoding a E2A protein, and a nucleic acid sequence encoding a VA protein, wherein each nucleic acid sequences encoding any one of E4, E2A, and VA RNA is operatively linked to a regulatable promoter; and a nucleic acid sequence encoding a zinc finger (ZF) transcriptional activator operatively linked to an inducible promoter, wherein the inducible promoter is a E4-responsive promoter, or a E2-responsive promoter; and a nucleic acid construct comprising a Rep protein operatively linked to a promoter comprising a target site for binding of a zinc-finger transcriptional activator (ZF-TA).

Another aspect provided herein is a cell having transient expression of a nucleic acid construct comprising a nucleic acid sequence encoding a toxic protein and a recombinase recognition sequence (RRS) located 3′ of the nucleic acid sequence encoding the toxic protein.

Another aspect provided herein is a cell having transient expression of a nucleic acid construct comprising a nucleic acid sequence encoding a Cap protein and a recombinase recognition sequence (RRS) located 3′ of the nucleic acid sequence encoding the Cap protein.

Another aspect provided herein is a cell having transient expression of a nucleic acid construct comprising a first nucleic acid construct comprising in a 5′ to 3′ direction: a promoter, a stop nucleic acid sequence flanked by a first pair of recombinase recognition sequences (RRS), and nucleic acid sequence encoding a Rep protein, wherein the promoter is operatively linked to the nucleic acid encoding the Rep protein, and a second nucleic acid construct comprising, in a 5′ to 3′ direction: a promoter, a stop nucleic acid sequence flanked by a second pair of recombinase recognition sequences (RRSs), and nucleic acid sequence encoding one or more of E2A, E4, and VA RNA, wherein the promoter is operatively linked to the nucleic acid encoding the one or more of E2A, E4, and VA RNA.

If a cell expresses at least two nucleic acid constructs, the type of expression, i.e., stable or transient, can be the same or different for each of the at least two nucleic acid constructs. For example, if a cell expresses at least two nucleic acid constructs, the expression of the at least two nucleic acid constructs can both be stable expression. If a cell expresses at least two nucleic acid constructs, the expression of the at least two nucleic acid constructs can both be transient expression. Alternatively, if a cell expresses at least two nucleic acid constructs, the expression of at least one of the two nucleic acid constructs is stable expression, e.g., a cell can have both stable expression of a nucleic acid construct, and transient expression of a different nucleic acid construct.

Cell culture systems used for viral vector propagation, such as viral vector producer cells—which include primary cells, semi-continuous cell lines, and continuous cell lines—can express any of the inducible promoters described herein to control a gene product required for viral vector propagation. Primary cell lines are cell lines derived from animal tissues, such as human, mouse, canine, monkey, and the like, and may be passaged once or twice to generate secondary cultures (i.e., subculture of the primary culture), but grow only for limited time. Secondary cultures are similar to primary cultures in both morphology and viral susceptibility. Semi-continuous cell lines, e.g., human diploid cells, are derived from fetal tissues, and can be sub-cultured for ˜50 passages. Continuous cell lines are cancer or other immortalized cell lines, which multiply rapidly and may be cultured indefinitely. These cells may become heteroploid on serial passage. Continuous cell lines typically have a narrower range of viral susceptibility as compared to primary cells and semi-continuous cell lines, but are easy to adapt for viral propagation.

Cell lines for propagating viral vectors are known in the art, and include, but are not limited to, the exemplary cell lines presented in Table 2. In one embodiment, the cell line for viral propagation is selected from Table 2, In one embodiment, the cell line for viral propagation is derived from a cell line selected from Table 2.

TABLE 2
Cell lines for propagating viral vectors.

| CELL LINE | ORIGIN | EXEMPLARY VIRUSES PROPAGATED |
|---|---|---|
| HEK293 | human embryonic kidney | Adenovirus, Adeno-associated virus (AAV), |
| | cells | lentivirus, retrovirus, influenza-like, |
| CHO | Chinese hamster ovary | lentivirus, retrovirus, |
| A549 | human lung carcinoma | Adenovirus, HSV, influenza, measles, mumps, |
| | | parainfluenza, poliovirus, respiratory syncytial |
| | | virus (RSV), rotavirus, Varicella zoster virus |
| | | (VZV), metapneumovirus (MPV) |
| BHK 21 | Syrian hamster kidney | Human adenovirus D, reovirus 3, vesicular |
| (clone 13) | | stomatitis virus (Indiana strain), Dengue, influenza, |
| | | rabies, foot and mouth, |
| | | rubellahttps://www.sigmaaldrich.com/technical- |
| | | documents/articles/biology/cell-culture/virus- |
| | | cultivation.html - ref |
| CV-1 | African green monkey | RSV, measles, HSV, VZV |
| | kidney fibroblast | |
| HeLa | human cervix | Poliovirus type I, adenovirus type 3, CMV, |
| | adenocarcinoma | echovirus, HSV, poliovirus, |
| | | rhinovirus, https://www.sigmaaldrich.com/technical- |
| | | documents/articles/biology/cell-culture/virus- |
| | | cultivation.html - ref vesicular stomatitis (Indiana |
| | | Strain) virus, |
| | | VZVhttps://www.sigmaaldrich.com/technical- |
| | | documents/articles/biology/cell-culture/virus- |
| | | cultivation.html - ref |
| LLCMK2 | Rhesus monkey kidney | Poliovirus type 1, enterovirus, rhinovirus, poxvirus |
| | | groups |
| McCoy | Mouse fibroblast | HSV |
| MDCK | Madin-Darby canine | Influenza A, influenza B, some types of adenovirus, |
| | kidney | reoviruses |
| MRC-5 | human fetal lung | CMV, https://www.sigmaaldrich.com/technical- |
| | | documents/articles/biology/cell-culture/virus- |
| | | cultivation.html - ref HSV, adenovirus, influenza, |
| | | mumps, https://www.sigmaaldrich.com/technical- |
| | | documents/articles/biology/cell-culture/virus- |
| | | cultivation.html - ref echovirus, poliovirus, |
| | | rhinovirus, RSV, VZV |
| NCI-H292 | Human lung, | Vaccinia |
| | mucoepidermoid | virus, https://www.sigmaaldrich.com/technical- |
| | | documents/articles/biology/cell-culture/virus- |
| | | cultivation.html - ref HSV, adenovirus, measles |
| | carcinoma | virus, https://www.sigmaaldrich.com/technical- |
| | | documents/articles/biology/cell-culture/virus- |
| | | cultivation.html - ref reoviruses, BK polyomavirus, |
| | | RSV, some strains of influenza A, most |
| | | enteroviruses, and rhinoviruses |
| Vero | African green monkey | Coxsackie B, HSV, measles, mumps, poliovirus |
| | kidney | type 3, https://www.sigmaaldrich.com/technical- |
| | | documents/articles/biology/cell-culture/virus- |
| | | cultivation.html - ref rotavirus, rubella |
| Vero76 | African green monkey | Coxsackie B, HSV, West Nile virus |
| | kidney | |
| Wi 38 | Human fetal lung | Adenovirus, |
| | | CMV, https://www.sigmaaldrich.com/technical- |
| | | documents/articles/biology/cell-culture/virus- |
| | | cultivation.html - ref |
| | | echovirus, https://www.sigmaaldrich.com/technical- |
| | | documents/articles/biology/cell-culture/virus- |
| | | cultivation.html - ref |
| | | HSV, https://www.sigmaaldrich.com/technical- |
| | | documents/articles/biology/cell-culture/virus- |
| | | cultivation.html - ref |
| | | mumps, https://www.sigmaaldrich.com/technical- |
| | | documents/articles/biology/cell-culture/virus- |
| | | cultivation.html - ref |
| | | influenza, https://www.sigmaaldrich.com/technical- |
| | | documents/articles/biology/cell-culture/virus- |
| | | cultivation.html - ref |
| | | rhinovirus, https://www.sigmaaldrich.com/technical- |
| | | documents/articles/biology/cell-culture/virus- |
| | | cultivation.html - ref |
| | | RSV, https://www.sigmaaldrich.com/technical- |
| | | documents/articles/biology/cell-culture/virus- |
| | | cultivation.html - ref VZV |
| A549 | human lung carcinoma | Adenovirus, HSV, influenza, measles, mumps, |
| | | parainfluenza, poliovirus, respiratory syncytial |
| | | virus (RSV), |
| | | rotavirus, https://www.sigmaaldrich.com/technical- |
| | | documents/articles/biology/cell-culture/virus- |
| | | cultivation.html - ref Varicella zoster virus (VZV), |
| | | metapneumovirus (MPV) |
| Sf9 | Insect cells | Baculovirus, AAV |
| HepG2 | Human hepatocellular | lentivirus |
| | carcinoma | |
| MCF-7 | Human invasive breast | Rubella virus |
| | ductal carcinoma | |
| MEF | Mouse embryonic | mouse cytomegalovirus |
| | fibroblast | |
| NS0 | nonsecreting murine | lentivirus |
| | myeloma | |
| HUVEC | Human umbilical vein | Human cytomegalovirus, zika virus, Kaposi's |
| | endothelial cells | sarcoma-associated herpesvirus (KSHV) |
| Jurkat | human T lymphocyte | retrovirus |
| Cos-7 | CV-1 African green | SV-40 monkey virus, Simian immunodeficiency |
| | monkey fibroblast | virus |
| 3T3 | Swiss albino mouse | Retrovirus, murine stem cell virus |
| | embryo tissue | |
| HL60 | Human leukemia | HIV, Epstein-Barr virus, lentivirus, poliovirus |
| ML-1 | Human acute | Lentivirus, poliovirus |
| | myeloblastic leukemia | |
| KG-1 | Human bone marrow | Retrovirus, poliovirus, HIV, dengue virus (DENV) |
| | aspirate | |
| U-937 | Human histiocytic | Poliovirus, HIV |
| | lymphoma | |
| THP-1 | Human acute monocytic | Poliovirus, HIV |
| | leukemia | |
| K-562 | Human immortalized | poliovirus |
| | myelogenous leukemia | |
| Molt-4 | Human T-cell acute | Poliovirus, feline immunodeficiency virus |
| | lymphoblastic leukemia | |
| TF-1 | Human Erythroleukemia | HIV |

In one embodiment, the cell line is an HEK293 cell line that has been modified such that it is no longer an adhesive cell line. In one embodiment, the cell line is a HEK293 cell line which grows in suspension. In one embodiment, the cell line is the Pro10 cell line. The Pro10 cell line is described, e.g., in U.S. Pat. No. 9,441,206, which is incorporated herein by reference.

Further provided herein is a method of producing any of the stable cell lines described herein comprising (a) transforming a population of cells with at least one nucleic acid cassette containing an inducible promoter operatively linked to a heterologous gene that encodes a toxic protein, or a nucleic acid described herien (b) culturing the population of cells of (a) under conditions and for a time sufficient to permit expression of the nucleic acid cassette or construct, (c) selecting for a cell that stably expresses the nucleic acid cassette, and (d) growing the cell of (c) to produce the cell line.

Techniques and methods for making stable cell lines are known in the art, and can be readily identified by a skilled person. In one embodiment, the parent cells were co-introduced with a vector comprising a gene that confers antibiotic resistant to the cell. For example, the bsr, bls, or BSD genes confer resistance to Blasticidin; the Sh ble gene confers resistance to Zeocin™; the pac gene confers resistance to Puromycin; the neo gene confers resistance to G418 (Geneticin); the hph gene confers resistance to Hygromycin B; and the Sh ble gene confers resistance to Phleomycin. For the initial selection, cells expressing a gene that confers resistance to an antibiotic are cultured in the presence of the antibiotic. For example, a cell expressing the neo gene is cultured in a geneticin-containing medium. After initial selection, surviving cells are isolated, cultured under conditions permitting expression of the nucleic acid cassette, harvested, and assayed to confirm expression of the nucleic acid cassette.

In one embodiment, the method further comprises culturing the cells under conditions and for a time sufficient to induce expression of the at least one toxic protein, for example, Rep, Cap, or a helper protien. Western blotting or other suitable assays may be used to assess expression of the nucleic acid cassette or at least one toxic protein. Inducing expression suitable involved applying at least one inducer to said cells. The inducer is applied for a suitable period of time to induce expression from the inducible promoter. As discussed herein, the inducer can be an agent which is administered to said cells, or can be a condition to which the cells are subjected. Suitable inducers are discussed herein.

Clones producing the nucleic acid cassette are expanded to produce the inventive stable cell lines (the term “cell line” is intended to include progeny (subclones) of an original line). In one embodiment, stability is confirmed by the ability to produce the at least one toxic protein over at least about 12 months, and for greater than about 50 passages. In one embodiment, stability is confirmed by the ability to produce the at least one toxic protein over at least about 1, 2, 3, 4, 5, 6, 7, 8, 9, 10, 11, 13, 14, 15, 16, 17, 18, 19, 20, 21, 22, 23, 24, or more months, and for greater than about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more passages.

### Regulatable Transcriptional Activators

The or each nucleic acid sequence encoding a Rep protein, is under the control of a second regulatable promoter or a regulatable transcriptional activator. In one embodiment of any aspect, the second regulatable promoter operatively linked to the or each nucleic acid encoding the or each Rep protein comprises a binding site for a regulatable transcriptional activator.

Exemplary transcriptional activators include homeodomain transcriptional activator, zinc-finger transcriptional activator, winged-helix (Forkhead) transcriptional activator, leucine-zipper transcriptional activator, and helix-loop-helix transcriptional activator. In one embodiment, the regulatable transcriptional activator is a zinc-finger transcriptional activator (ZF-TA). In one embodiment of any aspect, the second regulatable promoter comprises a binding site for a zinc-finger transcriptional activator (ZF-TA).

In one embodiment of any aspect, the zinc-finger transcriptional activator (ZF-TA) is expressed from a nucleic acid construct encoding a zinc finger (ZF) transcriptional activator operatively linked to an inducible promoter, optionally wherein the inducible promoter is a E4-responsive promoter, or a E2-responsive promoter or other helper-gene responsive promoter.

In one embodiment of any aspect, the second regulatable promoter operatively linked to the or each nucleic acid encoding the or each Rep protein comprises a binding site for a zinc finger (ZF) transcriptional activator.

In one embodiment of any aspect, the zinc-finger transcriptional activator (ZF-TA) is encoded by the following nucleic acid sequence:

(SEQ ID NO: 47)
ATGCTGGAGCCCGGCGAGAAGCCCTACAAGTGCCCCGAGTGCGGCAAGAG
CTTCAGCAGCCCCGCCGACCTGACCAGGCACCAGAGGACCCACACCGGCG
AGAAGCCCTACAAGTGCCCCGAGTGCGGCAAGAGCTTCAGCGACAAGAAG
GACCTGACCAGGCACCAGAGGACCCACACCGGCGAGAAGCCCTACAAGTG
CCCCGAGTGCGGCAAGAGCTTCAGCAGGAACGACGCCCTGACCGAGCACC
AGAGGACCCACACCGGCGAGAAGCCCTACAAGTGCCCCGAGTGCGGCAAG
AGCTTCAGCGAGAGGAGCCACCTGAGGGAGCACCAGAGGACCCACACCGG
CGAGAAGCCCTACAAGTGCCCCGAGTGCGGCAAGAGCTTCAGCGAGAGGA
GCCACCTGAGGGAGCACCAGAGGACCCACACCGGCGAGAAGCCCTACAAG
TGCCCCGAGTGCGGCAAGAGCTTCAGCGAGAGGAGCCACCTGAGGGAGCA
CCAGAGGACCCACACCGGCGAGAAGCCCTACAAGTGCCCCGAGTGCGGCA
AGAGCTTCAGCGACTGCAGGGACCTGGCCAGGCACCAGAGGACCCACACC
GGCGAGAAGCCCTACAAGTGCCCCGAGTGCGGCAAGAGCTTCAGCCACAG
GACCACCCTGACCAACCACCAGAGGACCCACACCGGCGAGAAGCCCTACA
AGTGCCCCGAGTGCGGCAAGAGCTTCAGCACCCACCTGGACCTGATCAGG
CACCAGAGGACCCACACCGGCGAGAAGCCCTACAAGTGCCCCGAGTGCGG
CAAGAGCTTCAGCGACTGCAGGGACCTGGCCAGGCACCAGAGGACCCACA
CCGGCGAGAAGCCCTACAAGTGCCCCGAGTGCGGCAAGAGCTTCAGCAGG
AGCGACAAGCTGGTGAGGCACCAGAGGACCCACACCGGCGAGAAGCCCTA
CAAGTGCCCCGAGTGCGGCAAGAGCTTCAGCAGGAGGGACGAGCTGAACG
TGCACCAGAGGACCCACACCGGCGAGAAGCCCTACAAGTGCCCCGAGTGC
GGCAAGAGCTTCAGCCAGAAGAGCAGCCTGATCGCCCACCAGAGGACCCA
CACCGGCAAGAAGACCAGCCCGAAAAAGAAACGCAAAGTTGGGCGCGCCG
ACGCGCTGGACGATTTCGATCTCGACATGCTGGGTTCTGATGCCCTCGAT
GACTTTGACCTGGATATGTTGGGAAGCGACGCATTGGATGACTTTGATCT
GGACATGCTCGGCTCCGATGCTCTGGACGATTTCGATCTCGATATGTTAA
TTAACTAA

In one embodiment, the inducible promoter is a E4-responsive promoter, or a E2-responsive promoter or other helper-gene responsive promoter.

In one embodiment, the binding site for a zinc-finger transcriptional activator (ZF-TA) binding sites comprises the nucleic acid sequence:

(SEQ ID NO: 48)
TCTAGAGGGTATATAATGGGGGCCACTAGTGCCAGCAGCAGCCTGACCAC
ATCTCATCCTCCAGCCACC

In one embodiment, the second regulatable promoter comprising a ZF-TA binding site operatively linked to the or each nucleic acid encoding the or each Rep protein comprises the following nucleic acid sequence, wherein the ZF-TA binding site is shown in bold:

(SEQ ID NO: 49)
TCTAGAGGGTATATAATGGGGGCCACTAGTGCCAG
CAGCAGCCTGACCACATCTCATCCTCCAGCCACC
ATGCCGGGGTTTTACGAGATTGTGATTAAGGTCCCCAGCGACCTTGACGA
GCATCTGCCCGGCATTTCTGACAGCTTTGTGAACTGGGTGGCCGAGAAGG
AGTGGGAGTTGCCGCCAGATTCTGACTTGGATCTGAATCTGATTGAGCAG
GCACCCCTGACCGTGGCCGAGAAGCTGCAGCGCGACTTTCTGACGGAGTG
GCGCCGTGTGAGTAAGGCCCCGGAGGCCCTTTTCTTTGTGCAATTTGAGA
AGGGAGAGAGCTACTTCCACTTACACGTGCTCGTGGAAACCACCGGGGTG
AAATCCTTAGTTTTGGGACGTTTCCTGAGTCAGATTCGCGAAAAACTGAT
TCAGAGAATTTACCGCGGGATCGAGCCGACTTTGCCAAACTGGTTCGCGG
TCACAAAGACCAGAAACGGCGCCGGAGGCGGGAACAAGGTGGTGGACGAG
TGCTACATCCCCAATTACTTGCTCCCCAAAACCCAGCCTGAGCTCCAGTG
GGCGTGGACTAATATAGAACAGTATTTAAGCGCCTGTTTGAATCTCACGG
AGCGTAAACGGTTGGTGGCGCAGCATCTGACGCACGTGTCGCAGACGCAG
GAGCAGAACAAAGAGAATCAGAATCCCAATTCTGACGCGCCGGTGATCAG
ATCAAAAACTTCAGCCAGGTACATGGAGCTGGTCGGGTGGCTCGTGGACA
AGGGGATTACCTCGGAGAAGCAGTGGATCCAGGAGGACCAGGCCTCATAC
ATCTCCTTCAATGCGGCCTCCAACTCGCGGTCCCAAATCAAGGCTGCCTT
GGACAATGCGGGAAAGATTATGAGCCTGACTAAAACCGCCCCCGACTACC
TGGTGGGCCAGCAGCCCGTGGAGGACATTTCCAGCAATCGGATTTATAAA
ATTTTGGAACTAAACGGGTACGATCCCCAATATGCGGCTTCCGTCTTTCT
GGGATGGGCCACGAAAAAGTTCGGCAAGAGGAACACCATCTGGCTGTTTG
GGCCTGCAACTACCGGGAAGACCAACATCGCGGAGGCCATAGCCCACACT
GTGCCCTTCTACGGGTGCGTAAACTGGACCAATGAGAACTTTCCCTTCAA
CGACTGTGTCGACAAGATGGTGATCTGGTGGGAGGAGGGGAAGATGACCG
CCAAGGTCGTGGAGTCGGCCAAAGCCATTCTCGGAGGAAGCAAGGTGCGC
GTGGACCAGAAATGCAAGTCCTCGGCCCAGATAGACCCGACTCCCGTGAT
CGTCACCTCCAACACCAACATGTGCGCCGTGATTGACGGGAACTCAACGA
CCTTCGAACACCAGCAGCCGTTGCAAGACCGGATGTTCAAATTTGAACTC
ACCCGCCGTCTGGATCATGACTTTGGGAAGGTCACCAAGCAGGAAGTCAA
AGACTTTTTCCGGTGGGCAAAGGATCACGTGGTTGAGGTGGAGCATGAAT
TCTACGTCAAAAAGGGTGGAGCCAAGAAAAGACCCGCCCCCAGTGACGCA
GATATAAGTGAGCCCAAACGGGTGCGCGAGTCAGTTGCGCAGCCATCGAC
GTCAGACGCGGAAGCTTCGATCAACTACGCAGACAGGTACCAAAACAAAT
GTTCTCGTCACGTGGGCATGAATCTGATGCTGTTTCCCTGCAGACAATGC
GAGAGAATGAATCAGAATTCAAATATCTGCTTCACTCACGGACAGAAAGA
CTGTTTAGAGTGCTTTCCCGTGTCAGAATCTCAACCCGTTTCTGTCGTCA
AAAAGGCGTATCAGAAACTGTGCTACATTCATCATATCATGGGAAAGGTG
CCAGACGCTTGCACTGCCTGCGATCTGGTCAATGTGGATTTGGATGACTG
CATCTTTGAACAATAA

### Regulatable Promoters

The at least one regulatable is an inducible promoter. The inducible promoter may be a promoter induced by the presence of an inducer, the absence of a repressor, or any other suitable physical or chemical condition that induces transcription from the inducible promoter. The terms “inducer”, “inducing conditions” and suchlike should be understood accordingly.

By way of non-limiting example, an inducible promoter for use in embodiments of the invention may be a forskolin-inducible promoter, a hypoxia-inducible promoter, a small molecule-inducible promoter, a tetracycline-regulatable (e.g. inducible or repressible) promoter, an alcohol-inducible promoter, a steroid-inducible promoter, a mifepristone (RU486)-inducible promoter, an ecdysone-inducible promoter, a rapamycin-inducible promoter, a metallothionein-inducible promoter, a hormone-inducible promoter, a cumate-inducible promoter, a temperature-inducible promoter, a pH-inducible promoter and a metal-inducible promoter. In one embodiment, an inducible promoter for use in embodiments of the invention is a forskolin-inducible promoter or a hypoxia-inducible promoter.

As will be discussed further below, various suitable inducible promoters have been described in the art and others are discussed herein. The person skilled in the art will be able to select one or more suitable inducible promoters for use in the various embodiments of the invention.

Furthermore, the person skilled in the art will be able to induce expression from said promoters as required based on the teachings herein or teachings present in the prior art.

Regulatable Intron

In some embodiments, the present invention may make use of at least one regulatable intron. The at least one regulatable intron may be operable to control expression of one or more proteins. The at least one regulatable intron may be used alone or in combination with one or more of the regulatable promoters described herein.

Suitably, the regulatable intron controls expression at the translation stage. By combining a regulatable promoter, such as an inducible promoter, and the regulatable intron of the present invention, a dual level of expression can be achieved, i.e. control at both the transcription level and at the translation level. This can allow for very tight control of expression of a gene, e.g. to avoid any expression “leakage”, i.e., expression in an unintended location, for example, a tissue or organ.

In one embodiment, said regulatable intron is an intron which comprises an excisable sequence which is capable of being spliced out of a transcript produced from the nucleic acid sequence via the unfolded protein response (UPR) system in the cell, thereby resulting in a transcript encoding a functional protein. The unfolded protein response (UPR) is a cellular coping mechanism for endoplasmic reticulum stress which is highly conserved across all eukaryotes.

In one embodiment, a nucleic acid construct of the present invention may comprise a sequence which encodes a regulatable intron, suitably a nucleic acid sequence which encodes a regulatable intron. In one embodiment, a nucleic acid sequence of the present invention which encodes a protein may comprise a sequence which encodes a regulatable intron. In one embodiment, a nucleic acid construct of the present invention may comprise a nucleic acid sequence encoding a protein, wherein the nucleic acid sequence encoding the protein comprises a sequence which encodes a regulatable intron.

A nucleic acid sequence encoding a regulatable intron may be present in any nucleic acid sequence encoding any protein described herein. In one embodiment the nucleic acid sequence encoding any of the following: an E4 protein, an E2A protein, a Rep protein, and a Cap protein may comprise a sequence which encodes a regulatable intron. In one embodiment, the nucleic acid sequence encoding the Rep protein may comprise a sequence encoding a regulatable intron. In one embodiment, there is provided a nucleic acid sequence encoding a Rep protein, wherein the nucleic acid sequence encoding the Rep protein comprises a sequence which encodes a regulatable intron.

In one embodiment, a cell of the present invention may comprise a nucleic acid sequence or a nucleic acid construct comprising a sequence which encodes a regulatable intron, as described above. In one embodiment the cell is a stable cell as described herein.

In one embodiment, a method of producing viral particles of the present invention may comprise providing and culturing a cell which comprises a nucleic acid sequence or a nucleic acid construct comprising a sequence which encodes a regulatable intron as described above. In one embodiment, said methods further comprise a step of treating the cell to induce UPR, to thereby induce splicing of the excisable sequence out of the regulatable intron. In one embodiment treating the cell to induce UPR comprises applying stress to the cell. For example, by administering a chemical agent to the cell, such as forskolin, dithiothreitol (DTT), tunicamycin, thapsigargin, a saturated fatty acid, an agent that is able to downregulate stearoyl-CoA desaturase enzyme activity, or by applying hypoxia, carbohydrate deprivation, or the like, to the cell.

Advantageously, the un-spliced transcript produced from the nucleic acid sequence encoding the protein encodes a truncated or otherwise defective version of the protein as a result of the presence of the regulatable intron, but when the transcript is processed by the UPR mechanism in the cell the excisable sequence of the intron is spliced out and the functional protein can be produced from the transcript. Thus, splicing out of the regulatable intron results in a functional mRNA encoding the functional protein expression product.

In one embodiment, the regulatable intron is capable of being spliced out by the IRE1 protein or a homologue or orthologue thereof, which is already present in the cell, (homologues or orthologues of IRE1 are present in all eukaryotes, including fungi, plants and mammals).

In some embodiments the regulatable intron is the XBP1 intron, the Hac1 intron, the bZIP60 intron, or a homologue thereof. By this it is meant that the intron can be a wild type form of the XBP1, Hac1 or bZIP60 intron, or a naturally occurring homologue thereof.

In one embodiment the regulatable intron comprises the sequence CNG/CNG-Xn-CNG/CNG, wherein Xn represents a sequence of length n bases, wherein/represents a cleavage site and wherein the sequence CNG-Xn-CNG is excised from the transcript. Thus, in other words, the regulatable intron suitably comprises a central sequence (Xn) flanked by two splice site target sequences, each having the sequence CNG/CNG, wherein/represents a cleavage site.

CNG/CNG is a consensus splice site sequence targeted in a highly-conserved manner by the UPR system in eukaryotic cells. As is known in the art, this splice site consensus sequence is targeted by the IRE1 protein (homologues or orthologues of which are present in all eukaryotes, including fungi, plants and mammals) when the UPR response is induced.

In various embodiments the regulatable intron or Xn can be from 10 to 500 nucleotides in length, 15 to 350 nucleotides in length, 15 to 100 nucleotides in length, 15 to 35 nucleotides in length, 20 to 25 nucleotides in length. The excisable sequence of the regulatory intron thus suitably has a length of 16 to 506 nucleotides, 21 to 356 nucleotides in length, 21 to 106 nucleotides in length, 21 to 41 nucleotides in length, 26 to 31 nucleotides in length.

There is considerable freedom regarding the specific sequence of Xn. Examples are set out below, but many other variants could of course be used provided that the regulatable intron remains functional, i.e. it is spliced out of the transcript by the UPR system at suitable levels.

In some embodiments, the regulatable intron comprises the sequence CNG/CNG-Xn-CNG/CNG[CG] (SEQ ID NO: 50), wherein Xn represents a sequence of length n nucleotides, wherein/represents the cleavage site such that the excisable sequence CNG-Xn-CNG (SEQ ID NO: 51) is excised from the transcript upon splicing, and wherein the nucleotide at the 5′ end of the sequence Xn is a C or G.

In some embodiments Xn comprises or consists of the sequence CACUCAGACUACGUGCACCU (SEQ ID NO: 52) or a sequence which is at least 60% identical thereto at least 70% identical thereto, at least 80% identical thereto, at least 90% identical thereto, at least 95%, 96%, 97%, 98% or 99% identical thereto.

In some embodiments Xn comprises or consists of one of the following sequences:

(SEQ ID NO: 53)
CACUCAGACUACGUGCACCU;
(SEQ ID NO: 54)
CACUCAGACUACGUGCUCCU;
(SEQ ID NO: 55)
CACUCAGACUACGUGCCCCU;
(SEQ ID NO: 56)
CACUCAGACUACGUGCGCCU;
and
(SEQ ID NO: 57)
CACUCAGACUAUGUGCACCU.

In other embodiments Xn comprises or consists of the sequence ACGGGCAACUUUACACGACG (SEQ ID NO: 58) or a sequence which is at least 60% identical thereto, at least 70% identical thereto, at least 80% identical thereto, at least 90% identical thereto, at least 95%, 96%, 97%, 98% or 99% identical thereto.

In one embodiment the regulatable intron comprises or consists of the sequence CNG/CNGCACUCAGACUACGUGCACCUCNG/CNGC (SEQ ID NO: 59), or a sequence which is at least 60% identical thereto, at least 70% identical thereto, at least 80% identical thereto, at least 90% identical thereto, at least 95%, 96%, 97%, 98% or 99% identical thereto, wherein/represents a cleavage site. In variant sequences according to the above sequence identity levels, the splice site target sequence may remain as CNG/CNGC, and sequence variation occurs in the other regions.

In one embodiment, the regulatable intron comprises or consists of the sequence CAG/CAGCACUCAGACUACGUGCACCUCUG/CUGC (SEQ ID NO: 60), or a sequence which is at least 60% identical thereto, at least 70% identical thereto, at least 80% identical thereto, at least 90% identical thereto, at least 95%, 96%, 97%, 98% or 99% identical thereto, wherein/represents a cleavage site. In variant sequences according to the above sequence identity levels, the splice site target sequence may remain as CAG/CUGC, and sequence variation occurs in the other regions.

In some embodiments the regulatable intron comprises or consists of one of the following sequences:

(SEQ ID NO: 61)
CNG/CAGCACUCAGACUACGUGCACCUCUG/CNG;
(SEQ ID NO: 62)
CNG/CAGCACUCAGACUACGUGCUCCUCUG/CNG;
(SEQ ID NO: 63)
CNG/CAGCACUCAGACUACGUGCCCCUCUG/CNG;
(SEQ ID NO: 64)
CNG/CAGCACUCAGACUACGUGCGCCUCUG/CNG;
and
(SEQ ID NO: 65)
CNG/CAGCACUCAGACUAUGUGCACCUCUG/CNG.

In other embodiments, the regulatable intron comprises or consists of one of the following sequences:

(SEQ ID NO: 66)
CAG/CAGCACUCAGACUACGUGCACCUCUG/CUGC;
(SEQ ID NO: 67)
CAG/CAGCACUCAGACUACGUGCUCCUCUG/CUGC;
(SEQ ID NO: 68)
CAG/CAGCACUCAGACUACGUGCCCCUCUG/CUGC;
(SEQ ID NO: 69)
CAG/CAGCACUCAGACUACGUGCGCCUCUG/CUGC;
and
(SEQ ID NO: 70)
CAG/CAGCACUCAGACUAUGUGCACCUCUG/CUGC.

In another embodiment the regulatable intron comprises the sequence CAG/CUGCAGCACUCAGACUACGUGCACCUCUG/CAG (SEQ ID NO: 71) or CAG/CUGCAGCACUCAGACUACGUGCACCUCUG/CUGG (SEQ ID NO: 72), wherein/represents a cleavage site. This sequence results from the addition of the trinucleotide CUG to the mammalian XBP1 intron sequence. Addition of this trinucleotide is believed to slightly de-optimise splicing of the intron to reduce any undesirable splicing (and hence background expression of the expression product) in cells.

Accordingly, in some embodiments Xn comprises or consists of

(SEQ ID NO: 73)
CAGCACUCAGACUACGUGCACCU.

In another embodiment the regulatable intron comprises the sequence: CNG/CAGACGGGCAACUUUACACGACGCUG/CNG (SEQ ID NO: 74), or a sequence which is at least 60% identical thereto, at least 70% identical thereto, at least 80% identical thereto, at least 90% identical thereto, at least 95%, 96%, 97%, 98% or 99% identical thereto, wherein/represents a cleavage site. In variant sequences according to the above sequence identity levels, the splice site target sequence may remain as CNG/CNG, and sequence variation occurs in the other regions.

In some embodiments it may be that the splice site target sequence (i.e. comprising the sequence CNG/CNG) in the transcript is flanked by sequences which are able to interact to form a stem-loop structure. Thus, the splice site target sequence is preferably flanked by sequences which are complementary to one another, such that they will hybridize with each other to form a stem-loop structure in which the splice site target sequence is located at least partially, or entirely, within the loop region of the stem-loop structure that is formed in the transcript.

In certain embodiments, where a stem-loop structure is to be formed, the stem-loop structure formed by the transcript preferably comprises a loop which comprises from 6 to 9 nucleotides, and a stem which is from 3 to 10 nucleotides in length. In some embodiments, the stem-loop structure comprises a loop which comprises from 7 to 8 nucleotides, and a stem which is from 4 to 8 nucleotides in length.

In certain embodiments, where a stem-loop structure is to be formed, the intron may suitably comprise a sequence at the splice target site as follows:

(SEQ ID NO: 214)
-Yn-CNG/CNG-A-Zn-

wherein A is an sequence having a length of from 0 to 3 nucleotides, in some embodiments 1 or 2 nucleotides,
wherein/represents the cleavage site,
and wherein Yn and Zn represent sequences that are complementary in nucleotide sequence when read in opposite directions, and are thus are able to hybridize to form the stem of the stem-loop structure. Yn and Zn are preferably from 3 to 10 nucleotides in length, in some embodiments from 4 to 8 nucleotides in length.

In some embodiments the intron may suitably comprise a sequence at the splice target site as follows:
Zn-CNG/CNG[CG]-A-Yn- (SEQ ID NO: 215), where the components have the same meaning as above. In this case A preferably has a length of 0, 1 or 2 nucleotides.

It will be apparent that providing suitable complementary sequences (e.g., Yn and Zn in the structures above) to provide the stem structure can be achieved by adapting the sequence of the intron to provide a suitable region which is complementary to the corresponding sequence in the adjacent coding (i.e. exon) sequence.

Further information regarding regulatable introns which may be used in the present invention is found in PCT/GB2018/052387 which is incorporated herein by reference.

Hypoxia-Inducible Promoters:

In some embodiments the hypoxia-inducible promoter is a synthetic hypoxia-inducible promoter. In some embodiments the synthetic hypoxia-inducible promoter comprises at least one hypoxia-responsive element (HRE) that is capable of being bound and activated by a hypoxia-inducible factor (HIF).

HIF is a family of transcription factors which are activated by decrease in the oxygen level in a cell. Under normal oxygen conditions, HIF is degraded following hydroxylation. Hypoxic conditions stabilize HIF and prevent its degradation. This allows HIF to translocate to the nucleus, bind to the HRE and activate HRE-responsive genes.

The hypoxia-inducible promoter typically comprises an HRE that is capable of being bound and activated by HIF operably linked to a minimal promoter. However, in some cases the HRE that is capable of being bound and activated by HIF is operably linked to a promoter other than a minimal promoter (e.g. a proximal promoter, such as a tissue-specific proximal promoter). The particular promoter associated with the HRE can be selected depending on the circumstances, but typically minimal promoters are preferred, especially when it is desired to minimize background expression levels.

HREs are generally composed of multimers of short conserved sequences, termed HIF-binding sites (HBSs). As the name suggests, HBSs are bound by HIF, whereupon the HRE is activated to drive transcription. Accordingly, the HRE of the present invention comprises a plurality of HBS, preferably 3 or more HBS, more preferably from 3 to 10 HBS, more preferably from 3 to 8 HBS, more preferably from 4 to 8 HBS. In some preferred embodiments of the present invention the HRE comprises 5, 6 or 8 HBS.

A core consensus sequence for the HBS has been determined. The core consensus sequence is NCGTG (SEQ ID NO: 75, N represents any nucleotide). There is an indication that A or G is optimal in the first position, so a generally preferred consensus sequence is [AG]CGTG (SEQ ID NO: 76). It should be noted that the HBS is functional when it is present in either strand of the double-stranded DNA (i.e. in either orientation). Accordingly, for example, the HBS may be represented by the reverse complement consensus sequence CACG[CT] (SEQ ID NO: 77) in one strand, indicating the presence of the sequence [AG]CGTG (SEQ ID NO: 76) on the corresponding complementary strand (in such cases the HBS can be described as being in the “reverse orientation” or “opposite orientation”).

The HBSs contained in the HRE each preferably comprise the consensus sequence NCGTG (SEQ ID NO: 75), and optionally the consensus sequence [AG]CGTG (SEQ ID NO: 76). Additional sequences flanking the consensus sequence may be present, and these have some effect on the affinity of the HIF for the HBS. Preferred HBS for some embodiments of the invention are discussed below.

Adjacent HBSs are typically, but not always, separated by spacer sequences. The spacing between HBSs in an HRE can have a significant effect on the inducibility and/or overall power of the promoter. In some cases, it may be desirable to optimize spacing between adjacent HBSs in order to maximize inducibility and power of the promoter. In other cases, it may be desirable to use suboptimal spacing in order to provide a promoter with lower inducibility and/or overall power of the promoter. Specific spacing between HBSs present in preferred embodiment of the invention will be discussed below. However, in general, it is typically preferred that the spacing between adjacent core consensus sequences in adjacent HBSs is from 3 to 50 nucleotides. To contribute to high levels of expression, it is typically preferred that the spacing between core consensus sequences in adjacent HBSs is from 7 to 25 nucleotides, preferably about 8 to 22 nucleotides. For intermediate levels of expression, it is typically preferred that the spacing between core consensus sequences in adjacent HBSs is from 5 to 6 nucleotides or from 26 to 32 nucleotides. For low levels of expression, it is typically preferred that the spacing between core consensus sequences in adjacent HBSs is from 2 to 4 nucleotides or from 33 to 50 nucleotides. It will be appreciated that there is scope to vary the spacing between adjacent HBS and thereby tailor the properties of the HRE.

The HRE is typically spaced from the promoter (e.g. minimal promoter), though it need not be. The spacing can have an effect on the inducibility and/or overall power of the promoter. Generally, it is preferred that the spacing between the core consensus sequences in the final HBS (i.e. that which is most proximal to the minimal promoter) and the TATA box (or equivalent sequence if a TATA box is not present) of the minimal promoter is from 0 to 200 nucleotides, more preferably 10 to 100 nucleotides, yet more preferably 20 to 70 nucleotides, yet more preferably 20 to 50 nucleotides, and yet more preferably 20 to 30 nucleotides. To contribute to high levels of expression, it is typically preferred that the spacing between final HBS and the TATA box (or equivalent sequence if a TATA box is not present) of the minimal promoter is 20-30, with spacings significantly over and under this leading to weaker expression levels. It will be appreciated that there is scope to vary the spacing between the final HBS and the MP and thereby tailor the properties of the HRE.

In some embodiments, the HRE that is capable of being bound and activated by HIF comprises at least one HBS that comprises or consists of the HRE1 sequence. The HRE1 HBS sequence is ACGTGC (SEQ ID NO: 78). HRE1 of course may be present on either strand of the nucleic acid, and thus in such cases the reverse orientation HRE1 will be indicated by the presence of the reverse complement sequence GCACGT (SEQ ID NO: 79).

In some embodiments, all HBS present in the HRE comprise or consist of the HRE1 sequence. The HRE1 sequences that are present in the HRE may each independently be present in either orientation. In some embodiments it is preferred that all of the HRE1 sequences that are present in the HRE are in the same orientation.

In some embodiments, the HRE that is capable of being bound and activated by HIF comprises at least one HBS that comprises or consists of the HRE2 sequence. The sequence of HRE2 is CTGCACGTA (SEQ ID NO: 80). In HRE2 the HBS is present in the reverse orientation when compared with HRE1, and as such the HRE2 sequence contains the reverse complement of the HRE1 sequence. HRE2 may be present on either strand of the nucleic acid, and thus in such cases the reverse orientation HRE2 may be indicated by the presence of the reverse complement sequence TACGTGCAG (SEQ ID NO: 81).

The HRE2 sequence comprises additional flanking sequences and is considered to be an optimized HBS, which binds HIF more strongly than HRE1. Thus, in cases where a high level of promoter inducibility and power are desired, HRE2 may be considered to be preferable to HRE1.

In some embodiments, all of the HBS present in the HRE comprise or consist of the HRE2 sequence. As the HRE2 sequence effectively comprises the HRE1 sequence, it will be apparent that when the HRE2 is provided HRE1 will inevitably also be present. The HRE2 sequences that are present in the HRE may each independently be present in either orientation. In some embodiments it is preferred that all of the HRE2 sequences that are present in the HRE are in the same orientation.

In some embodiments, the HRE that is capable of being bound and activated by a HIF comprises at least one HBS that comprises or consists of the HRE3 sequence, or a functional variant thereof.

The HRE3 sequence is ACCTTGAGTACGTGCGTCTCTGCACGTATG (SEQ ID NO: 82, HBS underlined). HRE3 represents a composite HBS which comprises two individual HBSs (i.e. binding sites for HIF, underlined) separated by a spacer, and with further spacers at each end. It can be seen that HRE3 comprises one HBS in each orientation (one comprising HRE1 and one comprising HRE2, the HRE1 sequence being positioned 5′ with respect to the HRE2 sequence). Given that each HRE3 sequence comprises 2 individual HBS, for the purposes of the present invention, each HRE3 sequence or functional variant thereof contributes 2 individual HBS to the total number of HBS present in the HRE.

HRE3 or functional variants thereof may be present on either strand of the nucleic acid, and thus in such cases the reverse orientation of HRE3 may be indicated by the presence of the reverse complement sequence CATACGTGCAGAGACGCACGTACTCAAGGT (SEQ ID NO: 83).

As mentioned above, functional variants of HRE3 also form embodiments of the present invention. Such variants are functional if they retain the ability to be bound by HIF leading to activation. Preferred functional variants of HRE3 retain the same HBSs as HRE3 in substantially the same position and orientation, but contain different spacer sequences. Accordingly, in some preferred embodiments the functional variant of HRE3 suitably has the following sequence:

(SEQ ID NO: 84)
S1-ACGTG-S2-CTGCACGTA-S3;

where S1 is a spacer of length 8-10, preferably 9,

where S2 is a spacer of length 4-6, preferably 5,

where S3 is a spacer of length 1-3, preferably 2.

In some embodiments of the invention, the functional variant of HRE3 comprises the sequence NNNNNNNNNACGTGNNNNNCTGCACGTANN (SEQ ID NO: 85).

In some embodiments, the functional variant of HRE3 has an overall sequence identity to HRE3 of a least 80%, preferably at least 90%, more preferably at least 95% identical to HRE3, and wherein the HBS sequences are completely identical to HRE3.

HRE3 is considered to be a particularly optimal sequence, which binds HIF strongly. Thus, in cases where a high level of prompter inducibility and power are desired, the presence of HRE3 or functional variants thereof that maintain similar properties may be considered to be preferable.

In some embodiments, all HBS present in the HRE comprise or consist of the HRE3 sequence, or a functional variant thereof. The HRE3 sequences, or functional variants thereof, that are present in the HRE may each independently be present in either orientation. In some embodiments it is preferred that all of the HRE3 sequences, or functional variants thereof, that are present in the HRE are in the same orientation.

In some embodiments, the HRE may comprise a combination of two or more of HRE1, HRE2 and/or HRE3.

In some embodiments, the HRE that is capable of being bound and activated by HIF suitably comprises the following sequence:

[ACGTGC-S]ₙ-ACGTGC
(“ACGTGC” disclosed as SEQ ID NO: 86);

wherein S is a spacer and n is from 2 to 9, preferably from 3 to 7. It should be noted that the sequence of the spacer can vary; that is to say that the spacer in each repeat unit [ACGTGC-S]ₙ (“ACGTGC” disclosed as SEQ ID NO: 87) may or may not have the same sequence or length.

The length of the spacer can be varied depending on the desired inducibility and power of the promoter.

Accordingly, in embodiments where is desired to maximize inducibility and power of the promoter, spacers are provided such that spacing between core consensus sequences in adjacent HBSs is from 7 to 18 nucleotides, preferably about 8-12 nucleotides, more preferably about 10 nucleotides. While it is often desirable to maximize inducibility and power of the promoter, in some cases a lower level of inducibility and power may be desired. In embodiments where a somewhat lower level of inducibility and power is desired, spacers can be provided such that adjacent HBSs are spaced apart by lesser or greater amounts, e.g. by from 4-6 nucleotides or from 19-50 nucleotides. It will be apparent that the HRE1 HBS comprises one nucleotide flanking the core consensus sequence (underlined—ACGTGC, SEQ ID NO: 78), and as such the spacers in in these embodiments take this into account to provide the desired spacing.

In some embodiments, the HRE that is capable of being bound and activated by HIF suitably comprises the following sequence:

ACGTGC-S-ACGTGC-S-ACGTGC-S-ACGTGC-S-ACGTGC
(“ACGTGC” disclosed as SEQ ID NO: 88)

wherein S is a spacer.

Suitable lengths for the spacer are discussed above. In some embodiments of the invention the spacers each have a length of 30-50 nucleotides. In such a case, an exemplary, but non-limiting, spacer has the following sequence:

(SEQ ID NO: 89)
GATGATGCGTAGCTAGTAGTGATGATGCGTAGCTAGTAGT.

In one embodiment the HRE that is capable of being bound and activated by HIF suitably comprises the following sequence: ACGTGCGATGATGCGTAGCTAGTAGTGATGATGCGTAGCTAGTAGTACGTGCGATGAT GCGTAGCTAGTAGTGATGATGCGTAGCTAGTAGTACGTGCGATGATGCGTAGCTAGTA GTGATGATGCGTAGCTAGTAGTACGTGCGATGATGCGTAGCTAGTAGTGATGATGCGTA GCTAGTAGTACGTGC (SEQ ID NO: 90, HBSs underlined), or a functional variant that is that is at least 80% identical thereto, preferably 85%, 90%, 95% or 99% identical thereto. Typically, it is preferred that in such functional variants the HRE1 sequences are substantially or completely identical to the reference sequence, and substantially all sequence variation arises in the spacer sequences

Such an HRE generally displays a very low level of inducibility and low expression levels when induced. This may be desirable in situations where background expression is to be minimized, and a high level of expression when induced is not required. Optimized spacing of the HBS can of course lead to higher levels of inducibility and expression upon induction.

In some preferred embodiments the HRE that is capable of being bound and activated by HIF suitably comprises the following sequence:

[CTGCACGTA-S]ₙ-CTGCACGTA
(“CTGCACGTA” disclosed as SEQ ID NO: 91);

wherein S is an optional spacer and n is from 2 to 9, preferably from 3 to 7.

It should be noted that the sequence of the spacer, when present, can vary; that is to say that the spacer in each repeat unit [CTGCACGTA-S]ₙ (“CTGCACGTA” disclosed as SEQ ID NO: 92) may or may not have the same sequence or length.

Details of the suitable spacings between core consensus sequences in adjacent HBSs are discussed above for the preceding embodiments, and these considerations apply to these embodiments equally. It will be apparent that the HRE2 HBS comprises four nucleotides flanking the core consensus sequence (underlined—CTGCACGTA, SEQ ID NO: 80), and as such the spacers in these embodiments take this into account to provide the desired spacing.

In some embodiments of the present invention, the HRE that is capable of being bound and activated by HIF suitably comprises the following sequence:

CTGCACGTA-S-CTGCACGTA-S-CTGCACGTA-S-CTGCACGTA-S-CTGCACGTA-S-CTGCACGTA (“CTGCACGTA” disclosed as SEQ ID NO: 93); wherein S is a spacer. Suitable lengths for the spacer are discussed above.

In some embodiments of the invention the spacers each have a length of 20 nucleotides. In such a case, an exemplary, but non-limiting, spacer has the following sequence:

(SEQ ID NO: 94)
GATGATGCGTAGCTAGTAGT

In one embodiment of the invention the HRE that is capable of being bound and activated by HIF suitably comprises the following sequence:

CTGCACGTAGATGATGCGTAGCTAGTAGTCTGCACGTAGATGATGCGTAG CTAGTAGTCTGCACGTAGATGATGCGTAGCTAGTAGTCTGCACGTAGATGATGCGTAGCT AGTAGTCTGCACGTAGATGATGCGTAGCTAGTAGTCTGCACGTA (SEQ ID NO: 95, HBSs underlined), or a functional variant that comprises a sequence that is at least 80% identical thereto, preferably 85%, 90%, 95% or 99% identical thereto. Typically, it is preferred that in such functional variants the HRE2 sequences are substantially or completely identical to the reference sequence, and substantially all sequence variation arises in the spacer sequences. Such an HRE generally displays an intermediate level of inducibility and low expression levels when induced. This may be desirable in situations where background expression is to be minimized, and a moderate level of expression when induced is required. Further optimization of the spacing of the HBSs can of course lead to higher levels of inducibility and expression upon induction. Likewise, de-optimization can lead to lower levels of inducibility and expression upon induction.

In some embodiments, the HRE that is capable of being bound and activated by HIF suitably comprises the following sequence:
CTGCACGTACTGCACGTACTGCACGTACTGCACGTA (SEQ ID NO: 96, HBSs underlined), or a functional variant that comprises a sequence that is at least 80% identical thereto, preferably 85%, 90%, 95% or 99% identical thereto.

It can be seen that this HRE comprises no additional spacers between adjacent HRE2 elements. However, in view of the four flanking nucleotides surrounding the core consensus sequence of HRE2, the core consensus sequences have an effective spacing of 4 nucleotides.

Such an HRE generally displays an intermediate level of inducibility and low expression levels when induced. This may be desirable in situations where background expression is to be minimized, and a moderate level of expression when induced required. Further optimization of the spacing of the HBSs can of course lead to higher levels of inducibility and expression upon induction. Likewise, de-optimization can lead to lower levels of inducibility and expression upon induction.

In some preferred embodiments, the HRE that is capable of being bound and activated by HIF suitably comprises from 3 to 6 HRE3 sequences, preferably from 3 to 5, preferably 4 HRE3 sequences, or a functional variant thereof, wherein adjacent HRE3 sequences, or functional variants thereof, are separated from each other by a spacer having a length of from 4 to 20 nucleotides, preferably from 6 to 15 nucleotides, more preferably 9 nucleotides.

In a preferred embodiment, the HRE that is capable of being bound and activated by HIF suitably comprises the following sequence:

[ACCTTGAGTACGTGCGTCTCTGCACGTATG-S]ₙ-
ACCTTGAGTACGTGCGTCTCTGCACGTATG
(“ACCTTGAGTACGTGCGTCTCTGCACGTATG”
disclosed as SEQ ID NO: 97);

wherein S is an optional spacer and n is from 2 to 5, preferably from 2 to 4, preferably 3.

It should be noted that the sequence of the spacer, if present, can vary; that is to say that the spacer in each repeat unit [ACCTTGAGTACGTGCGTCTCTGCACGTATG-S]ₙ (“ACCTTGAGTACGTGCGTCTCTGCACGTATG” disclosed as SEQ ID NO: 98) may or may not have the same sequence or length. It will be appreciated that in the HRE3 sequence as set out above can, in some or all instances, may be replaced with a functional variant thereof.

Details of the suitable spacings between core consensus sequences in adjacent HBSs are discussed above for the preceding embodiments, and these considerations apply to these embodiments equally. It will be apparent that the HRE3 composite HBS comprises 11 nucleotides flanking the region containing the two core consensus sequence (underlined—ACCTTGAGTACGTGCGTCTCTGCACGTATG, SEQ ID NO: 99), and as such the spacers in in these embodiments take this into account to provide the desired spacing. In some embodiments the spacer, S, suitably has a length of from 4 to 20 nucleotides, preferably from 7 to 15 nucleotides, more preferably 9 nucleotides.

In some embodiments of the present invention, the HRE that is capable of being bound and activated by HIF suitably comprises the following sequence:

ACCTTGAGTACGTGCGTCTCTGCACGTATG-S-ACCTTGAGTACGTGCGTCTCTGCACGTATG-S-ACCTTGAGTACGTGCGTCTCTGCACGTATG-S-ACCTTGAGTACGTGCGTCTCTGCACGTATG-S; (“ACCTTGAGTACGTGCGTCTCTGCACGTATG” disclosed as SEQ ID NO: 100); wherein S is a spacer. Suitable lengths for the spacer are discussed above. It will be appreciated that in the HRE3 sequence as set out here can, in some or all instances, be replaced with a functional variant thereof.

In some embodiments, the HRE that is capable of being bound and activated by HIF suitably comprises the following sequence:

ACCTTGAGTACGTGCGTCTCTGCACGTATG-S-ACCTTGAGTACGTGCGTCTCTGCACGTATG-S-ACCTTGAGTACGTGCGTCTCTGCACGTATG-S-ACCTTGAGTACGTGCGTCTCTGCACGTATG-S-ACCTTGAGTACGTGCGTCTCTGCACGTATG-S; (“ACCTTGAGTACGTGCGTCTCTGCACGTATG” disclosed as SEQ ID NO: 101); wherein S is a spacer. Suitable lengths for the spacer are discussed above. It will be appreciated that in the HRE3 sequence as set out here can, in some or all instances, be replaced with a functional variant thereof.

In some embodiments of the invention the spacers each have a length of 9 nucleotides. In such a case, an exemplary, but non-limiting, spacer has the following sequence:

(SEQ ID NO: 102)
GCGATTAAG.

In one preferred embodiment of the invention the HRE that is capable of being bound and activated by HIF suitably comprises the following sequence:

ACCTTGAGTACGTGCGTCTCTGCACGTATGGCGATTAAGACCTTGAGTAC GTGCGTCTCTGCACGTATGGCGATTAAGACCTTGAGTACGTGCGTCTCTGCACGTATGGC GATTAAGACCTTGAGTACGTGCGTCTCTGCACGTATG (SEQ ID NO: 103, HBSs underlined), or a functional variant that comprises a sequence that is at least 80% identical thereto, preferably 85%, 90%, 95% or 99% identical thereto. Typically, it is preferred that in such functional variants the HRE1 and HRE2 sequences present in the HRE3 sequence are substantially or completely identical to the reference sequence, and substantially all sequence variation arises in the spacer sequences.

Such an HRE generally displays a high level of inducibility and high expression levels when induced. This may be desirable in situations where a high level of expression when induced is required. Further optimization of the spacing of the HBSs may potentially lead to higher levels of inducibility and expression upon induction. Likewise, de-optimization can lead to lower levels of inducibility and expression upon induction.

As mentioned previously, the hypoxia-inducible promoter typically comprises the HRE that is capable of being bound and activated by HIF operably linked to a minimal or proximal promoter. It is preferred that the prompter operably linked to the HRE is a minimal promoter.

The minimal promoter can be any suitable minimal promoter. A wide range of minimal promoters are known in the art. Without limitation, suitable minimal promoters include CMV minimal promoter (CMV-MP), YB-TATA minimal promoter (YB-TABA), HSV thymidine kinase minimal promoter (MinTK), and SV40 minimal promoter (SV40-MP). The minimal promoter can be a synthetic minimal promoter. Particularly preferred minimal promoters are the CMV minimal promoter (CMV-MP) and YB-TATA minimal promoter (YB-TABA).

The sequence of CMV-MP is:

(SEQ ID NO: 104)
AGGTCTATATAAGCAGAGCTCGTTTAGTGAACCGTCAGATCGCCTAGAT
ACGCCATCCACGCTGTTTTGACCTCCATAGAAGATCGCCACC.

The sequence of YB-TATA is:

(SEQ ID NO: 105)
TCTAGAGGGTATATAATGGGGGCCA.

However, a longer sequence containing the YB-TATA MP is used in some preferred embodiments of the invention. The sequence of this longer sequence of the YB-TATA MP (referred to herein as longYB-TATA) is GCGATTAATCCATATGCTCTAGAGGGTATATAATGGGGGCCACTAGTCTACTACCAGAA AGCTTGGTACCGAGCTCGGATCCAGCCACC (SEQ ID NO: 106). Accordingly, wherever YB-TATA is provided as a component of an inducible promoter herein, a substantially equivalent sequence with longYB-TATA substituted in place of YB-TATA is also considered to be an embodiment of the invention. The spacing between the last HBS and the TATA box of MP is preferably retained.

The sequence of MinTK is:

(SEQ ID NO: 107)
TTCGCATATTAAGGTGACGCGTGTGGCCTCGAACACCGAGCGACCCTGC
AGCGACCCGCTTAA.

The sequence of SV40-MP is:

(SEQ ID NO: 108)
GCATCTCAATTAGTCAGCAACCATAGTCCCGCCCCTAACTCCGCCCATC
CCGCCCCTAACTCCGCCCAGTTCCGCCCATTCTCCGCCCCATCGCTGAC
TAATTTTTTTTATTTATGCAGAGGCCGAGGCCGCCTCGGCCTCTGAGCT
ATTCCAGAAGTAGTGAGGAGGCTTTTTTGGAGGCCTAGGCTTTTGCAAA
AAGCTT.

The sequence of MP1 is:

(SEQ ID NO: 109)
TTGGTACCATCCGGGCCGGCCGCTTAAGCGACGCCTATAAAAAATAGGT
TGCATGCTAGGCCTAGCGCTGCCAGTCCATCTTCGCTAGCCTGTGCTGC
GTCAGTCCAGCGCTGCGCTGCGTAACGGCCGCC

Accordingly, preferred embodiments comprise the HRE that is capable of being bound and activated by HIF operably linked to one of the abovementioned minimal promoters, more preferably to CMV-MP or YB-TATA, and most preferably CMV-MP. CMV-MP, when combined with HREs of the present invention, has shown to provide extremely high levels of inducibility and high promoter strength. Low background expression levels have also been observed.

The HRE is preferably spaced from the minimal promoter (or other type of promoter, if used) by a spacer sequence. The spacing between the HRE and the minimal promoter can affect the inducibility and power of the hypoxia-inducible promoter. Generally, it is preferred that the spacing between the core consensus sequences in the final HBS (i.e. that which is most proximal to the minimal promoter) and the TATA box (or equivalent sequence if a TATA box is not present) of the minimal promoter is from 10 to 100 nucleotides, more preferably 20 to 70 nucleotides, yet more preferably 20 to 50 nucleotides, and yet more preferably 20 to 30 nucleotides.

In embodiments where is desired to optimize inducibility and power of the hypoxia-inducible promoter, the spacing between the final HBS and the TATA box (or equivalent sequence if a TATA box is not present) of the minimal promoter is preferably from 20 to 30 nucleotides. In embodiments where a somewhat lower level of inducibility and power is desired, the spacing between the final HBS and the TATA box (or equivalent sequence if a TATA box is not present) can be lesser or greater, e.g. from 0 to 10 nucleotides or from 31 to 100 nucleotides. While it is often desirable to maximize inducibility and power of the promoter, in some cases a lower level of inducibility and power may be desired.

In an exemplary, non-limiting embodiment, the hypoxia-inducible promoter comprises on of the following sequences (the HBS sequences are underlined and minimal promoter sequences are shown in bold):
ACGTGCGATGATGCGTAGCTAGTAGTGATGATGCGTAGCTAGTAGTACGTGCGAT GATGCGTAGCTAGTAGTGATGATGCGTAGCTAGTAGTACGTGCGATGATGCGTAG CTAGTAGTGATGATGCGTAGCTAGTAGTACGTGCGATGATGCGTAGCTAGTAGTG ATGATGCGTAGCTAGTAGTACGTGCTTGGTACCATCCGGGCCGGCCGCTTAAG CGACGCCTATAAAAAATAGGTTGCATGCTAGGCCTAGCGCTGCCAGTCCATC TTCGCTAGCCTGTGCTGCGTCAGTCCAGCGCTGCGCTGCGTAACGGCCGCC (Synp-RTV-015; SEQ ID NO: 1), or a functional variant that comprises a sequence that is at least 80% identical thereto, preferably 85%, 90%, 95% or 99% identical thereto; and
CTGCACGTACTGCACGTACTGCACGTACTGCACGTATGGGTACCGTCGACGATAT CGGATCCAGGTCTATATAAGCAGAGCTCGTTTAGTGAACCGTCAGATCGCCT AGATACGCCATCCACGCTGTTTTGACCTCCATAGAAGATCGCCACC (Synp-HYP-001, SEQ ID NO: 3) or a functional variant that comprises a sequence that is at least 80% identical thereto, preferably 85%, 90%, 95% or 99% identical thereto;

Typically, it is preferred that in such functional variants the HRE1, HRE2 and MP sequences are substantially identical to the reference sequence, and substantially all sequence variation arises in the spacer sequences.

In some preferred embodiments, upon induction via rendering cells hypoxic (e.g. after cells are exposed to 5% oxygen for 5h, having previously been normoxic, e.g. exposed to 20% oxygen) the expression level of the transgene is increased by at least a 5-fold, more preferably a 10-, 15-, 20-, 30-, or 50-fold.

In some preferred embodiments, upon induction (e.g. after cells are exposed to 5% oxygen for 5h, having previously been normoxic, e.g. exposed to 20% oxygen) the expression level of the transgene is at least 50% of that provided by the CMV-IE promoter (i.e. an otherwise identical vector in the same cells under the same conditions, but in which expression of the transgene is under control of CMV-IE rather than the hypoxia inducible promoter). More preferably the expression level of the transgene is at least 75%, 100%, 150%, 200%, 300%, 400% or 500% of that provided by the CMV-IE promoter.

Other naturally-occurring hypoxia-inducible promoters are described in US20110158947A1, which is incorporated herein by reference. Accordingly, in some embodiments the hypoxia-inducible promoter can be selected from the group consisting of: Adenosine A2B receptor (A2BR) promoter, Plasminogen activator receptor (uPAR) VEGF receptors (VEGFR1 and VEGFR2) promoter, Platelet-derived endothelial cell growth factor/thymidine phosphorylase (PDECGF/TP) promoter, nitric oxide synthase (NOS) promoter, Phosphoglycerate kinase-1 (PGK-1) promoter, Pyruvate kinase M (PK-M) promoter, Glucose transporter 1 (GLUT1) promoter, Hypoxia-inducible factor (HIF-1) promoter, Early growth response 1 (Egr-1) promoter, Nuclear factor kB (NFkB) promoter, Hepatocyte growth factor activator (HGFA) promoter, Vascular endothelial growth factor (VEGF) promoter, CXCL8 promoter, CCL11 promoter, Transforming growth factor-beta (TGF-β) promoter, procollagen promoter, Integrin-linked kinase (ILK) promoter, K1PDC1 promoter, Erythropoietin (EPO) promoter, Serine/Threonine Kinase-15 (STK15) promoter, the histone demethylase Jumonji domain containing 1A (JMJD1A) promoter, Endothelin-2 (EDN2) promoter, choline kinase (Chk) promoter, Sphingosine kinase 1 promoter, Carcinoembryonic antigen (CEA, ceacam5) promoter, Monocyte chemoattractant protein-1 (MCP-1/CCL2) promoter, MCP-5 (Ccll 2) promoter, prostate-specific antigen (PSA) promoter, c-Met promoter, Matrix metalloproteinases Class III beta-tubulin (TUBB3) promoter, Glutamine: fructose-6-phosphate amidotransferase (GFAT) promoter, Protein phosphatase 1 nuclear targeting subunit beta-secretase (BACE1) promoter and Plasminogen activator inhibitor-1 (PAI-1) promoter.

Other naturally-occurring hypoxia-inducible promoters are described in WO2016/146819, which is incorporated herein by reference. See, for example, Table 4.

Hypoxia responsive elements have been described in L. Marignol, M. Lawler, M. Coffey & D. Hollywood (2005) Achieving hypoxia inducible gene expression in tumours, Cancer Biology & Therapy, 4:4, 365-370; U.S. Pat. No. 6,218,179; Madan et al, PNAS 90: 3928, 1993; JP2005095173A, US 2006/0099709; and WO1999/048916. A murine hypoxia response element is disclosed in U.S. Pat. No. 5,942,434.

Induction of a hypoxia-inducible promoter can be achieved by making the cells hypoxic, i.e. treating said population of cells so as to induce hypoxia in the cells, such that expression from the transgene linked to the hypoxia-inducible promoter is induced and the expression product is produced. Suitable approaches will be apparent to the skilled person for any particular cell type. Generally, eukaryotic cells are cultured under aerobic conditions, and many approaches are known in the art to achieve this for various cell and culture types. Hypoxic conditions can be achieved by reducing the amount of oxygen supplied to the cell. For example, cells can be grown under normoxic conditions (e.g. approximately 20% oxygen), before being switched to a gas mix comprising less oxygen or no oxygen to induce hypoxia. For example, a gas containing 5% oxygen can be used to induce hypoxia in cells. An exemplary suitable gas mix for use to induce hypoxic conditions in cell culture is 5% oxygen, 10% carbon dioxide and 85% nitrogen, but other gas mixes can be used. In an alternative approach, hypoxia in cell culture can be induced by introduction of an agent that can induce hypoxia in the cells. For example, CoCl2 can be used at suitable concentrations, e.g. a final concentration of approximately 100 μM in cell culture media, to induce hypoxia. Generally, it is preferred in the present invention that hypoxia is achieved without the addition of such an agent as it adds to costs, and in many cases the agent will be undesirable and may be hard to remove.

In some cases, it may be desirable to alter the amount of oxygen supplied to the cells while they are in a hypoxic condition to optimize or otherwise modulate expression of the desired expression product. For example, it may be desirable to establish highly hypoxic conditions initially to strongly induce hypoxic conditions, followed by a period of culturing the cells under less hypoxic conditions that are less detrimental to the health and activity of the cells. Thus, methods disclosed herein may comprise varying the level of hypoxia to which the cells are subject.

Expression from a hypoxia-inducible promoter as discussed herein can be adjusted (e.g. uninduced, repressed or modulated) by altering the level of oxygen to which cells comprising the promoter are exposed. For example, induced expression through hypoxia can be switched off (uninduced) by exposing the cells to normoxic conditions (e.g. exposed to 20% oxygen).

In one embodiment, the synthetic hypoxia-inducible promoter does not comprise or consist of one of the following structures:
HRE2-S₂₀-HRE2-S₂₀-HRE2-S₂₀-HRE2-S₂₀-HRE2-S₂₀-HRE2-S₅₉-CMV-MP;
HRE3-S₉-HRE3-S₉-HRE3-S₉-HRE3-S₁₇-YB-TABA-MP; and
HRE3-S₉-HRE3-S₉-HRE3-S₉-HRE3-S₁₇-CMV-MP.

Herein, S, represents a spacer of length X nucleotides.

In one embodiment, the synthetic hypoxia-inducible promoter does not comprise or consist of one of the following sequences:

(Synp-RTV-016, SEQ ID NO: 2)
CTGCACGTAGATGATGCGTAGCTAGTAGTCTGCACGTAGATGATGCGTAG
CTAGTAGTCTGCACGTAGATGATGCGTAGCTAGTAGTCTGCACGTAGATG
ATGCGTAGCTAGTAGTCTGCACGTAGATGATGCGTAGCTAGTAGTCTGCA
CGTAGTAGTCGTATGCTGATGCGCAGTTAGCGTAGCTGAGGTACCGTCGA
CGATATCGGATCCAGGTCTATATAAGCAGAGCTCGTTTAGTGAACCGTCA
GATCGCCTAGATACGCCATCCACGCTGTTTTGACCTCCATAGAAGATCGC
CACC;
(Synp-HYBT, SEQ ID NO: 4)
ACCTTGAGTACGTGCGTCTCTGCACGTATGGCGATTAAGACCTTGAGTAC
GTGCGTCTCTGCACGTATGGCGATTAAGACCTTGAGTACGTGCGTCTCTG
CACGTATGGCGATTAAGACCTTGAGTACGTGCGTCTCTGCACGTATGGCG
ATTAATCCATATGCTCTAGAGGGTATATAATGGGGGCCA;
and
(Synp-HV3C, SEQ ID NO: 5)
ACCTTGAGTACGTGCGTCTCTGCACGTATGGCGATTAAGACCTTGAGTAC
GTGCGTCTCTGCACGTATGGCGATTAAGACCTTGAGTACGTGCGTCTCTG
CACGTATGGCGATTAAGACCTTGAGTACGTGCGTCTCTGCACGTATGGCG
ATTAATCCATATGCAGGTCTATATAAGCAGAGCTCGTTTAGTGAACCGTC
AGATCGCCTAGATACGCCATCCACGCTGTTTTGACCTCCATAGAAGATCG
CCACC.

Forskolin-Inducible Promoters:

In some embodiments the inducible promoter is a forskolin-inducible promoter.

In some embodiments the forskolin-inducible promoter is a synthetic forskolin-inducible promoter. In some embodiments, the synthetic forskolin-inducible promoter comprises a synthetic forskolin-inducible cis-regulatory element (CRE) that is capable of being bound by CREB and/or AP1.

In one embodiment, the forskolin responsive enhancer element has a sequence comprising

(SEQ ID NO: 110)
TGACGTCACGATTTGACGTCAACCATTGACGTCACGATTTGACGTCA
ACCATTGACGTCACGATTTGACGTCACGATTTGACGTCAACCAT
TGACTCACGATTTGACTCAACCATTGACTCACGATTTGACTCA
ACCATTGACTCACGATTTGACTCA.

In the sequence for SEQ ID NO: 107, cAMPREs are indicated in underlined text, and AP-3 sites are indicated in bolded, underlined text. The space between sites is neutral DNA. As can be seen from this sequence the new enhancer element is comprised of 7-CRE sites and 6 AP-3 sites with a 5 bp spacer between elements. This enhancer was combined with the following minimal promoters, with a 5 bp spacer between the enhancer and the minimal promoter: YB-Tata (FORNYB-REP), CMV (FORNCMV), CMV53 (FORNCMV53), MinTK (FORNMinTK), MLP (FORNMLP), SV40 (FORNSV40), pJB42 (FORNJB42). Sequences of the forskolin responsive enhancer element combined with the minimal promoter are provided herein, e.g., in Example 7.

The enhancer element was further combined with the minimal promoter, TATA-m6A. The minimal promoter TATA-m6A has a sequence comprising of (SEQ ID NO: 111)

(SEQ ID NO: 111)
TATAAAAGGCAGAGCTCGTTTAGTGAACCGaagctt
ggactaaagcggacttgtctcgag

SEQ ID NO: 111 comprises a consensus sequence TATA box highlighted in underlined text and an m6a sequence highlighted in bolded, underlined text. The TATA box is the minimal sequence required to stablize transcription from an enhancer, whereas the m6A sequence that is signal for the mRNA to be methylated. This and other chemical modifications, of which there are at least 160 known, of mRNA are believed to create another layer of post-transcriptional control during gene expression. Of these the m6a is the best understood and research has shown that is involved in a large number of mRNA functions e.g. splicing, export, translation and stability. It has been observed that ˜¼ of all eukaryotic mRNAs have at least one m6a site and is therefore the most prevalent form of mRNA modification (Han et al, 2020). In one such study of m6a methylation it was shown that by placing methylations sequences at the 5′ end of the mRNA, before the ATG, could enhance translation of the transcript (Meyer et al, 2015). We have modified these findings to add a m6a sequence to our TATA minimal promoter to boost translational efficiency from a weak but very small minimal promoter. This should allow us to achieve high expression while recuing the overall size of the promoter and generate a novel minimal promoter.

One aspect provided herein is a synthetic forskolin inducible promoter comprising a sequence according to SEQ ID NO: 110, or a functional variant thereof.

In one embodiment, the synthetic forskolin inducible promoter comprising a sequence which is at least 60%, 65%, 70%, 75%, 80%, 90%, 95%, 96%, 97%, 98%, or 99% identical to SEQ ID NO: 110.

In one embodiment, the functional variant of the synthetic forskolin inducible promoter retains at least 25%, 50%, 75%, 80%, 85%, 80%, 95% or 100% of the activity of the reference promoter.

In one embodiment, the synthetic forskolin inducible promoter is operatively linked to a nucleic acid sequence to drive expression of the same, for example, a nucleic acid seqeunce that encodes a toxic gene described herein. However, the nucleic acid need not be a nucleic acid sequence described herein; any sequence can be operatively linked to the synthetic forskolin inducible promoter.

While the CRE/promoter is referred to as forskolin-inducible, it may also be induced by other agents, as discussed in more detail below. The mechanism of induction by forskolin is via the activation of adenylyl cyclase and the resultant increase of intracellular cAMP. Accordingly, the CRE/promoter is also inducible by other activators of adenylyl cyclase or factors that increase intracellular cAMP.

Preferably the CRE comprises at least 2, more preferably at least 3, transcription factor binding sites (TFBS) for CREB and/or AP1 (as used herein, the term “TFBS for X” means a TFBS which is capable of being bound by transcription factor X).

Preferably the CRE comprises at least 4 TFBS for CREB and/or API. Suitably the CRE comprises 3, 4, 5, 6, 7, 8, 9, 10, 11, 12, 13, 14, or 15 TFBS for CREB and/or AP1.

While there is no specific upper limit for the number TFBS for CREB and/or AP1, in general it is preferred that the CRE comprises 15 or fewer TFBS for CREB and/or AP1, optionally 10 or fewer TFBS for CREB and/or AP1.

In some embodiments the CRE comprises at least 1 TFBS for each of CREB and AP1. In some embodiments the CRE comprises at least 2, 3, 4, 5, 6 or 7 TFBS for each of CREB and AP1.

TFBS for CREB typically comprise or consist of the highly conserved consensus sequence TGACGTCA (SEQ ID NO: 112). This sequence is known as the cAMP Responsive Element (or cAMPRE or CRE; the abbreviation cAMPRE will be used herein to avoid confusion with the abbreviation for cis-regulatory element). Other cAMPREs that can be used are discussed below.

TFBS for AP1 typically comprise or consist of the consensus sequence TGA[GC]TCA (SEQ ID NO: 113). In specific examples of the present invention the sequences TGAGTCA (named AP1(1), SEQ ID NO: 114), TGACTCAG (named AP1(2), SEQ ID NO: 115) and TGACTCA (named AP1(3), SEQ ID NO: 116) were used, and thus AP1(1), AP1(3) and AP1(2) can be viewed as preferred TFBS for AP1. The generic term AP1 in respect of a TFBS refers to a TFBS comprising the above consensus sequence, and it encompasses both AP1(1), AP1(3) and AP1(2).

In some preferred embodiments the CRE comprises at least one TFBS for a transcription factor other than CREB and/or AP1. In some preferred embodiments of the present invention the CRE comprises at least one TFBS for ATF6 and/or hypoxia inducible factor (HIF). The CRE may comprise 2, 3, 4, 5, 6, 7, 8, 9, or 10 TFBS for a transcription factor other than CREB and/or AP1, for example for ATF6 and/or HIF. In some embodiments of the invention the CRE comprises at least 1 TFBS for each of ATF6 and HIF.

TFBS for HIF comprise or consist of the consensus sequence NCGTG (SEQ ID NO: 75), more preferably [AG]CGTG (SEQ ID NO: 76). This sequence is referred to as the HIF binding sequence (HBS). In specific examples of the present invention the HBS sequence CTGCACGTA (named HRE1, SEQ ID NO: 80) was used, and thus HRE1 can be viewed as a preferred TFBS for HIF. However, other TFBS for HIF are known and can be used in the present invention, for example ACGTGC (SEQ ID NO: 78) or

(SEQ ID NO: 82)
ACCTTGAGTACGTGCGTCTCTGCACGTATG.

TFBS for ATF6 comprise or consist of the consensus sequence TGACGT (SEQ ID NO: 117), more preferably TGACGTG (SEQ ID NO: 118). In specific examples of the present invention the TFBS sequence TGACGTGCT (SEQ ID NO: 119) was used and this can be viewed as preferred TFBS for ATF6. However, in general, any sequence comprising the consensus sequence TGACGT (SEQ ID NO: 117), more preferably TGACGTG (SEQ ID NO: 118), can be used.

Each of the TFBS discussed above can be present in either orientation (i.e. they can be functional when present on either strand of the double-stranded DNA). Thus, it will be apparent that any of the TFBS may be represented by the reverse complement consensus sequence in one strand, which indicates the presence of the TFBS sequence on the corresponding complementary strand (in such cases the TFBS can be described as being in the “reverse orientation” or “opposite orientation”). In general, a reference to a TFBS, whether by name or by reciting the sequence of a TFBS, should be considered to refer to the presence of the TFBS in either orientation. When a sequence of a TFBS is recited it should be appreciated that the orientation shown represents a specifically disclosed, and typically a preferred, embodiment.

In some embodiments, the CRE comprises:
5 TFBS for CREB and 3 TFBS for AP1;
5 TFBS for CREB and 4 TFBS for AP1;
8 TFBS for AP1;
3 TFBS for ATF6, 4 TFBS for AP1 and 3 TFBS for HIF; or
7 TFBS for CREB and 6 TFBS for AP1;

wherein adjacent TFBS are optionally, but preferably, separated by spacer sequences.

The spacer sequence can be any suitable length. Typically, the spacer is from 2 to 100 nucleotides in length, from 5 to 50 nucleotides in length, from 6 to 40 nucleotides in length, from 7 to 30 nucleotides in length, from 8 to 25 nucleotides in length or from 10 to 20 nucleotides in length. Spacers of 5, 10, 20 nucleotides in length have been used in some specific embodiments of the invention, and these function well, but other lengths of spacers can be used. In some embodiments it is preferred that the spacer is a multiple of 5 nucleotides in length. The skilled person can readily determine suitable lengths of spacers.

It should be noted that the sequence and length of the of the spacers can vary; that is to say that each spacer in a sequence need not have the same sequence or length as any other. For convenience, some or all ofthe spacers between TFBS in a CRE often do have the same sequence and length, so, while this may be preferred, it is not required.

The TFBS can suitably be in any order, but in preferred embodiments they are provided in the order in which they are recited, i.e. in the first embodiment in the list above there would be 4 TFBS for cAMPRE and then 3 TFBS for AP1 in an upstream to downstream direction.

In some embodiments, the CRE consists of:
5 TFBS for CREB and 3 TFBS for AP1;
5 TFBS for CREB and 4 TFBS for AP1;
8 TFBS for AP1;
3 TFBS for ATF6, 4 TFBS for AP1 and 3 TFBS for HIF; or
7 TFBS for CREB and 6 TFBS for AP1;

wherein adjacent TFBS are optionally, but preferably, separated by spacer sequences.

Suitable lengths for the spacers are discussed above.

Again, the TFBS can suitably be in any order, but in preferred embodiments they are provided in the order in which they are recited.

In some embodiments, the CRE comprises one of the following structures:
cAMPRE-S-cAMPRE-S-cAMPRE-S-cAMPRE-S-cAMPRE-S-AP1-S-AP1-S-AP1 (CRE comprising 5×cAMPRE and 3×AP1 TFBS);
cAMPRE-S-cAMPRE-S-cAMPRE-S-cAMPRE-S-cAMPRE-S-AP1(2)-S-AP1(2)-S-AP1(2) (CRE comprising 5×cAMPRE and 3×AP1(2) TFBS);
cAMPRE-S-cAMPRE-S-cAMPRE-S-cAMPRE-S-cAMPRE-S-AP1-S-AP1-S-AP1-S-AP1 (CRE comprising 5×cAMPRE and 4×AP1 TFBS);
cAMPRE-S-cAMPRE-S-cAMPRE-S-cAMPRE-S-cAMPRE-S-AP1(2)-S-AP1(2)-S-AP1(2)-S-AP1(2) (CRE comprising 5×cAMPRE and 4×AP1(2) TFBS);
AP1-S-AP1-S-AP1-S-AP1-S-AP1-S-AP1-S-AP1-S-AP1 (CRE comprising 8×AP1 TFBS);
AP1(1)-S-AP1(1)-S-AP1(1)-S-AP1(1)-S-AP1(1)-S-AP1(1)-S-AP1(1)-S-AP1(1) (CRE comprising 8×AP1(1) TFBS);
ATF6-S-ATF6-S-ATF6-S-AP1-S-AP1-S-AP1-S-AP1-S—HIF-S-HIF—S-HIF (CRE comprising 3×ATF6, 4×AP1 and 3×HIF TFBS); and
ATF6-S-ATF6-S-ATF6-S-AP1(1)-S-AP1(1)-S-AP1(1)-S-AP1(1)-S-HRE-S-HRE1-S-HRE1 (CRE comprising 3×ATF6, 4×AP1(1) and 3×HRE1 TFBS);
cAMPRE-S-cAMPRE-S-cAMPRE-S-cAMPRE-S-cAMPRE-S-AP1-S-AP1-S-AP1-S-AP1 (CRE comprising 5×cAMPRE 4×AP1 TFBS); and
cAMPRE-S-cAMPRE-S-cAMPRE-S-cAMPRE-S-cAMPRE-S-AP1(1)-S-AP1(1)-S-AP1(1)-S-AP1(1) (CRE comprising 5×cAMPRE 4×AP1(1) TFBS); and
cAMPRE-S-cAMPRE-S-cAMPRE-S-cAMPRE-S-cAMPRE-S-cAMPRE-S-cAMPRE-S-AP1(3)-S-AP1(3)-S-AP1(3)-S-AP1(3)-S-AP1(3)-S-AP1(3) (CRE comprising 7×cAMPRE and 6×AP1(3));
wherein S represents an optional, but preferable, spacer sequence. Suitable lengths for the spacers are discussed above.

In these structures, reference to a TF represents the presence of the TFBS for that TF. cAMPRE is used to refer to the TFBS for CREB.

In some specific embodiments, the CRE comprises one of the following structures:
cAMPRE-S₁₀-cAMPRE-S₁₀-cAMPRE-S₁₀-cAMPRE-S₁₀-cAMPRE-S₁₀-AP1-S₁₀-AP1-S₁₀-AP1 (CRE comprising 5×cAMPRE and 3×AP1 TFBS);
cAMPRE-S₁₀-cAMPRE-S₁₀-cAMPRE-S₁₀-cAMPRE-S₁₀-cAMPRE-S₁₀-AP1(2)-S₁₀-AP1(2)-S₁₀-AP1(2) (CRE comprising 5×cAMPRE and 3×AP1(2) TFBS);
cAMPRE-S₁₀-cAMPRE-S₁₀-cAMPRE-S₁₀-cAMPRE-S₁₀-cAMPRE-S₁₀-AP1-S₁₀-AP1-S₁₀-AP1-S₁₀-AP1 (CRE comprising 5×cAMPRE and 4×AP1 TFBS);
cAMPRE-S₁₀-cAMPRE-S₁₀-cAMPRE-S₁₀-cAMPRE-S₁₀-cAMPRE-S₁₀-AP1(2)-S₁₀-AP1(2)-S₁₀-AP1(2)-S₁₀-AP1(2) (CRE comprising 5×cAMPRE and 4×AP1(2) TFBS);
AP1-S₂₀-AP1-S₂₀-AP1-S₂₀-AP1-S₂₀-AP1-S₂₀-AP1-S₂₀-AP1-S₂₀-AP1 (CRE comprising 8×AP1 TFBS);
AP1(1)-S₂₀-AP1(1)-S₂₀-AP1(1)-S₂₀-AP1(1)-S₂₀-AP1(1)-S₂₀-AP1(1)-S₂₀-AP1(1)-S₂₀-AP1(1) (CRE comprising 8×AP1(1) TFBS);
ATF6-S₂₀-ATF6-S₂₀-ATF6-S₂₀-AP1-S₂₀-AP1-S₂₀-AP1-S₂₀-AP1-S₂₀-HIF—S₂₀-HIF—S₂₀-HIF (CRE comprising 3×ATF6, 4×AP1 and 3×HIF TFBS); and
ATF6-S₂₀-ATF6-S₂₀-ATF6-S₂₀-AP1(1)-S₂₀-AP1(1)-S₂₀-AP1(1)-S₂₀-AP1(1)-S₂₀-HRE1-S₂₀-HRE1-S₂₀-HRE1 (CRE comprising 3×ATF6, 4×AP1(1) and 3×HRE1 TFBS);
cAMPRE-S₁₀-cAMPRE-S₁₀-cAMPRE-S₁₀-cAMPRE-S₁₀-cAMPRE-S₁₀-AP1-S₁₀-AP1-S₁₀-AP1-S₁₀-AP1 (CRE comprising 5×cAMPRE 4×AP1 TFBS); and
cAMPRE-S₁₀-cAMPRE-S₁₀-cAMPRE-S₁₀-cAMPRE-S₁₀-cAMPRE-S₁₀-AP1(1)-S₁₀-AP1(1)-S₁₀-AP1(1)-S₁₀-AP1(1) (CRE comprising 5×cAMPRE 4×AP1(1) TFBS); and
cAMPRE-S₅-cAMPRE-S₅-cAMPRE-S₅-cAMPRE-S₅-cAMPRE-S₅-cAMPRE-S₅-cAMPRE-S₅-AP1(3)-S₅-AP1(3)-S₅-AP1(3)-S₅-AP1(3)-S₅-AP1(3)-S₅-AP1(3) (CRE comprising 7×cAMPRE and 6×AP1(3));
wherein Sₓ represents a spacer sequence of length X nucleotides.

The lengths of spacers specified above have been found to be effective in the specific examples discussed below. While other lengths of spacers are also expected to be functional, these represent preferred spacer lengths; this applies with respect to all aspects and embodiments of the invention comprising these TFBS as set out below.

In some specific embodiments, the CRE comprises one of the following sequences:
TGACGTCA-S-TGACGTCA-S-TGACGTCA-S-TGACGTCA-S-TGACGTCA-S-TGA[GC]TCA-S-TGA[GC]TCA-S-TGA[GC]TCA (“TGACGTCA” and “TGA[GC]TCA” disclosed as SEQ ID NOS 120 and 122, CRE comprising 5×cAMPRE and 3×AP1 TFBS);
TGACGTCA-S-TGACGTCA-S-TGACGTCA-S-TGACGTCA-S-TGACGTCA-S-TGA[GC]TCA-S-TGA[GC]TCA-S-TGA[GC]TCA-S-TGA[GC]TCA (“TGACGTCA” and “TGA[GC]TCA” disclosed as SEQ ID NOS 121-122, CRE comprising 5×cAMPRE and 4×AP1 TFBS);
TGA[GC]TCA-S-TGA[GC]TCA-S-TGA[GC]TCA-S-TGA[GC]TCA-S-TGA[GC]TCA-S-TGA[GC]TCA-S-TGA[GC]TCA-S-TGA[GC]TCA (“TGA[GC]TCA” disclosed as SEQ ID NO: 122, CRE comprising 8×AP1 TFBS); and
TGACGT-S-TGACGT-S-TGACGT-S-TGA[GC]TCA-S-TGA[GC]TCA-S-TGA[GC]TCA-S-TGA[GC]TCA-S-[AG]CGTG-S-[AG]CGTG-S-[AG]CGTG (“TGACGT”, “TGA[GC]TCA”, and “[AG]CGTG” disclosed as SEQ ID NOS 123, 122, and 216, CRE 3×ATF6, 4×AP1 and 3×HIF TFBS);
wherein S represents an optional, but preferable, spacer sequence. Suitable lengths for the spacers are discussed above.

In some specific embodiments, the CRE comprises one of the following sequences:
TGACGTCA-S-TGACGTCA-S-TGACGTCA-S-TGACGTCA-S-TGACGTCA-S-TGACTCAG-S-TGACTCAG-S-TGACTCAG (“TGACGTCA” disclosed as SEQ ID NO: 124, CRE from Synp-FORCSV-10, comprising 5×cAMPRE and 3×AP1(2) TFBS);
TGACGTCA-S-TGACGTCA-S-TGACGTCA-S-TGACGTCA-S-TGACGTCA-S-TGACTCAG-S-TGACTCAG-S-TGACTCAG-S-TGACTCAG (“TGACGTCA” disclosed as SEQ ID NO: 125, CRE from Synp-FORCMV-09 comprising 5×cAMPRE and 4×AP1(2) TFBS);
TGAGTCA-S-TGAGTCA-S-TGAGTCA-S-TGAGTCA-S-TGAGTCA-S-TGAGTCA-S-TGAGTCA-S-TGAGTCA (“TGAGTCA” disclosed as SEQ ID NO: 126, CRE from Synp-FMP-02, and Synp-FLP-01 comprising 8×AP1(1) TFBS);
TGACGTGCT-S-TGACGTGCT-S-TGACGTGCT-S-TGAGTCA-S-TGAGTCA-S-TGAGTCA-S-TGAGTCA-S-CTGCACGTA-S-CTGCACGTA-S-CTGCACGTA (“TGACGTGCT”, “TGAGTCA”, and “CTGCACGTA” SEQ ID NOS 127, 126, and 91, CRE comprising 3×ATF6, 4×AP1(1) and 3×HRE1 TFBS); and
TGACGTCA-S-TGACGTCA-S-TGACGTCA-S-TGACGTCA-S-TGACGTCA-S-TGACTCA-S-TGACTCA-S-TGACTCA-S-TGACTCA (“TGACGTCA” and “TGACTCA” disclosed as SEQ ID NOS+128 and 116, CRE comprising 5×cAMPRE and 4×AP1(1) TFBS); and
TGACGTCA-S-TGACGTCA-S-TGACGTCA-S-TGACGTCA-S-TGACGTCA-S-TGACGTCA-S-TGACGTCA-S-TGA[GC]TCA-S-TGA[GC]TCA-S-TGA[GC]TCA-S-TGA[GC]TCA-S-TGA[GC]TCA-S-TGA[GC]TCA (“TGACGTCA” and “TGA[GC]TCA” disclosed as SEQ ID NOS+129 and 122, CRE comprising 7×cAMPRE and 6×AP1); and
TGACGTCA-S-TGACGTCA-S-TGACGTCA-S-TGACGTCA-S-TGACGTCA-S-TGACGTCA-S-TGACGTCA-S-TGACTCA-S-TGACTCA-S-TGACTCA-S-TGACTCA-S-TGACTCA-S-TGACTCA (“TGACGTCA” and “TGACTCA” disclosed as SEQ ID NO: 130 and 116, CRE from FORNEW, FORNCMV, FORNCMV53, FORNMinTK, FORNMLP, FORNSV40, FORNpJB42, FORNTATAm6a comprising 7×cAMPRE and 6×AP1(3));
wherein S represents an optional, but preferable, spacer sequence. Suitable lengths for the spacers are discussed above.

In some specific preferred embodiments, the CRE comprises one of the following sequences:
TGACGTCACGATTACCATTGACGTCACGATTACCATTGACGTCACGATTACCATT GACGTCACGATTACCATTGACGTCAGCGATTAAGATGACTCAGCGATTAAGATGA CTCAGCGATTAAGATGACTCAG (SEQ ID NO: 131, CRE from Synp-FORCSV-10, comprising 5×cAMPRE and 3×AP1(2) TFBS);
TGACGTCACGATTACCATTGACGTCACGATTACCATTGACGTCACGATTACCATT GACGTCACGATTACCATTGACGTCAGCGATTAAGATGACTCAGCGATTAAGATGA CTCAGCGATTAAGATGACTCAGCGATTAAGATGACTCAG (SEQ ID NO: 132, CRE from Synp-FORCMV-09 comprising 5×cAMPRE and 4×AP1(2) TFBS);
TGAGTCAGATGATGCGTAGCTAGTAGTTGAGTCAGATGATGCGTAGCTAGTAGTT GAGTCAGATGATGCGTAGCTAGTAGTTGAGTCAGATGATGCGTAGCTAGTAGTTG AGTCAGATGATGCGTAGCTAGTAGTTGAGTCAGATGATGCGTAGCTAGTAGTTGA GTCAGATGATGCGTAGCTAGTAGTTGAGTCA (SEQ ID NO: 133, CRE from Synp-FMP-02, and Synp-FLP-01 comprising 8×AP1(1) TFBS);
TGACGTGCTGATGATGCGTAGCTAGTAGTTGACGTGCTGATGATGCGTAGCTAGT AGTTGACGTGCTGATGATGCGTAGCTAGTAGTTGAGTCAGATGATGCGTAGCTAG TAGTTGAGTCAGATGATGCGTAGCTAGTAGTTGAGTCAGATGATGCGTAGCTAGT AGTTGAGTCAGATGATGCGTAGCTAGTAGTCTGCACGTAGATGATGCGTAGCTAG TAGTCTGCACGTAGATGATGCGTAGCTAGTAGTCTGCACGTA (SEQ ID NO: 134, CRE comprising 3×ATF6, 4×AP1(1) and 3×HRE1 TFBS); and
TGACGTCACGATTACCATTGACGTCACGATTACCATTGACGTCACGATTACCATT GACGTCACGATTACCATTGACGTCAGCGATTAAGATGACTCAGCGATTAAGATGA CTCAGCGATTAAGATGACTCAGCGATTAAGATGACTCA (SEQ ID NO: 135, CRE comprising 5×cAMPRE and 4×AP1(1) TFBS);
AATAAAATATCTTTATTTTCATTACATCTGTGTGTTGGTTTTTTGTGTGCCATTGAC GTCACGATTTGACGTCAACCATTGACGTCACGATTTGACGTCAACCATTGACGTC ACGATTTGACGTCACGATTTGACGTCAACCATTGACTCACGATTTGACTCAACCA TTGACTCACGATTTGACTCAACCATTGACTCACGATTTGACTCACGATT (SEQ ID NO: 136, CRE from FORNEW, FORNCMV, FORNCMV53, FORNMinTK, FORNMLP, FORNSV40, FORNpJB42, FORNTATAm6a comprising 7×cAMPRE and 6×AP1(3));
or a functional variant of any of said sequences that comprises a sequence that is at least 80% identical thereto, preferably 85%, 90%, 95% or 99% identical thereto.

Typically, it is preferred that in such functional variants the TFBS sequences present are identical to the reference sequence, and substantially all variation arises in the spacer sequences lying therebetween.

The abovementioned CREs have been shown to provide good levels of inducibility and powerful expression upon induction, and low levels of background expression, when combined with a minimal promoter to for an inducible promoter. Thus, they are all useful for the provision of forskolin inducible promoters. The CREs demonstrate some degree of variation in terms of inducibility and expression levels upon induction, and this allows a promoter to be selected which has desired properties.

A CRE having the following structure cAMPRE-S-cAMPRE-S-cAMPRE-S-cAMPRE-S-cAMPRE-S-AP1-S-AP1-S-AP1-S-AP has been shown to provide excellent properties in terms of inducibility and expression when coupled to a minimal promoter. Accordingly, such a CRE represents a particularly preferred embodiment of the invention.

A CRE having the following structure ATF6-S-ATF6-S-ATF6-S-AP1-S-AP1-S-AP1-S-AP1-S—HIF-S-HIF—S-HIF has been shown to provide exceptional properties in terms of inducibility and expression when coupled to a minimal promoter. Accordingly, such a CRE represents a particularly preferred embodiment of the invention. It is surprising that such a CRE should perform so well, given that it includes several TFBS that are not known or expected to be induced by forskolin, and fewer TFBS that are induced by forskolin than some other CREs that are less inducible and powerful. It seems that an unexpected synergy has arisen in view of the combination of TFBS present in the CRE.

A synthetic forskolin-inducible promoter comprising the structure cAMPRE-S-cAMPRE-S-cAMPRE-S-cAMPRE-S-cAMPRE-S-cAMPRE-S-cAMPRE-S-AP1(3)-S-AP1(3)-S-AP1(3)-S-AP1(3)-S-AP1(3)-S-AP1(3) has been shown to provide exceptional properties in terms of inducibility and expression. Accordingly, such a promoter represents a particularly preferred embodiment of the invention. It seems that a particular synergy has arisen in view of the combination of TFBS present in the CRE.

In a further embodiment the promoter may comprise a cis-regulatory module (CRM) comprising a CRE according to the first aspect of the invention. Other CREs in the CRM can be forskolin-inducible CREs, or they can have any other function.

Preferably the synthetic forskolin-inducible promoter comprises a CRE (or CRM) as discussed above linked to a minimal promoter or a proximal promoter, preferably a minimal promoter.

The minimal promoter can be any suitable minimal promoter. A wide range of minimal promoters are known in the art. Without limitation, suitable minimal promoters include CMV minimal promoter (CMV-MP), YB-TATA minimal promoter (YB-TABA), HSV thymidine kinase minimal promoter (MinTK), SV40 minimal promoter (SV40-MP), or G6PC-MP (which is a liver-derived non-TATA box MP). The minimal promoter can be a synthetic minimal promoter.

The sequence of CMV-MP is:

(SEQ ID NO: 104)
AGGTCTATATAAGCAGAGCTCGTTTAGTGAACCGTCAGATCGCCTAGATA
CGCCATCCACGCTGTTTTGACCTCCATAGAAGATCGCCACC.

The sequence of YB-TATA is:

(SEQ ID NO: 137)
GCGATTAATCCATATGCTCTAGAGGGTATATAATGGGGGCCACTAGTCTA
CTACCAGAAAGCTTGGTACCGAGCTCGGATCCAGCCACC.

However, a shorter version of the YB-TATA MP is known in the art and this is should provide an effective alternative to the YB-TATA MP sequence recited above. The sequence of this shorter YB-TATA MP (referred to as sYB-TATA) is TCTAGAGGGTATATAATGGGGGCCA (SEQ ID NO: 10 5). Accordingly, wherever YB-TATA is referred to as a component of an inducible promoter herein, an equivalent sequence with sYB-TATA substituted in place of YB-TATA is also considered to be an alternative embodiment of the invention. In other words, in such an alternative the sequence of sYB-TATA is retained, while the remaining portions of YB-TATA can be replaced with other sequences, typically spacer sequences.

The sequence of the MinTK MP is:

(SEQ ID NO: 107)
TTCGCATATTAAGGTGACGCGTGTGGCCTCGAACACCGAGCGACCCTGCA
GCGACCCGCTTAA.

The sequence of SV40-MP is:

(SEQ ID NO: 207)
TGCATCTCAATTAGTCAGCAACCATAGTCCCGCCCCTAACTCCGCCCATC
CCGCCCCTAACTCCGCCCAGTTCCGCCCATTCTCCGCCCCATCGCTGACT
AATTTTTTTTATTTATGCAGAGGCCGAGGCCGCCTCGGCCTCTGAGCTAT
TCCAGAAGTAGTGAGGAGGCTTTTTTGGAGGCCTAGGCTTTTGCAAA.

The sequence of G6PC-MP is:

(SEQ ID NO: 208)
GGGCATATAAAACAGGGGCAAGGCACAGACTCATAGCAGAGCAATCACCA
CCAAGCCTGGAATAACTGCAGCCACC

In some embodiments, the synthetic forskolin-inducible promoter comprises any one of the CRE sequences set out above operably linked to a minimal promoter or a proximal promoter, preferably a minimal promoter. The CRE is preferably coupled to the MP via a spacer, but in some cases, there may be another CRE provided therebetween. The CRE may also be operably linked to the MP without a spacer.

The spacer sequence between the CRE and the minimal promoter can be of any suitable length. Typically, the spacer is from 5 to 100 nucleotides in length, from 20 to 80 nucleotides in length, or from 30 to 70 nucleotides in length. For example, spacers of 5, 10, 18, 20, 21, 42, 50, 59, 65 and 66 nucleotides in length have been used in specific non-limiting examples of the invention, and these function well. However, other lengths of spacers can be used, and the skilled person can readily determine suitable lengths of spacers.

In some preferred embodiments, the synthetic forskolin-inducible promoter comprises one of the following structures:
cAMPRE-S-cAMPRE-S-cAMPRE-S-cAMPRE-S-cAMPRE-S-AP1-S-AP1-S-AP1-S-MP (i.e. a CRE comprising 5×cAMPRE and 3×ATF6TFBS and a MP);
cAMPRE-S-cAMPRE-S-cAMPRE-S-cAMPRE-S-cAMPRE-S-AP1-S-AP1-S-AP1-S-AP1-S-MP (i.e. a CRE comprising 5×cAMPRE and 4×AP1 TFBS and a MP);
AP1-S-AP1-S-AP1-S-AP1-S-AP1-S-AP1-S-AP1-S-AP1-S-MP (i.e. a CRE comprising 8×AP1 TFBS and a MP);
ATF6-S-ATF6-S-ATF6-S-AP1-S-AP1-S-AP1-S-AP1-S—HIF-S-HIF—S-HIF-S-MP (i.e. a CRE comprising 3×ATF6, 4×AP1 and 3×HIF TFBS and a MP);
cAMPRE-S-cAMPRE-S-cAMPRE-S-cAMPRE-S-cAMPRE-S-AP1-S-AP1-S-AP1-S-AP1-S-MP (i.e. a CRE comprising 5×cAMPRE 4×AP1 TFBS and MP); and
cAMPRE-S-cAMPRE-S-cAMPRE-S-cAMPRE-S-cAMPRE-S-cAMPRE-S-cAMPRE-S-AP1(3)-S-AP1(3)-S-AP1(3)-S-AP1(3)-S-AP1(3)-S-AP1(3)-S-MP (CRE comprising 7×cAMPRE and 6×AP1(3) and MP);
and wherein S represents an optional, but preferable, spacer sequence and MP represents a minimal promoter. Suitable lengths for the spacers are discussed above.

In a particularly preferred embodiment, the synthetic forskolin-inducible promoter comprises the following structure ATF6-S-ATF6-S-ATF6-S-AP1-S-AP1-S-AP1-S-AP1-S—HIF-S—HIF—S-HIF-S-MP, wherein S represents an optional, but preferable, spacer sequence and MP represents a minimal promoter. More preferably, the MP is CMV-MP.

In some preferred embodiments, the synthetic forskolin-inducible promoter comprises one of the following structures:
cAMPRE-S-cAMPRE-S-cAMPRE-S-cAMPRE-S-cAMPRE-S-AP1-S-AP1-S-AP1-S-SV40-MP (i.e. a CRE comprising 5×cAMPRE and 3×AP1 TFBS and SV40-MP);
cAMPRE-S-cAMPRE-S-cAMPRE-S-cAMPRE-S-cAMPRE-S-AP1-S-AP1-S-AP1-S-AP1-S-CMV-MP (i.e. a CRE comprising 5×cAMPRE and 4×AP1 TFBS and CMV-MP);
AP1-S-AP1-S-AP1-S-AP1-S-AP1-S-AP1-S-AP1-S-AP1-S-(Min-TK or G6PC MP or CMV-MP) (i.e. a CRE comprising 8×AP1 TFBS and Min-TK or G6PC MP or CMV-MP);
ATF6-S-ATF6-S-ATF6-S-AP1-S-AP1-S-AP1-S-AP1-S—HIF-S-HIF—S-HIF-S-CMV-MP (i.e. CRE comprising 3×ATF6, 4×AP1 and 3×HIF TFBS and CMV-MP);
cAMPRE-S-cAMPRE-S-cAMPRE-S-cAMPRE-S-cAMPRE-S-AP1-S-AP1-S-AP1-S-AP1-S-YB-TATA (i.e. a CRE from comprising 5×cAMPRE 4×AP1 TFBS and YB-TATA); and
cAMPRE-S-cAMPRE-S-cAMPRE-S-cAMPRE-S-cAMPRE-S-cAMPRE-S-cAMPRE-S-AP1(3)-S-AP1(3)-S-AP1(3)-S-AP1(3)-S-AP1(3)-S-AP1(3)-S-(YB TATA or CMV-MP or CMV53 or MinTK or MLP or SV40 or pJV42 or TATAm6a) (i.e. CRE comprising 7×cAMPRE and 6×AP1(3) and YB TATA or CMV-MP or CMV53 or MinTK or MLP or SV40 or pJV42 or TATAm6a),
wherein S represents an optional, but preferable, spacer sequence. Suitable lengths for the spacers are discussed above.

In some preferred embodiments, the synthetic forskolin-inducible promoter comprises one of the following sequences:
TGACGTCA-S-TGACGTCA-S-TGACGTCA-S-TGACGTCA-S-TGACGTCA-S-TGA[GC]TCA-S-TGA[GC]TCA-S-TGA[GC]TCA-S-TGCATCTCAATTAGTCAGCAACCATAGTCCCGCCCCTAACTCCGCCCATCCCGCC CCTAACTCCGCCCAGTTCCGCCCATTCTCCGCCCCATCGCTGACTAATTTTTTTTA TTTATGCAGAGGCCGAGGCCGCCTCGGCCTCTGAGCTATTCCAGAAGTAGTGAGG AGGCTTTTTTGGAGGCCTAGGCTTTTGCAAA (SEQ ID NOS 138, 138, 138, 138, 138, 122, 122, 122, and 207 respectively, CRE comprising 5×cAMPRE and 3×AP1 TFBS and SV40-MP);
TGACGTCA-S-TGACGTCA-S-TGACGTCA-S-TGACGTCA-S-TGACGTCA-S-TGA[GC]TCA-S-TGA[GC]TCA-S-TGA[GC]TCA-S-TGA[GC]TCA-S-AGGTCTATATAAGCAGAGCTCGTTTAGTGAACCGTCAGATCGCCTAGATACGCCA TCCACGCTGTTTTGACCTCCATAGAAGATCGCCACC (SEQ ID NOS 139, 139, 139, 139, 139, 122, 122, 122, 122, and 10⁴, respectively, CRE comprising 5×cAMPRE and 4×AP1 TFBS and CMV-MP);
TGA[GC]TCA-S-TGA[GC]TCA-S-TGA[GC]TCA-S-TGA[GC]TCA-S-TGA[GC]TCA-S-TGA[GC]TCA-S-TGA[GC]TCA-S-TGA[GC]TCA-S-(TTCGCATATTAAGGTGACGCGTGTGGCCTCGAACACCGAGCGACCCTGCAGCGA CCCGCTTAA or GGGCATATAAAACAGGGGCAAGGCACAGACTCATAGCAGAGCAATCACCACCAA GCCTGGAATAACTGCAGCCACC or AGGTCTATATAAGCAGAGCTCGTTTAGTGAACCGTCAGATCGCCTAGATACGCCA TCCACGCTGTTTTGACCTCCATAGAAGATCGCCACC) (SEQ ID NO: 140, 140, 140, 140, 140, 140, 140, 140, 107, 208, and 10⁴, respectively, CRE comprising 8×AP1 TFBS and Min-TK or G6PC MP or CMV-MP);
TGACGT-S-TGACGT-S-TGACGT-S-TGA[GC]TCA-S-TGA[GC]TCA-S-TGA[GC]TCA-S-TGA[GC]TCA-S-[AG]CGTG-S-[AG]CGTG-S-[AG]CGTG-S-AGGTCTATATAAGCAGAGCTCGTTTAGTGAACCGTCAGATCGCCTAGATACGCCA TCCACGCTGTTTTGACCTCCATAGAAGATCGCCACC (SEQ ID NO: 141, 141, 141, 113, 113, 113, 113, 216, 216, 216, and 104, respectively, CRE 3×ATF6, 4×AP1 and 3×HIF TFBS and CMV-MP); and
TGACGTCA-S-TGACGTCA-S-TGACGTCA-S-TGACGTCA-S-TGACGTCA-S-TGA[GC]TCA-S-TGA[GC]TCA-S-TGA[GC]TCA-S-TGA[GC]TCA-S-GCGATTAATCCATATGCTCTAGAGGGTATATAATGGGGGCCACTAGTCTACTACC AGAAAGCTTGGTACCGAGCTCGGATCCAGCCACC (SEQ ID NO: 142, 142, 142, 142, 142, 113, 113, 113, 113, and 106, respectively, CRE comprising 5×cAMPRE and 4×AP1 TFBS and YB-TATA);
wherein S represents an optional, but preferable, spacer sequence. Suitable lengths for the spacers are discussed above.

In some preferred embodiments, the synthetic forskolin-inducible promoter comprises one of the following sequences (the TFBS sequences are underlined and minimal promoter sequences are shown in bold):

(SEQ ID NO: 6; core sequence of Synp-FORCSV-10)
TGACGTCACGATTACCATTGACGTCACGATTACCATTGACGTCA
CGATTACCATTGACGTCACGATTACCATTGACGTCAGCGATTAAGA
TGACTCAGCGATTAAGATGACTCAGCGATTAAGATGACTCAG
CGATTAAGATGACTCACTAGCCCGGGCTCGAGATCTGCGATC
TGCATCTCAATTAGTCAGCAACCATAGTCCCGCCCCT
AACTCCGCCCATCCCGCCCCTAACTCCGCCCAGTTCC
GCCCATTCTCCGCCCCATCGCTGACTAATTTTTTTTA
TTTATGCAGAGGCCGAGGCCGCCTCGGCCTCTGAGCT
ATTCCAGAAGTAGTGAGGAGGCTTTTTTGGAGGCCTA
GGCTTTTGCAAA;
(SEQ ID NO: 7; core sequence of Synp-FORCMV-09)
TGACGTCACGATTACCATTGACGTCACGATTACCATTGACGTCA
CGATTACCATTGACGTCACGATTACCATTGACGTCAGCGATTAAGA
TGACTCAGCGATTAAGATGACTCAGCGATTAAGATGACTCAG
CGATTAAGATGACTCAGCGATTAATCCATATGC
AGGTCTATATAAGCAGAGCTCGTTTAGTGAACCGTCA
GATCGCCTAGATACGCCATCCACGCTGTTTTGACCTC
CATAGAAGATCGCCACC;
(SEQ ID NO: 8; Synp-FMP-02)
TGAGTCAGATGATGCGTAGCTAGTAGTTGAGTCAGATGATGCGTA
GCTAGTAGTTGAGTCAGATGATGCGTAGCTAGTAGTTGAGTCA
GATGATGCGTAGCTAGTAGTTGAGTCAGATGATGCGTAGCTAG
TAGTTGAGTCAGATGATGCGTAGCTAGTAGTTGAGTCAGATGA
TGCGTAGCTAGTAGTTGAGTCAGTAGTCGTATGCTGATGCGCA
GTTAGCGTAGCTGAGGTACCGTCGACGATATCGGATCC
TTCGCATATTAAGGTGACGCGTGTGGCCTCGAACACC
GAGCGACCCTGCAGCGACCCGCTTAA;
and
(SEQ ID NO: 9; Synp-FLP-01)
TGAGTCAGATGATGCGTAGCTAGTAGTTGAGTCAGATGATGCGTA
GCTAGTAGTTGAGTCAGATGATGCGTAGCTAGTAGTTGAGTCA
GATGATGCGTAGCTAGTAGTTGAGTCAGATGATGCGTAGCTAG
TAGTTGAGTCAGATGATGCGTAGCTAGTAGTTGAGTCAGATGA
TGCGTAGCTAGTAGTTGAGTCAGTAGTCGTATGCTGATGCGCA
AGTTGCGTAGCTGAGGTACCGTCGACGATATCGGATCC
GGGCATATAAAACAGGGGCAAGGCACAGACTCATAGC
AGAGCAATCACCACCAAGCCTGGAATAACTGCAGCCA
CC
(SEQ ID NO: 143; FORNYB)
CCATTGACGTCACGATTTGACGTCAACCATTGACGTCACGATTTGA
CGTCAACCATTGACGTCACGATTTGACGTCACGATTTGACGTCAAC
CATTGACTCACGATTTGACTCAACCATTGACTCACGATTTGACTCA
ACCATTGACTCACGATTTGACTCACGATTGCGATTAATCCATATGC
TCTAGAGGGTATATAATGGGGGCCACTAGTCTACTACCAGAAAGCT
TGGTACCGAGCTCGGATCCAGCCACC
(SEQ ID NO: 144; FORNTATAm6a)
CCATTGACGTCACGATTTGACGTCAACCATTGACGTCACGATTTGA
CGTCAACCATTGACGTCACGATTTGACGTCACGATTTGACGTCAAC
CATTGACTCACGATTTGACTCAACCATTGACTCACGATTTGACTCA
ACCATTGACTCACGATTTGACTCACGATTTATAAAAGGCAGAGCTC
GTTTAGTGAACCGaagcttggactaaagcggact
(SEQ ID NO: 210; FORNpJB42)
CCATTGACGTCACGATTTGACGTCAACCATTGACGTCACGATTTGA
CGTCAACCATTGACGTCACGATTTGACGTCACGATTTGACGTCAAC
CATTGACTCACGATTTGACTCAACCATTGACTCACGATTTGACTCA
ACCATTGACTCACGATTTGACTCACGATTCTGACAAATTCAGTATA
AAAGCTTGGGGCTGGGGCCGAGCACTGGGGACTTTGAGGGTGGCCA
GGCCAGCGTAGGAGGCCAGCGTAGGATCCTGCTGGGAGCGGGGAAC
TGAGGGAAGCGACGCCGAGAAAGCAGGCGTACCACGGAGGGAGAGA
AAAGCTCCGGAAGCCCAGCAGCG

or a functional variant of any of said sequences that comprises a sequence that is at least 80% identical thereto, preferably 85%, 90%, 95% or 99% identical thereto.

Typically, it is preferred that in such functional variants the TFBS and MP sequences present are identical to the reference sequence, and substantially all sequence variation arises in the spacer sequences lying therebetween.

The abovementioned forskolin-inducible promoters have been shown to provide good levels of inducibility and powerful expression upon induction, and low levels of background expression. The promoters demonstrate a degree of variation in terms of inducibility and expression levels upon induction, and this allows a promoter to be selected which has desired properties.

A synthetic forskolin-inducible promoter comprising the structure ATF6-S-ATF6-S-ATF6-S-AP1-S-AP1-S-AP1-S-AP1-S-HIF—S-HIF-S—HIF-S-MP, preferably wherein the MP is CMV-MP, has been shown to provide exceptional properties in terms of inducibility and expression.

Accordingly, such a promoter represents a particularly preferred embodiment of the invention. As mentioned above, it is surprising that such a CRE should perform so well, given that it includes several TFBS that are not known or expected to be induced by forskolin, and fewer TFBS that are induced by forskolin than some other CREs that are less inducible and powerful. It seems that an unexpected synergy has arisen in view of the combination of TFBS present in the CRE.

In preferred embodiments of the invention, the inducibility of the promoter is such that upon induction (e.g. after cells, e.g. HEK293 cells, are exposed to 18 μM forskolin for 5h) the expression level of the transgene which is under the control of the promoter is increased by at least a 3-fold, more preferably a 5-, 10-, 15-, 20-, 30-, or 50-fold.

In some embodiments of the invention, upon induction (e.g. after cells, e.g. HEK293 cells, are exposed to 18 μM forskolin for 5h) the expression level of the transgene which is under the control of the promoter is at least 50% of that provided by the CMV-IE promoter (i.e. an otherwise identical vector in the same cells under the same conditions, but in which expression of the transgene is under control of CMV-IE rather than the forskolin inducible promoter). More preferably the expression level of the transgene is at least 75%, 100%, 150%, 200%, 300%, 400%, 500%, 750% or 1000% of that provided by the CMV-IE promoter.

The consensus cAMP response element TGACGTCA was described in JBC vol 272, No. 31, Issue of August 1, pp. 19158-19164, 1997, which is incorporated herein by reference. A novel cAMP response element CACTTGATC was described in J. Neurochem, 63 (1), 28-40 July 1994, which is incorporated herein by reference; this element can be used in the forskolin-inducible promoters as discussed above.

The cAMP response element (CRE)/cAMP autoregulatory response element (CARE) are as discussed in Molecular Endocrinology, Volume 13, Issue 7, 1 Jul. 1999, Pages 1207-1217.

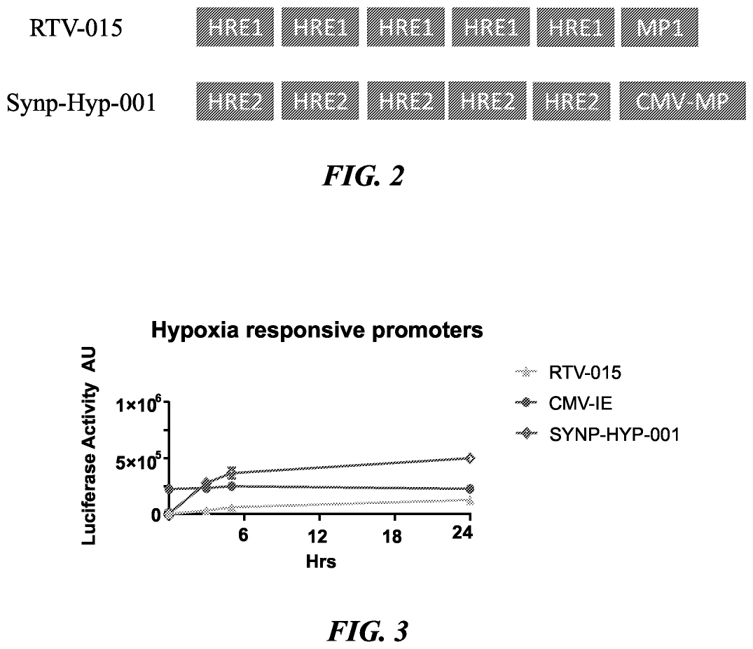

cAMP response elements are also discussed in Gut 3 May 2005, 54(9):1309-1317, e.g. in FIG. 2. Such elements can be used in the forskolin-inducible promoters as discussed above.

Another cAMP-inducible promoter is described in U.S. Pat. No. 6,596,508, which is incorporated herein by reference, see for example SEQ ID NO: 3 of U.S. Pat. No. 659,650. Such promoters can be used in the present invention. cAMP response elements from the VIP promoter are also disclosed, e.g. SEQ ID NO: 1 and 2 of U.S. Pat. No. 659, 650.

Further cAMP response elements are also described in U.S. Pat. No. 8,986,937, which is incorporated herein by reference. Exemplary naturally occurring cAMP-inducible promoters described therein include the PEPCK promoter (Roesler et al. (1998) The Journal of Biological Chemistry, 273, 14950-14957); promoters containing the cAMP-responsive element (CRE) that is located at position-294 with respect to the translation initiation site of the human cyclin D2 promoter (Munfiz et al. (2006) Biology of Reproduction 75(2): 279-288); and promoters containing the cAMP-responsive element (CRE) of the lactate dehydrogenase A subunit promoter (Welfeld et al. (1989) J. Biol. Chem. 264(12):6941-7. Exemplary cAMP-inducible promoters comprise a 236-nucleotide glycoprotein hormone alpha subunit promoter, which contains a cyclic AMP (cAMP) regulatory element (CRE) (AF401991), as described in U.S. Pat. Appl. Publication no. US2008-0187942, published on Aug. 7, 2008, which is incorporated herein by reference. Such elements can be used in forskolin-inducible promoters as described above.

U.S. Pat. No. 9,060,310, which is incorporated herein by reference, describes further cAMP response elements, e.g. various CRE-palindromes and hairpins of SEQ ID NOs: 2, 3, 8, 9, 10 and 11 of U.S. Pat. No. 9,060,310. Such cAMP response elements can be used in forskolin-inducible promoters as described above.

US20070036810 and Mayr B, Montminy M., Nat Rev Mol Cell Biol 2001 August; 2(8):599-609 (both incorporated by reference) disclose cAMP responses elements which include a palindromic sequence of TGACGTCA or asymmetric variations which include a CRE half site with the core sequence TGAC. Such elements can be used in forskolin-inducible promoters as described above.

Various cAMP induced genes/well-known targets of cAMP are also discussed in US20070036810 (incorporated herein by reference), e.g. TSHalpha, phosphoenol pyruvate carboxykinase (PEPCK), crystallin alpha-B, and EGF-like molecule amphiregulin. Promoters from such genes may also be relevant in the present context.

Suitable inducers that are able to activate adenylyl cyclase include, but are not limited to:
Forskolin (a potent adenylyl cyclase activator (CAS Number 66575-29-9));
NKH 477 (a water-soluble analogue of forskolin (CAS Number 138605-00));
PACAP-27 (a neuropeptide that stimulates adenylate cyclase (CAS Number 127317-03-7));
PACAP-38 (a neuropeptide that stimulates adenylate cyclase (CAS Number 137061-48-4));
Pertussis toxin CAS Number 70323-44-3; and
Cholera toxin (CAS Number 9012-63-9).

All of the above are commercially available from Sigma-Aldrich, Inc (now part of Merck KGaA).

In some preferred embodiments of the invention the inducer comprises forskolin or NKH 477.

Forskolin is classified as generally regarded as safe (GRAS), which is generally desirable from a safety point of view. Forskolin (also known as coleonol) is a labdane diterpene that is produced by the Indian Coleus plant (Plectranthus barbatus). Forskolin is a commonly used in material research to increase levels of cyclic AMP. Forskolin is also used in traditional medicine. Since forskolin is GRAS, it is a preferred inducer for the promoters according to this invention in gene therapy applications.

NKH 477 is a water-soluble analogue of forskolin and therefore may be advantageous in terms of ease of use in cell culture in particular. Since NKH477 is water-soluble, it can be a preferred inducer.

The inducer can be administered to the cells in any suitable manner. For example, the inducer can be added to the culture medium, if necessary with a suitable carrier, surfactant or suchlike.

A suitable dosage rate for any given inducer can be readily determined by the person skilled in the art. The person skilled in the art can thus readily determine for any inducer an appropriate way to deliver the inducer to the cells, and a suitable concentration to use. In general terms, the inducer may be administered at any suitable concentration in the range of from 1 nM to 1000 μM, optionally in the range of from 0.1 μM to 100 μM.

Forskolin may suitably be administered to the cells at a concentration of from 0.1 μM to 1000 μM, more preferable from 1 μM to 100 μM, yet more preferably from 5 μM to 30 μM.

For example, administration of a concentration of about 18 μM to cells was determined to be optimum to induce expression in HEK-293 cells.

NKH 477 may suitably be administered to the cells at a concentration of from 0.1 μM to 1000 μM, more preferable from 1 μM to 100 μM, yet more preferably from 2 μM to 20 μM. For example, administration of a concentration of about 8 μM to cells was determined to be optimum to induce expression in HEK-293 cells.

The method may suitably comprise ceasing to administer the inducer. Ceasing to administer the inducer will lead to at least a reduction of expression of the expression product. Typically, expression of the expression product will return to a baseline level over time.

The method may suitably comprise varying the concentration of the inducer administered to the cells over time. This can be used to modulate the level of expression of the expression product.

In some embodiments methods disclosed herein may involve administering to the cells an inhibitor of adenylyl cyclase, which acts to reduce or turn off expression of the expression product. Inhibitors of adenylyl cyclase include, but are not limited to:
NB001—inhibitor of adenylyl cyclase 1 (AC1);
9-Cyclopentyladenine monomethanesulfonate—stable, cell-permeable, non-competitive adenylyl cyclase inhibitor;
SQ 22,536—cell-permeable adenylyl cyclase inhibitor;
MDL-12,330A hydrochloride—adenylyl cyclase inhibitor;
2′,5′-Dideoxyadenosine—cell-permeable adenylyl cyclase inhibitor;
2′,5′-Dideoxyadenosine 3′-triphosphate tetrasodium salt—potent inhibitor of adenylyl cyclase;
MANT-GTPγS—potent and competitive adenylyl cyclase inhibitor;
2′,3′-Dideoxyadenosine—specific adenylyl cyclase inhibitor;
NKY80—selective adenylyl cyclase-V inhibitor; and
KH7—selective inhibitor of soluble adenylyl cyclase.

All of the above are commercially available from Sigma-Aldrich, Inc (now part of Merck KGaA).

Administration of inhibitors of adenylyl cyclase can thus be used to switch off or reduce expression of a toxic protein.

In one embodiment, the synthetic forskolin-inducible promoter does not comprise or consist of one of the following structures:
AP1-S₂₀-AP1-S₂₀-AP1-S₂₀-AP1-S₂₀-AP1-S₂₀-AP1-S₂₀-AP1-S₂₀-AP1-S₅₉-CMV-MP (i.e. structure of RTV-17 comprising 8×AP1 TFBS and a CMV-MP);
ATF6-S₂₀-ATF6-S₂₀-ATF6-S₂₀-AP1-S₂₀-AP1-S₂₀-AP1-S₂₀-AP1-S₂₀-HRE1-S₂₀-HRE1-S₂₀-HRE1-S₅₉-CMV-MP (i.e. structure of Synp-RTV-019 comprising 3×ATF6, 4×AP1 and 3×HIF TFBS and a CMV-MP);
cAMPRE-S₁₀-cAMPRE-S₁₀-cAMPRE-S₁₀-cAMPRE-S₁₀-cAMPRE-S₁₀-AP1-S₁₀-AP1-S₁₀-AP1-S₁₀-AP1-S₀-YB-TATA-MP (i.e. structure of FORCYB1 comprising 5×cAMPRE and 4×AP1 TFBS and YB-TATA-MP).

Herein, Sₓ represents a spacer of length X nucleotides.

In one embodiment, the synthetic forskolin-inducible promoter does not comprise or consist of one of the following sequences:

(SEQ ID NO: 145; Synp-RTV-017)
TGAGTCAGATGATGCGTAGCTAGTAGTTGAGTCAGATGATGCGTAGCTAG
TAGTTGAGTCAGATGATGCGTAGCTAGTAGTTGAGTCAGATGATGCGTAG
CTAGTAGTTGAGTCAGATGATGCGTAGCTAGTAGTTGAGTCAGATGATGC
GTAGCTAGTAGTTGAGTCAGATGATGCGTAGCTAGTAGTTGAGTCAGTAG
TCGTATGCTGATGCGCAGTTAGCGTAGCTGAGGTACCGTCGACGATATCG
GATCCAGGTCTATATAAGCAGAGCTCGTTTAGTGAACCGTCAGATCGCCT
AGATACGCCATCCACGCTGTTTTGACCTCCATAGAAGATCGCCACC;
(SEQ ID NO: 146; Synp-RTV-019)
TGACGTGCTGATGATGCGTAGCTAGTAGTTGACGTGCTGATGATGCGTAG
CTAGTAGTTGACGTGCTGATGATGCGTAGCTAGTAGTTGAGTCAGATGAT
GCGTAGCTAGTAGTTGAGTCAGATGATGCGTAGCTAGTAGTTGAGTCAGA
TGATGCGTAGCTAGTAGTTGAGTCAGATGATGCGTAGCTAGTAGTCTGCA
CGTAGATGATGCGTAGCTAGTAGTCTGCACGTAGATGATGCGTAGCTAGT
AGTCTGCACGTAGATGATGCGTAGCTAGTAGTGCAGTTAGCGTAGCTGAG
GTACCGTCGACGATATCGGATCCAGGTCTATATAAGCAGAGCTCGTTTAG
TGAACCGTCAGATCGCCTAGATACGCCATCCACGCTGTTTTGACCTCCAT
AGAAGATCGCCACC;
and
(SEQ ID NO: 147; Synp-FORCYB1)
TGACGTCACGATTACCATTGACGTCACGATTACCATTGACGTCACGATTA
CCATTGACGTCACGATTACCATTGACGTCAGCGATTAAGATGACTCAGCG
ATTAAGATGACTCAGCGATTAAGATGACTCAGCGATTAAGATGACTCAGC
GATTAATCCATATGCTCTAGAGGGTATATAATGGGGGCCACTAGTCTACT
ACCAGAAAGCTTGGTACCGAGCTCGGATCCAGCCACC.

Temperature-Inducible Promoters:

The inducible promoter may be induced by reduction of temperature, e.g. a cold-shock responsive promoter. In some embodiments, the inducible promoter is a synthetic cold-shock responsive promoter derived from the S1006a gene (calcyclin) of CHO cells. The temperature sensitivity of the S1006a gene (calcyclin) promoter was identified by Thaisuchat et al., 2011 (Thaisuchat, H. et al. (2011) ‘Identification of a novel temperature sensitive promoter in cho cells’, BMC Biotechnology, 11. doi: 10.1186/1472-6750-11-51), which is incorporated herein by reference. In some embodiments, the inducible promoter is one of the synthetic cold-shock responsive promoters shown in FIG. 2 of Thaisuchat et al., 2011. These promoters are induced by decrease of temperature as shown in FIG. 3 of Thaisuchat et al., 2011. Most of these synthetic promoter constructs show expression similar to the known promoter SV40 at 37° C. and are induced by 2-3 times when the temperature is reduced to 33° C. In some embodiments, the inducible promoter is sps5 from FIG. 2 of Thaisuchat et al., 2011. In some preferred embodiments, the inducible promoter is sps8 from FIG. 2 of Thaisuchat et al., 2011.

pH-Inducible Promoters:

The inducible promoter may be induced by reduction or increase of pH to which cells comprising the promoter are exposed. Suitably, the inducible promoter may be induced by reduction of pH, i.e. a promoter inducible under acidic conditions. Suitable acid-inducible promoters are described in Hou et al., 2016 (Hou, J. et al. (2016) ‘Isolation and functional validation of salinity and osmotic stress inducible promoter from the maize type-II H+-pyrophosphatase gene by deletion analysis in transgenic tobacco plants’, PLoS ONE, 11(4), pp. 1-23. doi: 10.1371/journal.pone.0154041), which is incorporated herein by reference.

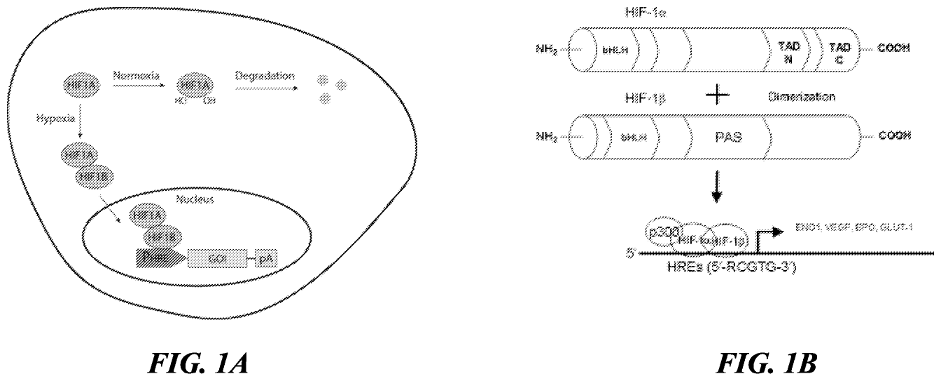

In some embodiments, the inducible promoter is a synthetic promoter inducible under acidic conditions derived from the YGP1 gene or the CCW14 gene. The inducibility by acidic conditions of the YGP1 gene or the CCW14 gene was studied and improved by modifying transcription factor binding sites by Rajkumar et al., 2016 (Rajkumar, A. S. et al. (2016) ‘Engineering of synthetic, stress-responsive yeast promoters’, 44(17). doi: 10.1093/nar/gkw553), which is incorporated herein by reference. In some embodiments, the inducible promoter is one of the synthetic promoter inducible under acidic conditions in FIG. 1A, 2A, 3A and 4A of Rajkumar et al., 2016. These promoters are induced by decrease of pH as shown in FIG. 1B, 2B, 3B and 4B of Rajkumar et al., 2016. Most of these synthetic promoters are induced by up to 10-15 times when the reduced from pH 6 to pH 3. In some preferred embodiments, the inducible promoter is YGP1pr from FIG. 1 of Rajkumar et al., 2016. In other preferred embodiments, the inducible promoter is YGP1pr from FIG. 1 of Rajkumar et al., 2016.

Osmolarity-Inducible Promoters:

The inducible promoter may be osmolarity-induced. Suitable promoters induced by osmolarity are described in Zhang et al. (Molecular Biology Reports volume 39, pages 7347-7353(2012)) which is incorporated herein by reference.

Carbon Source-Inducible Promoters:

The inducible promoter may be induced by addition of a specific carbon source, e.g. a non-sugar carbon source. Alternatively, the inducible promoter may be induced by withdrawal or the absence of a carbon source. Suitable promoters induced by the presence or absence of various carbon sources are described in Weinhandl et al., 2014 (Weinhandl, K. et al. (2014) ‘Carbon source dependent promoters in yeasts’, Microbial Cell Factories, 13(1), pp. 1-17. doi: 10.1186/1475-2859-13-5), which is incorporated herein by reference.

Alcohol (e.g. Ethanol)-Inducible Promoters:

The inducible promoter may be induced by addition of ethanol. Suitable promoters induced by ethanol are described in Matsuzawa et al. (Applied Microbiology and Biotechnology volume 97, pages 6835-6843(2013)), which is incorporated herein by reference.

Amino Acid-Inducible Promoters:

The inducible promoters may be induced by addition of one or more amino acids.

Suitably, the amino acid may be an aromatic amino acid. Suitably, the amino acid may be GABA (gamma aminobutyric acid), which is also a neurotransmitter. Suitable promoter induced by aromatic amino acids and GABA are described in Kim et al. (Applied Microbiology and Biotechnology, volume 99, pages 2705-2714(2015)) which is incorporated herein by reference.

Hormone (e.g. Ecdysone)-Inducible Promoters:

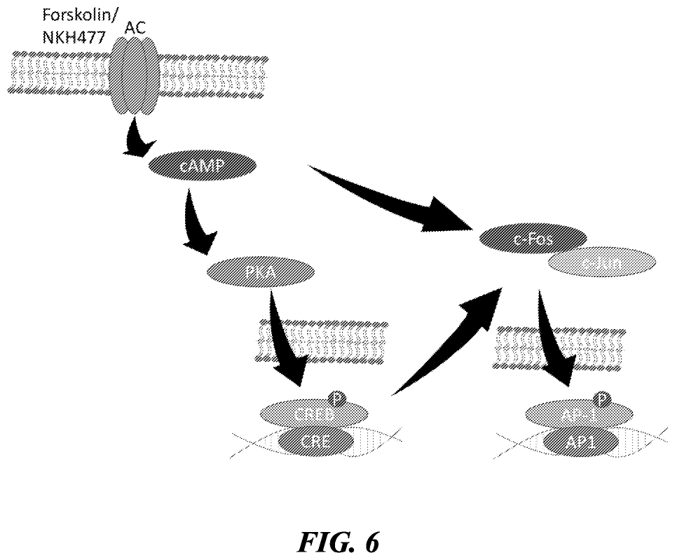

The inducible promoter may be the induced by a steroid hormone. Suitably, the steroid hormone may be ecdysone. A mammalian ecdysone-inducible system was created by No, Yao and Evans (No, D., Yao, T. P. and Evans, R. M. (1996) ‘Ecdysone-inducible gene expression in mammalian cells and transgenic mice’, Proceedings of the National Academy of Sciences of the United States of America, 93(8), pp. 3346-3351. doi: 10.1073/pnas.93.8.3346), which is incorporated herein by reference. Expression of a modified ecdysone receptor in mammalian cells allows expression from an ecdysone responsive promoter to be induced upon addition of ecdysone as shown in FIG. 2 of No, Yao and Evans, 1996. This system showed lower basal activity and higher inducibility than the tetracycline-inducible system as shown in FIG. 6 of No, Yao and Evans, 1996. A suitable commercially available inducible system is available from Agilent technologies and is described in Agilent Technologies (2015) ‘Complete Control Inducible Mammalian Expression System Instruction Manual’, 217460, which is incorporated herein by reference.

Tetracycline-Regulated Promoters:

In some embodiments, the promoter may be induced by the presence or absence of tetracycline or its derivatives.

A suitable promoter induced in the absence of tetracycline or its derivatives is the promoter in the tet-OFF system. In the tet-OFF system, tetracycline-controlled transactivator (tTA) allows transcriptional activation of a tTA-dependent promoter in the absence of tetracycline or its derivatives. tTA and the tTA-dependent promoter were initially created by Gossen and Bujard, 1992 (Gossen, M. and Bujard, H. (1992) ‘Tight control of gene expression in mammalian cells by tetracycline-responsive promoters’, Proceedings of the National Academy of Sciences of the United States of America, 89(12), pp. 5547-5551. doi: 10.1073/pnas.89.12.5547), which is incorporated herein by reference. tTA was created by fusion of the tetracycline resistance operon (tet repressor) encoded in Tn10 of Escherichia coli with the activating cycline-controlled transactivator (tTA) and the tTA-dependent promoter was created by combining the tet operator sequence and a minimal promoter from the human cytomegalovirus promoter IE (hCMV-IE). When tetracycline or its derivatives are added, tTA can no longer bind its target sequence within the tTA-dependent promoter and there is no expression from the tTA-dependent promoter. This is shown in FIG. 1A and explained on page s96 of Jaisser, 2000 (Jaisser, F. (2000) ‘Inducible gene expression and gene modification in transgenic mice’, Journal of the American Society ofNephrology, 11(SUPPL. 16), pp. 95-100), which is incorporated herein by reference. The mechanism of the conformational change brought by binding of tetracycline or its derivatives to tTA is described in Orth et al., 2000 (Orth, P. et al. (2000) ‘Structural basis of gene regulation by the tetracycline inducible Tet repressor-operator system’, Nature Structural Biology, 7(3), pp. 215-219. doi: 10.1038/73324), which is incorporated herein by reference. Binding of tetracycline to TetR increases the separation of the attached DNA binding domains which abolishes the affinity of TetR for its operator DNA.

A suitable promoter induced by presence of tetracycline or its derivatives is the promoter in the tet-ON system. In the tet-ON system, a reverse tetracycline-controlled transactivator (rtTA) allows transcriptional activation of a tTA-dependent promoter in the presence of tetracycline or its derivatives as described in Gossen et al (Science 23 Jun. 1995: Vol. 268, Issue 5218, pp. 1766-1769 DOI: 10.1126/science.7792603), which is incorporated herein by reference. In the absence of tetracycline or its derivatives, tTA can no longer bind its target sequence within the tTA-dependent promoter and there is no expression from the tTA-dependent promoter. This is shown in FIG. 1B and explained on page s96 of Jaisser, 2000 (Jaisser, F. (2000) ‘Inducible gene expression and gene modification in transgenic mice’, Journal of the American Society ofNephrology, 11(SUPPL. 16), pp. 95-100), which is incorporated herein by reference.

Suitably, an improved variant of the reverse tetracycline-controlled transactivator (rtTA) is used.

Suitable improved variants are described in table 1 of Urlinger et al., 2000 (Urlinger, S. et al. (2000) ‘Exploring the sequence space for tetracycline-dependent transcriptional activators: Novel mutations yield expanded range and sensitivity’, Proceedings of the National Academy of Sciences of the United States of America, 97(14), pp. 7963-7968. doi: 10.1073/pnas.130192197), which is incorporated herein by reference. Variants rtTA-S2 and rtTA-M2 were shown to have lower basal activity in FIG. 3 of Urlinger et al., 2000, which indicates minimal background expression from the tTA-dependent promoter in the absence of tetracycline or its derivatives. Additionally rtTA-M2 showed an increased sensitivity towards tetracycline and its derivatives as shown in in FIG. 3 of Urlinger et al., 2000 and functions at 10 fold lower concentrations than rtTA. In some preferred embodiments, the improved variant of rtTA is rtTA-M2 from of Urlinger et al., 2000.

Alternative improved variants are described in Table 1 of Zhou et al., 2006 (Zhou, X. et al. (2006) ‘Optimization of the Tet-On system for regulated gene expression through viral evolution’, Gene Therapy, 13(19), pp. 1382-1390. doi: 10.1038/sj.gt.3302780), which is incorporated herein by reference. The majority of these variants were shown to have higher transcriptional activity and doxycycline sensitivity than rtTA as described in FIG. 3 of Zhou et al., 2006. The highest performing variants were seven-fold more active and 100 times more sensitive to doxycycline. In some preferred embodiments, the improved variant of rtTA is V14, V15 or V16 from Zhou et al., 2006.

Suitable commercially available tetracycline-inducible system is the T-Rex system from Life-Technologies (see e.g. Life-Technologies (2014) ‘Inducible Protein Expression Using the T-REx™ System’, 1, pp. 1-12. Available at:
www.lifetechnologies.com/de/de/home/references/protocols/proteins-expression-isolation-and-analysis/protein-expression-protocol/inducible-protein-expression-using-the-trex-system.reg.us.html/).

Induction Via, e.g. Tetracycline Absence and Estrogen Presence:

The inducible promoter may be induced by absence of a molecule and presence of a different molecule. In some embodiments, the inducible promoter may be induced by removal of tetracycline and addition of estrogen as described in Iida et al., 1996 (Iida, A. et al. (1996) ‘Inducible gene expression by retrovirus-mediated transfer of a modified tetracycline-regulated system.’, Journal of virology, 70(9), pp. 6054-6059. doi: 10.1128/jvi.70.9.6054-6059.1996), which is incorporated herein by reference. This specific inducibility was achieved by the addition of the ligand-binding domain of the estrogen receptor to the carboxy terminal of the tTA transactivator. Such modified transactivator was shown result in high expression of the gene of interest in the absence of tetracycline and the presence of estrogen as shown in FIG. 3 of Iida et al., 1996.

Induction Via Small Molecule Enhancers:

The inducible promoter may be induced by small molecule enhancers. Suitable promoters induced by small molecule enhancers such as aromatic carboxylic acids, hydroxamic acids and acetamides are described in Allen et al. (Biotechnol. Bioeng. 2008; 100: 1193-1204), which is incorporated herein by reference.

Mifepristone (RU-486)-Inducible Promoters:

The inducible promoter may be induced by a synthetic steroid. In some embodiments, the inducible promoter may be induced by mifepristone, also known as RU-486. A hybrid mifespristone-responsive transcription factor, LexPR transactivator, was created by Emelyanov and Parinov, 2008 (Emelyanov, A. and Parinov, S. (2008) ‘Mifepristone-inducible LexPR system to drive and control gene expression in transgenic zebrafish’, Developmental Biology, 320(1), pp. 113-121. doi: 10.1016/j.ydbio.2008.04.042, which is incorporated herein by reference) by fusion of the DNA-binding domain of the bacterial LexA repressor, a truncated ligand-binding domain of the human progesterone receptor and the activation domain of the human NF-kB/p65 protein. Upon addition of mifepristone, LexPR induces expression from a promoter sequence harbouring LexA binding sites as shown in FIG. 1 and FIG. 2 of Emelyanov and Parinov, 2008. Suitable commercially available mifepristone-inducible system is the GeneSwitch System (see e.g., Fisher, T. (1994) ‘Inducible Protein Expression Using GeneSwitch™ Technology’, pp. 1-25).

Cumate-Inducible Promoters:

In some embodiments, the inducible promoter may be induced by the presence or the absence of cumate.

In the cumate switch system from Mullick et al., 2006 (Mullick, A. et al. (2006) ‘The cumate gene-switch: A system for regulated expression in mammalian cells’, BMC Biotechnology, 6, pp. 1-18. doi: 10.1186/1472-6750-6-43, which is incorporated herein by reference), a repressor CymR blocks transcription from a promoter comprising CuO sequence placed downstream of the promoter. Once cumate is added, the CymR repressor is unable to bind to CuO and transcription from a promoter comprising CuO can proceed. This is shown in FIG. 1B and FIG. 2 from Mullick et al., 2006.

In an alternative cumate switch system, a chimeric transactivator (cTA) created from the fusion of CymR with the activation domain of VP16 does not prevent transcription from a promoter comprising CuO sequence upstream of a promoter in the presence of cumate. In the absence of cumate, the chimeric transactivator (cTA) binds to the CuO sequence and prevents transcription. This is shown in FIG. 1C and FIG. 3 from Mullick et al., 2006.

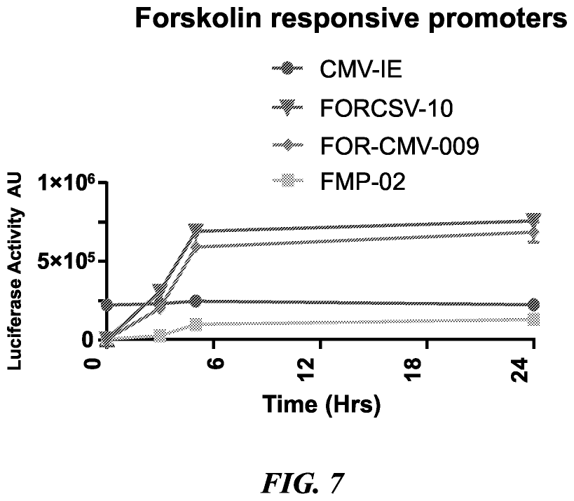

In a third configuration, a reverse chimeric transactivator (rcTA) prevents transcription from a promoter comprising CuO sequence upstream of a promoter in the absence of cumate. In the presence of cumate, the rcTA binds to the CuO sequence and transcription from the promoter comprising CuO sequence can proceed. This is shown in FIG. 1D and FIG. 7 from Mullick et al., 2006.

Suitable commercially available cumate-inducible systems is found from SBI Biosciences (see SBI (2020) ‘Cumate-inducible Systems For the ultimate in gene expression control, use SBI's cumate-CUMATE-INDUCIBLE SYSTEMS’, pp. 1-13, which is incorporated herein by reference).

### 4-hydroxytamoxifen (OHT)-Inducible Promoters

The inducible promoter may be induced by 4-hydroxytamoxifen (OHT). Suitable 4-hydroxytamoxifen inducible promoters are described by Feil et al. (Biochemical and Biophysical Research Communications Volume 237, Issue 3, 28 Aug. 1997, Pages 752), which is incorporated herein by reference.

Gas-Inducible Promoters:

The inducible promoter may be a gas-inducible promoter, e.g. acetaldehyde-inducible. Suitable gas-inducible promoters are described in Weber et al., 2004 (Weber, W. et al. (2004) ‘Gas-inducible transgene expression in mammalian cells and mice’, Nature Biotechnology, 22(11), pp. 1440-1444. doi: 10.1038/nbt1021), which is incorporated herein by reference. The native acetaldehyde-inducible AlcR-PalcA system from Asperigillus nidulans has been adapted for mammalian use by introducing an AlcR-specific operator module to a human minimal promoter, together called P_{AIR}, as shown in FIG. 1A. When AlcR is constitutively expressed in the cell of interest, upon introduction of acetaldehyde, acetaldehyde binds to AlcR and, in turn, the gene of interest which is under the control of the PAIR promoter is expressed, as shown in FIG. 1C, FIG. 2 and FIG. 3. In the absence of acetaldehyde, there is no expression of the gene of interest.

Riboswitch, Ribozyme and Aptazyme-Inducible Promoters:

The inducible promoter may be induced by the presence or absence of a ribozyme. The ribozyme can, in turn be, be induced by a ligand.

The inducible promoter may be induced in the absence of a metabolite. In some embodiments, the metabolite may be glucosamine-6-phosphate-responsive. Suitable ribozyme which acts as a glucosamine-6-phosphate-responsive gene repressor is described by Winkler et al., 2004 (Winkler, W. C. et al. (2004) ‘Control of gene expression by a natural metabolite-responsive ribozyme’, Nature, 428(6980), pp. 281-286. doi: 10.1038/nature02362), which is incorporated herein by reference. The ribozyme is activated by glucosamine-6-phosphate in a concentration dependent manner as shown in FIG. 2C and cleaves the messenger RNA of the glmS gene. Upon modification, it is possible that this natural system may be applied to control of a gene of interest other than the glmS gene.

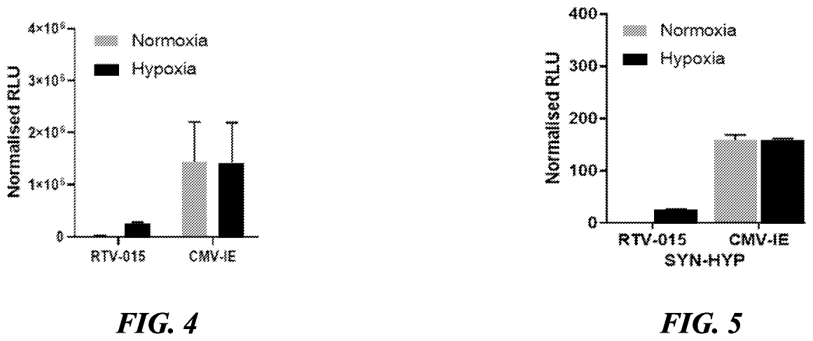

Protein expression can also be downregulated by ligand-inducible aptazyme. Protein expression can be downregulated by aptazyme which downregulate protein expression by small molecule-induced self-cleavage of the ribozyme resulting in mRNA degradation Zhong et al., 2016 (Zhong, G. et al. (2016) ‘Rational design of aptazyme riboswitches for efficient control of gene expression in mammalian cells’, eLife, 5(November 2016). doi: 10.7554/eLife.18858), which is incorporated herein by reference. Suitable aptazymes are shown in FIG. 4A of (Zhong et al., 2016). These aptazymes reduce relative expression of a gene of interest as shown in FIG. 4 of (Zhong et al., 2016).

Protein expression can also be upregulated by a small-molecule dependent ribozyme. The ribozyme may be tetracycline-dependent. Suitable tetracycline-dependent ribozymes which can switch on protein expression by preventing ribozyme cleavage which otherwise cleaves mRNA in the absence of ligand is described in Beilstein et al. (ACS Synth. Biol. 2015, 4, 5, 526-534), which is incorporated herein by reference.

Protein expression can also be regulated by a guanine dependent aptazyme as described by Nomura et al. (Chem. Commun., 2012, 48, 7215-7217) which is incorporated herein by reference.

Additionally, an RNA architecture that combines a drug-inducible allosteric ribozyme with a microRNA precursor analogue that allows chemical induction of RNAi in mammalian cells is described in Kumar et al (J. Am. Chem. Soc. 2009, 131, 39, 13906-13907), which is incorporated herein by reference.

Metallothionein-Inducible Promoters:

Metallothionein-inducible promoters have been described in the literature. See for example Shinichiro Takahashi “Positive and negative regulators of the metallothionein gene” Molecular Medicine Reports Mar. 9, 2015, P795-799, which is incorporated herein by reference.

Rapamycin-Inducible Promoters

The inducible promoter may be induced by a small molecule drug such as rapamycin. A humanized system for pharmacologic control of gene expression using rapamycin is described in Rivera et al., 1996 (Rivera et al Nature Medicine volume 2, pages 1028-1032(1996)), which is incorporated herein by reference. The natural ability of rapamycin to bind to FKBP12 and, in turn, for this complex to bind to FRAP was used by Rivera et al., 1996 to induce rapamycin-specific expression of a gene of interest. This was achieved by fusing one of the FKBP12/FRAP proteins to a DNA binding domain and the other protein to an activator domain. If the FKBP is fused with a DNA binding domain and FRAP is fused to an activator domain, there would be no transcription of the gene of interest in the absence of rapamycin since FKBP and FRAP do not interact, as shown in FIG. 1b. In the presence of rapamycin, FKBP and FRAP interact and the DNA binding domain and the activator domain are brought into close contact, resulting in transcription of the gene of interest as shown in FIG. 2 and FIG. 3.

Chemically-Induced Proximity-Inducible Promoters

The inducible promoter may be controlled by the chemically induced proximity. Suitable small molecule-based systems for controlling protein abundance or activities is described in Liang et al. (Sci Signal. 2011 Mar. 15; 4(164):rs2. doi: 10.1126/scisignal.2001449), which is incorporated herein by reference.

Gene expression may be induced by chemically induced proximity by a molecule combining two protein binding surfaces as shown in Belshaw et al., 1996 (Belshaw, P. J. et al. (1996) ‘Controlling protein association and subcellular localization with a synthetic ligand that induces heterodimerization of proteins’, Proceedings of the National Academy of Sciences of the United States of America, 93(10), pp. 4604-4607), which is incorporated herein by reference. Transcriptional activation of a gene of interest by chemically induced proximity by a molecule combining two protein binding surfaces is shown in FIG. 3 of Belshaw et al.

Rheoswitch® Inducible Promoters:

The inducible promoter may be induced by small synthetic molecules. In some embodiments, these small synthetic molecules may be diacylhydrazine ligands. Suitable systems for inducible up- and down-regulation of gene expression is described in Cress et al. (Volume 66, Issue 8 Supplement, pp. 27) or Barrett et al. (Cancer Gene Therapy volume 25, pages 106-116(2018)), which are incorporated herein by reference. The RheoSwitch® system consists of two chimeric proteins derived from the ecdysone receptor (EcR) and RXR that are fused to a DNA-binding domain and an acidic transcriptional activation domain, respectively. The nuclear receptors can heterodimerize to create a functional transcription factor upon binding of a small molecule synthetic ligand and activate transcription from a responsive promoter linked to a gene of interest.

CRISPR-Inducible Promoters:

Gene expression may be induced by a on CRISPR-based transcription regulators. A nuclease-deficient Cas9 can be directed to a sequence of interest by designing its associated single guide RNA (sgRNA) and it can modulate the gene expression by tethering of effector domains on the sgRNA-Cas9 complex as shown in FIG. 1A of Ferry, Lyutova and Fulga, 2017 (Ferry, Q. R. V., Lyutova, R. and Fulga, T. A. (2017) ‘Rational design of inducible CRISPR guide RNAs for de novo assembly of transcriptional programs’, Nature Communications. Nature Publishing Group, 8, pp. 1-10. doi: 10.1038/ncomms14633), which is incorporated herein by reference. Suitable versatile inducible-CRISPR-TR platform based on minimal engineering of the sgRNA is described in Ferry, Lyutova and Fulga, 2017.

The CRISPR-based transcriptional regulation may in turn be induced by drugs. Suitable drug inducible CRISPR-based transcription regulators systems are shown in Zhang et al., 2019 (Zhang, J. et al. (2019) ‘Drug Inducible CRISPR/Cas Systems’, Computational and Structural Biotechnology Journal. Elsevier B. V., 17, pp. 1171-1177. doi: 10.1016/j.csbj.2019.07.015), which is incorporated herein by reference.

In one embodiment, contacting the cell with an inducer or applying a suitable inducing condition to the cell results in expression of the at least one toxic protein.

Inducible promoters described herein can further control expression of an inducer or repressor of an inducible promoter, e.g., an inducer or repressor of a second, different promoter, or an inducer or repressor of itself. In one embodiment, the cell comprises a first inducible promoter that is operatively linked to a repressible element that can stop protein expression.

In one embodiment, the first inducible promoter that further encodes a protein that represses expression of the first inducible promoter.

In one embodiment, the cell comprises a first inducible promoter that further encodes a protein that induces expression of a second inducible promoter.

### Toxic Genes

Various aspects of the invention described herein relate to the use of any of the inducible promoters presented herein to control expression of a toxic protein in a cell, such as a viral vector producer cell. As used herein, “toxic protein” refers to a gene product that, when expressed in a cell, exert toxic effects on the cell, which negatively impact the physiology of the cell, and can result in decreased cellular propagation, apoptosis and finally cell death. The toxic protein is not initially highly toxic, but over time its expression can, e.g., kill the cell.

Certain viral proteins required for propagation of viral vectors are toxic when expressed in a cell, thus is it desirable to tightly control the expression of these genes during production of viral vectors. For example, the replication (rep) and capsid (cap) proteins required for propagation of an AAV viral vector are toxic when expressed in an AAV producer cell. Rep and cap each express a family of related proteins from separate open reading frames and are produced by alternative mRNA splicing and different transcriptional and translational start sites. Rep proteins (Rep78, Rep68, Rep52, and Rep40) are involved in replication, rescue and integration of the AAV genome. Rep78 and Rep68 have the same amino-terminal sequence and share the same native promoter, p5, but Rep78 contains an exon that is alternatively spliced out in rep68. Similarly, Rep52 and Rep40 have the same amino-terminal sequence and share the native p19 promoter, which is downstream from the p5 promoter, but rep52 contains an exon that is alternatively spliced out in rep68. The Cap gene encodes three capsid proteins (VP1, VP2, and VP3) and assembly-activating protein (AAP) that promotes capsid formation, forming the virion capsid. Cap gene transcription is driven by the p40 promoter. Helper virus genes used in the production of AAV vectors can also produce toxic products. Helper genes conventionally used include in AAV production include E1 (E1A and E1B), E2A, E4 and VA RNA. Further, the polymerase (pol), Group antigen (gag), and envelope (env) proteins required for propagation of, e.g., a lentivirus vector or adenovirus vector, are toxic over time when expressed in a viral vector producer cell. Env protein is essential for the formation of the viral envelope. Gag is an acronym for group antigens (ag). The group antigens form the viral core structure, RNA genome binding proteins, and are the major proteins comprising the nucleoprotein core particle. Pol, a reverse transcriptase is the essential enzyme that carries out the reverse transcription process that take the RNA genome to a double-stranded DNA preintegrate form. The pol gene further encodes an Integrase activity and an RNase H activity that functions during genome reverse transcription. The tat and rev proteins can also be toxic to the cell when expressed over time.

As noted in the above aspects, expression of the Rep protein is under the control of a second regulatable promoter or regulatable transcription factor. In one embodiment, the second regulatable promoter is an inducible promoter, or comprises a binding site for a regulatable transcriptional activator.

In one embodiment, the or each nucleic acid encoding the Rep protein is operatively linked to an inducible promoter. Suitable inducible promoters are described elsewhere herein.

In one embodiment, the or each nucleic acid encoding the Rep protein is operatively linked to a promoter comprising a target site for binding of a regulatable transcriptional activator. Suitable regulatable transcriptional activators are described elsewhere herein e.g., a zinc-finger transcriptional activator (ZF-TA).

In one embodiment, a first and a second Rep protein are encoded by the nucleic acid sequence, optionally by one or more nucleic acid sequences. In one embodiment, the first Rep protein is Rep78 and the second Rep protein is Rep 52. In one embodiment, the or each nucleic acid encoding the first and second Rep proteins is under the control of a second regulatable promoter or regulatable transcriptional activator. In one embodiment, each nucleic acid encoding the first and second Rep proteins is under the control of a regulatable transcriptional activator. In one embodiment, each nucleic acid encoding the first and second Rep proteins is under the control of the same regulatable transcriptional activator. In one embodiment, the transcriptional activator is a zinc-finger transcriptional activator (ZF-TA).

In one embodiment, the nucleic acid encoding the Rep protein comprises a modified p19 promoter. In one embodiment, the nucleic acid encoding the Rep78 protein comprises a modified p19 promoter. In one embodiment, the modified p19 promoter is modified to reduce expression. In one embodiment, the modified p19 promoter comprises one or more mutations.

In one embodiment, the modified p19 promoter comprises the following nucleic acid sequence:

(SEQ ID NO: 149)
acgcAAtGcCGCCGGgGGaGcGAACAAaGTtGTtGACGAGTGCTACATCC
CCAATTACTTGCTCCCCAAAACCCAGCCTGAGCTCCAaTGGGgaTGGACa
AAcATAGAACAGTAcctg

In one embodiment, the nucleic acid sequence encoding the Rep protein comprises a modified start codon. In one embodiment, the nucleic acid sequence encoding the large Rep protein (e.g. Rep78) protein comprises a modified start codon. In one embodiment, the nucleic acid sequence encoding the large Rep protein (e.g. Rep78) protein comprises a modified start codon selected from: ACC, AUC, CUG, and AGG. In such embodiments, the nucleic acid sequence encoding the small Rep (e.g. Rep52) protein comprises atypical start codon. In such embodiments, the nucleic acid sequence encoding the small Rep (e.g. Rep52) protein comprises and ATG start codon.

In one embodiment, the nucleic acid sequence encoding the large Rep protine, e.g., Rep78 protein, comprises a CUG start codon.

In one embodiment, a nucleic acid encoding a modified Rep protein comprising a modified start codon comprises the following sequence, where the modified start codon is in bold:

(SEQ ID NO: 150)
cTGCCGGGGTTTTACGAGATTGTGATTAAGGTCCCCAGCGACCTTGACGA
GCATCTGCCCGGCATTTCTGACAGCTTTGTGAACTGGGTGGCCGAGAAGG
AGTGGGAGTTGCCGCCAGATTCTGACTTGGATCTGAATCTGATTGAGCAG
GCACCCCTGACCGTGGCCGAGAAGCTGCAGCGCGACTTTCTGACGGAGTG
GCGCCGTGTGAGTAAGGCCCCGGAGGCCCTTTTCTTTGTGCAATTTGAGA
AGGGAGAGAGCTACTTCCACTTACACGTGCTCGTGGAAACCACCGGGGTG
AAATCCTTAGTTTTGGGACGTTTCCTGAGTCAGATTCGCGAAAAACTGAT
TCAGAGAATTTACCGCGGGATCGAGCCGACTTTGCCAAACTGGTTCGCGG
TCACAAAGACCAGAAACGGCGCCGGAGGCGGGAACAAGGTGGTGGACGAG
TGCTACATCCCCAATTACTTGCTCCCCAAAACCCAGCCTGAGCTCCAGTG
GGCGTGGACTAATATAGAACAGTATTTAAGCGCCTGTTTGAATCTCACGG
AGCGTAAACGGTTGGTGGCGCAGCATCTGACGCACGTGTCGCAGACGCAG
GAGCAGAACAAAGAGAATCAGAATCCCAATTCTGACGCGCCGGTGATCAG
ATCAAAAACTTCAGCCAGGTACATGGAGCTGGTCGGGTGGCTCGTGGACA
AGGGGATTACCTCGGAGAAGCAGTGGATCCAGGAGGACCAGGCCTCATAC
ATCTCCTTCAATGCGGCCTCCAACTCGCGGTCCCAAATCAAGGCTGCCTT
GGACAATGCGGGAAAGATTATGAGCCTGACTAAAACCGCCCCCGACTACC
TGGTGGGCCAGCAGCCCGTGGAGGACATTTCCAGCAATCGGATTTATAAA
ATTTTGGAACTAAACGGGTACGATCCCCAATATGCGGCTTCCGTCTTTCT
GGGATGGGCCACGAAAAAGTTCGGCAAGAGGAACACCATCTGGCTGTTTG
GGCCTGCAACTACCGGGAAGACCAACATCGCGGAGGCCATAGCCCACACT
GTGCCCTTCTACGGGTGCGTAAACTGGACCAATGAGAACTTTCCCTTCAA
CGACTGTGTCGACAAGATGGTGATCTGGTGGGAGGAGGGGAAGATGACCG
CCAAGGTCGTGGAGTCGGCCAAAGCCATTCTCGGAGGAAGCAAGGTGCGC
GTGGACCAGAAATGCAAGTCCTCGGCCCAGATAGACCCGACTCCCGTGAT
CGTCACCTCCAACACCAACATGTGCGCCGTGATTGACGGGAACTCAACGA
CCTTCGAACACCAGCAGCCGTTGCAAGACCGGATGTTCAAATTTGAACTC
ACCCGCCGTCTGGATCATGACTTTGGGAAGGTCACCAAGCAGGAAGTCAA
AGACTTTTTCCGGTGGGCAAAGGATCACGTGGTTGAGGTGGAGCATGAAT
TCTACGTCAAAAAGGGTGGAGCCAAGAAAAGACCCGCCCCCAGTGACGCA
GATATAAGTGAGCCCAAACGGGTGCGCGAGTCAGTTGCGCAGCCATCGAC
GTCAGACGCGGAAGCTTCGATCAACTACGCAGACAGGTACCAAAACAAAT
GTTCTCGTCACGTGGGCATGAATCTGATGCTGTTTCCCTGCAGACAATGC
GAGAGAATGAATCAGAATTCAAATATCTGCTTCACTCACGGACAGAAAGA
CTGTTTAGAGTGCTTTCCCGTGTCAGAATCTCAACCCGTTTCTGTCGTCA
AAAAGGCGTATCAGAAACTGTGCTACATTCATCATATCATGGGAAAGGTG
CACGACGCTTGCACTGCCTGCGATCTGGTCAATGTGGATTTGGATGACTG
CATCTTTGAACAATAA

In one embodiment, the Rep protein is a modified Rep protein. A modified Rep protein Rep78. In one embodiment, the modified Rep protein has a lysine to arginine mutation at amino acid 84. One skilled in the art can generate a modified Rep protein using a standard single-site mutagenesis PCR-based assay. DNA sequencing can be used to detect a Rep protein having, e.g., an amino acid substitution.

In one embodiment, the modified Rep protein is modified Rep78 and comprises the following sequence, where the lysine to arginine mutation is in bold:

(SEQ ID NO: 151)
MPGFYEIVIKVPSDLDEHLPGISDSFVNWVAEKEWELPPDSDLDLNLIEQ
APLTVAEKLQRDFLTEWRRVSKAPEALFFVQFERGESYFHLHVLVETTGV
KSLVLGRFLSQIREKLIQRIYRGIEPTLPNWFAVTKTRNGAGGGNKVVDE
CYIPNYLLPKTQPELQWAWTNIEQYLSACLNLTERKRLVAQHLTHVSQTQ
EQNKENQNPNSDAPVIRSKTSARYMELVGWLVDKGITSEKQWIQEDQASY
ISFNAASNSRSQIKAALDNAGKIMSLIKTAPDYLVGQQPVEDISSNRIYK
ILELNGYDPQYAASVFLGWATKKFGKRNTIWLFGPATTGKTNIAEAIAHT
VPFYGCVNWTNENFPFNDCVDKMVIWWEEGKMTAKVVESAKAILGGSKVR
VDQKCKSSAQIDPTPVIVTSNTNMCAVIDGNSTTFEHQQPLQDRMFKFEL
TRRLDHDFGKVTKQEVKDFFRWAKDHVVEVEHEFYVKKGGAKKRPAPSDA
DISEPKRVRESVAQPSTSDAEASINYADRYQNKCSRHVGMNLMLFPCRQC
ERMNQNSNICFTHGQKDCLECFPVSESQPVSVVKKAYQKLCYIHHIMGKV
PDACTACDLVNVDLDDCIFEQ

In one embodiment, the nucleic acid encoding the Rep protein further comprises the nucleic acid encoding a ribozyme protein at its 3′ end.

In one embodiment of any aspect, the or each Rep protein may comprise any combination of the above-mentioned features to optimize expression.

In one embodiment, the Rep protein comprises a modified p19 promoter and an amino acid substitution mutation. In one embodiment of any aspect, the Rep protein comprises a modified p19 promoter and comprises a nucleic acid encoding a ribozyme at its 3′ end. In one embodiment, the Rep protein comprises a modified start codon, and a modified p19 promoter. In one embodiment, the Rep protein comprises a modified start codon, and a nucleic acid encoding a ribozyme at its 3′ end. In one embodiment, the Rep protein comprises a modified start codon, and an amino acid substitution. In one embodiment, the Rep protein comprises an amino acid substitution, and comprises a nucleic acid encoding a ribozyme at its 3′ end.

In one embodiment of any aspect, the Rep protein comprises an amino acid substitution, is a modified Rep protein, and further comprises a nucleic acid encoding a ribozyme at its 3′ end. In one embodiment of any aspect, the Rep protein comprises a modified start codon, an amino acid substitution, and further comprises a nucleic acid encoding a ribozyme at its 3′ end. In one embodiment of any aspect, the Rep protein comprises an amino acid substitution, comprises a modified p19 promoter, and comprises a modified start codon. In one embodiment of any aspect, the Rep protein comprises a modified p19 promoter, a modified start codon, and a nucleic acid encoding a ribozyme at its 3′ end.

In one embodiment, a nucleic acid encoding a Rep protein comprising a modified start codon, an amino acid substitution, and a modified p19 promoter comprises the following sequence, wherein these features are consecutively shown in bold:

(SEQ ID NO: 152)
cTGCCGGGGTTTTACGAGATTGTGATTAAGGTCCCC
AGCGACCTTGACGAGCATCTGCCCGGCATTTCTGAC
AGCTTTGTGAACTGGGTGGCCGAGAAGGAGTGGGAG
TTGCCGCCAGATTCTGACTTGGATCTGAATCTGATT
GAGCAGGCACCCCTGACCGTGGCCGAGAAGCTGCAG
CGCGACTTTCTGACGGAGTGGCGCCGTGTGAGTAAG
GCCCCGGAGGCCCTTTTCTTTGTGCAATTTGAGAga
GGAGAGAGCTACTTCCACTTACACGTGCTCGTGGAA
ACCACCGGGGTGAAATCCTTAGTTTTGGGACGTTTC
CTGAGTCAGATTCGCGAAAAACTGATTCAGAGAATT
TACCGCGGGATCGAGCCGACTTTGCCAAACTGGTTC
GCGGTCACAAAGACacgcAAtGcCGCCGGgGGaGcG
AACAAaGTtGTtGACGAGTGCTACATCCCCAATTAC
TTGCTCCCCAAAACCCAGCCTGAGCTCCAaTGGGga
TGGACaAAcATAGAACAGTAcctgAGCGCCTGTTTG
AATCTCACGGAGCGTAAACGGTTGGTGGCGCAGCAT
CTGACGCACGTGTCGCAGACGCAGGAGCAGAACAAA
GAGAATCAGAATCCCAATTCTGACGCGCCGGTGATC
AGATCAAAAACTTCAGCCAGGTACATGGAGCTGGTC
GGGTGGCTCGTGGACAAGGGGATTACCTCGGAGAAG
CAGTGGATCCAGGAGGACCAGGCCTCATACATCTCC
TTCAATGCGGCCTCCAACTCGCGGTCCCAAATCAAG
GCTGCCTTGGACAATGCGGGAAAGATTATGAGCCTG
ACTAAAACCGCCCCCGACTACCTGGTGGGCCAGCAG
CCCGTGGAGGACATTTCCAGCAATCGGATTTATAAA
ATTTTGGAACTAAACGGGTACGATCCCCAATATGCG
GCTTCCGTCTTTCTGGGATGGGCCACGAAAAAGTTC
GGCAAGAGGAACACCATCTGGCTGTTTGGGCCTGCA
ACTACCGGGAAGACCAACATCGCGGAGGCCATAGCC
CACACTGTGCCCTTCTACGGGTGCGTAAACTGGACC
AATGAGAACTTTCCCTTCAACGACTGTGTCGACAAG
ATGGTGATCTGGTGGGAGGAGGGGAAGATGACCGCC
AAGGTCGTGGAGTCGGCCAAAGCCATTCTCGGAGGA
AGCAAGGTGCGCGTGGACCAGAAATGCAAGTCCTCG
GCCCAGATAGACCCGACTCCCGTGATCGTCACCTCC
AACACCAACATGTGCGCCGTGATTGACGGGAACTCA
ACGACCTTCGAACACCAGCAGCCGTTGCAAGACCGG
ATGTTCAAATTTGAACTCACCCGCCGTCTGGATCAT
GACTTTGGGAAGGTCACCAAGCAGGAAGTCAAAGAC
TTTTTCCGGTGGGCAAAGGATCACGTGGTTGAGGTG
GAGCATGAATTCTACGTCAAAAAGGGTGGAGCCAAG
AAAAGACCCGCCCCCAGTGACGCAGATATAAGTGAG
CCCAAACGGGTGCGCGAGTCAGTTGCGCAGCCATCG
ACGTCAGACGCGGAAGCTTCGATCAACTACGCAGAC
AGGTACCAAAACAAATGTTCTCGTCACGTGGGCATG
AATCTGATGCTGTTTCCCTGCAGACAATGCGAGAGA
ATGAATCAGAATTCAAATATCTGCTTCACTCACGGA
CAGAAAGACTGTTTAGAGTGCTTTCCCGTGTCAGAA
TCTCAACCCGTTTCTGTCGTCAAAAAGGCGTATCAG
AAACTGTGCTACATTCATCATATCATGGGAAAGGTG
CCAGACGCTTGCACTGCCTGCGATCTGGTCAATGTG
GATTTGGATGACTGCATCTTTGAACAATAA

In one embodiment, a Rep protein comprising a modified start codon, an amino acid substitution, and a modified p19 promoter comprises the following sequence:

(SEQ ID NO: 153)
LPGFYEIVIKVPSDLDEHLPGISDSFVNWVAEKEWELPPDSDLDLNLIEQ
APLTVAEKLQRDFLTEWRRVSKAPEALFFVQFERGESYFHLHVLVETTGV
KSLVLGRFLSQIREKLIQRIYRGIEPTLPNWFAVTKTRNAAGGANKVVDE
CYIPNYLLPKTQPELQWGWTNIEQYLSACLNLTERKRLVAQHLTHVSQTQ
EQNKENQNPNSDAPVIRSKTSARYMELVGWLVDKGITSEKQWIQEDQASY
ISFNAASNSRSQIKAALDNAGKIMSLTKTAPDYLVGQQPVEDISSNRIYK
ILELNGYDPQYAASVFLGWATKKFGKRNTIWLFGPATTGKTNIAEAIAHT
VPFYGCVNWTNENFPFNDCVDKMVIWWEEGKMTAKVVESAKAILGGSKVR
VDQKCKSSAQIDPTPVIVTSNTNMCAVIDGNSTTFEHQQPLQDRMFKFEL
TRRLDHDFGKVTKQEVKDFFRWAKDHVVEVEHEFYVKKGGAKKRPAPSDA
DISEPKRVRESVAQPSTSDAEASINYADRYQNKCSRHVGMNLMLFPCRQC
ERMNQNSNICFTHGQKDCLECFPVSESQPVSVVKKAYQKLCYIHHIMGKV
PDACTACDLVNVDLDDCIFEQ

In one embodiment of any aspect, the Rep protein comprises a modified start codon, a modified p19 promoter, is a modified Rep protein, and further comprises a nucleic acid encoding a ribozyme at its 3′ end.

In one embodiment of any aspect, the Rep protein comprises a CUG start codon, a modified p19 promoter, a lysine to arginine mutation at amino acid 84, and further comprises a nucleic acid encoding a ribozyme at its 3′ end.

In one embodiment, the large Rep, e.g., Rep 78, and the small Rep, e.g., Rep 52, are under control of separate or distinct regulatable elements so that the expression of each Rep proteins is independent of the other. In this way, small Rep expression can be readily controlled.

In one embodiment, the toxic protein is any viral protein known in the art, for example, any component or product of a virus. Viral proteins include, but are not limited to, structural proteins, nonstructural proteins, regulatory proteins, and accessory proteins for any virus.

In one embodiment, the toxic protein is a capsid protein, an envelope protein, a membrane fusion protein, a nonstructural protein, or a viral accessory protein. In one embodiment, the toxic protein is a Rep protein as explained above.

Viral membrane fusion proteins are grouped into four different classes, and each class is identified by characteristic structural conformations: (Class I) Post-fusion conformation has a distinct central coiled-coil structure composed of signature trimer of α-helical hairpins, e.g., HIV glycoprotein, gp41; (Class II) Lack the central coiled-coil structure and contains a characteristic elongated β-sheet ectodomain structure that refolds to give a trimer of hairpins; e.g., dengue virus E protein, and the west nile virus E protein; (Class III) Structural conformation is a combination of features from Class I and Class II viral membrane fusion proteins; e.g., rabies virus glycoprotein, G; and (Class IV) viral fusion proteins are fusion-associated small transmembrane (FAST) proteins and do not form trimers of hairpins or hairpin structures themselves. FAST proteins are coded for by members of the nonenveloped reoviridae family of viruses.

Viral nonstructural proteins are proteins coded for by the genome of the virus and are expressed in infected cells. However, these proteins are not assembled in the virion, but rather, viral nonstructural proteins carry out important functions that affect the replication and assembly processes. Some viral nonstructural protein functions are replicon formation, immunomodulation, and transactivation of viral structural protein encoding genes. For example, in the hepatitis C virus, viral nonstructural proteins interact with cellular vesicle membrane transport protein, hVAP-33, to assemble the replicon. Viral nonstructural 4b (NS4B) protein alter the host cell's membrane and starts the formation process of the replication complex. Other viral nonstructural proteins, such as NS5A, NS5B, and NS3, are also recruited to the complex, and NS4B interacts with them and binds to viral RNA. Exemplary immunomodulatory proteins include viral nonstructural protein NS1 in the West Nile virus, which prevents complement activation through its binding to a complement control protein, factor H.

Viral accessory proteins, also known as auxiliary proteins, are coded for by the genome of retroviruses. Most viral accessory proteins only carry out their functions in specific types of cells and do not have much influence on the replication of the virus. In certain instances, however, maintaining the replication of viruses would require the function of viral accessory proteins.

In one embodiment, the toxic gene is a Cas protein. Exemplary Cas proteins include, but are not limited to, Cpf1, C2c1, C2c3, Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, C2c1, C2c3, Cas12a, Cas12b, Cas12c, Cas12d, Cas12e, Cas13a, Cas13b, and Cas13c.

In one embodiment, the Cas protein is Cas9 or Cas9 variant, e.g. isolated from the bacterium Streptococcus pyogenes (SpCas9). The CRISPR-associate nuclease associates with guide RNA (gRNA) that guides the nuclease to the desired target sequence, e.g. having a protospacer adjacent motif (PAM) sequence, downstream of the target sequence for its cutting action. Once Cas9 recognizes the PAM sequence (5′-NGG-3 in case of SpCas9, where N is any nucleotide), it creates a double-strand break (DSB) at the target locus. Cas9 activity is a collective effort of two parts of the protein: the recognition lobe that senses the complementary sequence of gRNA and the nuclease lobe that cleaves the DNA.

In one embodiment, the Cas protein is an enhanced specificity spCas9 (eSpCas9) variant. eSpCas9 variants are further described in Slaymaker, et al. Science. 2016; 351(6268): 84-88, which is incorporated herein by reference in its entirety.

In one embodiment the Cas protein is a natural variant of Cas. Cas9 variants include e.g. Staphylococcus aureus (SaCas9), Streptococcus thermophilus (StCas9), Neisseria meningitidis (NmCas9), Francisella novicida (FnCas9), and Campylobacter jejuni (CjCas9), to name a few, in CRISPR experiments. The nuclease can be determined based on preferred PAM sequence or size, e.g. the SaCas9 nuclease is about 1 kb smaller in size than SpCas9 so it can be packaged into viral vectors more easily; and e.g. CasX and CasY (Burstein, David, et al. New CRISPR-Cas systems from uncultivated microbes. Nature 542.7640 (2017): 237, incorporated by reference in its entirety) are two of the most compact naturally occurring CRISPR variants.

Sequences for Cas9 for various species are known in the art. For example, S. aureus Cas9 (saCas9) has the sequence of SEQ ID NO: 154.

SEQ ID NO: 154 is an amino acid sequence encoding S. aureus Cas9.

(SEQ ID NO: 154)
MKRNYILGLD IGITSVGYGI IDYETRDVID AGVRLFKEAN
VENNEGRRSK RGARRLKRRR RHRIQRVKKL LFDYNLLTDH
SELSGINPYE ARVKGLSQKL SEEEFSAALL HLAKRRGVHN
VNEVEEDTGN ELSTKEQISR NSKALEEKYV AELQLERLKK
DGEVRGSINR FKTSDYVKEA KQLLKVQKAY HQLDQSFIDT
YIDLLETRRT YYEGPGEGSP FGWKDIKEWY EMLMGHCTYF
PEELRSVKYA YNADLYNALN DLNNLVITRD ENEKLEYYEK
FQIIENVEKQ KKKPTLKQIA KEILVNEEDI KGYRVTSTGK
PEFTNLKVYH DIKDITARKE IIENAELLDQ IAKILTIYQS
SEDIQEELTN LNSELTQEEI EQISNLKGYT GTHNLSLKAI
NLILDELWHT NDNQIAIENR LKLVPKKVDL SQQKEIPTTL
VDDFILSPVV KRSFIQSIKV INAIIKKYGL PNDIIIELAR
EKNSKDAQKM INEMQKRNRQ TNERIEEIIR TTGKENAKYL
IEKIKLHDMQ EGKCLYSLEA IPLEDLLNNP FNYEVDHIIP
RSVSFDNSFN NKVLVKQEEN SKKGNRTPFQ YLSSSDSKIS
YETFKKHILN LAKGKGRISK TKKEYLLEER DINRFSVQKD
FINRNLVDTR YATRGLMNLL RSYFRVNNLD VKVKSINGGF
TSFLRRKWKF KKERNKGYKH HAEDALIIAN ADFIFKEWKK
LDKAKKVMEN QMFEEKQAES MPEIETEQEY KEIFITPHQI
KHIKDFKDYK YSHRVDKKPN RELINDTLYS TRKDDKGNTL
IVNNLNGLYD KDNDKLKKLI NKSPEKLLMY HHDPQTYQKL
KLIMEQYGDE KNPLYKYYEE TGNYLTKYSK KDNGPVIKKI
KYYGNKLNAH LDITDDYPNS RNKVVKLSLK PYRFDVYLDN
GVYKFVTVKN LDVIKKENYY EVNSKCYEEA KKLKKISNQA
EFIASFYNND LIKINGELYR VIGVNNDLLN RIEVNMIDIT
YREYLENMND KRPPRIIKTI ASKTQSIKKY STDILGNLYE
VKSKKHPQII KKG

In one embodiment, the Cas protein is a Cas 9 derived from Campylobacter jejuni (C. jejuni). This C. jejuni Cas9 (CjCas9) is further described in, e.g., International patent application WO2016021973A1, which is incorporated herein by reference in its entirety.

SEQ ID NO: 155 is an amino acid sequence encoding CjCas9.

(SEQ ID NO: 155)
MARILAFDIG ISSIGWAFSE NDELKDCGVR IFTKVENPKT
GESLALPRRL ARSARKRLAR RKARLNHLKH LIANEFKLNY
EDYQSFDESL AKAYKGSLIS PYELRFRALN ELLSKQDFAR
VILHIAKRRG YDDIKNSDDK EKGAILKAIK QNEEKLANYQ
SVGEYLYKEY FQKFKENSKE FTNVRNKKES YERCIAQSFL
KDELKLIFKK QREFGFSFSK KFEEEVLSVA FYKRALKDFS
HLVGNCSFFT DEKRAPKNSP LAFMFVALTR IINLLNNLKN
TEGILYTKDD LNALLNEVLK NGTLTYKQTK KLLGLSDDYE
FKGEKGTYFI EFKKYKEFIK ALGEHNLSQD DLNEIAKDIT
LIKDEIKLKK ALAKYDLNON QIDSLSKLEF KDHLNISFKA
LKLVTPLMLE GKKYDEACNE LNLKVAINED KKDFLPAFNE
TYYKDEVTNP VVLRAIKEYR KVLNALLKKY GKVHKINIEL
AREVGKNHSQ RAKIEKEQNE NYKAKKDAEL ECEKLGLKIN
SKNILKLRLF KEQKEFCAYS GEKIKISDLQ DEKMLEIDHI
YPYSRSFDDS YMNKVLVFTK QNQEKLNQTP FEAFGNDSAK
WQKIEVLAKN LPTKKQKRIL DKNYKDKEQK NFKDRNLNDT
RYIARLVLNY TKDYLDFLPL SDDENTKLND TQKGSKVHVE
AKSGMLTSAL RHTWGFSAKD RNNHLHHAID AVIIAYANNS
IVKAFSDFKK EQESNSAELY AKKISELDYK NKRKFFEPFS
GFRQKVLDKI DEIFVSKPER KKPSGALHEE TFRKEEEFYQ
SYGGKEGVLK ALELGKIRKV NGKIVKNGDM FRVDIFKHKK
TNKFYAVPIY TMDFALKVLP NKAVARSKKG EIKDWILMDE
NYEFCFSLYK DSLILIQTKD MQEPEFVYYN AFTSSTVSLI
VSKHDNKFET LSKNQKILFK NANEKEVIAK SIGIQNLKVF
EKYIVSALGE VTKAEFRQRE DFKK

In one embodiment the Cas protein is Cas12a (also known as Cpf1). As Cas9 requires guanine-rich PAM sequence of NGG, it is not well suited for targeting AT-rich sequences. Zetsche et al. characterized a nuclease (see e.g. US Patent Application US 2016/0208243 for sequence and variants, incorporated by reference in its entirety), CRISPR from Prevotella and Francisella 1 (Cpf1; now classified as Cas12a) can be used when targeting AT-rich DNA sequences. Cpf1 creates a staggered double-stranded cut, rather than blunt-end cut generated by SpCas9, in the target DNA, and is useful for experiments relying on the HDR repair outcome. Also, Cpf1 is smaller than SpCas9 and does not require a tracer RNA. The guide RNA required by Cfp1 is therefore shorter in length, making it more economical to produce.

Sequences for Cpf1 for various species are known in the art. For example, Acidaminococcus sp. Cpf1 has the sequence of SEQ ID NO: 156.

SEQ ID NO: 156 is an amino acid sequence encoding Acidaminococcus sp. Cpf1.

(SEQ ID NO: 156)
MTQFEGFTNLYQVSKTLRFELIPQGKTLKHIQEQGFIEEDKARNDHYKEL
KPIIDRIYKTYADQCLQLVQLDWENLSAAIDSYRKEKTEETRNALIEEQA
TYRNAIHDYFIGRTDNLTDAINKRHAEIYKGLFKAELFNGKVLKQLGTVT
TTEHENALLRSFDKFTTYFSGFYENRKNVFSAEDISTAIPHRIVQDNFPK
FKENCHIFTRLITAVPSLREHFENVKKAIGIFVSTSIEEVFSFPFYNQLL
TQTQIDLYNQLLGGISREAGTEKIKGLNEVLNLAIQKNDETAHIIASLPH
RFIPLFKQILSDRNTLSFILEEFKSDEEVIQSFCKYKTLLRNENVLETAE
ALFNELNSIDLTHIFISHKKLETISSALCDHWDTLRNALYERRISELTGK
ITKSAKEKVQRSLKHEDINLQEIISAAGKELSEAFKQKTSEILSHAHAAL
DQPLPTTLKKQEEKEILKSQLDSLLGLYHLLDWFAVDESNEVDPEFSARL
TGIKLEMEPSLSFYNKARNYATKKPYSVEKFKLNFQMPTLASGWDVNKEK
NNGAILFVKNGLYYLGIMPKQKGRYKALSFEPTEKTSEGFDKMYYDYFPD
AAKMIPKCSTQLKAVTAHFQTHTTPILLSNNFIEPLEITKEIYDLNNPEK
EPKKFQTAYAKKTGDQKGYREALCKWIDFTRDFLSKYTKTTSIDLSSLRP
SSQYKDLGEYYAELNPLLYHISFQRIAEKEIMDAVETGKLYLFQIYNKDF
AKGHHGKPNLHTLYWTGLFSPENLAKTSIKLNGQAELFYRPKSRMKRMAH
RLGEKMLNKKLKDQKTPIPDTLYQELYDYVNHRLSHDLSDEARALLPNVI
TKEVSHEIIKDRRFTSDKFFFHVPITLNYQAANSPSKFNQRVNAYLKEHP
ETPIIGIDRGERNLIYITVIDSTGKILEQRSLNTIQQFDYQKKLDNREKE
RVAARQAWSVVGTIKDLKQGYLSQVIHEIVDLMIHYQAVVVLENLNFGFK
SKRTGIAEKAVYQQFEKMLIDKLNCLVLKDYPAEKVGGVLNPYQLTDQFT
SFAKMGTQSGFLFYVPAPYTSKIDPLTGFVDPFVWKTIKNHESRKHFLEG
FDFLHYDVKTGDFILHFKMNRNLSFQRGLPGEMPAWDIVFEKNETQFDAK
GTPFIAGKRIVPVIENHRFTGRYRDLYPANELIALLEEKGIVFRDGSNIL
PKLLENDDSHAIDTMVALIRSVLQMRNSNAATGEDYINSPVRDLNGVCFD
SRFQNPEWPMDADANGAYHIALKGQLLLNHLKESKDLKLONGISNQDWLA
YIQELRN

In one embodiment, the Cas protein is an engineered Cas9 Variant, e.g. a Cas9 Nickase, or a dead Cas9 for use in CRISPRi or CRISPRa systems. For example, variants that nick a single DNA strand instead of creating a double-strand break. (See e.g. Cong, Le, et al. Multiplex genome engineering using CRISPR/Cas systems. Science (2013): 1231143; Mali, Prashant, et al. CAS9 transcriptional activators for target specificity screening and paired nickases for cooperative genome engineering. Nature biotechnology 31.9 (2013): 833; Ran, F. Ann, et al. Double nicking by RNA-guided CRISPR Cas9 for enhanced genome editing specificity. Cell 154.6 (2013): 1380-1389; Cho, Seung Woo, et al. Analysis of off-target effects of CRISPR/Cas-derived RNA-guided endonucleases and nickases. Genome research 24.1 (2014): 132-141, each of which incorporated by reference in their entirety). In some embodiments two guide RNAs are used with the nCAS9. Alternatively, eSpCas9 that uses a single gRNA can be used. Although nickases show high specificity, they rely on two guide RNAs to reach the target sites, thereby reducing the number of potential target sites in the genome. An alternative was created by engineering versions of Cas9 that improved fidelity using a single guide RNA; (see e.g. Qi, Lei S., et al. Repurposing CRISPR as an RNA-guided platform for sequence-specific control of gene expression.” Cell 152.5 (2013): 1173-1183, incorporated by reference in its entirety).

In one embodiment the Cas protein is SpCas9-HF1 or HypaCas9Kleinstiver (See e.g. Benjamin P., et al. High-fidelity CRISPR-Cas9 nucleases with no detectable genome-wide off-target effects.” Nature 529.7587 (2016): 490; Chen, Janice S., et al. Enhanced proofreading governs CRISPR-Cas9 targeting accuracy. Nature 550.7676 (2017): 407, each of which are incorporated by reference in their entirety).

In one embodiment, the Cas protein is the xCas9 nuclease recognizes a broad range of PAM sequences, increasing the target sites to 1 in 4 in the genome, (See e.g. Hu, Johnny H., et al. Evolved Cas9 variants with broad PAM compatibility and high DNA specificity. Nature (2018), incorporated by reference in its entirety).

In one embodiment, the Cas protein is a split Cas9 fusions with fluorescent proteins like GFP can be made. This would allow imaging of genomic loci (see “Dynamic Imaging of Genomic Loci in Living Human Cells by an Optimized CRISPR/Cas System” Chen B et al. Cell 2013), but in an inducible manner. As such, in some embodiments, one or more of the Cas9 parts may be associated (and in particular fused with) a fluorescent protein, for example GFP. In general, any use that can be made of a Cas9, whether wt, nickase or a dead-Cas9 (with or without associated functional domains) can be pursued using the split Cas9 approach.

In one embodiment, the Cas protein is a dimeric CRISPR RNA-guided Fokl nuclease (see., e.g., Tsai S G, et al. Nat Biotechnol. 2014. 32(6):569-576, which is incorporated herein by reference in its entirety).

In one embodiment the Cas protein is Inactive Cas9, Dead Cas9 (also referred to as dCAS9). The dead Cas9 (dCas9) CRISPR variant is made by simply inactivating the catalytic nuclease domains while maintaining the recognition domains that allow guide RNA-mediated targeting to specific DNA sequences (Komor, Alexis C., et al. Programmable editing of a target base in genomic DNA without double-stranded DNA cleavage. Nature 533.7603 (2016): 420, incorporated by reference in its entirety). dCas9 is known to silence gene expression by physically blocking the transcription. dCas9 has also been fused to other proteins and used in various applications. For instance, gene activators or inhibitors can be fused to the dCas9 to activate or repress gene expression (CRISPRa and CRISPRi). Also, tagging a fluorescent dye to the dCas9 has enabled visualization of specific DNA fragments the genome (Gaudelli, Nicole M., et al. Programmable base editing of A⋅T to G⋅C in genomic DNA without DNA cleavage Nature 551.7681 (2017): 464, incorporated by reference in its entirety). In one embodiment, FokI fused dCas9 is used (Abudayyeh, Omar O., et al. C2c2 is a single-component programmable RNA-guided RNA-targeting CRISPR effector Science 353.6299 (2016): aaf557314, incorporated by reference in its entirety).

In one embodiment, the deactivated Cas protein is a functional gene-editing nuclease by serving as a base editor. Base editor enzymes consist of a dead Cas9 domain fused with catalytic enzyme cytidine aminase that converts GC to AT or for example, a tRNA adenosine deaminase fused with Cas9 to convert AT to GC, thus allowing for a complete range of nucleotide exchanges in the genome: See e.g. Komor, Alexis C., et al. Programmable editing of a target base in genomic DNA without double-stranded DNA cleavage Nature 533.7603 (2016): 420; Gaudelli, Nicole M., et al. Programmable base editing of A⋅T to G⋅C in genomic DNA without DNA cleavage.” Nature 551.7681 (2017): 464; incorporated by reference in their entirety).

### Gene Regulation System

Various aspects of the invention described herein relate to the use of any of regulatable promoters such as the inducible or repressible promoters presented herein, or any of the regulatable transcriptional activators described herein to control expression of genes. It is also envisaged that regulatable repressors may be used to control the expression of genes.

In embodiment of any aspect, a cell of the invention may further comprise a synthetic gene regulation system, wherein the synthetic gene regulation system comprises a targeted DNA binding protein or a nucleic acid sequence encoding a targeted DNA binding protein operably linked to a promoter; and a nucleic acid sequence encoding a gene of interest operably linked to a target promoter wherein the targeted DNA binding protein is capable of binding to a target sequence, wherein the target sequence is located within the target promoter and/or the nucleic acid sequence encoding the gene of interest to thereby moderate or prevent expression of the gene of interest.

In one embodiment, the gene regulation system is provided on one or more vectors. In one embodiment, the vectors are comprised in the cell. Suitable vectors are described elsewhere herein.

The nucleic acid sequences of the synthetic gene regulation system may be part of the same nucleic acid molecule or different nucleic acid molecules. Thus, accordingly, the nucleic acid sequence or the nucleic acid sequences of the synthetic gene regulation system may be comprised on the same vector or different vectors.

The synthetic gene regulation system may comprise different components based on the desired target and outcome, for example, the DNA binding protein may be provided as a protein or encoded in a nucleic acid, the gene of interest may be provided encoded in a nucleic acid or already present within the cell, the target sequence may be within the target promoter or the nucleic acid sequence encoding the gene of interest.

In one embodiment, the gene of interest is a gene which it is desired to repress. Accordingly, a gene for which it is desired to prevent expression of in the producer cell line. In one embodiment, the gene of interest may be therapeutic gene as described elsewhere herein. Accordingly, in such an embodiment, the gene of interest may be provided encoded in a nucleic acid, i.e. exogenous to the cell.

Alternatively, the gene of interest may be an unwanted gene, of which the expression product is not required or desired when expressing the nucleic acids encoding viral proteins/RNA, and Rep proteins of the invention. In one embodiment, the unwanted gene may be toxic to the cell or an organism containing the cell when expressed, as described elsewhere herein. In one embodiment, the unwanted gene may cause a metabolic burden on the cell or an organism containing the cell when expressed. Accordingly, in such an embodiment, the unwanted gene may already be present within the cell, i.e. endogenous to the cell.

In one embodiment where the nucleic acid sequences of the synthetic gene regulation system are provided in an AAV vector, the targeted DNA binding protein moderates or prevents expression of the gene of interest from the AAV vector. Therefore, the targeted DNA binding protein downregulates expression of the gene of interest from the AAV vector. Accordingly, the gene of interest may be termed a ‘payload gene’. Accordingly, the targeted DNA binding protein moderates or prevents expression of a payload gene from the AAV vector. Accordingly, the targeted DNA binding protein downregulates expression of a payload gene from the AAV vector. Accordingly, the ‘payload’ gene may be a therapeutic gene.

In one embodiment, the targeted DNA binding protein may prevent or silence the expression of the gene of interest by preventing transcription of the gene of interest (transcriptional gene silencing). In such an embodiment, the targeted DNA binding protein may be a regulatable transcriptional repressor. Alternatively, the targeted DNA binding protein may prevent or silence expression of the gene of interest by preventing elongation of the mRNA transcript resulting in an incomplete mRNA transcript and an incomplete, truncated or non-functional expression product of the gene of interest. In such an embodiment, the targeted DNA binding protein may be a regulatable translational repressor.

In one embodiment, the targeted DNA binding protein comprises a ligand binding domain. Accordingly, the targeted DNA binding protein may comprise a plurality of DNA binding domains. The plurality of DNA binding domains may optionally be linked by linkers. In one embodiment, targeted DNA binding protein may consist of a plurality of DNA binding domains. In one embodiment, the targeted DNA binding protein may consist of a plurality of linked DNA binding domains. In one embodiment, the targeted DNA binding protein may comprise one or more DNA binding domains with no other functional domains. In one embodiment, therefore, the targeted DNA binding protein may not have any other functional domains apart from the one or more DNA binding domains. In one embodiment, the targeted DNA binding protein may consist solely of one or more DNA binding domains which may optionally be linked by non-functional linkers. In one embodiment, the targeted DNA binding protein may comprise one or more DNA binding domains with no other functionality.

The DNA binding domain recognizes the target sequence and binds to the target sequence. In one embodiment, the DNA binding domain may comprise at least one of: bacterial helix-turn-helix protein, homeodomain, winged helix-turn-helix, ETS domain, Helix-turn-helix, basic region-leucine zipper, basic region helix-loop-helix, Zinc finger, Zinc binding domain, beta sheet recognition, TATA binding protein, Rel homology domain (Garvie & Wolberger, 2001). Alternatively, the DNA binding domain may comprise a TAL effector central repeat domain (DNA binding domain), DNA binding domain from a restriction enzyme, and PAM-interactive domain from cas9 loaded with guide RNA.

In one embodiment, the DNA binding domain comprises at least one zinc finger (ZF) domain. In one embodiment, the DNA binding domain may comprise at least six ZF domains. In one embodiment, the ZF domains may be linked by linkers. In one embodiment, the ZF domain may be Cys2-His2 domain. Alternatively, the DNA binding domain may comprise a mixture of domains selected from the group of: bacterial helix-turn-helix protein, homeodomain, winged helix-turn-helix, ETS domain, Helix-turn-helix, basic region-leucine zipper, basic region helix-loop-helix, Zinc finger, Zinc binding domain, beta sheet recognition, TATA binding protein, Rel homology domain, a TAL effector central repeat domain (DNA binding domain), DNA binding domain from a restriction enzyme, DNA binding domain from a transcription factor, and PAM-interactive domain from cas9 loaded with guide RNA.

In one embodiment, the targeted DNA binding domain consists of six ZF domains linked by linkers. In another embodiment, the targeted DNA binding domain consists of nine ZF domains linked by linkers. In yet another embodiment, the targeted DNA binding domain consists of ten ZF domains linked by linkers. In a further embodiment, the targeted DNA binding domain consists of thirteen ZF domains linked by linkers. In a yet further embodiment, the targeted DNA binding domain consists of fourteen ZF domains linked by linkers.

In one embodiment, the targeted DNA binding protein binds to a target sequence within the target promoter operably linked to a nucleic acid sequence encoding a gene of interest and/or within the nucleic acid sequence encoding a gene of interest.

In one embodiment, the targeted DNA binding protein may be adapted to recognise the target sequence and, upon binding of the targeted DNA binding protein to the target sequence, to prevent expression of the gene of interest.

In one embodiment, the target sequence is within the target promoter. Without wishing to be bound by theory, binding of the targeted DNA binding protein to the target sequence within the target promoter is expected to prevent transcription of the gene of interest as the targeted DNA binding protein will prevent binding and function of DNA polymerase, transcription factors and/or transcription machinery, i.e. transcription will not be initiated.

In one embodiment, the target sequence may be within the nucleic acid sequence encoding the gene of interest. Without wishing to be bound by theory, the binding of the targeted DNA binding protein to the target sequence within the gene of interest is expected to prevent elongation of the mRNA of the gene of interest (prevent the elongation stage of transcription) resulting in incomplete, non-functional mRNA of the gene of interest. Regardless of whether the target sequence is within the target promoter of the gene of interest and/or within the nucleic acid sequence encoding the gene of interest itself, binding of the targeted DNA binding protein to the target sequence is expected to result in downregulation of the expression of the gene of interest.

Additionally, the target sequence may be within an untranslated region, such as a 5′ UTR. The target sequence may span a region of the 3′ end of the target promoter and the 5′ end of the nucleic acid sequence encoding the gene of interest. The target sequence may span a region of the 3′ end of the target promoter, 5′ UTR and the 5′ end of the nucleic acid sequence encoding the gene of interest. In one embodiment, the target sequence may be located partially within the target promoter and partially within an untranslated region or partially within the nucleic acid sequence encoding the gene of interest.

In one embodiment, the targeted DNA binding protein may moderate expression of the gene of interest by downregulating the expression of the gene of interest. This can be achieved by preventing some, but not all, of the expression of the gene of interest.

In one embodiment, the targeted DNA binding protein downregulates expression of the gene of interest by at least 10%, 20%, 30%, 40%, 50%, 60%, 70%, 80%, 90%, 95%, 99% or more as compared to an appropriate control. As described herein, an appropriate control refers to the expression of the gene of interest in a cell not contacted by the targeted DNA binding protein.

Alternatively, the targeted DNA binding protein may silence the expression of the gene of interest. This can be achieved, e.g., by preventing any expression of the gene of interest.

The promoter operably linked to the nucleic acid sequence encoding the targeted DNA binding protein may be different from the target promoter or, alternatively, the promoter operably linked to the nucleic acid sequence encoding the targeted DNA binding protein may be the target promoter. When the promoter operably linked to the nucleic acid sequence encoding the targeted DNA binding protein is a different promoter to the target promoter, it is referred to herein as the ‘expression promoter’.

In one embodiment, the expression promoter may be a constitutive promoter. The constitutive expression promoter may be selected from: CMV-IE, EF1a, SV40, PGK1, CAG, human beta actin, T7, T7lac, SP6, LP1, TTR, CK8, Synapsin, Glial fibrillary acidic protein (GFAP), CaMKII, TBG, and albumin promoter.

In one embodiment, the expression promoter may be an inducible promoter. The inducible expression promoter may be selected from: alcohol-inducible promoter, steroid-inducible promoter, tetracycline-inducible promoter, lactose-inducible promoter, forskolin-inducible promoter, hypoxia-inducible promoter, tetracycline-inducible, isopropyl β-D-1-thiogalactopyranoside-inducible, mifepristone-inducible, cumate-inducible, and phenobarbital-inducible promoter.

When the expression promoter is an inducible promoter, the synthetic gene regulation system may further comprise an inducer. The inducer may allow expression of the targeted DNA binding protein in a concentration dependent manner. In some cases, an inducible promoter can be induced by applying a suitable condition to a cell, e.g. hypoxia, hat shock, pH, etc.). In such cases the “inducer” can be a suitable condition to which the cells are exposed.

In one embodiment, the level of expression of the gene of interest can be fine-tuned by varying the concentration of the targeted DNA binding protein provided in the cell or population of cells. In one embodiment, the concentration of targeted DNA binding protein provided in the cell or population of cells is controlled by the promoter operably linked to the nucleic acid sequence encoding the targeted DNA binding protein.

In embodiments wherein the promoter operably linked to the nucleic acid sequence encoding the targeted DNA binding protein is an inducible promoter, the concentration of the targeted DNA binding protein may be varied by adjusting the concentration of the inducer or altering the condition to which cells containing the system are exposed.

In embodiments wherein the promoter operably linked to the nucleic acid sequence encoding the targeted DNA binding protein is a constitutive promoter, the concentration of the targeted DNA binding protein may be varied by choosing a constitutive promoter of desired strength.

### Recombinant Viral Vector Production

Provided herein is a method for producing viral particles comprising (a) providing any of the stable cell line described herein, e.g., a cell line having stable expression of a heterologous toxic protein under the control of an inducible promoter, or any nucleic acid construct described herein, in a viral expression system; (b) culturing the cells for a time sufficient and/or under conditions in which at least one toxic protein or construct is expressed, wherein the at least one toxic protein is operatively linked to at least one inducible promoter; (c) culturing the cells time sufficient and/or under conditions in which viral particles are produced; and (d) optionally isolating the viral particles.

Protocols for producing recombinant viral vectors and for using viral vectors for nucleic acid delivery can be found, e.g., in Current Protocols in Molecular Biology, Ausubel, F. M. et al. (eds.) Greene Publishing Associates, (1989) and other standard laboratory manuals (e.g., Vectors for Gene Therapy. In: Current Protocols in Human Genetics. John Wiley and Sons, Inc.: 1997). Further, production of AAV vectors is further described, e.g., in U.S. Pat. No. 9,441,206, the contents of which is incorporated herein by reference in its entirety.

Viral vectors produced in a viral expression system can be released (i.e. set free from the cell that produced the vector) using any standard technique. For example, viral vectors can be released via mechanical methods, for example microfluidization, centrifugation, or sonication, or chemical methods, for example lysis buffers and detergents. Released viral vectors are then recovered (i.e., collected) and purified to obtain a pure population using standard methods in the art. For example, viral vectors can be recovered from a buffer they were released into via purification methods, including a clarification step using depth filtration or Tangential Flow Filtration (TFF). As described herein in the examples, viral vectors can be released from the cell via sonication and recovered via purification of clarified lysate using column chromatography.

Expression of toxic genes required for viral vector production can be detrimental to the cell expressing the toxic gene. Nucleic acids described herein are designed to temporally control the expression of a toxic gene during viral vector production such that the viral particle is efficiently produced, with the least amount of damage to the cell. Ideally, expression of a toxic gene is induced, or its repression is removed, at the proper time in the viral vector production protocol. For example, during the 72 hours viral vector protocol described in U.S. Pat. No. 9,441,206, the Large Rep proteins are required for replication and amplification of the genome from hour 12 to hour 46 of the protocol. Expression of Small Rep proteins are required for packaging and pre-assembly of viral particles during the last 24 hours of the protocol. Prior to providing a cell into a viral vector production system described herein, no expression of Rep should be detected in the cell. Further, Cap proteins should be highly expressed, for example, having higher expression than Rep, for the first 24 hours of the protocol, followed by a gradual decrease in Cap protein expression for the remainder of the 72 hour protocol. It is preferable to have Cap expression at the start of the protocol.

Another aspect herein provides a method of producing viral particles, comprising (a) providing the cell line expressing a nucleic acid sequence comprising at least one of: a nucleic acid sequence encoding a E4 protein, a nucleic acid sequence encoding a E2A protein, and a nucleic acid sequence encoding a VA protein, wherein each nucleic acid sequences encoding any one of E4, E2A, and VA RNA is operatively linked to a first regulatable promoter; and a nucleic acid sequence encoding a Rep protein, wherein the nucleic acid encoding a Rep protein is under the control of a second regulatable promoter or transcriptional activator, wherein the first and the second regulatable promoters are different; (b) culturing the cells for a time sufficient and under conditions in which at least the nucleic acid sequence encoding a E4 protein, the nucleic acid sequence encoding a E2A protein, or the nucleic acid sequence encoding a VA protein is expressed at hour 10 of viral vector production protocol; (c) culturing the cells for a time sufficient and under conditions in which the nucleic acid sequence encoding a Rep protein is expressed at hour 12 of viral vector production protocol; (d) culturing the cells under conditions in which viral particles are produced; and (e) optionally isolating the viral particles.

In one embodiment, culturing in step (b) is culturing with an inducer of the first regulatable promoter. In one embodiment, culturing in step (c) is culturing with an inducer of the second regulatable promoter.

Another aspect herein provides a method of producing viral particles, comprising (a) providing the cell line expressing at least one of a nucleic acid construct comprising a nucleic acid sequence encoding a zinc finger (ZF) transcriptional activator operatively linked to an inducible promoter, wherein the inducible promoter is a E4-responsive promoter, or a E2-responsive promoter or other helper-gene responsive promoter; nucleic acid construct comprising a toxic protein operatively linked to a promoter comprising a target site for binding of a zinc-finger transcriptional activator (ZF-TA); nucleic acid construct comprising a Rep protein operatively linked to a promoter comprising a target site for binding of a zinc-finger transcriptional activator (ZF-TA); a nucleic acid sequence encoding at least one helper protein, wherein each nucleic acid construct is operatively linked to a regulatable promoter; a nucleic acid encoding the toxic protein is under the control of a second regulatable promoter or a zinc-finger transcriptional activator; a nucleic acid sequence encoding a E4 protein, a nucleic acid sequence encoding a E2A protein, and a nucleic acid sequence encoding a VA protein, wherein each nucleic acid sequences encoding any one of E4, E2A, and VA RNA is operatively linked to a regulatable promoter; or a nucleic acid encoding the Rep protein is under the control of a second regulatable promoter or a zinc-finger transcriptional activator; (b) culturing the cells for a time sufficient and under conditions in which at least the nucleic acid sequence encoding the E4 protein, the nucleic acid sequence encoding the E2A protein, or the nucleic acid sequence encoding the VA protein is expressed at hour 10 of viral vector production protocol; (c) culturing the cells for a time sufficient and under conditions in which the nucleic acid sequence encoding a toxic protein or Rep protein is expressed at hour 12 of viral vector production protocol; (d) culturing the cells under conditions in which viral particles are produced; and (e) optionally isolating the viral particles.

In one embodiment, culturing in step (b) is culturing with an inducer of the regulatable promoter operatively linked to at least E4, E2A, or VA protein. In one embodiment, culturing in step (c) is culturing with the ZF-TA or an inducer of a second regulatable promoter. In one embodiment, expression of the zinc-finger transcriptional activator (ZF-TF) is induced by expression of E4 or E2.

Another aspect provides a method of producing viral particles, comprising (a) providing the cell line expressing a nucleic acid sequence encoding a tetracycline-responsive transactivator protein operatively linked to a constitutive promoter; a nucleic acid sequence comprising at least one of: a nucleic acid sequence encoding a E4 protein, a nucleic acid sequence encoding a E2A protein, and a nucleic acid sequence encoding a VA protein, wherein each nucleic acid sequences encoding any one of E4, E2A, and VA RNA is operatively linked to a regulatable promoter; a nucleic acid sequence encoding a zinc finger (ZF) transcriptional activator operatively linked to an inducible promoter, wherein the inducible promoter is a E4-responsive promoter, or a E2-responsive promoter; and a nucleic acid construct comprising a Rep protein operatively linked to a promoter comprising a target site for binding of a zinc-finger transcriptional activator (ZF-TA) (b) culturing the cells for a time sufficient and under conditions in which at least the nucleic acid sequence encoding the E4 protein, the nucleic acid sequence encoding the E2A protein, or the nucleic acid sequence encoding the VA protein is expressed at hour 10 of viral vector production protocol; (c) culturing the cells for a time sufficient and under conditions in which the nucleic acid sequence encoding a toxic protein or Rep protein is expressed at hour 12 of viral vector production protocol; (d) culturing the cells under conditions in which viral particles are produced; and (e) optionally isolating the viral particles.

In one embodiment, culturing in step (b) is culturing with an inducer of the regulatable promoter operatively linked to at least E4, E2A, or VA protein. In one embodiment, culturing in step (c) is culturing with the ZF-TA. In one embodiment, expression of the zinc-finger transcriptional activator (ZF-TF) is induced by expression of E4 or E2.

Another aspect provides a method of producing viral particles, comprising (a) providing the cell line expressing a nucleic acid construct comprising a nucleic acid sequence encoding a Cap protein and a recombinase recognition sequence (RRS) located 3′ of the nucleic acid sequence encoding the Cap protein; (b) culturing the cells for a time sufficient and under conditions in which at least the nucleic acid sequence encoding the Cap protein is highly expressed for the first 24 hours of the viral vector production protocol, and is moderately expressed for the remaining 48 hours of the viral vector production protocol; (c) culturing the cells for a time sufficient and under conditions in which viral particles are produced; and (d) optionally isolating the viral particles. In one embodiment, such construct can be used to remove the nucleic acid sequence encoding the Cap protein and replace the sequence with another, for example, a different nucleic acid sequence encoding a different Cap protein.

In one embodiment, culturing in step (b) is culturing with an inducer of the regulatable promoter operatively linked to the Cap protein.

Another aspect provides a method of producing viral particles, comprising (a) providing the cell expressing a first nucleic acid construct comprising in a 5′ to 3′ direction: a promoter, a stop nucleic acid sequence flanked by a first pair of recombinase recognition sequences (RRS), and nucleic acid sequence encoding a Rep protein, wherein the promoter is operatively linked to the nucleic acid encoding the Rep protein, and a second nucleic acid construct comprising, in a 5′ to 3′ direction: a promoter, a stop nucleic acid sequence flanked by a second pair of recombinase recognition sequences (RRSs), and nucleic acid sequence encoding one or more of E2A, E4, and VA RNA, wherein the promoter is operatively linked to the nucleic acid encoding the one or more of E2A, E4, and VA RNA; (b) culturing the cells for a time sufficient and under conditions in which at least the nucleic acid sequence encoding the E4 protein, the nucleic acid sequence encoding the E2A protein, or the nucleic acid sequence encoding the VA protein is expressed at hour 10 of viral vector production protocol; (c) culturing the cells for a time sufficient and under conditions in which the nucleic acid sequence encoding a toxic protein or Rep protein is expressed at hour 12 of viral vector production protocol; (d) culturing the cells under conditions in which viral particles are produced; and (e) optionally isolating the viral particles.

In one embodiment, culturing in step (b) is culturing with a recombinase specific for the first pair of recombinase recognition sequences (RRSs). In one embodiment, culturing in step (c) is culturing with a recombinase specific for the second pair of recombinase recognition sequences (RRSs).

Current methods for recombinant viral particle production can result in an undesired amount of empty particle that are not capable of infection. In one embodiment, production of recombinant viral vectors using any of the constructs described herein results in an at least 10% decrease in empty particles as compared to an appropriate control. In one embodiment, production of recombinant viral vectors using any of the constructs described herein results in an at least 1%, 2%, 3%, 4%, 5%, 6%, 7%, 8%, 9%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more decrease in empty particles as compared to an appropriate control. As used herein, an appropriate control refers to any method for producing viral vectors that does not utilize any of the constructs described herein, e.g., the triple transfection method that does not utilize an inducible promoter. One skilled in the art can apply standard methods to assess the amount of empty particles produced.

In one embodiment, production of recombinant viral vectors using any of the constructs described herein results in an at least 10% increase in full particles as compared to an appropriate control. In one embodiment, production of recombinant viral vectors using any of the constructs described herein results in an at least 1%, 2%, 3%, 4%, 5%, 6%, 7%, 8%, 9%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more, or an at least 5×, 10×, 50×, 100×, 200×, 300×, 400×, 500×, 600×, 700×, 800×, 900×, 1000×, 1100×, 1200×, 1300×, 1400×, 1500×, 1600×, 1700×, 1800×, 1900×, 2000×, 2100×, 2200×, 2300×, 2400×, 2500×, 2600×, 2700×, 2800×, 2900×, 3000×, 3100×, 3200×, 3300×, 3400×, 3500×, 3600×, 3700×, 3800×, 3900×, 4000×, 4100×, 4200×, 4300×, 4400×, 4500×, 4600×, 4700×, 4800×, 4900×, 5000× or more increase in full particles as compared to an appropriate control. As used herein, an appropriate control refers to any method for producing viral vectors that does not utilize any of the constructs described herein, e.g., the triple transfection method that does not utilize an inducible promoter. One skilled in the art can apply standard methods to assess the amount of full particles produced.

In one embodiment, the vector can be, but is not limited to a nonviral vector or a viral vector. In one embodiment of any aspect, the vector is a DNA or RNA virus. Nonlimiting examples of a viral vector of this invention include an AAV vector, an adenovirus vector, a lentivirus vector, a retrovirus vector, a herpesvirus vector, an alphavirus vector, a poxvirus vector, a baculovirus vector, and a chimeric virus vector.

Any viral vector that is known in the art can be used in the present invention. Examples of such viral vectors include, but are not limited to vectors derived from: Adenoviridae; Birnaviridae; Bunyaviridae; Caliciviridae, Capillovirus group; Carlavirus group; Carmovirus virus group; Group Caulimovirus; Closterovirus Group; Commelina yellow mottle virus group; Comovirus virus group; Coronaviridae; PM2 phage group; Corcicoviridae; Group Cryptic virus; group Cryptovirus; Cucumovirus virus group Family ([PHgr]6 phage group; Cysioviridae; Group Carnation ringspot; Dianthovirus virus group; Group Broad bean wilt; Fabavirus virus group; Filoviridae; Flaviviridae; Furovirus group; Group Germinivirus; Group Giardiavirus; Hepadnaviridae; Herpesviridae; Hordeivirus virus group; Illarvirus virus group; Inoviridae; Iridoviridae; Leviviridae; Lipothrixviridae; Luteovirus group; Marafivirus virus group; Maize chlorotic dwarf virus group; icroviridae; Myoviridae; Necrovirus group; Nepovirus virus group; Nodaviridae; Orthomyxoviridae; Papovaviridae; Paramyxoviridae; Parsnip yellow fleck virus group; Partitiviridae; Parvoviridae; Peaenation mosaic virus group; Phycodnaviridae; Picornaviridae; Plasmaviridae; Prodoviridae; Polydnaviridae; Potexvirus group; Potyvirus; Poxviridae; Reoviridae; Retroviridae; Rhabdoviridae; Group Rhizidiovirus; Siphoviridae; Sobemovirus group; SSV 1-Type Phages; Tectiviridae; Tenuivirus; Tetraviridae; Group Tobamovirus; Group Tobravirus; Togaviridae; Group Tombusvirus; Group Torovirus; Totiviridae; Group Tymovirus; and Plant virus satellites.

Viral vectors produced by the method of the invention may comprise the genome, in part or entirety, of any naturally occurring and/or recombinant viral vector nucleotide sequence (e.g., AAV, adeno virus, lentivirus, etc.) or variant. Viral vector variants may have genomic sequences of significant homology at the nucleic acid and amino acid levels, produce viral vector which are generally physical and functional equivalents, replicate by similar mechanisms, and assemble by similar mechanisms.

Variant viral vector sequences can be used to produce viral vectors in the viral expression system described herein. For example, or more sequences having at least about 70%, at least about 75%, at least about 80%, at least about 85%, at least about 90%, at least about 95%, at least about 99%, or more nucleotide and/or amino acid sequence identity (e.g., a sequence having about 75-99% nucleotide sequence identity) to a given vector (for example, AAV, adeno virus, lentivirus, etc.).

It is to be understood that a viral expression system will further be modified to include any necessary elements required to complement a given viral vector during its production using methods described herein. For example, in certain embodiment, the nucleic acid cassette is flanked by terminal repeat sequences. In one embodiment, for the production of rAAV vectors, the AAV expression system will further comprise at least one of a recombinant AAV plasmid, a plasmid expressing Rep, a plasmid expressing Cap, and an adenovirus helper plasmid. Complementary elements for a given viral vector are well known the art and a skilled practitioner would be capable of modifying the viral expression system described herein accordingly.

A viral expression system for manufacturing an AAV vector (e.g., an AAV expression system) could further comprise Replication (Rep) genes and/or Capsid (Cap) genes in trans, for example, under the control of an inducible promoter. Expression of Rep and Cap can be under the control of one inducible promoter, such that expression of these genes are turned “on” together, or under control of two separate inducible promoters that are turned “on” by distinct inducers. On the left side of the AAV genome there are two promoters called p5 and p19, from which two overlapping messenger ribonucleic acids (mRNAs) of different length can be produced. Each of these contains an intron which can be either spliced out or not, resulting in four potential Rep genes; Rep78, Rep68, Rep52 and Rep40. Rep genes (specifically Rep 78 and Rep 68) bind the hairpin formed by the ITR in the self-priming act and cleave at the designated terminal resolution site, within the hairpin. They are necessary for the AAVS1-specific integration of the AAV genome. All four Rep proteins were shown to bind ATP and to possess helicase activity. The right side of a positive-sensed AAV genome encodes overlapping sequences of three capsid proteins, VP1, VP2 and VP3, which start from one promoter, designated p40. The cap gene produces an additional, non-structural protein called the Assembly-Activating Protein (AAP). This protein is produced from ORF2 and is essential for the capsid-assembly process. Necessary elements for manufacturing AAV vectors are known in the art, and can further be reviewed, e.g., in U.S. Pat. Nos. 5,478,745A; 5,622,856A; 5,658,776A; 6,440,742B1; 6,632,670B1; 6,156,303A; 8,007,780B2; 6,521,225B1; 7,629,322B2; 6,943,019B2; 5,872,005A; and U.S. Patent Application Numbers US 2017/0130245; US20050266567A1; US20050287122A1; the contents of each are incorporated herein by reference in their entireties.

A viral expression system for manufacturing a lentivirus using methods described herein would further comprise long terminal repeats (LTRs) flanking the nucleic acid cassette. LTRs are identical sequences of DNA that repeat hundreds or thousands of times at either end of retrotransposons or proviral DNA formed by reverse transcription of retroviral RNA. The LTRs mediate integration of the retroviral DNA via an LTR specific integrase the host chromosome. LTRs and methods for manufacturing lentiviral vectors are further described, e.g., in U.S. Pat. Nos. 7,083,981B2; 6,207,455B1; 6,555,107B2; 8,349,606B2; 7,262,049B2; and U.S. Patent Application Numbers US20070025970A1; US20170067079A1; US20110028694A1; the contents of each are incorporated herein by reference in their entireties.

A viral expression system for manufacturing an adenovirus using methods described herein would further comprise identical Inverted Terminal Repeats (ITR) of approximately 90-140 base pairs (exact length depending on the serotype) flanking the nucleic acid cassette. The viral origins of replication are within the ITRs exactly at the genome ends. The adenovirus genome is a linear double-stranded DNA molecule of approximately 36000 base pairs. Often, adenoviral vectors used in gene therapy have a deletion in the E1 region, where novel genetic information can be introduced; the E1 deletion renders the recombinant virus replication defective. ITRs and methods for manufacturing adenovirus vectors are further described, e.g., in U.S. Pat. Nos. 7,510,875B2; 7,820,440B2; 7,749,493B2; 7,820,440B2; U.S. Ser. No. 10/041,049B2; International Patent Application Numbers WO2000070071A1; and U.S. Patent Application Numbers WO2000070071A1; US20030022356A1; US20080050770A1 the contents of each are incorporated herein by reference in their entireties.

In one embodiment, the viral expression system can be a host cell, such as a virus, a mammalian cell or an insect cell. Exemplary insect cells include but are not limited to Sf9, Sf21, Hi-5, and S2 insect cell lines. For example, a viral expression system for manufacturing an AAV vector could further comprise a baculovirus expression system, for example, if the viral expression system is an insect cell. The baculovirus expression system is designed for efficient large-scale viral production and expression of recombinant proteins from baculovirus-infected insect cells.

Baculovirus expression systems are further described in, e.g., U.S. Pat. Nos. 6,919,085B2; 6,225,060B1; 5,194,376A; the contents of each are incorporated herein by reference in their entireties.

In another embodiment, the viral expression system is a cell-free system. Cell-free systems for viral vector production are further described in, for example, Cerqueira A., et al. Journal of Virology, 2016; Sheng J., et al. The Royal Society of Chemistry, 2017; and Svitkin Y. V., and Sonenberg N. Journal of Virology, 2003; the contents of which are incorporated herein by reference in their entireties.

Recombinant AAV Vector Production

Further provided herein is a method of producing adeno associate virus (AAV) particles comprising (a) providing the any of the cell lines described herein, e.g., a cell line having stable expression of at least one heterologous toxic protein required for AAV vector production, such as rep or cap, under the control of an inducible promoter, or any nucleic acid construct described herein, in an AAV expression system, (b) culturing the cells for a time sufficient and/or under conditions in which the at least one toxic protein or construct is expressed, (c) culturing the cells for a time sufficient and/or under conditions in which AAV particles are produced, and (d) optionally isolating the AAV particles.

In one embodiment, the step of culturing the cells for a time sufficient and/or under conditions in which AAV particles are produced occurs only after the toxic protein or construct is sufficiently expressed. For example, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or more hours after the cell is contacted with the inducer or applying suitable inducing conditions to the cell. As used herein, “sufficient expression” refers to the level of expression a protein required for proper function, e.g., the level of rep protein needed in the cell to induce replication.

If a cell comprises more than one distinct regulatable promoter, e.g., inducible promoter, the more than one inducible promoters can be induced to drive expression on the protein at substantially the same time, or at different times. Alternatively, if a cell comprises more than one distinct inducible promoter, the more than one inducible promoters can be induced to drive expression on the protein induced for the same period of time, or for different periods of time. In one embodiment, the cells are cultured with at least two inducers at substantially the same time, and for the same duration. In one embodiment, culturing with a first inducer is occurring when culturing with a second inducer begins, such that there is overlap in terms of culturing. This is sometimes referred to herein as “simultaneous” or “concurrent culturing.” In other embodiments, culturing with the first inducer ends prior to culturing with the second inducer beginning. When culturing occurs at substantially the same time or simultaneously, the first and second inducer can be provided in the same culture medium. Alternatively, when culturing occurs at substantially the same time or simultaneously, the first and second inducer can be provided in different culture mediums.

In one embodiment, the cells are cultured in suspension. In another embodiment, the cells are cultured in animal component-free conditions. The animal component-free medium can be any animal component-free medium (e.g., serum-free medium) compatible with a given cell line, for example, HEK293 cells. Examples include, without limitation, SFM4Transfx-293 (Hyclone), Ex-Cell 293 (JRH Biosciences), LC-SFM (Invitrogen), and Pro293-S(Lonza).

Conditions sufficient for the replication and packaging of the AAV particles can be, e.g., the presence of AAV sequences sufficient for replication of an AAV template and encapsidation into AAV capsids (e.g., AAV rep sequences and AAV cap sequences) and helper sequences from adenovirus and/or herpesvirus. In particular embodiments, the AAV template comprises two AAV ITR sequences, which are located 5′ and 3′ to the heterologous nucleic acid sequence, although they need not be directly contiguous thereto.

In some embodiments, the AAV template comprises an ITR that is not resolved by Rep to make duplexed AAV vectors as described in international patent publication WO 01/92551.

The AAV template and AAV Rep and/or Cap sequences are provided under conditions such that virus vector comprising the AAV template packaged within the AAV capsid is produced in the cell. The method can further comprise the step of collecting the virus vector from the culture. In one embodiment, the virus vector can be collected by lysing the cells, e.g., after removing the cells from the culture medium, e.g., by pelleting the cells. In another embodiment, the virus vector can be collected from the medium in which the cells are cultured, e.g., to isolate vectors that are secreted from the cells. Some or all of the medium can be removed from the culture one time or more than one time, e.g., at regular intervals during the culturing step for collection of rAAV (such as every 12, 18, 24, or 36 hours, or longer extended time that is compatible with cell viability and vector production), e.g., beginning about 48 hours post-transfection. After removal of the medium, fresh medium, with or without additional nutrient supplements, can be added to the culture. In one embodiment, the cells can be cultured in a perfusion system such that medium constantly flows over the cells and is collected for isolation of secreted rAAV. Collection of rAAV from the medium can continue for as long as the cells, e.g., the transfected cells, remain viable, e.g., 48, 72, 96, or 120 hours or longer post-transfection, or in the case of the use of an inducible promoter to express a toxic protein or construct, e.g., 48, 72, 96, or 120 hours or longer post-induction. In certain embodiments, the collection of secreted rAAV is carried out with serotypes of AAV (such as AAV8 and AAV9), which do not bind or only loosely bind to the producer cells. In other embodiments, the collection of secreted rAAV is carried out with heparin binding serotypes of AAV (e.g., AAV2) that have been modified so as to not bind to the cells in which they are produced. Examples of suitable modifications, as well as rAAV collection techniques, are disclosed in U.S. Publication No. 2009/0275107, which is incorporated by reference herein in its entirety.

In the event that a cell line described herein (stable or transient) does not stably or transiently express Rep or Cap, these sequences are to be provided to the AAV expression system. AAV Rep or Cap sequences may be provided by any method known in the art. Current protocols typically express the AAV Rep or Cap genes on a single plasmid. The AAV replication and packaging sequences need not be provided together, although it may be convenient to do so. The AAV Rep and/or Cap sequences may be provided by any viral or non-viral vector. For example, the rep/cap sequences may be provided by a hybrid adenovirus or herpesvirus vector (e.g., inserted into the Ela or E3 regions of a deleted adenovirus vector). EBV vectors may also be employed to express the AAV Rep and/or Cap genes. One advantage of this method is that EBV vectors are episomal, yet will maintain a high copy number throughout successive cell divisions (i.e., arc stably integrated into the cell as extra-chromosomal elements, designated as an “EBV based nuclear episome,” see Margolski, Curr. Top. Microbial. Immun. 158:67 (1992)).

Typically, the AAV Rep/Cap sequences will not be flanked by the TRs, to prevent rescue and/or packaging maintain of these sequences.

The AAV template can be provided to the cell using any method known in the art. For example, the template can be supplied by a non-viral (e.g., plasmid) or viral vector. In particular embodiments, the AAV template is supplied by a herpesvirus or adenovirus vector (e.g., inserted into the Ela or E3 regions of a deleted adenovirus). As another illustration, Palombo et al., J. Virol. 72:5025 (1998), describes a baculovirus vector carrying a reporter gene flanked by the AAV TRs. EBV vectors may also be employed to deliver the template, as described above with respect to the rep/cap genes.

In another representative embodiment, the AAV template is provided by a replicating rAAV virus. In still other embodiments, an AAV provirus comprising the AAV template is stably integrated into the chromosome of the cell.

To enhance virus titers, helper virus functions (e.g., adenovirus or herpesvirus) that promote a productive AAV infection can be provided to the cell. Helper virus sequences necessary for AAV replication are known in the art. In the event that a cell line described herein (stable or transient) does not stably or transiently express a helper protein, these sequences are to be provided to the AAV expression system. Typically, these sequences will be provided by a helper adenovirus or herpesvirus vector. Alternatively, the adenovirus or herpesvirus sequences can be provided by another non-viral or viral vector, e.g., as a non-infectious adenovirus miniplasmid that carries all of the helper genes that promote efficient AAV production as described by Ferrari et al., Nature Med. 3:1295 (1997), and U.S. Pat. Nos. 6,040,183 and 6,093,570, which is incorporated herein by reference.

Further, the helper virus functions may be provided by a packaging cell with the helper sequences embedded in the chromosome or maintained as a stable extrachromosomal element. Generally, the helper virus sequences cannot be packaged into AAV virions, e.g., are not flanked by TRs.

Those skilled in the art will appreciate that it may be advantageous to provide the AAV Cap and Rep sequences and the helper virus sequences (e.g., adenovirus sequences) on a single helper construct. In one embodiment, expression of at least one gene product encoded by the single helper construct is controlled by an inducible promoter. This helper construct may be a non-viral or viral construct. As one nonlimiting illustration, the helper construct can be a hybrid adenovirus or hybrid herpesvirus comprising the AAV Rep and/or Cap genes.

In one particular embodiment, the AAV Rep and/or Cap sequences and the adenovirus helper sequences are supplied by a single adenovirus helper vector. This vector can further comprise the AAV template. The AAV Rep and/or Cap sequences and/or the AAV template can be inserted into a deleted region (e.g., the E1 a or E3 regions) of the adenovirus. In one embodiment, expression of at least one gene product encoded by the AAV template is controlled by an inducible promoter.

In a further embodiment, the AAV Rep and/or Cap sequences and the adenovirus helper sequences are supplied by a single adenovirus helper vector. According to this embodiment, the AAV template can be provided as a plasmid template.

In another illustrative embodiment, the AAV Rep and/or Cap sequences and adenovirus helper sequences are provided by a single adenovirus helper vector, and the AAV template is integrated into the cell as a provirus. Alternatively, the AAV template is provided by an EBV vector that is maintained within the cell as an extrachromosomal element (e.g., as an EBV based nuclear episome).

Use of the inducible and repressible promoters described herein can be used to achieve temporal regulation of any of the toxic proteins required for viral vector production, for example, Rep and Cap. In one embodiment, inducible and/or repressible promoters provide for careful fine tuning of expression of a toxic protein, such that one can tailor the start and stop of the expression to achieve the desired level of expression, and at the desired timing during production.

In a further exemplary embodiment, the AAV Rep and/or Cap sequences and adenovirus helper sequences are provided by a single adenovirus helper. The AAV template can be provided as a separate replicating viral vector. For example, the AAV template can be provided by an AAV particle or a second recombinant adenovirus particle.

According to the foregoing methods, the hybrid adenovirus vector typically comprises the adenovirus 5′ and 3′ cis sequences sufficient for adenovirus replication and packaging (i.e., the adenovirus terminal repeats and PAC sequence). The AAV rep and/or cap sequences and, if present, the AAV template are embedded in the adenovirus backbone and are flanked by the 5′ and 3′ cis sequences, so that these sequences may be packaged into adenovirus capsids. As described above, the adenovirus helper sequences and the AAV rep and/or cap sequences are generally not flanked by TRs so that these sequences are not packaged into the AAV virions. Zhang et al., Gene Ther. 18:704 ((2001)) describe a chimeric helper comprising both adenovirus and the AAV rep and/or cap genes.

Herpesvirus may also be used as a helper virus in AAV packaging methods. Hybrid herpesviruses encoding the AAV Rep protein(s) may advantageously facilitate scalable AAV vector production schemes. A hybrid herpes simplex virus type I (HSV-1) vector expressing the AAV-2 rep and cap genes has been described (Conway et al., Gene Ther. 6:986 (1999) and WO 00/17377).

AAV vector stocks free of contaminating helper virus may be obtained by any method known in the art. For example, AAV and helper virus may be readily differentiated based on size. AAV may also be separated away from helper virus based on affinity for a heparin substrate (Zolotukhin et al. Gene Ther. 6:973 (1999)). Deleted replication-defective helper viruses can be used so that any contaminating helper virus is not replication competent. As a further alternative, an adenovirus helper lacking late gene expression may be employed, as only adenovirus early gene expression is required to mediate packaging of AAV. Adenovirus mutants defective for late gene expression are known in the art (e.g., ts100K and ts149 adenovirus mutants).

In various embodiments, the method of the invention is completely scalable, so it can be carried out in any desired volume of culture medium, e.g., from 10 ml (e.g., in shaker flasks) to 10 L, 50 L, 100 L, or more (e.g., in bioreactors such as wave bioreactor systems and stirred tanks).

The method is suitable for production of all serotypes and chimeras of AAV, e.g., AAV1, AAV2, AAV3, AAV4, AAV5, AAV6, AAV7, AAV8, AAV9, AAV 10, AAV11, AAV12, AAV13, and any chimeras thereof.

In certain embodiments, the method provides at least about 1×10⁴ vector genome-containing particles per cell prior to purification, e.g., at least about 2×10⁴ is 3×10⁴, 4×10⁴, 5×10⁴, 6×10⁴, 7×10⁴, 8×10⁴, 9×10⁴, or 1×10⁵ or more vector genome-containing particles per cell priorto purification. In other embodiments, the method provides at least about 1×10¹² purified vector genome-containing particles per liter of cell culture, e.g., at least about 5×10¹², 1×10¹³, 5×10¹³, or 1×10¹⁴ or more purified vector genome-containing particles per liter of cell culture.

In general, the recombinant viral vector of the present invention can be employed to deliver a nucleic acid, e.g., a therapeutic gene, encoding a polypeptide or functional RNA to treat and/or prevent any disease state for which it is beneficial to deliver a therapeutic polypeptide or functional RNA. Illustrative disease states include, but are not limited to those listed in Table 3.

TABLE 3
Exemplary diseases and disorders.

Amyotrophic Lateral Sclerosis; endotoxemia; atherosclerotic vascular disease is coronary
artery disease; stent restenosis; carotid metabolic disease; stroke; acute myocardial infarction;
heart failure; peripheral arterial disease; limb ischemia; vein graft failure; AV fistula failure;
Crohn's disease; ulcerative colitis; ileitis and enteritis; vaginitis; psoriasis and inflammatory
dermatoses such as dermatitis; eczema; atopic dermatitis; allergic contact dermatitis; urticaria;
vasculitis; spondyloarthropathies; scleroderma; respiratory allergic diseases such as asthma;
allergic rhinitis; hypersensitivity lung diseases; arthritis (e.g. rheumatoid and psoriatic);
eczema; psoriasis; osteoarthritis; multiple sclerosis; systemic lupus erythematosus; diabetes
mellitus; glomerulonephritis; graft rejection (including allograft rejection and graft-v-host
disease) or rejection of an engineered tissue; infectious diseases; myositis; inflammatory CNS
disorders; stroke; closed-head injuries; neurodegenerative diseases; Alzheimer's disease;
encephalitis; meningitis; osteoporosis; gout; hepatitis; hepatic veno-occlusive disease (VOD);
hemorrhagic cystitis; nephritis; sepsis; sarcoidosis; conjunctivitis; otitis; chronic obstructive
pulmonary disease; sinusitis; Bechet's syndrome; graft-versus-tumor effect; mucositis;
appendicitis; ruptured appendix; peritonitis; aortic valve disease; mitral valve disease; Rett's
syndrome; tuberous sclerosis; phenylketonuria; Smith-Lemli-Opitz syndrome and fragile X
syndrome; Parkinson's disease; Aicardi-Goutierès Syndrome; Alexander Disease; Allan-
Hemdon-Dudley Syndrome; POLG-Related Disorders; Alpha-Mannosidosis (Type II and III);
Alström Syndrome; Angelman Syndrome; Ataxia-Telangiectasia; Neuronal Ceroid-
Lipofuscinoses; Beta-Thalassemia; Bilateral Optic Atrophy and (Infantile) Optic Atrophy Type
1; Retinoblastoma (bilateral); Canavan Disease; Cerebrooculofacioskeletal Syndrome 1
[COFS1]; Cerebrotendinous Xanthomatosis; Cornelia de Lange Syndrome; MAPT-Related
Disorders; Genetic Prion Diseases; Dravet Syndrome; Early-Onset Familial Alzheimer Disease;
Friedreich Ataxia [FRDA]; Fryns Syndrome; Fucosidosis; Fukuyama Congenital Muscular
Dystrophy; Galactosialidosis; Gaucher Disease; Organic Acidemias; Hemophagocytic
Lymphohistiocytosis; Hutchinson-Gilford Progeria Syndrome; Mucolipidosis II; Infantile Free
Sialic Acid Storage Disease; PLA2G6-Associated Neurodegeneration; Jervell and Lange-
Nielsen Syndrome; Junctional Epidermolysis Bullosa; Huntington Disease; Krabbe Disease
(Infantile); Mitochondrial DNA-Associated Leigh Syndrome and NARP; Lesch-Nyhan
Syndrome; LIS1-Associated Lissencephaly; Lowe Syndrome; Maple Syrup Urine Disease;
MECP2 Duplication Syndrome; ATP7A-Related Copper Transport Disorders; LAMA2-
Related Muscular Dystrophy; Arylsulfatase A Deficiency; Mucopolysaccharidosis Types I; II
or III; Peroxisome Biogenesis Disorders; Zellweger Syndrome Spectrum; Neurodegeneration
with Brain Iron Accumulation Disorders; Acid Sphingomyelinase Deficiency; Niemann-Pick
Disease Type C; Glycine Encephalopathy; ARX-Related Disorders; Urea Cycle Disorders;
COL1A1/2-Related Osteogenesis Imperfecta; Mitochondrial DNA Deletion Syndromes; PLP1-
Related Disorders; Perry Syndrome; Phelan-McDermid Syndrome; Glycogen Storage Disease
Type II (Pompe Disease) (Infantile); MAPT-Related Disorders; MECP2-Related Disorders;
Rhizomelic Chondrodysplasia Punctata Type 1; Roberts Syndrome; Sandhoff Disease;
Schindler Disease-Type 1; Adenosine Deaminase Deficiency; Smith-Lemli-Opitz Syndrome;
Spinal Muscular Atrophy; Infantile-Onset Spinocerebellar Ataxia; Hexosaminidase A
Deficiency; Thanatophoric Dysplasia Type 1; Collagen Type VI-Related Disorders; Usher
Syndrome Type I; Congenital Muscular Dystrophy; Wolf-Hirschhorn Syndrome; Lysosomal
Acid Lipase Deficiency; and Xeroderma Pigmentosum.

In one embodiment, the AAV plasmids or templates described herein can further comprise a transgene, for example, a therapeutic transgene. The therapeutic transgene described herein modulates, e.g., increases or decreases, the expression of a disease gene. In one embodiment, the therapeutic gene alters (e.g., increases or decreases) the expression of a disease gene or gene product therefrom. For example, expression of the therapeutic gene in a cell increases the expression of a disease gene by at least 5%, 10%, 15%, 20%, 25%, 30%, 35%, 40%, 45%, 50%, 55%, 60%, 65%, 70%, 75%, 80%, 85%, 90%, 95%, 99%, or more as compared to a reference level, or at least 10-fold, 20-fold, 30-fold, 40-fold, 50-fold, 60-fold, 70-fold, 80-fold, 90-fold, 100-fold, 150-fold, 200-fold, 250-fold, 300-fold, 350-fold, 400-fold, 450-fold, 500-fold, or more as compared to a reference level. Alternatively, expression of the therapeutic gene in a cell decreases the expression of a disease gene by at least 500,10%, 15%, 20%,25%, 30%, 35%, 40%, 45%, 50%, 55%, 60%, 65%, 70%, 75%, 80%, 85%, 90%, 95%, 99%, or more as compared to a reference level. As used herein, “reference level” refers to the expression level of the disease gene in an otherwise identical sample that is not co-administered the viral vector and antibiotic, or is administered the viral vector without co-administration of the antibiotic. One skilled in the art can assess the level ofa disease gene or gene product therefrom using standard techniques, for example, using PCR-based assays or western blotting to measure mRNA or protein levels, respectively. Illustrative disease genes include, but are not limited to those listed in Table 4.

In one embodiment, the transgene is not under control of an inducible promoter. In one embodiment, the transgene is under control of a different inducible promoter as the toxic gene to prevent transgene expression in the producer cell line. In an alternate embodiment, the transgene is under control of a repressible promoter such that its expression can be repressed in the producer cell line. In one embodiment, the transgene is the gene of interest within the gene regulation system described hereinabove. In such an embodiment, the transgene is under the control of atargeted DNA binding protein which is capable of binding to a target sequence within the target promoter of, and/or the nucleic acid sequence encoding the transgene to thereby moderate or prevent expression of the transgene. In one embodiment, the target promoter may be regarded as a ‘repressible promoter’. In one embodiment, the targeted DNA binding protein may be regarded as a regulatable repressor, or a regulatable transcriptional repressor

TABLE 4
Exemplary disease genes.

1p36; 18p; 6p21.3; 14q32; AAAS; FGD1; EDNRB; CP (3p26.3); LMBR1; COL2A1
(12q13.11); 4p16.3; HMBS; ADSL; ABCD1; JAG1; NOTCH2; TP63; TREX1; RNASEH2A;
RNASEH2B; RNASEH2C; SAMHD1; ADAR; IFIH1; GFAP; HGD; 10q26.13; ATP1A3;
ALMS1; ALAD; FGFR2; VPS33B; ATM; PITX2; FOXO1A; FOXC1; PAX6; 10q26; FGFR2;
IGF-2; CDKN1C; H19; KCNQ1OT1; BTD; BCS1L; 15q26.1; 17 FLCN; ATP2A1; MAOA;
NOTCH3; HTRA1; X 17q24.3-q25.1; ASPA; RAB23; SNAP29; FTR (7q31.2); PMP22;
MFN2; CHD7; LYST; RUNX2; ERCC6; ERCC8; X RPS6KA3; COH1; COL11A1;
COL11A2; COL2A1; NTRK1; PTEN; CPOX; 14q13-q21; 5p; 16q12; FGFR2; FGFR3;
FGFR3; ATP2A2; Xp11.22 CLCN5; OCRL; WT1; 18q; 22q11.2; HSPB8; HSPB1; HSPB3;
GARS; REEP1; IGHMBP2; SLC5A7; DCTN1; TRPV4; SIGMAR1; COL1A1; COL1A2;
COL3A1; COL5A1; COL5A2; TNXB; ADAMTS2; PLOD1; B4GALT7; DSE; EMD; LMNA;
SYNE1; SYNE2; FHL1; TMEM43; FECH; FANCA; FANCB; FANCC; FANCD1; FANCD2;
FANCE; FANCF; FANCG; FANCI; FANCJ; FANCL; FANCM; FANCN; FANCP; FANCS;
RAD51C; XPF; GLA (Xq22.1); APC; IKBKAP; MYCN; MED12; FXN; GALT; GALK1;
GALE; GBA (1); PAX6; GCDH; ETFA; ETFB; ETFDH; BCS1L; MYO5A; RAB27A;
MLPH; ATP2C1 (3); ABCA12; HFE; HAMP; HFE2B; TFR2; TF; CP ; FVIII; UROD; 3q12;
ENG; ACVRL1; MADH4; GNE; MYHC2A; VCP; HNRPA2B1; HNRNPA1; EXT1; EXT2;
EXT3; HPS1; HPS3; HPS4; HPS5; HPS6; HPS7; AP3B1; PMP22; NODAL; NKX2-5; ZIC3;
CCDC11; CFC1; SESN1; CBS (gene); HD; IDS; IDUA; AASS; AGXT; GRHPR; DHDPSL;
ABCA1; COL2A1; FGFR3 (4p16.3); 20q11.2; IKBKG (Xq28); TBX4; 15q11-14; FGFR2;
INPP5E; TMEM216; AHI1; NPHP1; CEP290; TMEM67; RPGRIP1L; ARL13B; CC2D2A;
OFD1; TMEM138; TCTN3; ZNF423; AMRC9; ALS2; COL2A1; PDGFRB; GAL; ATP13A2;
LCAT; HPRT (X); TP53; MSH2; MLH1; MSH6; PMS2; PMS1; TGFBR2; MLH3; RYR1
(19q13.2); BCKDHA; BCKDHB; DBT; DLD; ARSB; 20 q13.2-13.3; XK (X); AP1S1; MEFV;
ATP7A (Xq21.1); MMAA; MMAB; MMACHC; MMADHC; LMBRD1; MUT; RAB3GAP
(2q21.3); ASPM (1q31); GALNS; GLB1; ZEB2 (2); FGFR3; MEN1; RET; MSTN; DMPK;
CNBP; HYAL1; 17q11.2; SMPD1; NPA; NPB; NPC1; NPC2; GLDC; AMT; GCSH; PTPN11;
KRAS; SOS1; RAF1; NRAS; HRAS; BRAF; SHOC2; MAP2K1; MAP2K2; CBL; RELN;
RAG1; RAG2; COL1A1; COL1A2; IFITM5; PANK2 (20p13-p12.3); UROD; PDS; STK11;
FGFR1; FGFR2; PAH; AASDHPPT; TCF4 (18); PKD1 (16) or PKD2 (4); DNAI1; DNAH5;
TXNDC3; DNAH11; DNAI2; KTU; RSPH4A; RSPH9; LRRC50; PROC; PROS1; ABCC6;
RP1; RP2; RPGR; PRPH2; IMPDH1; PRPF31; CRB1; PRPF8; TULP1; CA4; HPRPF3;
ABCA4; EYS; CERKL; FSCN2; TOPORS; SNRNP200; PRCD; NR2E3; MERTK; USH2A;
PROM1; KLHL7; CNGB1; TTC8; ARL6; DHDDS; BEST1; LRAT; SPARA7; CRX ;
MECP2; ESCO2; CREBBP; HEXB; SGSH; NAGLU; HGSNAT; GNS; HSPG2; COL2A1;
FBN1; 11p15; Xp11.22; PHF8; ABCB7; SLC25A38; GLRX5; GUSB; DHCR7; 17p11.2;
ATXN1; ATXN2; ATXN3; PLEKHG4; SPTBN2; CACNA1A; ATXN7; ATXN8OS;
ATXN10; TTBK2; PPP2R2B; KCNC3; PRKCG; ITPR1; TBP; KCND3; FGF14; FGFR3;
ABCA4; CNGB3; ELOVL4; PROM1; COL11A1; COL11A2; COL2A1; COL9A1; COL2A1;
HEXA (15); GCH1; PCBD1; PTS; QDPR; MTHFR; DHFR; FGFR3; 5q32-q33.1 (TCOF1;
POLR1C; or POLR1D); TSC1; TSC2; MYO7A; USH1C; CDH23; PCDH15; USH1G;
USH2A; GPR98; DFNB31; CLRN1; PPOX; VHL; PAX3; MITF; WS2B; WS2C; SNAI2;
EDNRB; EDN3; SOX10; COL11A2; ATP7B; C2ORF37 (2q22.3-q35); 4p16.3; 15 ERCC4;
CENPVL1; CENPVL2; GSPT2; MAGED1; ALAS2 (X); PEX1; PEX2; PEX3; PEX5; PEX6;
PEX10; PEX12; PEX13; PEX14; PEX16; PEX19; and PEX26

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.”

In one respect, the present invention relates to the herein described compositions, methods, and respective component(s) thereof, as essential to the invention, yet open to the inclusion of unspecified elements, essential or not (“comprising). In some embodiments, other elements to be included in the description of the composition, method or respective component thereof are limited to those that do not materially affect the basic and novel characteristic(s) of the invention (“consisting essentially of”). This applies equally to steps within a described method as well as compositions and components therein. In other embodiments, the inventions, compositions, methods, and respective components thereof, described herein are intended to be exclusive of any element not deemed an essential element to the component, composition or method (“consisting of”).

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The invention is further illustrated by the following examples, which should not be construed as further limiting.
1. The invention described herein can further be described in the following numbered paragraphs:
2. A stable cell line for recombinant viral vector production, comprising at least one inducible promoter operatively linked to a heterologous gene that encodes a toxic protein.
3. The stable cell of paragraph 1, wherein the regulatable promoter is an inducible or repressible promoter.
4. The stable cell of any preceding paragraph, wherein the regulatable promoter is an inducible promoter.
5. The stable cell line of paragraph 1, wherein the toxic protein is a viral protein.
6. The stable cell line of any preceding paragraph, wherein the toxic protein is associated with nucleic acid transcription.
7. The stable cell line of any preceding paragraph, wherein the toxic protein is associated with capsid or envelope production.
8. The stable cell line of any preceding paragraph, wherein the viral protein is selected from the group consisting of replication (rep), capsid (cap), envelope (env), and polymerase (pol).
9. The stable cell line of any preceding paragraph, wherein the inducible promoter is selected from the group selected from a forskolin inducible promoter, a hypoxia inducible promoter, a tetracycline inducible promoter, an alcohol inducible promoter, a steroid inducible promoter, an RU486 inducible promoter, an ecdysone inducible promoter, a rapamycin inducible promoter, a metallothionein inducible promoter, a hormone inducible promoter and a metal inducible promoter.
10. The stable cell line of any preceding paragraph, wherein the cell comprises at least two inducible promoters, and wherein the at least two inducible promoters are induced by different compositions, and the at least two inducible promoters are operatively linked to distinct heterologous genes that encode different toxic proteins.
11. The stable cell line of any preceding paragraph, wherein the cell comprises a first inducible promoter that is operatively linked to a repressible element that can stop protein expression.
12. The stable cell line of any preceding paragraph, wherein the first inducible promoter that further encodes a protein that represses expression of the first inducible promoter.
13. The stable cell line of any preceding paragraph, wherein the cell comprises a first inducible promoter that further encodes a protein that induces expression of a second inducible promoter.
14. The stable cell line of any preceding paragraph, wherein the cell is a eukaryotic cell or a prokaryotic cell.
15. The stable cell line of any preceding paragraph, wherein the cell is selected from the cell types listed in Table 2.
16. The stable cell line of any preceding paragraph, wherein the cell is derived from a cell type selected from the cell types listed in Table 2.
17. The stable cell line of any preceding paragraph, wherein contacting the cell with an inducer results in expression of the at least one toxic protein.
18. The stable cell line of any preceding paragraph, for use in production of a viral particles selected from the group consisting of: an adenovirus vector, a lentivirus vector, a retrovirus vector, a herpesvirus vector, an alphavirus vector, a poxvirus vector, a baculovirus vector, and a chimeric virus vector.
19. A stable cell line for recombinant AAV vector production, comprising at least one inducible promoter, wherein the inducible promoter is operatively linked to a heterologous rep gene that encodes a rep protein.
20. The stable cell line of any preceding paragraph, wherein the inducible promoter is further operatively linked to a heterologous cap gene that encodes a cap protein.
21. The stable cell line of any preceding paragraph, further comprising a second inducible promoter operatively linked to a heterologous cap gene that encodes a cap protein, wherein the second inducible promoter is induced by a compound different from the first inducible promoter.
22. A stable cell line for recombinant AAV vector production, comprising at least one inducible promoter, wherein the inducible promoter is operatively linked to a heterologous cap gene that encodes a cap protein.
23. A method of producing the stable cell line of any preceding paragraph, the method comprising:
a. transforming a population of cells with at least one nucleic acid cassette containing an inducible promoter operatively linked to a heterologous gene that encodes a toxic protein;
b. culturing the population of cells of (a) under conditions and for a time sufficient to permit expression of the nucleic acid cassette;
c. selecting for a cell that stably expresses the nucleic acid cassette; and
d. growing the cell of (c) to produce the cell line.
24. A method of producing adeno associate virus (AAV) particles, comprising;
a. providing the stable cells of any preceding paragraph in an AAV expression system;
b. culturing the cells under conditions in which the at least one toxic protein is expressed;
c. culturing the cells under conditions in which AAV particles are produced; and
d. optionally isolating the AAV particles.
25. The method of any preceding paragraph, wherein the cells are cultured in suspension.
26. The method of any preceding paragraph, wherein the cells are cultured in animal component-free conditions.
27. The method of any preceding paragraph, wherein step (c) comprises isolating the AAV particles from the cells.
28. The method of any preceding paragraph, wherein step (c) comprises isolating the AAV particles from medium in which the cells are cultured.
29. The method of any preceding paragraph, wherein the cells are cultured in shaker flasks.
30. The method of any preceding paragraph, wherein the cells are cultured in bioreactors.
31. The method of any preceding paragraph, wherein step (c) occurs after the toxic protein is expressed.
32. The method of any preceding paragraph, wherein if the stable cell comprises at least two inducible promoters, the at least two inducible promoters are induced at substantially the same time.
33. The method of any preceding paragraph, wherein if the stable cell comprises at least two inducible promoters, the at least two inducible promoters are induced at different time and/or for different durations.
34. The method of any preceding paragraph, wherein the method is capable of producing all serotypes, chimeras, and hybrids of AAV.
35. The method of any preceding paragraph, wherein said AAV is selected from the group consisting ofAAV1, AAV2, AAV3, AAV4, AAV5, AAV6, AAV7, AAV8, AAV9, AAV 10, AAV11, AAV12, and AAV13 or a chimeric AAV that is composed of AAV1-13 2.5, 218, 9.45 and other chimeric or hybrid capsids.
36. The method of any preceding paragraph, wherein the AAV particle comprises a rational haploid capsid.
37. The method of any preceding paragraph, wherein said AAV expression system comprises at least one of a recombinant AAV plasmid, a plasmid expressing Rep, a plasmid expressing Cap, and an adenovirus helper plasmid.
38. The method of any preceding paragraph, wherein the recombinant AAV plasmid encodes a transgene.
39. The method of any preceding paragraph, wherein the transgene is a therapeutic transgene.
40. The method of any preceding paragraph, wherein the method provides at least about 4×10⁴ vector genome-containing particles per cell prior to purification.
41. The method of any preceding paragraph, wherein the method provides at least about 1×10⁵ vector genome-containing particles per cell prior to purification.
42. The method of any preceding paragraph, wherein the method provides at least about 1×10¹² purified vector genome-containing particles per liter of cell culture.
43. The method of any preceding paragraph, wherein the method provides at least about 1×10¹³ purified vector genome-containing particles per liter of cell culture.
44. A method of producing viral particles, comprising;
a. providing the stable cell line of any preceding paragraph in a viral expression system;
b. culturing the cells under conditions in which at least one toxic protein is expressed, wherein the at least one toxic protein is operatively linked to at least one inducible promoter;
c. culturing the cells under conditions in which viral particles are produced; and
d. optionally isolating the viral particles.
45. The method of any preceding paragraph, wherein the viral particles are selected from the group consisting of: an adenovirus vector, a lentivirus vector, a retrovirus vector, a herpesvirus vector, an alphavirus vector, a poxvirus vector, a baculovirus vector, and a chimeric virus vector.
46. A cell line for recombinant viral vector production, comprising transient expression of at least one inducible promoter, wherein the inducible promoter is operatively linked to a heterologous gene that encodes a toxic protein.
47. A stable cell line for recombinant viral vector production, comprising at least one inducible promoter operatively linked to a heterologous gene that encodes a toxic protein,
i. wherein the at least one inducible promoter is selected from the group consisting of: a forskolin inducible promoter, a hypoxia inducible promoter, a tetracycline inducible promoter, an alcohol inducible promoter, a steroid inducible promoter, an RU486 inducible promoter, an ecdysone inducible promoter, a rapamycin inducible promoter, a metallothionein inducible promoter, a hormone inducible promoter and a metal inducible promoter.
48. A stable cell line for recombinant viral vector production, comprising at least one forskolin inducible promoter operatively linked to at least one heterologous gene that encodes a toxic protein.
49. A stable cell line for recombinant viral vector production, comprising at least one hypoxia inducible promoter operatively linked to at least one heterologous gene that encodes a toxic protein.
50. A stable cell line for recombinant viral vector production, comprising at least one forskolin inducible promoter operatively linked to at least one heterologous gene that encodes a toxic protein, and
i. at least one inducible promoter operatively linked to at least one heterologous gene that encodes a toxic protein,
ii. wherein the at least one inducible promoter is selected from the group consisting of: a forskolin inducible promoter, a hypoxia inducible promoter, a tetracycline inducible promoter, an alcohol inducible promoter, a steroid inducible promoter, an RU486 inducible promoter, an ecdysone inducible promoter, a rapamycin inducible promoter, a metallothionein inducible promoter, a hormone inducible promoter and a metal inducible promoter.
51. A stable cell line for recombinant viral vector production, comprising at least one hypoxia inducible promoter operatively linked to at least one heterologous gene that encodes a toxic protein, and
i. at least one inducible promoter operatively linked to at least one heterologous gene that encodes a toxic protein,
ii. wherein the at least one inducible promoter is selected from the group consisting of: a forskolin inducible promoter, a hypoxia inducible promoter, a tetracycline inducible promoter, an alcohol inducible promoter, a steroid inducible promoter, an RU486 inducible promoter, an ecdysone inducible promoter, a rapamycin inducible promoter, a metallothionein inducible promoter, a hormone inducible promoter and a metal inducible promoter.
52. A stable cell line for recombinant viral vector production, comprising at least one forskolin inducible promoter operatively linked to at least one heterologous gene that encodes a toxic protein, and at least one hypoxia inducible promoter operatively linked to at least one heterologous gene that encodes a toxic protein.
53. A stable cell line for recombinant viral vector production, comprising at least one inducible promoter having a sequence of SEQ ID NO: 1 or SEQ ID NO: 3 operatively linked to at least one heterologous gene that encodes a toxic protein.
54. A stable cell line for recombinant viral vector production, comprising at least one inducible promoter having a sequence of any one of SEQ ID NO: 6 to SEQ ID NO: 9 operatively linked to at least one heterologous gene that encodes a toxic protein.
55. A stable cell line for recombinant viral vector production, comprising at least one inducible promoter having a sequence of SEQ ID NO: 1 or SEQ ID NO: 3 operatively linked to at least one heterologous gene that encodes a toxic protein, and
i. at least one inducible promoter operatively linked to at least one heterologous gene that encodes a toxic protein,
ii. wherein the at least one inducible promoter is selected from the group consisting of: a forskolin inducible promoter, a hypoxia inducible promoter, a tetracycline inducible promoter, an alcohol inducible promoter, a steroid inducible promoter, an RU486 inducible promoter, an ecdysone inducible promoter, a rapamycin inducible promoter, a metallothionein inducible promoter, a hormone inducible promoter and a metal inducible promoter.
56. A stable cell line for recombinant viral vector production, comprising at least one inducible promoter having a sequence of any one of SEQ ID NO: 6 to SEQ ID NO: 9 operatively linked to at least one heterologous gene that encodes a toxic protein, and
i. at least one inducible promoter operatively linked to at least one heterologous gene that encodes a toxic protein,
ii. wherein the at least one inducible promoter is selected from the group consisting of: a forskolin inducible promoter, a hypoxia inducible promoter, a tetracycline inducible promoter, an alcohol inducible promoter, a steroid inducible promoter, an RU486 inducible promoter, an ecdysone inducible promoter, a rapamycin inducible promoter, a metallothionein inducible promoter, a hormone inducible promoter and a metal inducible promoter.
57. A stable cell line for recombinant viral vector production, comprising at least one inducible promoter having a sequence of SEQ ID NO: 1 or SEQ ID NO: 3 operatively linked to at least one heterologous gene that encodes a toxic protein, and at least one inducible promoter having a sequence of any one of SEQ ID NO: 6 to SEQ ID NO: 9 operatively linked to at least one heterologous gene that encodes a toxic protein.
58. A stable cell line for rAAV production, comprising at least one inducible promoter operatively linked to a heterologous gene that encodes a toxic protein,
i. wherein the at least one inducible promoter is selected from the group consisting of: a forskolin inducible promoter, a hypoxia inducible promoter, a tetracycline inducible promoter, an alcohol inducible promoter, a steroid inducible promoter, an RU486 inducible promoter, an ecdysone inducible promoter, a rapamycin inducible promoter, a metallothionein inducible promoter, a hormone inducible promoter and a metal inducible promoter.
59. A stable cell line for rAAV production, comprising at least one forskolin inducible promoter operatively linked to at least one heterologous gene that encodes a toxic protein.
60. A stable cell line for rAAV production, comprising at least one hypoxia inducible promoter operatively linked to at least one heterologous gene that encodes a toxic protein.
61. A stable cell line for rAAV production, comprising at least one forskolin inducible promoter operatively linked to at least one heterologous gene that encodes a toxic protein, and
i. at least one inducible promoter operatively linked to at least one heterologous gene that encodes a toxic protein,
ii. wherein the at least one inducible promoter is selected from the group consisting of: a forskolin inducible promoter, a hypoxia inducible promoter, a tetracycline inducible promoter, an alcohol inducible promoter, a steroid inducible promoter, an RU486 inducible promoter, an ecdysone inducible promoter, a rapamycin inducible promoter, a metallothionein inducible promoter, a hormone inducible promoter and a metal inducible promoter.
62. A stable cell line for rAAV production, comprising at least one hypoxia inducible promoter operatively linked to at least one heterologous gene that encodes a toxic protein, and
i. at least one inducible promoter operatively linked to at least one heterologous gene that encodes a toxic protein,
ii. wherein the at least one inducible promoter is selected from the group consisting of: a forskolin inducible promoter, a hypoxia inducible promoter, a tetracycline inducible promoter, an alcohol inducible promoter, a steroid inducible promoter, an RU486 inducible promoter, an ecdysone inducible promoter, a rapamycin inducible promoter, a metallothionein inducible promoter, a hormone inducible promoter and a metal inducible promoter.
63. A stable cell line for rAAV production, comprising at least one forskolin inducible promoter operatively linked to at least one heterologous gene that encodes a toxic protein, and at least one hypoxia inducible promoter operatively linked to at least one heterologous gene that encodes a toxic protein.
64. A stable cell line for rAAV production, comprising at least one inducible promoter having a sequence of SEQ ID NO: 1 or SEQ ID NO: 3 operatively linked to at least one heterologous gene that encodes a toxic protein.
65. A stable cell line for rAAV production, comprising at least one inducible promoter having a sequence of any one of SEQ ID NO: 6 to SEQ ID NO: 9 operatively linked to at least one heterologous gene that encodes a toxic protein.
66. A stable cell line for rAAV production, comprising at least one inducible promoter having a sequence of SEQ ID NO: 1 or SEQ ID NO: 3 operatively linked to at least one heterologous gene that encodes a toxic protein, and
i. at least one inducible promoter operatively linked to at least one heterologous gene that encodes a toxic protein,
ii. wherein the at least one inducible promoter is selected from the group consisting of: a forskolin inducible promoter, a hypoxia inducible promoter, a tetracycline inducible promoter, an alcohol inducible promoter, a steroid inducible promoter, an RU486 inducible promoter, an ecdysone inducible promoter, a rapamycin inducible promoter, a metallothionein inducible promoter, a hormone inducible promoter and a metal inducible promoter.
67. A stable cell line for rAAV production, comprising at least one inducible promoter having a sequence of any one of SEQ ID NO: 6 to SEQ ID NO: 9 operatively linked to at least one heterologous gene that encodes a toxic protein, and
i. at least one inducible promoter operatively linked to at least one heterologous gene that encodes a toxic protein,
ii. wherein the at least one inducible promoter is selected from the group consisting of: a forskolin inducible promoter, a hypoxia inducible promoter, a tetracycline inducible promoter, an alcohol inducible promoter, a steroid inducible promoter, an RU486 inducible promoter, an ecdysone inducible promoter, a rapamycin inducible promoter, a metallothionein inducible promoter, a hormone inducible promoter and a metal inducible promoter.
68. A stable cell line for rAAV production, comprising at least one inducible promoter having a sequence of SEQ ID NO: 1 or SEQ ID NO: 3 operatively linked to at least one heterologous gene that encodes a toxic protein, and at least one inducible promoter having a sequence of any one of SEQ ID NO: 6 to SEQ ID NO: 9 operatively linked to at least one heterologous gene that encodes a toxic protein.
69. The stable cell line of any preceding paragraph, further comprising at least one repressible element operatively linked to at least one heterologous gene that encodes a toxic protein.
70. The stable cell line of any preceding paragraph, wherein the stable cell line has at least two inducible promoters, and the at least two inducible promoters are the same.
71. The stable cell line of any preceding paragraph, wherein the stable cell line has at least two inducible promoters, and the at least two inducible promoters are different.

The invention described herein can further be described in the additional numbered paragraphs below:
1. A nucleic acid construct comprising:
a nucleic acid sequence comprising at least one of: a nucleic acid sequence encoding a E4 protein, a nucleic acid sequence encoding a E2A protein, and a nucleic acid sequence encoding a VA RNA, wherein each nucleic acid sequences encoding any one of E4 protein, E2A protein, and VA RNA is operatively linked to a first regulatable promoter; and
a nucleic acid sequence encoding a Rep protein, wherein the nucleic acid encoding a Rep protein is under the control of a second regulatable promoter or heterologous transcriptional activator,
wherein the first and the second regulatable promoters are different.
2. The nucleic acid construct of paragraph 1, wherein the Rep protein is a modified Rep protein.
3. The nucleic acid construct of any preceding paragraph, wherein the modified Rep protein has a lysine to arginine mutation at amino acid 84.
4. The nucleic acid construct of any preceding paragraph, wherein the nucleic acid encoding the Rep protein further comprises the nucleic acid encoding a ribozyme at its 3′ end.
5. The nucleic acid construct of any preceding paragraph, wherein the regulatable promoter operatively linked to the nucleic acid encoding the Rep protein is an inducible promoter or comprises a binding site for the heterologous transcriptional activator.
6. The nucleic acid construct of any preceding paragraph, wherein the inducible promoter is selected from the group consisting of a forskolin inducible promoter, a hypoxia inducible promoter, a tetracycline inducible promoter, an alcohol inducible promoter, a steroid inducible promoter, an RU486 inducible promoter, an ecdysone inducible promoter, a rapamycin inducible promoter, a metallothionein inducible promoter, a hormone inducible promoter and a metal inducible promoter
7. The nucleic acid construct of any preceding paragraph, wherein the inducible promoter lacks a minimal promoter.
8. The nucleic acid construct of any preceding paragraph, wherein the inducible promoter further comprises a TATA box sequence, or a p5 replication sequence, or both a TATA box sequence and p5 replication sequence.
9. A nucleic acid construct comprising a nucleic acid sequence encoding a zinc finger (ZF) transcriptional activator operatively linked to an inducible promoter, wherein the inducible promoter is a E4-responsive promoter, or a E2-responsive promoter or other helper-gene responsive promoter.
10. A nucleic acid construct comprising a toxic protein operatively linked to a promoter comprising a target site for binding of a zinc-finger transcriptional activator (ZF-TA).
11. A nucleic acid construct comprising a Rep protein operatively linked to a promoter comprising a target site for binding of a zinc-finger transcriptional activator (ZF-TA).
12. The nucleic acid construct of any preceding paragraph, wherein the zinc-finger transcriptional activator (ZF-TA) is expressed from the nucleic acid construct of any preceding paragraph.
13. The nucleic acid construct of any preceding paragraph, further comprising at least one of a nucleic acid sequence encoding at least one helper protein, wherein each nucleic acid construct is operatively linked to a regulatable promoter;
a nucleic acid encoding the toxic protein is under the control of a second regulatable promoter or a zinc-finger transcriptional activator;
a nucleic acid sequence encoding a E4 protein, a nucleic acid sequence encoding a E2A protein, and a nucleic acid sequence encoding a VA RNA, wherein each nucleic acid sequences encoding any one of E4, E2A, and VA RNA is operatively linked to a regulatable promoter; or
a nucleic acid encoding the Rep protein is under the control of a second regulatable promoter or a zinc-finger transcriptional activator.
14. A nucleic acid construct comprising:
a nucleic acid sequence encoding a tetracycline-responsive transactivator protein operatively linked to a constitutive promoter;
a nucleic acid sequence comprising at least one of: a nucleic acid sequence encoding a E4 protein, a nucleic acid sequence encoding a E2A protein, and a nucleic acid sequence encoding a VA protein, wherein each nucleic acid sequences encoding any one of E4, E2A, and VA RNA is operatively linked to a regulatable promoter; and
a nucleic acid sequence encoding a zinc finger (ZF) transcriptional activator operatively linked to an inducible promoter, wherein the inducible promoter is a E4-responsive promoter, or a E2-responsive promoter;
and a nucleic acid construct comprising a Rep protein operatively linked to a promoter comprising a target site for binding of a zinc-finger transcriptional activator (ZF-TA).
15. A cell comprising the nucleic acid construct of any preceding paragraph.
16. A cell comprising the nucleic acid construct of any preceding paragraph.
17. A cell comprising the nucleic acid construct of any preceding paragraph.
18. A cell comprising the nucleic acid construct of any preceding paragraph.
19. A cell comprising the nucleic acid construct of any preceding paragraph.
20. A cell comprising at least one nucleic acid construct selected from those in any preceding paragraph.
21. The cell of any preceding paragraph, comprising at least two nucleic acid constructs selected from any of those in any preceding paragraph.
22. The cell of any preceding paragraph, comprising at least three nucleic acid constructs selected from any of those in any preceding paragraph.
23. The cell of any of any preceding paragraph, further comprising a nucleic acid construct comprising a nucleic acid sequence encoding a tetracycline-responsive transactivator protein operatively linked to a constitutive promoter.
24. The cell of any of any preceding paragraph, further comprising a nucleic acid construct comprising a nucleic acid sequence encoding a marker protein.
25. The cell of any preceding paragraph, wherein the nucleic sequence encoding a marker protein is flanked by recombinase recognition sequences (RRSs) in the same orientation with respect to each other.
26. The cell of any preceding paragraph, wherein expression of the nucleic acid construct is a stable expression.
27. The cell of any preceding paragraph, wherein expression of the nucleic acid construct is a transient expression.
28. The cell of any preceding paragraph, wherein the cell comprises at least two nucleic acid constructs, and the expression of the at least two nucleic acid constructs is a stable expression.
29. The cell of any preceding paragraph, wherein the cell comprises at least two nucleic acid constructs, and the expression of the at least two nucleic acid constructs is a transient expression.
30. The cell of any preceding paragraph, wherein the cell comprises at least two nucleic acid constructs, and the expression of at least one nucleic acid construct is a stable expression.
31. A stable cell comprising stable expression of at least one nucleic acid construct selected from those in any preceding paragraph.
32. A cell comprising transient expression of at least one nucleic acid construct selected from those in any preceding paragraph.
33. A nucleic acid construct comprising a nucleic acid sequence encoding a Cap protein and a recombinase recognition sequence (RRS) 3′ of the nucleic acid sequence encoding the Cap protein.
34. The nucleic acid construct of any preceding paragraph, wherein the nucleic acid sequence encoding a Cap protein is operatively linked to a constitutive promoter.
35. The nucleic acid construct of any preceding paragraph, wherein the nucleic acid sequence encoding a Cap protein is operatively linked a regulatable promoter.
36. The nucleic acid construct of any preceding paragraph, wherein the regulatable promoter in an inducible promoter.
37. The nucleic acid construct of any preceding paragraph, wherein the RRS is a Flippase-responsive RRSs.
38. The nucleic acid construct of any preceding paragraph, further comprising a nucleic acid encoding a recombinase protein operatively linked to an inducible promoter.
39. A cell comprising the nucleic acid construct of any preceding paragraph.
40. The cell of any preceding paragraph, further comprising a nucleic acid construct comprising a nucleic acid encoding a recombinase protein operatively linked to an inducible promoter.
41. A nucleic acid construct comprising
a first nucleic acid construct comprising in a 5′ to 3′ direction: a promoter, a stop nucleic acid sequence flanked by a first pair of recombinase recognition sequences (RRS), and nucleic acid sequence encoding a Rep protein, wherein the promoter is operatively linked to the nucleic acid encoding the Rep protein, and
a second nucleic acid construct comprising, in a 5′ to 3′ direction: a promoter, a stop nucleic acid sequence flanked by a second pair of recombinase recognition sequences (RRSs), and nucleic acid sequence encoding one or more of E2A, E4, and VA RNA, wherein the promoter is operatively linked to the nucleic acid encoding the one or more of E2A, E4, and VA RNA.
42. The nucleic acid construct of any preceding paragraph, wherein the nucleic acid construct further comprises a nucleic acid encoding one or more selection markers flanked between a third pair of recombinase recognition sequences (RRSs), wherein the pair of RRS are in the same orientation with respect to each other, and wherein the nucleic acid encoding the one or more selection markers is operatively linked to one or more promoters from any preceding paragraph.
43. The nucleic acid construct of any preceding paragraph, wherein the first pair of RRS and the second pair of RRS are in the same orientation with respect to each other.
44. The nucleic acid construct of any preceding paragraph, wherein the first pair of RRS and the second pair of RRS are in the inverse orientation with respect to each other.
45. The nucleic acid construct of any preceding paragraph, wherein the first pair of RRS, second pair of RRS, and third pair of RRS are each responsive to different tyrosine recombinase or serine integrase enzymes.
46. The nucleic acid construct of any preceding paragraph, wherein the first pair of RRS and second pair of RRS, are responsive to the same tyrosine recombinase or serine integrase enzyme.
47. The nucleic acid construct of any preceding paragraph, wherein the first pair of RRS, or second pair of RRS, or both are Cre-responsive RRS.
48. The nucleic acid construct of any preceding paragraph, wherein the third pair of RRSs are Flipase-responsive RRS.
49. A cell comprising the nucleic acid construct of any preceding paragraph.
50. A cell comprising the nucleic acid construct of any preceding paragraph and a nucleic acid encoding a Cre recombinase protein operatively linked to an inducible promoter.
51. A method of producing viral particles, comprising;
providing any cell line of any preceding paragraph, any stable cell line of any preceding paragraph, or any transient cell line of any preceding paragraph in a viral expression system;
culturing the cells for a time sufficient and under conditions in which the at least one nucleic acid under the control of an regulatable promoter is expressed;
culturing the cells under conditions in which viral particles are produced; and
optionally isolating the viral particles.
52. A method of producing viral particles, comprising;
   a. providing the cell line of any preceding paragraph;
   b. culturing the cells for a time sufficient and under conditions in which at least the nucleic acid sequence encoding a E4 protein, the nucleic acid sequence encoding a E2A protein, or the nucleic acid sequence encoding a VA protein is expressed first in a viral vector production protocol;
   c. culturing the cells for a time sufficient and under conditions in which the nucleic acid sequence encoding a Rep protein is expressed second in the viral vector production protocol;
   d. culturing the cells under conditions in which viral particles are produced; and
   e. optionally isolating the viral particles.
53. The method of any preceding paragraph, wherein culturing in step (b) is culturing with an inducer of the first regulatable promoter.
54. The method of any preceding paragraph, wherein culturing in step (c) is culturing with an inducer of the second regulatable promoter.
55. A method of producing viral particles, comprising;
   a. providing the cell line of any preceding paragraph;
   b. culturing the cells for a time sufficient and under conditions in which at least the nucleic acid sequence encoding the E4 protein, the nucleic acid sequence encoding the E2A protein, or the nucleic acid sequence encoding the VA protein is expressed first in a viral vector production protocol;
   c. culturing the cells for a time sufficient and under conditions in which the nucleic acid sequence encoding a toxic protein or Rep protein is expressed second in the viral vector production protocol;
   d. culturing the cells under conditions in which viral particles are produced; and
   e. optionally isolating the viral particles.
56. The method of any preceding paragraph, wherein culturing in step (b) is culturing with an inducer of the regulatable promoter operatively linked to at least E4, E2A, or VA protein.
57. The method of any preceding paragraph, wherein culturing in step (c) is culturing with the ZF-TA.
58. The method of any preceding paragraph, wherein expression of the zinc-finger transcriptional activator (ZF-TF) is induced by expression of E4 or E2.
59. A method of producing viral particles, comprising;
   a. providing the cell line of any preceding paragraph;
   b. culturing the cells for a time sufficient and under conditions in which at least the nucleic acid sequence encoding the E4 protein, the nucleic acid sequence encoding the E2A protein, or the nucleic acid sequence encoding the VA protein is expressed first in a viral vector production protocol;
   c. culturing the cells for a time sufficient and under conditions in which the nucleic acid sequence encoding a toxic protein or Rep protein is expressed second in the viral vector production protocol;
   d. culturing the cells under conditions in which viral particles are produced; and
   e. optionally isolating the viral particles.
60. The method of any preceding paragraph, wherein culturing in step (b) is culturing with an inducer of the regulatable promoter operatively linked to at least E4, E2A, or VA protein.
61. The method of any preceding paragraph, wherein culturing in step (c) is culturing with the ZF-TA.
62. The method of any preceding paragraph, wherein expression of the zinc-finger transcriptional activator (ZA-TF) is induced by expression of E4 or E2.
63. A method of producing viral particles, comprising;
   a. providing the cell line of any preceding paragraph;
   b. culturing the cells for a time sufficient and under conditions in which at least the nucleic acid sequence encoding the Cap protein is highly expressed for the first 24 hours of the viral vector production protocol, and is moderately expressed for the remaining 48 hours of the viral vector production protocol;
   c. culturing the cells for a time sufficient and under conditions in which viral particles are produced; and
   d. optionally isolating the viral particles.
64. The method of any preceding paragraph, wherein culturing in step (b) is culturing with an inducer of the regulatable promoter operatively linked to the Cap protein.
65. A method of producing viral particles, comprising;
   a. providing the cell line of any preceding paragraph;
   b. culturing the cells for a time sufficient and under conditions in which at least the nucleic acid sequence encoding the E4 protein, the nucleic acid sequence encoding the E2A protein, or the nucleic acid sequence encoding the VA RNA is expressed first a viral vector production protocol;
   c. culturing the cells for a time sufficient and under conditions in which the nucleic acid sequence encoding a toxic protein or Rep protein is expressed second in the viral vector production protocol;
   d. culturing the cells under conditions in which viral particles are produced; and
   e. optionally isolating the viral particles.
66. The method of any preceding paragraph, wherein culturing in step (b) is culturing with a recombinase specific for the first pair of recombinase recognition sequences (RRSs).
67. The method of any preceding paragraph, wherein culturing in step (c) is culturing with a recombinase specific for the second pair of recombinase recognition sequences (RRSs).
68. The method of any preceding paragraph, wherein the transcriptional activator is a zinc finger transcriptional activator (ZF-TA).

### EXAMPLES

### Example 1

Materials and Methods

Derivation ofsuspension HEK293 cells from an adherentHEK293 QualifiedMaster Cell Bank. The derivation of the suspension cell line from the parental HEK293 Master Cell Bank (MCB), is performed in a Class 10,000 clean room facility. The derivation of the suspension cell line is carried out in a two phase process that involved first weaning the cells off of media containing bovine serum and then adapting the cells to serum free suspension media compatible with HEK293 cells. The suspension cell line is created as follows. First, a vial of qualified Master Cell Bank (MCB) is thawed and placed into culture in DMEM media containing 10% fetal bovine serum (FBS) and cultured for several days to allow the cells to recover from the freeze/thaw cycle. The MCB cells are cultured and passaged over a 4 week period while the amount of FBS in the tissue culture media is gradually reduced from 10% to 2.5%. The cells are then transferred from DMEM 2.5% FBS into serum free suspension media and grown in shaker flasks. The cells are then cultured in the serum-free media for another 3 weeks while their growth rate and viability is monitored. The adapted cells are then expanded and frozen down. A number of vials from this cell bank are subsequently thawed and used during process development studies to create a scalable manufacturing process using shaker flasks and wave bioreactor systems to generate rAAV vectors. Suspension HEK293 cells are grown in serum-free suspension media that supports both growth and high transfection efficiency in shaker flasks and wave bioreactor bags. Multitron Shaker Incubators (ATR) are used for maintenance of the cells and generation of rAAV vectors at specific rpm shaking speeds (based on cell culture volumes), 80% humidity, and 5% CO₂.

Transfection of suspension HEK293 cells. On the day of transfection, the cells are counted using a ViCell XR Viability Analyzer (Beckman Coulter) and diluted for transfection. To mix the transfection cocktail the following reagents are added to a conical tube in this order: plasmid DNA, OPTIMEM® I (Gibco) or OptiPro SFM (Gibco), or other serum free compatible transfection media, and then the transfection reagent at a specific ratio to plasmid DNA. The plasmid DNA has a sequence comprising a heterologous nucleic acid sequence of a rep gene operatively linked to a hypoxia-inducible promoter (i.e.

(Synp-HYP-001, SEQ ID NO: 3)
CTGCACGTACTGCACGTACTGCACGTACTGCACGTATGGGTACCGTCGAC
GATATCGGATCCAGGTCTATATAAGCAGAGCTCGTTTAGTGAACCGTCAG
ATCGCCTAGATACGCCATCCACGCTGTTTTGACCTCCATAGAAGATCGCC
ACC,

and a heterologous nucleic acid sequence of a cap gene operatively linked to a forskolin inducible promoter (i.e. TGACGTCACGATTACCATTGACGTCACGATTACCATTGACGTCACGATTACCATTGACG TCACGATTACCATTGACGTCAGCGATTAAGATGACTCAGCGATTAAGATGACTCAGCG ATTAAGATGACTCAGCGATTAAGATGACTCACTAGCCCGGGCTCGAGATCTGCGATCT GCATCTCAATTAGTCAGCAACCATAGTCCCGCCCCTAACTCCGCCCATCCCGCCCCTAA CTCCGCCCAGTTCCGCCCATTCTCCGCCCCATCGCTGACTAATTTTTTTTATTTATGCAG AGGCCGAGGCCGCCTCGGCCTCTGAGCTATTCCAGAAGTAGTGAGGAGGCTTTTTTGG AGGCCTAGGCTTTTGCAAA (Synp-FORCSV-10, SEQ ID NO: 6). The cocktail is inverted to mix prior to being incubated at room temperature. The transfection cocktail is then pipetted into the flasks and placed back in the shaker/incubator. All optimization studies are carried out at 30 mL culture volumes followed by validation at larger culture volumes. Cells are harvested 48 hours post-transfection.

Production of rAAV using wave bioreactor systems. Wave bags are seeded 2 days prior to transfection. Two days post-seeding the wave bag, cell culture counts are taken and the cell culture is then expanded/diluted before transfection. The wave bioreactor cell culture is then transfected. 48 hours post-transfection, wave bioreactor cell culture are cultured under conditions that induce expression of the rep and cap proteins. Such conditions for cap expression require administering NKH 477 at a concentration of from 1 μM to 100 μM, e.g. 8 μM in the wave bioreactor cell culture. Such conditions for rep expression require culturing the cells under hypoxic conditions, i.e. 5% oxygen. Cell culture is harvested from the wave bioreactor bag at least 48 hours post-induction.

Analyzing transfection efficiency using Flow Cytometry. Approximately 24 hours post-induction, 1 mL of cell culture is removed from each flask or wave bioreactor bag as well as an uninduced control. Samples are analyzed using a Dako Cyan flow cytometer to confirm that the plasmid DNA.

Harvesting suspension cells from shaker flasks and wave bioreactor bags. 48 hours post-induction, cell cultures are collected into 500 mL polypropylene conical tubes (Corning) either by pouring from shaker flasks or pumping from wave bioreactor bags. The cell culture is then centrifuged at 655×g for 10 min using a Sorvall RC3C plus centrifuge and H6000A rotor. The supernatant is discarded, and the cells are resuspended in 1×PBS, transferred to a 50 mL conical tube, and centrifuged at 655×g for 10 min. At this point, the pellet could either be stored in NLT-60° C. or continued through purification.

Titering rAAV from cell lysate using qPCR. 10 mL of cell culture is removed and centrifuged at 655×g for 10 min using a Sorvall RC3C plus centrifuge and H6000A rotor. The supernatant is decanted from the cell pellet. The cell pellet is then resuspended in 5 mL of DNase buffer (5 mM CaCl₂, 5 mM MgCl₂, 50 mM Tris-HCl pH 8.0) followed by sonication to lyre the cells efficiently. 300 ul is then removed and placed into a 1.5 mL microfuge tube. 140 units of DNase I is then added to each sample and incubated at 37° C. for 1 hour. To determine the effectiveness of the DNase digestion, 4-5 ug of plasmid DNA is spiked into a non-transfected cell lysate with and without the addition of DNase. 50 ul of EDTA/Sarkosyl solution (6.3% sarkosyl, 62.5 mM EDTA pH 8.0) is then added to each tube and incubated at 70° C. for 20 minutes. 50 ul of Proteinase K (10 mg/mL) is then added and incubated at 55° C. for at least 2 hours. Samples are then boiled for 15 minutes to inactivate the Proteinase K. An aliquot is removed from each sample to be analyzed by qPCR. Two qPCR reactions are carried out in order to effectively determine how much rAAV vector is generated per cell.

Purification of rAAV from crude lysate. Each cell pellet is adjusted to a final volume of 10 mL. The pellets are vortexed briefly and sonicated for 4 minutes at 30% yield in one second on, one second off bursts. After sonication, 550 U of DNase is added and incubated at 37° C. for 45 minutes. The pellets are then centrifuged at 9400×g using the Sorvall RCSB centrifuge and HS-4 rotor to pellet the cell debris and the clarified lysate is transferred to a Type70Ti centrifuge tube (Beckman 361625). In regard to harvesting and lysing the suspension HEK293 cells for isolation of rAAV, one skilled in the art could use mechanical methods such as microfluidization or chemical methods such as detergents, etc., followed by a clarification step using depth filtration or Tangential Flow Filtration (TFF).

AAV vector purification. Clarified AAV lysate is purified by column chromatography methods as one skilled in the art would be aware of and described in the following manuscripts (Allay et al., Davidoff et al., Kaludov et al., Zolotukhin et al., Zolotukin et al, etc).

Titering rAAV using dot blot. 100 ul of DNase buffer (140 units DNase, 5 mM CaCl₂, 5 mM MgCl₂, 50 mM Tris-HCl pH 8.0) is added to each well of a 96-well microtiter plate. 1-3 ul or serial dilutions of virus is added to each well and incubated at 37° C. for 30 min. The samples are then supplemented with 15 ul Sarkosyl/EDTA solution (6.3% sarkosyl, 62.5 mM EDTA pH 8.0) and placed at 70° C. for 20 min. Next, 15 ul of Proteinase K (10 mg/mL) is added and incubated at 50° C. for at least 2 hours. 125 ul of NaOH buffer (80 mM NaOH, 4 mM EDTA pH 8.0) is added to each well. A series of transgene specific standards are created through a dilution series. NaOH buffer is then added and incubated. Nylon membrane is incubated at RT in 0.4 M Tris-HCl, pH 7.5 and then set up on dot blot apparatus. After a 10-15 minute incubation in NaOH buffer, the samples and standards are loaded into the dot blot apparatus onto the GeneScreen PlusR hybridization transfer membrane (PerkinElmer). The sample is then applied to the membrane using a vacuum. The nylon membrane is soaked in 0.4 M Tris-HCl, pH 7.5 and then cross linked using UV strata linker 1800 (Stratagene) at 600 ujouls×100. The membrane is then pre-hybridized in CHURCH buffer (1% BSA, 7% SDS, 1 mM EDTA, 0.5 M Na₃PO₄, pH 7.5). After pre-hybridization, the membrane is hybridized overnight with a ³²P-CTP labeled transgene probe (Roche Random Prime DNA labeling kit). The following day, the membrane is washed with low stringency SSC buffer (1×SSC, 0.1% SDS) and high stringency (0.1×SSC, 0.1% SDS). It is then exposed on a phosphorimager screen and analyzed for densitometry using a STORM840 scanner (GE).

Analyzing rAAV vector purity using silver stain method. Samples from purified vector are loaded onto NuPage 10% Bis-Tris gels (Invitrogen) and run using 1×NuPage running buffer. Typically, 1×10¹⁰ particles are loaded per well. The gels are treated with SilverXpress Silver staining kit #LC6100 (Invitrogen).

Analysis of self-complementary genomes using alkaline gel electrophoresis and southern blot. Briefly, purified self-complementary rAAV is added to 200 ul, of DNase I buffer (140 units DNase, 5 mM CaCl₂, 5 mM MgCl₂, 50 mM Tris-HCl pH 8.0) and incubated at 37° C. for 60 minutes, followed by inactivation of the DNase by adding 30 ul, of EDTA Sarkosyl/EDTA solution (6.3% sarkosyl, 62.5 mM EDTA pH 8.0) and placed at 70° C. for 20 min. 20 ul of Proteinase K (10 mg/mL) is then added to the sample and incubated for a minimum of 2 hours at 50° C. Phenol/Chloroform is added in a 1:1 ratio, followed by ethanol precipitation of the viral vector DNA. The pelleted DNA is then resuspended in alkaline buffer (50 mM NaOH, 1 mM EDTA) for denaturation, loaded onto a 1% alkaline agarose gel, and run at 25V overnight. The gel is then equilibrated in alkaline transfer buffer (0.4 M NaOH, 1 M NaCl) and a southern blot is performed via an overnight transfer of the vector DNA to a GeneScreen PlusR hybridization transfer membrane (PerkinElmer). The membrane is then neutralized using 0.5 M Tris pH 7.5 with 1 M NaCl, and is hybridized overnight with a ³²P-CTP labeled transgene probe. After washing the membrane as previously described, the membrane is exposed to a phosphorimager screen and analyzed using a STORM840 scanner.

Transduction Assays. HeLaRC-32 cells (Chadeuf et al., J Gene Med. 2:260 (2000)) are plated at 2×10⁵ cells/well of a 24 well plate and incubated at 37° C. overnight. The cells are observed for 90-100% confluence. 50 mL of DMEM with 2% FBS, 1% Pen/Strep is pre-warmed, and adenovirus (d1309) is added at a MOI of 10. The d1309 containing media is aliquoted in 900 ul fractions and used to dilute the rAAV in a series of ten-fold dilutions. The rAAV is then plated at 400 μl and allowed to incubate for 48 hours at 37° C.

Concentration Assays. The starting vector stock is sampled and loaded onto a vivaspin column and centrifuged at 470×g (Sorvall H1000B) in 10 minute intervals. Once the desired volume/concentration had been achieved, both sides of the membrane are rinsed with the retentate, which is then harvested. Samples of the pre-concentrated and concentrated rAAV are taken to determine physical titers and transducing units.

Transmission electron microscopy (TEM) of negatively stained rAAV particles. Electron microscopy allows a direct visualization of the viral particles. Purified dialyzed rAAV vectors are placed on a 400-mesh glow-discharged carbon grid by inversion of the grid on a 20 ul drop of virus. The grid is then washed 2 times by inversion on a 20 ul drop of ddH₂O followed by inversion of the grid onto a 20 ul drop of 2% uranyl acetate for 30 seconds. The grids are blotted dry by gently touching Whatman paper to the edges of the grids. Each vector is visualized using a Zeiss EM 910 electron microscope.

### Example 2—Hypoxia-Inducible Promoters

Hypoxia and HIF:

The importance of the HIF signalling cascade is shown by knockout studies in mammals which leads to perinatal death. This is due to its role in the development of the vascular system and chondrocyte survival. In addition, HIF1 plays a central role in human metabolism as it is linked with respiration and energy generation. Furthermore, the cascade mediates the effects of hypoxia by upregulating genes important for survival in such conditions. For example, hypoxia promotes the formation of blood vessels, which is a normal response essential in development. However, in cancer, hypoxia can also lead to the vascularisation of tumours.

The main response element for the sensing and upregulation of genes involved in hypoxia stress response is the transcriptional complex HIF1. This complex is highly conserved across eukaryotes and is formed by the dimerization of 2 subunits, α and β. The β-subunit is constitutively expressed and is an aryl hydrocarbon receptor nuclear translocator (ARNT) essential for translocation of the complex to the nucleus. Both the α and β-subunits belong to the basic helix-loop-helix family of transcription factors and contain the following domains:
N-terminal: bHLH domain for DNA binding
Central heterodimerization domain: Per-ARNT-Sim (PAS) domain
C-terminal: recruits transcriptional coregulatory proteins

HIF Mechanism of Action:

Under normoxic conditions HIF1α subunits are hydroxylated at conserved proline residues. This hydroxylation by HIF prolyl-hydroxylases targets the subunits for recognition and ubiquitination by the VHL E3 ubiquitin ligase and subsequent degradation by the proteasome.

However, under hypoxic conditions, oxygen limitation inhibits the HIF prolyl-hydroxylase as oxygen is an essential co-substrate for this enzyme. Once stabilised, HIF-1α subunits can heterodimerise with HIF-1β subunits and translocate to the nucleus where they can upregulate the expression of a number of genes. This is achieved by the HIF complex's binding to HIF-responsive elements (HREs) in promoters that contain the HBS sequence NCGTG (SEQ ID NO: 75) (where N is preferably either an A or G) or its reverse complement. The genes upregulated by the HIF1 complex are involved in central metabolism, such as glycolysis enzymes which allow ATP synthesis in an oxygen-independent manner, or in angiogenesis such as vascular endothelial growth factor (VEGF).

Pseudohypoxia:

There are alternative ways to activate the HIF1 complex. Mutations to SDHB, one of four protein subunits forming succinate dehydrogenase, cause build-up of succinate by inhibiting electron transfer in the succinate dehydrogenase complex. This excess succinate inhibits HIF prolyl-hydroxylase, stabilizing HIF-la.

NF-κB can also directly modulate HIF1 regulation under normoxic conditions. It is believed that NF-κB can regulate basal HIF-1α expression as increased HIF-1α levels was correlated with increased NF-κB expression.

Hypoxia Responsive Elements:

Hypoxia-responsive elements tend to have a conserved HIF1 binding consensus sequence, NCGTG (SEQ ID NO: 75), where N is preferably either an A or G (Schödel, et al., 2011, Blood. 2011 Jun. 9; 117(23):e207-17.). The flanking sequence of this is notoriously variable but still contributes to the activity of the promoter.

The following exemplary HIF binding sequences (HBS) are used in the following examples:
HRE1 (ACGTGC (SEQ ID NO: 78)) which is a variant of the consensus sequence ([AG]CGTG, SEQ ID NO: 76) found in HIF binding sites of hypoxia-responsive elements (Schödel, et al., 2011).
HRE2 (CTGCACGTA (SEQ ID NO: 80)) was described as a superior and highly active hypoxia-inducible motif (Kaluz, et al., 2008, Biochem Biophys Res Commun. 2008 Jun. 13; 370(4):613-8).
HRE3 (ACCTTGAGTACGTGCGTCTCTGCACGTATG (SEQ ID NO: 82)) was described as a strongly induced element (Ede, et al., 2016, ACS Synth. Biol., 2016, 5 (5), pp 395-404). HRE3 is a composite HBS which comprises both HRE1 and HRE2 and it was hypothesised that it may be possible to increase the strength of induction by using this element.

Synthetic promoters comprising these HBS sequences were prepared and tested as described below.
Synp-HYP-001 construct (SEQ ID NO: 3) comprises 4 HRE2 elements without spacers, a spacer of 32 base pair length between the core of the last HRE2 and the TATA box of the CMV minimal promoter. This construct was designed with suboptimal spacing between the HRE2 elements and between the last HBS and the minimal promoter, and it was predicted to have relatively low inducibility and strength of expression.
Synp-RTV-015 construct (SEQ ID NO: 1) comprises 5 HRE1 elements spaced apart by 40 bp spacers, followed directly by a synthetic minimal promoter TATA box (MP1). This promoter was designed to be only weakly induced by hypoxia by its suboptimal spacing of 40 bp between the HRE1 elements and a spacing of 36 bp from the core of the last HRE1 HBS to the TATA box of MP1.

All promoters were placed upstream of the luciferase gene.

To compare across experiments, the strength of the inducible promoters was compared to CMV-IE promoter, which was driving the same gene as the other constructs.

The synthetic promoters were synthesised by Geneart. The promoter constructs were used to drive expression of luciferase in the pMQ plasmid, unless otherwise stated.

### Example 2A

The constructs were initially used to drive luciferase expression in HEK293-F and HEK293-T in hypoxia.

Transfection of HEK293-F Cells in 24 Well Format:

40 ml of cells were grown in a 250 ml vented Erlenmeyer flask (Sigma-Aldrich CLS431144) at 37° C., 20% O₂, 8% CO₂ with agitation at 100 rpm. Cells were seeded as described in the manufacturer's instructions (300,000 cells/ml). HEK293-F were obtained from Thermofisher, R79007.

One day before transfection, the cells were counted using a haemocytometer and split to 500,000 cells/ml.

On the day of transfection, the cells are seeded to 1,000,000 cells/ml in 500 μl of appropriate medium (Freestyle 293 expression medium, 12338002) in a 24 well plate. 0.625 μg of DNA per well was then added to 10 μl of OptiMem medium (Thermofisher; 11058021) and incubated for 5 minutes at room temperature.

Concurrently, 0.625 μl of Max reagent (Thermofisher, 16447100) was made up to 10 μl by addition of OptiMem and incubated for 5 minutes at room temperature. After this incubation, both mixes were added to the same tube and incubated at room temperature for 25-30 minutes. The DNA/Max reagent mix (20 μl/well) was then added directly to the cells and the cells incubated at 37° C., 8% CO₂ with agitation at 100 rpm. The transfected DNA was one of the vectors where the promoter constructs (RTV-015, Synp-HYP-001) or a control promoter (CMV-IE) were used to drive luciferase expression and β-galactosidase containing pcDNA6 plasmid. The β-galactosidase containing plasmid was used as internal control for transfection efficiency (Thermofisher, V22020).

After transfection the cells were incubated for 24 hrs in normoxia conditions (20% oxygen) before being switched to a gas mix of 5% oxygen, 10% carbon dioxide and 85% nitrogen (hypoxia). This was achieved by gas displacement in a sealed hypoxia chamber. Induction of the promoters was assessed by using luciferase activity after 3, 5 and 24 hrs in hypoxia. These results are shown in FIG. 3.

Transfection ofHEK293-T Cells:

HEK293-T are seeded at a density of 20%. Once they reached a confluence between 60 and 80%, the media is changed with DMEM (#21885-025—Thermo Scientific) supplemented with 10% FBS (Gibco, 26140). After 3 hours, the cells were transfected by a transfection mix. The transfection mix is prepared by adding DNA (2 μg per 6 well plate) and PEI 25 kDA (#23966-1-Polyscience) in a ratio of 1:3 in sterile DPBS (#14190169—ThermoFisher Scientific). After mixing, the transfection mix is incubated at room temperature for 30 minutes and then added directly to the cells. After 16 to 18 h post transfection, the media is changed to DMEM+2% FBS. The transfected DNA was one of the vectors where the promoter construct (RTV-015) or a control promoter (CMV-IE) were used to drive luciferase expression and β-galactosidase containing pcDNA6 plasmid. The β-galactosidase containing plasmid was used as internal control for transfection efficiency (Thermofisher, V22020).

After transfection the cells were incubated for 24 hrs in normoxic conditions (20% oxygen). Induction of the promoters was assessed by using luciferase activity after 24 hrs in normoxia or hypoxia (5% oxygen, 10% carbon dioxide and 85% nitrogen). Hypoxia was achieved by gas displacement in a sealed hypoxia chamber. Results are shown in FIG. 4.

Measurement of Luciferase Activity:

Luciferase activity was measured using LARII (Dual Luciferase Reporter 1000 assay system, Promega, E1980).

Media was removed from the cells at the respective time point (0, 3, 5, 24 hrs after induction). The cells were washed once in 300 μl of DPBS. Cells were lysed by adding 10 μl of passive lysis buffer to the cells and incubation with rocking for 15 minutes. The cell debris was pelleted by centrifugation of the plate at max speed in a benchtop centrifuge for 1 min. 10 μl of supernatant was pipetted into white 96-well plate and luminescence measured by addition of 50 μl of LARII substrate.

β-galactosidase activity was measured as per manufacturer's instructions (Mammalian βGalactosidase Assay Kit, 75707/75710, Thermo Scientific) using 25 μl of lysate. 25 μl of lysate was transferred into a microplate well and mixed with 25 μl of β-galactosidase Assay Reagent, equilibrated to room temperature. The mixture was incubated at 37° C. for 30 min and absorbance measured at 405 nm.

Luciferase readouts were normalised to β-galactosidase to produce normalised relative luminometer units (RLUs).

Results:

The described promoters were transiently transfected into either the suspension cell line HEK293-F (FIG. 3) or the adherent HEK293-T (FIG. 4) cell line and activity of the promoters was assessed using luciferase assay.

In FIG. 3, the promoters in HEK293-F cells showed a rapid increase in activity upon a switch to hypoxic conditions with an increase in luciferase activity observed after 3 hrs. Maximal activity was observed after 5 hrs for all of the promoters tested with no significant increase in activity at the 24 hr timepoint. The promoter's activities correlate with their designs, with RTV-015 being the weakest, and SYNP-HYP-001 being stronger.

In FIG. 4, the promoter's expression after 24 h in hypoxia was compared to their expression after 24 h in normoxia. In HEK293-T cells the pattern of expression is very similar to HEK293-F cells with RTV-015. Again, there is no change in the CMV-IE promoter between normoxic and hypoxic conditions.

These results seem to validate our design principals with the strength of the promoters correlating to their theoretical relative strength.

### Example 2B

Transient Transfection of CHO-GS Cells:

40 ml of cells were grown in a 250 ml vented Erlenmeyer flask (Sigma-Aldrich CLS431144) at 37° C., 20% O₂, 8% CO₂ with agitation at 100 rpm. Cells were seeded as at 300,000 cells/ml.

One day before transfection, the cells were counted using a haemocytometer and split to 500,000 cells/ml.

On the day of transfection, the cells are seeded to 1,000,000 cells/ml in 500 μl of appropriate medium (Thermofisher, CD-CHO 10743029) in a 24 well plate. 0.625 μg of DNA per well was then added to 10 μl of OptiMem medium (Thermofisher; 11058021) and incubated for 5 minutes at room temperature.

Concurrently, 0.625 μl of Freestyle Max reagent (Thermofisher, 16447100) was made up to 10 μl by addition of OptiMem and incubated for 5 minutes at room temperature. After this incubation, both mixes were added to the same tube and incubated at room temperature for 25-30 minutes. The DNA/Max reagent mix (20 μl/well) was then added directly to the cells and the cells incubated at 37° C., 8% CO₂ with agitation at 100 rpm.

The transfected DNA was one of the vectors where the promoter construct (RTV-015) or a control promoter (CMV-IE) were used to drive luciferase expression and β-galactosidase containing pcDNA6 plasmid. The β-galactosidase containing plasmid was used as internal control for transfection efficiency (Thermofisher, V22020).

After transfection the cells were incubated for 24 hrs in normoxic conditions (20% oxygen). Induction of the promoters was assessed by using luciferase activity after 24 hrs in normoxia or hypoxia (5% oxygen, 10% carbon dioxide and 85% nitrogen). Hypoxia was achieved by gas displacement in a sealed hypoxia chamber. Luciferase activity was measured as described above.

Results are shown in FIG. 5.

Results:

Luciferase expression form the promoter RTV-015 was assessed in the transiently transfected CHO suspension line CHO-GS cells in order to test their functionality.

As can be seen from FIG. 5, the promoters behave in a similar manner in these cells as they do in HEK293 cells. Again, the switch to hypoxia has no effect on the activity of the CMV-IE promoter and luciferase activity from this promoter does not differ in hypoxia or normoxia.

This demonstrates the robustness of the promoters across multiple cell lines and validates our design rules.

### Example 3—Forskolin-Inducible Promoters

The forskolin-inducible promoters FORCSV-10, FOR-CMV-009, FMP-02 and FLP-01 were used to drive expression of luciferase in a PM-RQ vector in the suspension cell line HEK293-F. The tested promoters were synthesized directly upstream of the ATG of PM-RQ plasmid and the suspension cell lines were transiently transfected with the PM-RQ plasmid.

Transfection of HEK293-F cells:

40 ml of cells were grown in a 250 ml vented Erlenmeyer flask (Sigma-Aldrich CLS431144) at 37° C., 20% O₂, 8% CO₂ with agitation at 100 rpm. Cells were seeded as described in the manufacturer's instructions (300,000 cells/ml). HEK293-F were obtained from Thermofisher, R79007.

One day before transfection, the cells were counted using a haemocytometer and split to 500,000 cells/ml.

On the day of transfection, the cells are seeded to 1,000,000 cells/ml in 500 μl of appropriate medium (Freestyle 293 expression medium, 12338002) in a 24 well plate. 0.625 μg of DNA per well was then added to 10 μl of OptiMem medium (Thermofisher; 11058021) and incubated for 5 minutes at room temperature.

Concurrently, 0.625 μl of Max reagent (Thermofisher, 16447100) was made up to 10 μl by addition of OptiMem and incubated for 5 minutes at room temperature. After this incubation, both mixes were added to the same tube and incubated at room temperature for 25-30 minutes. The DNA/Max reagent mix (20 μl/well) was then added directly to the cells and the cells incubated at 37 C, 8% CO₂ with agitation at 100 rpm.

24 hours after transfection, the promoters were induced by addition of 20 μM forskolin and luciferase activity was measured 0, 3, 5 and 24 hours after induction. Luciferase activity was measured as previously described.

Results:

The forskolin-inducible promoters were used to drive expression of luciferase in the suspension cell line HEK293-F (FIG. 10). In the time course after induction, the promoters show a low background with a rapid increase in activity with maximal activity seen after 5 hrs. This activity is maintained until 24 hrs. Fold induction for the promoters varies from 50 to 100-fold, with FMP-02 being the weakest and FORCSV-10 being the strongest. The dynamic range of the promoters is also very wide. These results show that the promoters may be promising in bioprocessing applications due to their tight control and wide dynamic range (the ratio of the strongest promoter strength to the weakest promoter strength).

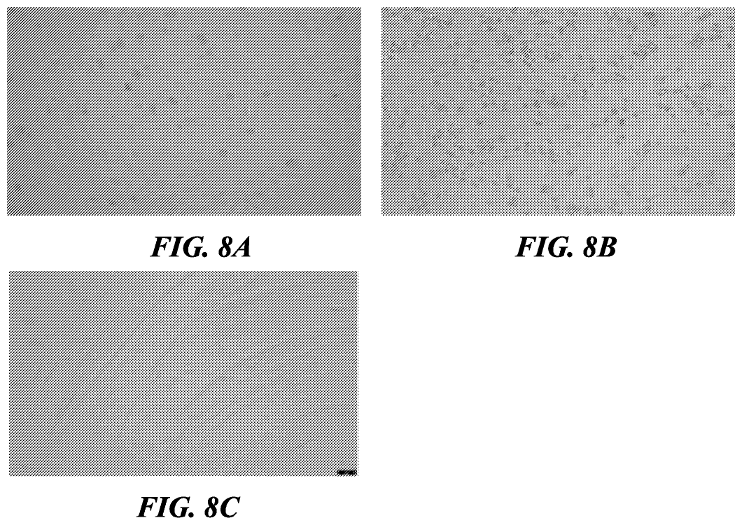

Similar performance was observed in CHO cells, in the Huh7 human liver cell line and in the C2C12 human muscle cell line. FIG. 8 shows the C2C12 cells as prepared prior to testing.

### Example 4—AAV Cell Line

Regulation of gene expression provides clear benefits over constitutive gene expression in both basic and applied research e.g. functional genomics, gene therapy, tissue engineering and bioproduction, this includes viral vector production. Inherently, regulatable systems provide a flexibility of use due to the ability to fine tune gene expression in a concentration and time dependent manner. In addition to this, these systems allow the expression of toxic proteins that cannot be expressed via constitutive promoters as this would lead to significant toxicity and cell death. This point is particularly salient in regards to generating AAV stable cell lines as many of the proteins required for AAV production are toxic. These include the replication proteins Rep78 and 52, both of which are required for replication of the AAV genome, and the adenovirus helper functions E2A and E4 which are required to co-opt the host cell machinery to make AAV particles and genomes. It is therefore, paramount that these components are under tight regulation to ensure viability of the host cell and to increase the chances of generating stable high-titer producing cell lines (Li et al, 2019).

AAV synthesis is a dynamic process requiring each of the multiple components to be expressed at specific times and levels during the process for optimal yield. This is evidenced by the observation that a wtAAV infection cycle produces a higher-titer, almost 100% full capsids and a near 1:1 ratio of viral genomes to infectivity. Whereas, rAAV has lower yields, a lower percentage of full particles, typically 20%, and an infectivity ratio of 1 in 10 to 1 in 40,000 particles. The answer to why is outlined below.

For manufacturing therapeutics there is one cell line that is used more often than any other and that is Hek293 cells. These cells, generated by Graham et at, 1977 are used because they have significant advantages over other cells, namely ease of growth, established bioprocesses as well as being highly amenable to plasmid transfection. A further advantage of these cells is to be found in how they were generated. Hek293 cells were made by transfecting embryonic kidney cells with a fragmented adenovirus 5 genome and selecting for immortalised cells. Once analysed it was shown that the cells had incorporated a 5 kb section of the adenovirus genome including the E1 gene. The E1 gene is essential for AAV replication therefore these cells already have 1 of the essential genes required for manufacture conferring a significant advantage over other systems. rAAV production in Hek293 cells involves transfection of 3 plasmids containing the genes required for replication of the therapeutic vector. However, each of the genes is controlled by a promoter that is in turn controlled by a master regulator, the E1 gene. In Hek293 transfections, this renders each of the E2A, E4, p5, 19 (Rep) and p40 (Cap) promoters active, as Hek293 continually expresses the E1 gene. In addition, native regulation by Rep of the p5, p19 and p40 promoters via interaction with cellular proteins and the ITRs only happens if the ITRs and the promoters are in a cis-configuration. However, in rAAV production the ITRs and the p5, p19 and p40 promoters are in trans and consequently the dynamics of gene expression are altered leading to a less efficient process. Replacing the endogenous promoters with regulatable alternatives would allow the process to be tailored to mimic wtAAV dynamics and increase the yield, quality and potency of AAV preparations.

Ascertaining the Level of Expression of E1A in Pro10 Cells

Hek293 cells were generated by integrating a 5 kb fragment of the adenovirus genome into fetal kidney tissue and selecting for immortalised cells (Graham et al, 1977). When analysed it was determined that the E1A gene was stably integrated into the cells genome and was constituvely expressed. This, in part, is the reason why Hek293 cells are a good candidate for rAAV production as E1A is an essential component of AAV replication. In fact, it is the master regulator from which all gene expression cascades to allow replication to occur.

Pro10 is a clonal isolate of these original Hek293 cells that have been adapted for growth in serum free medium and in suspension. Pro10 is currently the preeminent cell line for transient transfection production of rAAV. It's ability to produce rAAV to high titers (10·vg) with excellent quality has been documented previously and the cell line is patented. (Greiger et al, 2015). However, very little molecular characterization of the Pro10 cell line has been carried out. Work described herein sought to ascertain what, if any, differences could be seen between the parental Hek293 cell line and Pro10 in the expression of the master regulator E1A gene.

Description of the Experiment

Pro10 cells were maintained in Freestyle F17 expression medium (Thermofisher: A1383501) plus 10 mM Glutamax (Thermofisher: 35050061) supplement unless otherwise stated. Cells were incubated in a shaking incubator (120 rpm) at 37 C, 5% CO2.

Hek293 cells (ATCC-CRL-1573) were maintained in DMEM (Gibco: 10566016) supplemented with 10% heat inactivated FBS. Cells were incubated in a static incubator at 37 C, 5% CO2.

Both cell types were grown to 4×10\hat{ }6 cells/ml and harvested by centrifugation at 900×g for 5 minutes. RNA was extracted from the cell pellets using the RNeasy mini-kit(Qiagen: 74104) using the manufacturer's instructions.

cDNA was generated from lug of total RNA using the High capacity reverse transcription kit (Applied Biosystems: 4368813) using the manufacturer's instructions.

E1A levels were ascertained using the Tagman qPCR protocol.

E1A rimers and robe P set.

(SEQ ID NO: 157)
F: tgtgtctgaacctgagcctg (10 uM)
(SEQ ID NO: 158)
P: agcccgagccagaaccggagcctgcaa (5 uM)
(SEQ ID NO: 159)
R: atagcaggcgccattttagg (10uM)

GAPDH was used as a control and was assessed using the following kit as per the manufacturers' instructions.

TaqMan™ Gene Expression Assay (FAM) (Thermofisher: 4351370). Reactions multiplexed and were setup as described below:

| | | |
|---|---|---|
| 2× Taqman Fast Univ. PCR Master Mix | 5 | ul |
| (Applied Biosystems: 4351891) | | |
| E1 PP mix | 1 | ul |
| GAPDH PP mix | 0.5 | ul |
| cDNA *** | 2 | ul |
| H2O | 1.5 | ul |
| Final reaction vol. | 10 | ul |

Fluorophore and quencher chemistry for the probes

| | | |
|---|---|---|
| E1 | JOE (VIC) | TAMRA |
| GAPDH | FAM | MGB |

Samples were run on a Quantstudio 7 (Applied Biosystems) using the following program.

| | | | | |
|---|---|---|---|---|
| Hold | 50 | 2 | min | 1x |
| | 95 | 10 | min | |
| PCR Stage | 95 | 15 | sec | 40x |
| | 60 | 1 | min | |

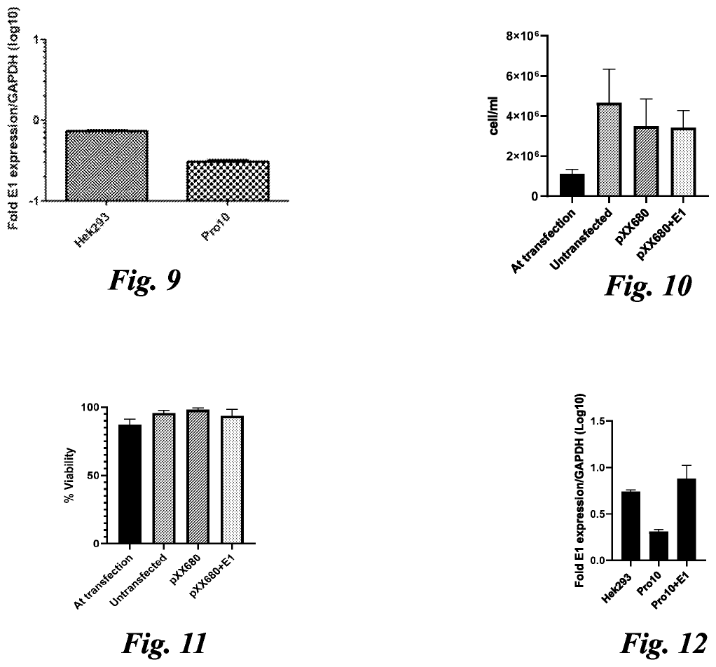

Samples were analyzed using the Comparative Ct (ΔΔCt) analysis. Pro10 cells express E1A RNA at ˜½ of the level of the parental Hek293 cells (FIG. 9). It is unclear why this disparity exists; this may be a function of the suspension adaption or serum depletion. What is clear however is that this level of E1A is enough to produce high titer rAAV. The obvious question then arises, what would happen if the level of E1A increased in Pro10 cells, would this provide a boost in rAAV production. To determine this, an experiment was designed in which rAAV production in Pro10 was supplemented with extra E1A.

Increasing the Concentration of E1A in Pro10 Cells During rAAV Production

AAV Production

For the production of AAV vectors, low-passage Pro10 cells were used. These were maintained in FreeStyle™ F17 Expression Medium supplemented with 10 mM GlutaMax (ThermoFisher, UK) in a shaking incubator set at 37° C., 5% CO2. Shaking speeds vary depending on the tissue culture container used for their culturing (120-200 rpm). This is a generic protocol used for all AAV productions in this document, deviations were marked at the appropriate juncture.

On the day of transfection for the production of AAV vectors, the Pro10 cells were seeded at an optimal density (1e6 cells/ml) in appropriate tissue culture containers. Three plasmid transfections were immediately performed; Plasmids that were transfected into the Pro10 cells for production of AAV vectors include an Adenovirus helper (XX680-Kan_JMS_11jan.2018) or pHelper (Takara Biotech, 6230) and RepCap (differs according to the AAV being produced) plasmids as well as the transgene of interest (pAAV-ZsGreen, Takara Biotech, 6231) at a ratio of 2:1.66:1. High potency linear polyethylenimine (PEI “Max” (Mw 40,000); Polysciences, 24765) is used as the transfecting agent at a ratio of 2:1 (PEI:DNA), while the final concentration of DNA used for transfection is 1.5 ug/1e6 cells. The transfection mix is prepared by adding the plasmid DNA followed by the F17+ media followed by the PEI; it is vortexed for 15 secs and incubated at room temperature for 7 mins. The mix is then pipetted drop-by-drop onto the cell suspension sample. The transfected cells were placed back into the incubator. 3 hours post-transfection, HyClone CDM4HEK293 media (Fisher Scientific, UK, 11506341) supplemented with 10 mM Glutamax (ThermoFisher, UK) is added to each well at an equal volume to the transfection mix and the transfected cells were placed back into the shaking incubator for a total of 3 days.

On day 4 cells were harvested in Eppendorf tubes via centrifuged in a tabletop centrifuge at 300 g for 5 min, supernatants were discarded, and cell pellets were resuspended in 1×DPBS. The pellets then undergo five cycles of freeze/thawing. They were centrifuged in a tabletop centrifuge at 4700 rpm for 5 min to remove any cell debris and the supernatants were transferred to new tubes. These then undergo DNAse digestion to remove cellular DNA; In brief, DNAse I enzyme (10 mg/ml; ThermoFisher, UK, 18047019) plus 10×DNAse buffer (NEB, UK, B0303S) were added to each sample. Samples were mixed and incubated for 1 hour at 37° C. followed by 10 mins at 95° C. in a thermal cycler. EDTA (0.5M; ThermoFisher, UK, AM9260S) is then added to each sample and mixed.

Extraction of DNA from the Virus

Following DNAse I treatment, the samples undergo Proteinase K digestion, to remove the protein capsid, following the manufacturer's protocol (NEB, UK, HH4501). In brief, Proteinase K enzyme (10 mg/ml) enzyme plus 10×Proteinase K buffer were added to each sample. Samples were mixed and incubated for 1 hour at 55° C. followed by 10 mins at 95° C. in a thermal cycler. Following incubation, the samples were frozen until further use/analysis.

qPCR—General Protocol

Post-transfection and prior to viral genome extraction, cells were harvested and lysed. Total RNA is extracted from the cell pellets using a standard Trizol protocol (TRIzol™ Reagent; ThermoFisher, UK, 15596018). DNAse I treatment is performed following the manufacturer's instructions (DNA-free™ Kit; ThermoFisher, UK, AM1906). Complementary DNA (cDNA) is synthesized using the SuperScript™ VILO™ cDNA Synthesis Kit and following the manufacturer's protocol (ThermoFisher, UK, 117545050). Quantitative real-time polymerase chain reaction (qRT-PCR) is performed on a QuantStudio™ 7 Flex Real-Time PCR System using the Tagman assay system (ThermoFisher, UK).

qPCR—AAV Physical Titer

For determining the physical titer of the AAV vectors produced, the following alterations to the general qPCR protocol were performed; Post-transfection and after extracting the AAV genomes (as previously described herein above in the ‘AAV production’ protocol), two different dilutions of each sample were prepared: i) 1:10 and ii) 1:100 in nuclease-free H2O. A master mix containing 2×TaqMan™ Fast Advance qPCR mix (ThermoFisher, UK, 4444963), the forward and reverse primers and the fluorescent probe (details of primers/probe—AS_qPCR_zsGreenl-F: GTGTGCATCTGCAACGCCG (SEQ IDNO: 160) & AS-qPCR_zsGReenlR: GACTCGTGGTACATGCAGTTCTC (SEQ IDNO: 161)/ZsGreen probe: ACATCACCGTGAGCGTGGAG (SEQ IDNO: 162); 6FAM-TAMRA; 100 uM) is then prepared depending on the number of samples to be run. The master mix is pipetted into the appropriate number of wells, followed by the addition of the AAV samples, negative or positive controls. A pAAV-zsGreen in-house plasmid, at a concentration of 1e9 copies/ul, is used as the qPCR positive control standard. This is used to prepare 7×10-fold dilutions that will be used as the standard curve for analysis purposes. Untransfected cell samples, non-template control (NTC) and PBS-only wells were also set up, as negative controls. 3×replicates of each sample were run. Once the plate has been set up, a sticky film is placed over it and the plate is briefly spun for 1 min at 1000 rpm on a tabletop centrifuge to remove any bubbles. The plate is then run on a QuantStudio™ 7 Flex Real-Time PCR System (ThermoFisher, UK) following the manufacturer's instructions for the TaqMan™ protocol.

Results of the Experiment

In certain experiments described herein, the plasmid pGH-E1 was added to the transfection mix to create a 2:1.66:1:1 ratio while maintaining overall DNA concentration. An inert carrier DNA, pUC19, was added to the control without extra E1. The Rep-Cap construct was Rep-Cap2. All results were the mean of 3 experiments and error bars represent the standard deviation.

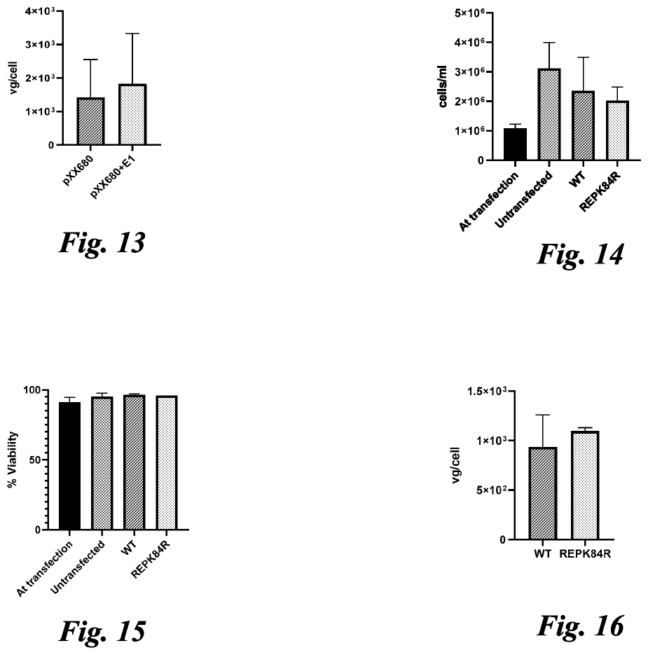

One concern with regards to increasing the levels of E1 in Pro10 cells is the possible toxicity to the cells, resulting in growth retardation or toxicity. However, as can be seen from FIGS. 10 and 11, no difference between the higher E1 concentration and the standard transfection without E1 supplementation was observed. This indicates that the cells were not more sensitive to E1 than the parental strain. After harvest, the cells were lysed and E1A levels and rAAV production was analysed using qPCR as described herein above. As can be seen from FIGS. 12 and 13, E1A levels were increased significantly when the plasmid pGH-E1 is added to the transfection with an ˜4 fold increase in mRNA. However, this increase in expression does not increase the amount of vg/ml produced from the Pro10 cells. This indicates that the level of E1A seen in Pro10 is not the limiting factor in production of rAAV and need not be supplemented to generate a stable cell line.

### Example 5-Testing an Unstable Rep Protein During rAAV Production in Pro10 Cells

As previously described, one hurdle to generating a stable AAV cell line is that continuous expression of some of the essential components is toxic and can result in cell death. This minimizes the chances of generating a cell line, and if a cell line is generated, it is likely to have undesirable characteristics for bioprocessing. There are a number of ways to circumvent these problems, e.g., 1) the use of regulatable promoters to control gene expression and 2) modifying the protein to be less toxic.

Generation of the K84R Mutation

A Rep protein having a lysine (K) to a an Arginine (R) mutation, which was originally show in Weger et al, 2004 to reduce the half-life of the protein, was generated using the Quickchange site directed mutagenesis kit (Agilent: 200518) following the manufacturer's instructions. Primers were designed as per manufacturer's instructions these are shown below:

Fwd primer:
(SEQ ID NO: 163)
gcccttttctttgtgcaatttgagaggggagagagctac
Rev Primer:
(SEQ ID NO: 164)
TCTCCCCTCTCAAATTGCACAAAGAAAAGGGCC

Testing the mutant in rAAV production

The Rep K84R mutant was tested for its effect on rAAV production in Pro10 cells. The protocol described herein above was slightly modified, with the modification being that the standard Rep2-Cap2 plasmid was replaced by the REPK84R plasmid in the triple transfection. The genome plasmid contained the Zs-green transgene. Viral titer was measured as described previously. Viable cell density and viability were calculated using Trypan blue exclusion in a Vicell XR Cell viability Analzyer™ as per the manufacturer's instructions (Beckman Coulter).

Results

These data presented in FIG. 14-16 represent the mean of 3 independent experiments. The error bars represent standard deviation.

As can be seen from FIGS. 14 and 15, the mutated Rep has no significant effect on cell viability or growth during production. This is not surprising as the expression is transient and any benefits of a less stable and therefore less toxic protein only become apparent in a cell line continually expressing the Rep protein. Measurement of viral titer from the production process using this mutant showed no significant difference in the amount of viral genomes replicated as compared to the standard Rep construct. There is a consistent trend to a small increase in the amount of virus produced, however this increase is not statistically significant. Data presented herein show that the K84R mutation creates a less stable Rep protein is not detrimental to AAV production or cell performance.

Determining the Expression Levels of Rep and Cap During rAAV and wtAAV Production

As discussed previously it is known that the AAV replication process is dynamic with many interconnected and interacting parts. AAVs' natural replication cycle involves a cell infected with both an AAV and an adenovirus. This is the most efficient way to produce AAV, however using co-infection with adenovirus to produce rAAV can present challenges, such as contamination of the AAV preparation with adenovirus due to the lytic nature of adenovirus infection, and could cause serious health issues if administered to a patient. For these reasons, the minimal required adenovirus genes required for AAV production, E1, E2A, E4 (all ORFs) and VA-RNA, were identified. These genes are either present in Hek293, such as E1, or in the case of the other genes supplied to the cell via a plasmid. This allows the production of rAAV without using a co-infection with adenovirus, and therefore removing the potential problem of adenovirus contamination. However, these methods make it difficult replicate the natural replication cycle of these gene in the cell. For example, it is known that adenovirus replication is split into an early and late phase, with the early phase being replication and the late phase being a lytic cycle. It should be noted that all genes required for AAV replication were associated with the early phase of adenovirus infection. Crisostomo et al, 2019 showed that during adenovirus 5 infection of human cells the expression profile for these genes was as follows, VA-RNA 3 hrs, E1 1.5 hrs, E2A 3 hrs, E4 ORFs 3 hrs. This would indicate that in Hek293 cells, the production protocol described herein is starting at the point of 6 hrs in the adenovirus replication cycle due to the constitutive expression of E1 in these cells.

In addition to the temporal dynamics of adenovirus gene expression, the Rep and Cap genes of AAV also show temporal gene expression profiles. This was demonstrated by Mouw et al, 2000, where they show that the p5, p19 and p40 promoters were upregulated at different timepoints and to different levels during AAV replication upon co-infection with adenovirus. In this case, the p40 promoter which regulates expression of the Cap gene is immediately upregulated to a high level, whereas the p5 and p19 were upregulated later and to a lesser degree. As p5 and p19 control Rep78 and Rep52, respectively, it appears that Rep52 is required at a higher level than Rep78 and at later timepoints. These timings make sense mechanistically, as the p40 promoter controls Cap expression, Rep78 is responsible for genome replication, and Rep52 is involved in packaging of the genomes into the pre-formed AAV capsids. Therefore, capsid formation is followed by genome replication and then packaging, p40>p5>p19. Furthermore, looking at the timing of expression of AAV promoters, a correlation with the timing of adenovirus early genes that were required for AAV replication is observed. In short, E1, E2A and E4 were activated at 2-3 hrs, p40 at 6 hrs, p5 at 7-8 hrs and p19 at 10 hrs indicating an activation of the promoters by the adenovirus genes.

Experimental Set-Up

The following experiments were designed to determine if differences in temporal gene expression of the E2A and E4 adenovirus genes, and the AAV genes during rAAV production were observed. Side by side production of rAAV-Zs-green and wtAAV were compared. For these experiments, wtAAV is generated using the plasmid, pGRG25-wtAAV2, which has Rep-Cap2 in cis with the ITRs. rAAV is made using Rep2-Cap2.

Deviations from the AAV production protocol described herein above were as follows. Expi293-F cells (Thermofisher A14527) a different 293 suspension cell line was used in lieu of Pro10 cells. Expi293-F cells were maintained in Freestyle F17 expression medium as per Pro10 cells. The transfection protocol is identical with the exception of wtAAV production being simulated by transfecting the pGRG25-wtAAV2 plasmid instead of the Zs-green genome plasmid. mRNA levels of E2A, E4, Rep52, Rep78 and Cap2 were determined by qPCR as outlined above using the following primers and robes.

| | | SEQ | | SEQ | | SEQ | |
|---|---|---|---|---|---|---|---|
| | | ID | | ID | | ID | Probe |
| Target | Forward | NO: | Reverse | NO: | Probe | NO: | chemistry |
| Ad5 E2 | Ad5-E2-72 kDaFw: | 166 | Ad5-E2-72 kDa Rv: | 212 | Ad5-E2-72kDa Pr: | 175 | FAM- |
| 72 kDa | CTTTGGTAGCTGC | | GTATCCTAACGCCC | | TAGTGGCATCAGAA | | BHQ1 |
| | CTTCCCA | | AGACCG | | GGTGA | | |
| Ad5 E4 | Ad5-E4-ORF6a Fw: | 167 | Ad5-E4-ORF6a Rv: | 171 | Ad5-E4-ORF6a Pr: | 176 | FAM- |
| ORF6 | AGGGATCGCCTAC | | AGTGTTACATTCGG | | TACCATACTGGAGG | | BHQ1 |
| | CTCCTTT | | GCAGCA | | ATCATC | | |
| Rep52 | AS_qPCR-Rep52F: | 168 | AS_qPCR-Rep52R: | 172 | Rep52-VIC: | 177 | VIC- |
| | GCCGAGGACTTGC | | TCGGCCAAAGCCAT | | TCCACGCGCACCTTG | | BHQ1 |
| | ATTTCTG | | TCTC | | CTTCCTC | | |
| Rep78 | AS_Rep78-5012: | 169 | Rep78 Rv: | 173 | Rep78-TAMRA: | 178 | FAM- |
| | CTGACAGCTTTGT | | GAATCTGGCGGCAA | | GTGGCCGAGAAGGA | | TAMRA |
| | GAACTGG | | CTC | | ATGG | | |
| Cap2 | Cap Fw: | 170 | Cap Rv: | 174 | Cap Probe: | 179 | FAM- |
| | CAGTTGCGCAGCC | | CAATTACAGATTAC | | TTCGACCACCTTCAG | | TAMRA |
| | AT | | GAGTCAGG | | TGCGGCA | | |
| ITR | CWR73 Aur. | 180 | CWR74 Aur. ITR2_Rv: | 181 | CWR-Probe: | 182 | FAM- |
| primers | ITR2_Fw: | | CGGCCTCAGTGAGC | | CACTCCCTCTCTGCG | | BHQ1 |
| | GGAACCCCTAGTG | | GA | | CGCTCG | | |
| | ATGGAGTT | | | | | | |

Viral titer was assessed using Zs-Green primers and probes previously described for rAAV and ITR primers for the wtAAV experiment.

Results of the Experiment

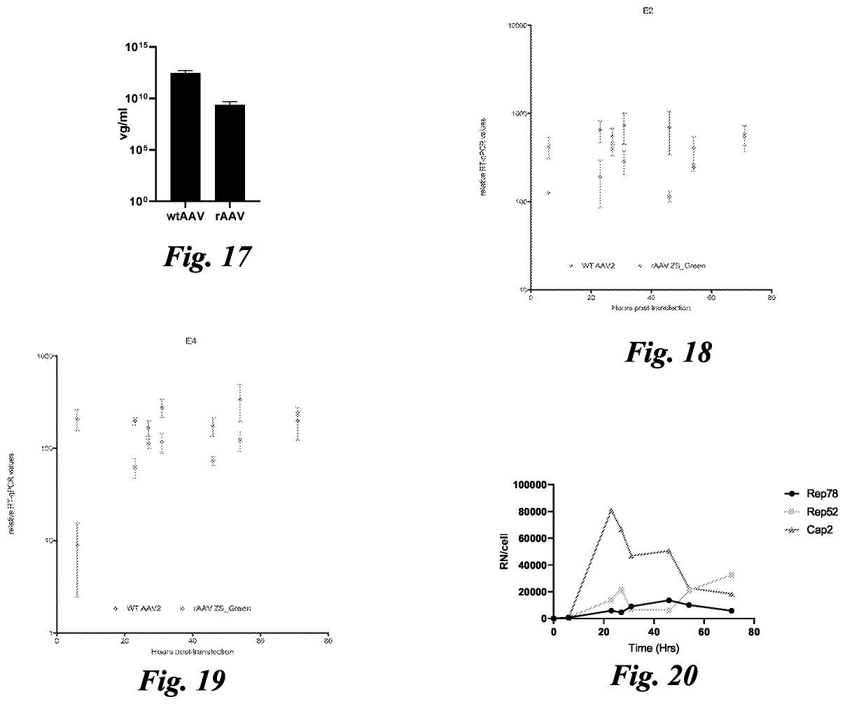

As can be seen from FIGS. 17-19 the differences in E2A and E4 gene expression between conditions is most pronounced at the earlier timepoints <24 hrs with the expression levels becoming almost identical from 24 hrs onwards. In the case of E2, there is a ˜5-fold difference at 12 hrs, whereas the there is greater than 10-fold change with E4. In both cases the expression during wtAAV is lower at this timepoint, indicating that the timing and level of expression may be important for optimal yield generation and that higher levels of Ad helper genes early in the process were detrimental.

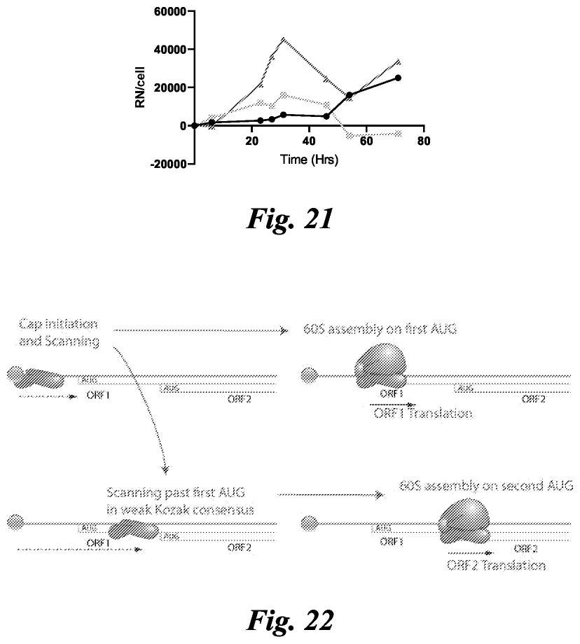

FIGS. 20 and 21 show differences in gene expression during AAV production. The most notable and important of these being that Rep78 and Rep52 appear to have switched expressions at later stages in the process. This may be indicative of inefficient packaging due to lack of Rep52 rAAV, which packages the genomes into capsids, and could explain the differences in the observed viral titers. It is therefore specifically contemplated that correcting this using regulated expression of Rep52 may lead to an increase in titer from rAAV.

The Use of Alternative Start Codons to Control the Ratio of Rep Proteins

As previously described, the ratio of the Rep78 and Rep52 proteins is of critical importance during AAV production. Co-infection of cells with AAV and adenovirus results in a clear preference for greater amounts of Rep52, particularly towards the end of the process, see, e.g., Mouw et al, 2000. However, during the rAAV production process it appears that the ratio of Rep78 to 52 is skewed with there being more Rep78 present than is required. One way to overcome this problem is to use a process inherent to virus biology called leaky scanning. Leaky scanning comprises skipping a weak translational start signal via the ribosome to prevent translation initiation. This can be either because the initiation codon, ATG, is present in a weak Kozak sequence or because it is a non-canonical start codon such as ACG. In this instance the 40S ribosomal subunit skips over the start codon and continues scanning for the next codon to begin translation. Viruses have evolved this mechanism to maximise the amount of information that can be stored in a minimal amount of DNA. By using this mechanism multiple proteins can be encoded on one mRNA if the ATG were not in frame. Similarly, proteins with different N-terminus and different lengths can be encoded if the ATG is in frame. See, e.g., FIG. 22.

Indeed, this mechanism is so well characterized that there is a hierarchy of start codons. See table 5 below.

TABLE 5

| Codon | % of ATG |
|---|---|
| ATG | 100 |
| ACG | 7 |
| ATC | 2 |
| CTG | 19 |
| AGG | 0.5 |

See, e.g., Kearse et al, 2017

In the context of AAV, both Rep78 and 52 are driven by the p5 and p19 promoters respectively, were initiated from ATG codons, indicating that these will have strong translational initiation. In an attempt to correct this imbalance and produce the “correct” ratio of Rep78 and Rep52 for rAAV production the initiation codon of Rep78 was engineered to be either, ACG, ATC, CTG or AGG. The initiation codon for Rep52 will remain ATG in all of these experiments as higher expression of this protein is preferred. However, one can eliminate the Rep52 start codon and insert a separate Rep52 operably linked to a different promoter, for example, a regulatable promoter such as an inducible promoter or one associated with a zinc finger.

Description of the Experiments

Between the start of the Rep78 and Rep52 CDS there is 9 potential ATG's that could be used as a translation initiation site. These were removed by having all the constructs synthesized. These ATG's were mutated while introducing no changes to the amino acid sequence of Rep78. All other synthesised constructs were identical with the exception of either ACG, ATC, CTG or AGG for Rep78 translation initiation.

Viruses were made in Expi-293-F cells using the Zs-Green genome plasmid, and vg/ml was calculated as previously described herein.

Removal of Potential Upstream ATGs does not Affect Viral Production

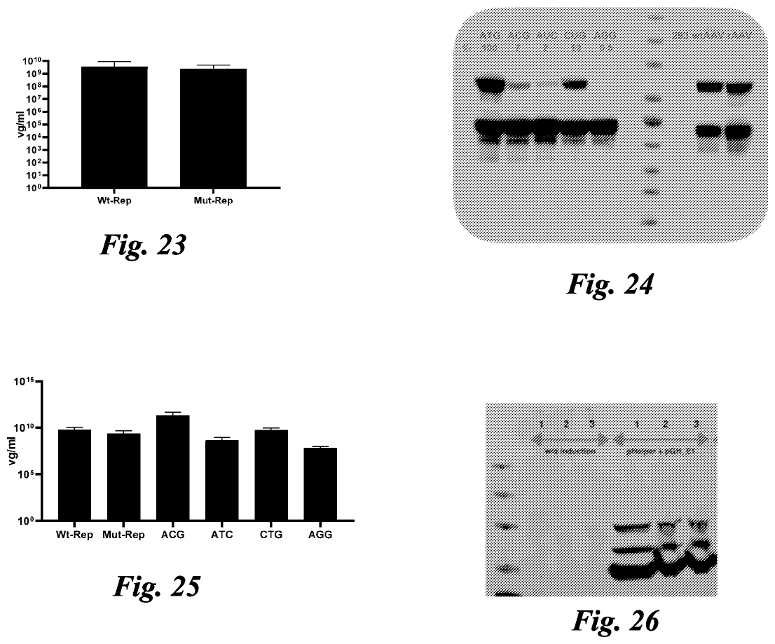

The mutation of the 9 ATGs in the coding sequence for Rep78 has the potential to cause problems during rAAV production, even when there were no predicted changes to the amino acid sequence. Expi293-F cells were triple transfected to assess the impact on these mutations on viral titer. This was done as described previously and using the Zs-green genome. The results can be seen in FIG. 23, which shows that the removal of the 9 potential ATG's that could be used as a translation initiation site from Rep78 had no effect its ability to make rAAV

Testing the Different Start Codons for Expression of Rep78

To assess the effect of the different translation initiation codons on the production of rAAV, AAV production was performed as previously described using the CodOptRep-Cap2 plasmid. In addition, cell lysate was retained and analysed for Rep78, 52 ratios via western blotting.

Western Blot Protocol

Cells were collected in Eppendorf tubes and centrifuged at 300 g for 5 mins in a tabletop centrifuge. Supernatants were discarded and cell pellets were lysed in 1× radioimmunoprecipitation assay (RIPA) buffer, which is made up of RIPA Lysis & Extraction Buffer (Thermofisher, UK, 89900), 100×Halt Protease Inhibitor (Thermofisher, UK, 78445). After addition of the cell lysis buffer all samples were constantly kept on ice. The samples were vortexed and either stored at −20° C. overnight or kept on ice for 30 mins prior to centrifugation at full speed (21,130 g) in the tabletop centrifuge for 20 mins at 4° C. for the removal of any insoluble material. Cell lysates were mixed with 4×LDS loading sample buffer and 10×sample reducing agent (ThermoFisher, UK, NP0007 and NP0004); they were briefly spun down, heated to 95° C. for 10 mins, allowed to cool to 10° C., briefly spun down again and then separated on polyacrylamide gels (NuPAGE Novex 4-12% Bis-Tris Gels, 1.0 mm; ThermoFisher UK, NP0324BOX), which were allowed to reach room temperature before use. The PageRuler™ Prestained NIR Protein Ladder (ThermoFisher, UK, 26635) is run alongside the cell lysates for size comparison purposes. Gels were run at 150V for 1 hour 15 mins using ThermoFisher's XCell SureLock Mini-Cell system. Gels were then transferred to nitrocellulose membranes using the iBlot 2 Dry Blotting System, p0 programme, (ThermoFisher, UK) according to the manufacturer's instructions. Initially, a total protein stain is performed following the manufacturer's instructions (Revert™ 700 Total Protein Stain for Western Blot Normalization; Licor, UK, 926-11011), and this is followed by immunoblotting of the membranes. The membranes were specifically transferred to falcon tubes where they were first blocked with the Licor Odyssey® blocking buffer at room temperature for 1 hour, followed by probing with suitable primary antibodies resuspended in the Licor Odyssey® blocking buffer in a cold room set at 4° C. overnight. The following day membranes were washed 3× in PBS-T (0.05%) for 5 mins each time and then probed with a suitable secondary antibody for 1.5 hours at room temperature. Secondary antibodies typically contain a fluorescent tag for visualisation purposes. Membranes were washed once again 2× in PBS-T (0.05%) for 5 mins each time followed by a final wash in 1×PBS for 5 mins. The membranes were imaged using an Odyssey@ Fc Licor imaging system.

The Ab's used in this experiment were: antiRep antibody 303.9 clone (Progen, 61069). Secondary Ab LICOR anti-mouse 800 nm (LICOR, 926-32210).

Western Blotting of Rep Proteins

The western blot of the Rep proteins using different start codons can be seen below in FIG. 24, which shows that different start codons do express Rep78 in differing amounts. These amounts correspond well with the published translation rates for each codon. From these results, one can see that the current configuration of ATG for Rep78 and 52 leads to an overabundance of Rep78 expression giving an almost 1:1 ratio with Rep52. Whereas, the mutated codons give a range of expression profiles and ratios which always have more Rep52 than Rep78. This is predicted to be favourable to rAAV production.

Viral Titer

From the western blot analysis, it can be seen that the ratio of the Rep78 and Rep52 proteins were altered to be more in line with wtAAV production results that were obtained in the previous section. The next step was to assess if these changes in Rep78 and 52 ratio and expression levels had any effect in viral titer. The results of this can be seen in FIG. 25, which shows that only the ACG mutation makes a significant improvement on viral titer. The use of this start codon gives rise to an ˜1-log increase in viral titer which is a significant improvement. The rest either showed no change or a decrease (AGG, which is not surprising as there was no detectable Rep78) in titer. This may indicate that the rate limiting step is not replication (Rep78, and it is decreased in this context) but packaging of the genomes (Rep52) and that this ratio of Rep78/52 improves this process. Accordingly, this Rep configuration has been included in further experiments described herein.

### Example 6—Inducible Promoters for Control of AAV Gene Expression

As has previously been discussed herein above, regulatable control of the essential proteins for rAAV production is highly desirable due to the dynamic nature of the process. The inventors have previously described promoters that were inducible with the compound forskolin (Sigma-Aldrich, F6886) or its water soluble analog NHK477 (Sigma-Aldrich, N3290). Upon investigation of these promoters, it was discovered that they were inducible by both E1 and the adenovirus helper functions. Using two of these promoters, stable cell lines expressing the Cap2 gene under inducible control were generated. rAAV was made using these stable cell lines, which also have inducible control of VP1, 2 and 3 gene expression.

Generation of Stable Cell Lines

HEK293-T (ATCC-CRL-3216) cell lines were maintained in DMEM plus 10% FBS as described for HEK293 cells. Briefly, cell lines were generated by linearizing the hypoxia-Cap2 (C8) or forskolin-Cap2 (C7) plasmid and transfecting using Lipofectamine 2000 (Invitrogen, 11668027) as per the manufacturer's instructions. The cells were then cultured for 48 hrs in non-selective medium before being switched to selective medium (standard medium plus Blasticidin 5 ug/ml (Thermofisher, A1113902). The cells were then cultured in this medium for 3 weeks before being tested for Cap expression and rAAV production.

Expression of Cap2 from Integrated Inducible Promoters

Forskolin and hypoxia promoter sequences were positioned upstream of the Cap2 gene, and have shown that the expression of the Cap constructs can be controlled via the adenovirus helper functions required for rAAV replication. Cell lines generated using either forskolin and hypoxia promoter sequences were transfected to make rAAV in an identical manner as described for Pro10 cells herein above. Cell lysates were then analysed for expression of the capsid proteins via western blot, anti-AAV VP1/VP2/VP3 mouse monoclonal, B1, (Progen, 61058). The addition of E1 was also tested to assess if the concentration of that protein made a difference to rAAV production; no effect was observed, see previous sections. Surprising, it was found that when adenovirus helper genes and E1 was added, full induction of the promoters and high expression of the capsid proteins was observed. In stark contrast, no expression was observed in the off state (FIG. 26). Though unexpected, biologically these results make sense as viral proteins are known to activate the AP1 and CRE pathways, and these TF binding sites correspond to the TF binding sites that the forskolin and hypoxia promoters are mostly comprised of.

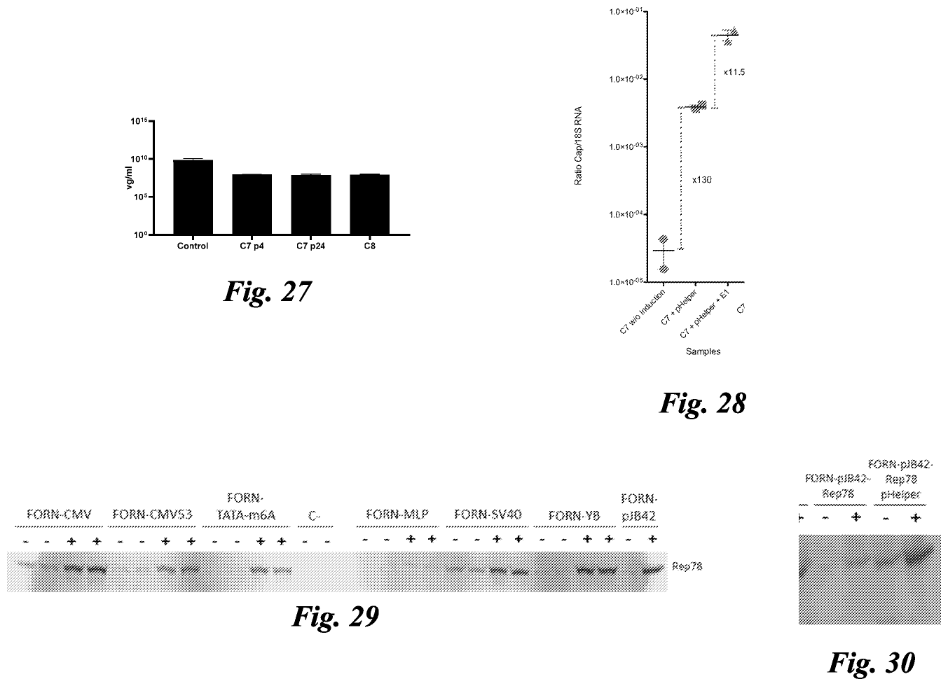

From this result it would be expected that these cells are able to produce rAAV. Therefore, it was not surprising virus was detected from these cell lines (FIG. 27). However, compared to the triple transfection control, the titer was ˜2 log lower. With the C7 cell line (forskolin-Cap2) virus was still detected after 24 passages, or approximately 2 months of continuous growth, showing that these were indeed stable cell lines. However, there were some dynamics of process that were note fully understand that reduces viral titers compared to the control experiment. One hypothesis is that Rep expression downregulates the forskolin and hypoxia promoters due to interactions with AP-1 transcription factors (Prasad et al, 2003). In which case, expression of Rep would decrease levels of the Cap protein and consequently reduce viral titer.

To further these studies, the contribution of the adenovirus helper functions and E1 to the activation of the promoters was assessed. To do this C7 cells were transfected with either the adenovirus helper functions or these functions plus E1 and assessed the effect on Cap mRNA production (FIG. 28). RT-PCR was carried out as previously described (Cap primers). As can be seen from FIG. 28, the adenovirus functions to activate the promoter by over 100-fold whereas addition of E1 boosts this expression by another ˜10-fold, giving an almost 1,000-fold increase in promoter activity. This indicates that the largest contributor to induction were the adenovirus helper functions, whereas excess E1 is required for a modest induction of the promoters.

Control of AAV genes may be via the production process itself, i.e. helper functions causing expression of the genes necessary for rAAV production. This indicates that system should be engineered such that a cascade of expression mirrors the previously described dynamics of wtAAV production.

Design New Control Mechanisms for Rep Expression

Described herein is how the forskolin and hypoxia promoters were significantly upregulated by the adenovirus helper functions, opening the possibility for the design of a cascaded production system. To this end, the promoters previously described were improved and their effectiveness for the control of Rep expression during rAAV production were shown.

Testing of the Promoters

Each of the promoters were tested for their ability to induce expression of the Rep78 protein. To eliminate the expression of Rep52 the ATG start codon in Rep52 was mutated to an AGG. All constructs were synthesized by Genewiz, see sequence files for full plasmid.

Pro10 cells were cultured and transfected using lipofectamine 2000 as previously described herein above. 24 hrs after transfection 10 uM forskolin was added to the cells. Control wells were given DMSO as forskolin was dissolved in this carrier. After another 24 hrs the cells were then lysed and lysates were analyzed for Rep78 expression via western blot (previously described). The best performing promoters were the TATA-m6a, YB and pJB42 (FIG. 29). These promoters have no Rep78 expression in the absence of forskolin but strong expression on the presence (FIG. 29). The rest of the promoters have some level of background which indicates leaky expression. This data strongly indicate that the best performing promoters can be used to tightly control the expression of Rep, increasing the probability of generating a cell line with this toxic protein while also allowing us to raise expression levels to the optimal level for rAAV production.

Activation of the Promoters by Adenovirus Helper Functions

The ability for the aforementioned helper functions to induce the activity of the best performing promoter FORN-pJB42 was next assessed. Transfections were carried out as previously described. Conditions were as follows: FORN-pJB42 controlling expression of Rep78 was transfected with and without the adenovirus helper functions. In addition, the transfected cells were also treated with forskolin at 10p M or vehicle DMSO control. Analysis of activity was as described in the previous section. The promoter is activated by the presence of the helper functions, even in the absence of forskolin, demonstrating that it can be induced by the helper functions (FIG. 30). However, it should be noted that full activity is not obtained until both the adenovirus helper functions and 10 μM forskolin were present. These results, as in a previous section, indicate that this and similar promoters are viable candidates to incorporate into a cascaded gene expression system.

Use of Cre-Recombinase and LoxP Sites to Control Gene Expression of the Rep Gene

In addition to the control of Rep gene expression via inducible promoters use of a different control mechanism was investigated. The inherent problem with controlling Rep-expression is that there are 2-4 proteins made from 2 separate promoters, p5 and p19. In all the Rep constructs described herein the 2 proteins are Rep78 and Rep52. The complication for inducible expression arises as the p19 promoter is found within the coding sequence of the larger of the 2 proteins, Rep78. The production of Rep52 can be stopped by mutating the start codon of this protein to encode a glycine as described above. However, this would require that both Rep proteins encode from separate expression cassettes.

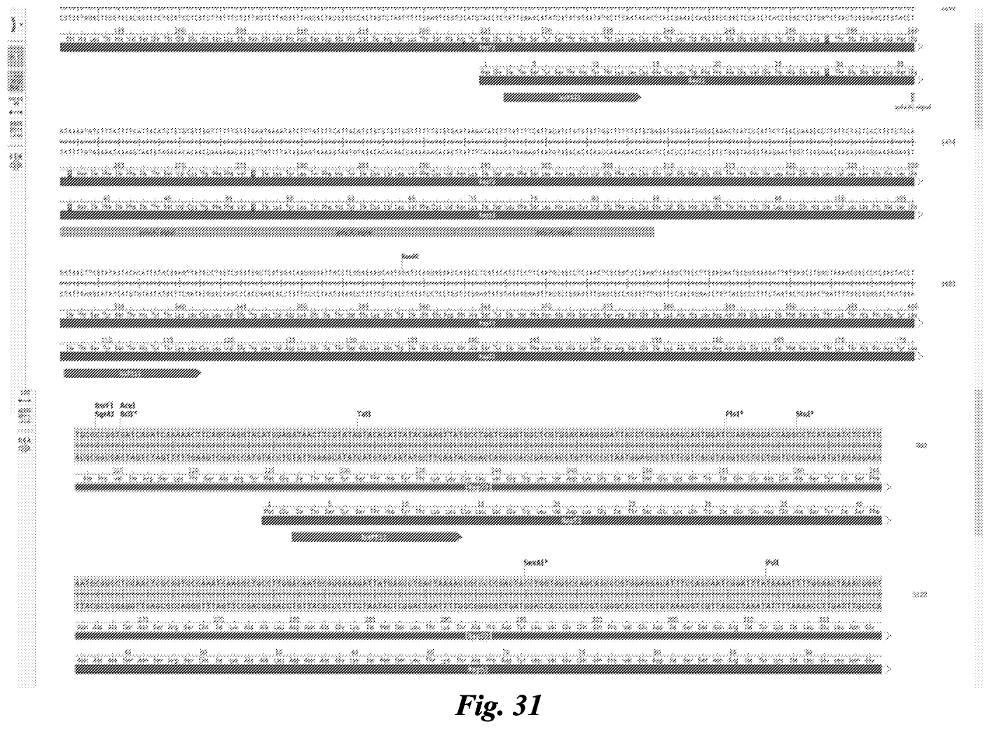

An alternative to this problem is to harness the use of recombinase technology. Accordingly, a Cre-recombinase system is utilized. A Rep construct was designed that placed 2×loxp sites flanking a stretch of 3×PolyA tails directly after the ATG of Rep52. This design should block production of both Rep78 and Rep52 protein without the need to modify either the p5 or p19 promoter. There is precedence for using Cre-Recombinase to make AAV. A similar but different system was used in Yuan et al, 2011. FIG. 31 shows configuration of LoxP-3×PolyA-LoxP block (Rep-loxP-Block) and after recombination. After recombination both Rep proteins should be produced.

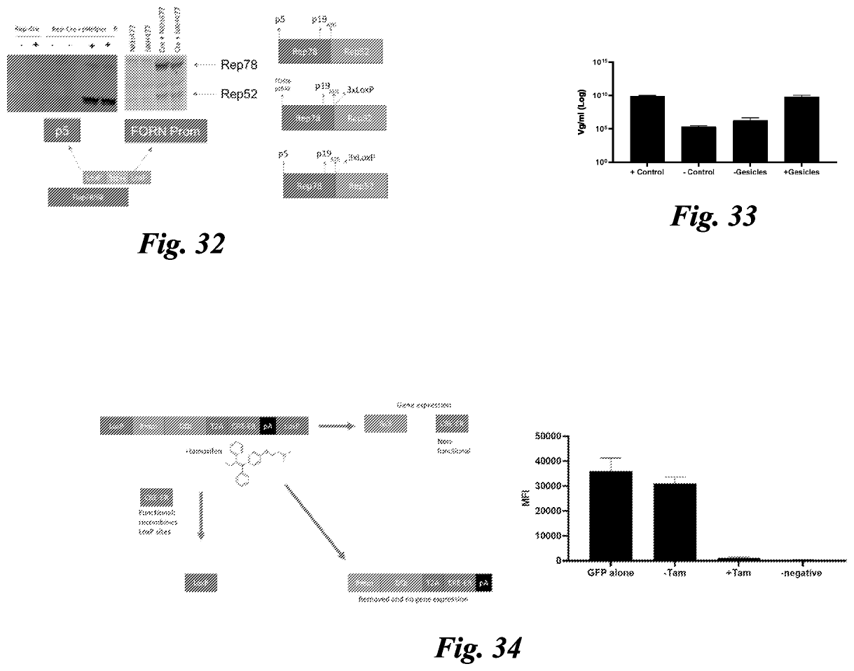

The Rep construct described above was synthesized in 2 ways: 1) with the p5 promoter driving expression of Rep78 (p5-rep-cre-loxp-ca), and 2) the FORN-pJB42 promoter driving Rep78. P19 was unaltered in both constructs. The constructs were tested by transfecting Pro10 as described previously. Cre-recombinase induction for removal of the LoxP flanked PolyA tails was achieved by treatment of the cells with Cre-recombinase gesicles (Takara: 631449) as per the manufacturer's instructions. Induction of FORN-pJB42 was done via 10 uM NHK477. All analysis was via western blot of cell lysates 24 hrs after induction and treatment with Cre-recombinase. It should also be noted that then p5 promoter is strongly upregulated by the adenovirus helper functions, therefore it was added to assess leakiness of the Loxp-PolyA-Loxp block. The results can be seen in, e.g., FIG. 32.

No expression of Rep protein in untreated samples for either promoter was observed (FIG. 32). In addition, both promoters show Rep protein expression even in the presence of the adenovirus helper genes or NHK477. This indicates that the Loxp-3×polyA-Loxp block is highly efficient at controlling expression. Rep protein production is only observed in the presence of Cre-recombinase in both cases with both Rep78 and 52 observed. It should be noted however that in the case of FORN-pBJ42 the expression of Rep78 may be too high for efficient AAV production as it appears to be present in larger quantities than Rep52 which is usually driven from the weaker p19 promoter. Using the p5 configuration, this method of control in AAV production was tested; the p5 Rep-Loxp-3×polyA-loxp construct was cloned into a Rep2-Cap8 plasmid. This vector was then assessed in AAV production in Pro10 as previously described with the only deviation being that Cre-recombinase gesicles were added 3 hrs after transfection. AAV production was measured as previously described. Genome plasmid was Zs-Green.

A vastly reduced AAV produced in the absence of Cre-recombinase was observed (FIG. 33). A small increase was observed as compared to negative control, possibly indicative of leaky expression from the construct in AAV production. However, upon addition of Cre-recombinase, a level of AAV production that corresponds to the positive control was observed. This indicates that this is a viable control mechanism for Rep expression and the stable introduction of this toxic protein into cells. Furthermore, using our inducible promoters an inducible Cre-recombinase expressing cell line could be created that could allow for tailoring of gene expression for optimal AAV yields.

Silencing of Gene Expression Using Cre-LoxP Sites

In tangential experiments to the AAV cell line the power of Cre-recombinase technology was used to demonstrate gene silencing. Gene silencing presents another tool for creating a regulatable stable cell line to optimize the dynamic interplay of genes required to generate high quality and high titer rAAV. In these experiments the construct outlined in FIG. 34 was designed. This construct contains 2 LoxP sites flanking a gene expression cassette containing a promoter driving expression of GFP and a Cre-Recombinase fused to the ligand binding domain of the estrogen receptor (CRE-ER). These 2 proteins are expressed from a single mRNA strand which is fully translated, however 2 separate proteins are generated as a T2A cleavage peptide is incorporated into the design (Liu et al, 2017). The Cre-ER is a ligand activated Cre-recombinase, it has no activity in the absence of the estrogen analogue tamoxifen whereas in the presence of the drug it will excise and rearrange LoxP sites (Donocoff et al, 2020).

In these experiments Hek293 cells were transfected with either the aforementioned construct or a PGK-GFP plasmid, as a control, using Lipofectamine 2000. 3 hrs after transfection either 500 nM (z) 4-hyrdoxy-Tamoxifen (Sigma-Aldrich: 68047-06-3) in ethanol or ethanol (negative control) was added to the cultures. 24 hrs later GFP expression was measured by FACs. The absence of tamoxifen the cells transfected with the Cre-inducible construct (GFP-Cre-ER) have high GFP expression, similar to the positive control (FIG. 34). However, upon addition of the Cre-inducer, 500 nM (z) 4-hyrdoxy-Tamoxifen for 24 hrs, there is very low to little GFP expression indicating removal of the expression cassette via the LoxP sites. There is still some expression which indicates that the excision is not complete, however, even this level is very low and comparable to the negative control. These data indicate that using Cre-recombinase is a viable method of gene silencing and that inducible versions of recombinases could be powerful tools in generating AAV cell lines.

Generation of a Highly Potent AAV by Vector Design

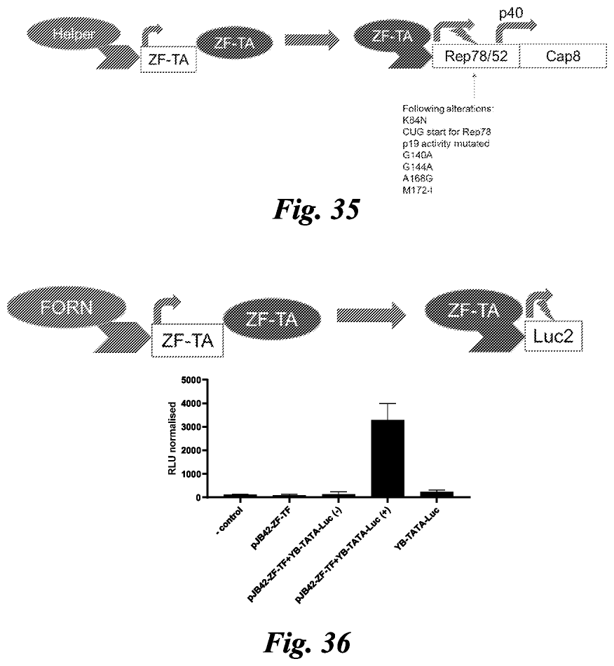

Previously discussed herein is the dynamic interplay of the genes required for rAAV production, as well as the concepts and tools that could be harnessed to replicate the wtAAV situation which are believe to be the optimal situation for AAV manufacture. Namely, expression of genes could be staggered, such as E2A and E4 are activated at 2-3 hrs, p40 (Cap) at 6 hrs, p5 (Rep78) at 7-8 hrs and p19 (Rep52) at 10 hrs. In addition, Rep52 would express at a higher level than Rep78. These conditions would mimic wt AAV kinetics as understood by experiments described herein and could enhance the manufacturing process. To achieve this, the strategy outlined in FIG. 35 was used. To summarize, a zinc-finger transcription activator was designed to bind to the DNA sequence of the minimal promoter YB-TATA. Below is the sequence of YB-TATA that the ZF is designed to bind to.
TCTAGAGGGTATATAATGGGGGCCACTAGTGCCAGCAGCAGCCTGACCACATCTC ATCCTCCAGCCACC (Zinc-Finger target site) (SEQ ID NO: 183)

The zinc finger was designed using the following website, e.g., on the world wide web at https://www.scripps.edu/barbas/zfdesign/zfdesignhome.php.

The DNA binding domain of the zinc-finger was then combined with a nuclear localization signal and a VP64 transcriptional activator domain making it a transcription like factor.

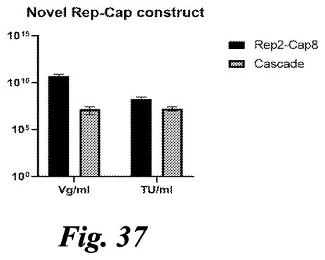

This ZF-TF (ZF-TA in figures) protein was then placed under the control of the adenovirus helper function inducible promoter FORN-pJB42 described previously (FORNpJV42-ZF). In initial experiments the ability of this TF-ZF protein to bind to its target site and activate transcription was assessed using a luciferase based assay. To assess synthesis of this construct, the Luc2 gene was placed downstream of the YB-TATA DNA sequence. If the ZF-TF is able to bind to the YB-TATA site and activate transcription, luciferase activity will only be observed in the presence of the ZF-TF. Indeed, this activity should only be present when the inducible promoter is activated in this case via forskolin activation. This experiment was performed in Pro10 and these were transfected as previously described, forskolin was added to the indicated wells at 10 μM 24 hrs after transfection and luciferase was measured 24 hrs later using the Dual luciferase reporter assay (Promega: E1910) as per the manufacturer's instructions (FIG. 36). ZF-TF functions as expected as the only luciferase expression observed is when both the inducer and the ZF-TF is present. In the absence of either inducer or TF-ZF there is little to no activity (FIG. 36). This indicates that the concept is functional.

However, a number of considerations were to be taken into consideration when used for AAV production. Most notably the activity of the p19 promoter, which is constitutively active and would therefore not be under the control of the cascade. To overcome this, a number of mutations that destroy the p19 promoter active sites were made; some of these mutations introduce amino acid changes, which are highlighted in FIG. 36. These were the most conservative changes that could be made, e.g. glycine to alanine and therefore should have a minimal effect on Rep78 function. Below is
a list of all the changes made to a standard Rep2-Cap8 plasmid.
a. No p5 replaced
b. Zf-binding site for Rep78 expression
c. Non-canonical start codon for Rep78 (ACG)
d. K84N mutation in Rep78 to reduce stability
e. Following mutations to p19

| | |
|---|---|
| i. | cAAP 140: |
| | (SEQ ID NO: 184) |
| | CCAGAAACGG to |
| | (SEQ ID NO: 185) |
| | CacgcAAtG (G140A mutation) |
| ii. | Sp1-130: |
| | (SEQ ID NO: 186) |
| | GAGGCGG to |
| | (SEQ ID NO: 187) |
| | GgGGaG (G144A mutation) |
| iii. | GGT-110: |
| | (SEQ ID NO: 188) |
| | GGTGGTG to |
| | (SEQ ID NO: 189) |
| | aGTtGTt (silent) |
| iv. | SP1-50: |
| | (SEQ ID NO: 190) |
| | AGTGGGCGTG to |
| | (SEQ ID NO: 191) |
| | AaTGGGgaTG (A168G mutation) |
| v. | TATA-35: |
| | (SEQ ID NO: 192) |
| | TAATAT to |
| | (SEQ ID NO: 213) |
| | aAAcAT (silent) |
| vi. | TATA-20: |
| | (SEQ ID NO: 193) |
| | TATTTA to |
| | (SEQ ID NO: 194) |
| | TAcctg (silent) |

vii. ATG for small Rep
viii. P40 intact with Cap8 downstream

These modifications would mean that the expression of both Rep proteins is controlled by the ZF-TF as p19 is destroyed with the levels of expression of both Reps controlled by the leaky scanning mechanism as previously described.

This new Rep2-Cap8 plasmid was tested for its ability to make rAAV in a transfection as previously described. The deviation from this was that the ZF-TF plasmid was added to the transfection for activation of the promoter. It was also compared to the standard Rep2-Cap8 plasmid. Viral titer was determined from a Zs-green genome plasmid.

It should be noted that in this experiment the ZF-TF will be active throughout the duration of the production as the FORN-pJB42 was activated by the adenovirus helper functions which were present in the transfection. In addition to viral titer transduction potential (potency) of the produced virus was performed using the following protocol.

Briefly, virus productions from Pro10 were titered by qPCR and added in serial dilution steps, this generates different MOIs, 1:10, 1:100, 1:1000 and 1:10,000 to a plate of Hek29H cells (Gibco: 11631017) in triplicate. These cells were maintained as per Hek293 cells. The cells were incubated for 48 hrs with the virus and then washed 3× in PBS. The cells were then measured for GFP fluorescence via FACs. The MFI was plotted against the dilutions to generate a standard curve and if the linear regression was greater than 0.95 the assay was accepted. The transducing units/ml was then calculated using from the MOI.

The results of both the virus titer and transducing units are presented in FIG. 37. The cascade Rep2-Cap8 plasmid (full cascade construct) was less efficient during the production of rAAV than the standard triple transfection; this difference was approximately 200-fold. (FIG. 37). However, when the potency of the viruses was assessed it was observed that the cascade produced a far more potent batch of virus, approximately 500-fold. When calculated it means that the cascaded expression produced a yield of 1 potent virus per 9 viruses. This is a significant improvement over the 1 in 4,500 produced by the standard procedure. The reasons for this are unclear, however it specifically hypothesized herein that it is due to the staggered gene expression introduced into the production process. This is a significant finding as high potency virus is a requirement for lowering the dose of AAV given to patients as the high doses currently being administered 1×10\hat{ }4 vg/ml have shown toxicity. It was contemplated that a reduction of the dose by 500-fold would mitigate the risk of this toxicity. Furthermore, it was specifically contemplated herein that incorporating inducible helper functions to fully control the activity of the cascade an optimal production protocol and increase the yields in production.

The foregoing is illustrative of the present invention, and is not to be construed as limiting thereof. The invention is defined by the following claims, with equivalents of the claims to be included therein.

### Example 7

Sequences—Hypoxia-Inducible Promoters:

Synp-RTV-015

Synp-RTV-015
(SEQ ID NO: 1)
ACGTGCGATGATGCGTAGCTAGTAGTGATGATGCGTAGCTAGTAGTACGTGCGATGATG
CGTAGCTAGTAGTGATGATGCGTAGCTAGTAGTACGTGCGATGATGCGTAGCTAGTAGTG
ATGATGCGTAGCTAGTAGTACGTGCGATGATGCGTAGCTAGTAGTGATGATGCGTAGCTA
GTAGTACGTGCTTGGTACCATCCGGGCCGGCCGCTTAAGCGACGCCTATAAAAAATA
GGTTGCATGCTAGGCCTAGCGCTGCCAGTCCATCTTCGCTAGCCTGTGCTGCGTCA
GTCCAGCGCTGCGCTGCGTAACGGCCGCC
HRE1 underlined, MP1 minimal promoter in bold.
Synp-HYP-001
(SEQ ID NO: 3)
CTGCACGTACTGCACGTACTGCACGTACTGCACGTATGGGTACCGTCGACGATATCGGAT
CCAGGTCTATATAAGCAGAGCTCGTTTAGTGAACCGTCAGATCGCCTAGATACGCC
ATCCACGCTGTTTTGACCTCCATAGAAGATCGCCACC
HRE2 underlined, minimal promoter bold
pMA-RQ luciferase vector - RTV-015
(SEQ ID NO: 195)
ACGTGCGATGAGCTCCCCGGGTTAATTAACATATGACTAGTGAATTCATTGATCATAATC
AGCCATACCACATTTGTAGAGGTTTTACTTGCTTTAAAAAACCTCCCACACCTCCCCCTG
AACCTGAAACATAAAATGAATGCAATTGTTGTTGTTAACTTGTTTATTGCAGCTTATAAT
GGTTACAAATAAAGCAATAGCATCACAAATTTCACAAATAAAGCATTTTTTTCACTGCAT
TCTAGTTGTGGTTTGTCCAAACTCATCAATGTATCTTATCATGTCTGGCGGCCGCGACCTG
CAGGCGCAGAACTGGTAGGTATGGAAGATCCCTCGAGATCCATTGTGCTGGCGGTAGGC
GAGCAGCGCCTGCCTGAAGCTGCGGGCATTCCCGATCAGAAATGAGCGCCAGTCGTCGT
CGGCTCTCGGCACCGAATGCGTATGATTCTCCGCCAGCATGGCTTCGGCCAGTGCGTCGA
GCAGCGCCCGCTTGTTCCTGAAGTGCCAGTAAAGCGCCGGCTGCTGAACCCCCAACCGTT
CCGCCAGTTTGCGTGTCGTCAGACCGTCTACGCCGACCTCGTTCAACAGGTCCAGGGCGG
CACGGATCACTGTATTCGGCTGCAACTTTGTCATGCTTGACACTTTATCACTGATAAACAT
AATATGTCCACCAACTTATCAGTGATAAAGAATCCGCGCCAGCACAATGGATCTCGAGG
TCGAGGGATCTCTAGAGGATCCTCTACGCCGGACGCATCGTGGCCGGCATCACCGGCGC
CACAGGTGCGGTTGCTGGCGCCTATATCGCCGACATCACCGATGGGGAAGATCGGGCTC
GCCACTTCGGGCTCATGAGCGCTTGTTTCGGCGTGGGTATGGTGGCAGGCCCCGTGGCCG
GGGGACTGTTGGGCGCCATCTCCTTGCATGCACCATTCCTTGCGGCGGCGGTGCTCAACG
GCCTCAACCTACTACTGGGCTGCTTCCTAATGCAGGAGTCGCATAAGGGAGAGCGTCGA
CCTCGGGCCGCGTTGCTGGCGTTTTTCCATAGGCTCCGCCCCCCTGACGAGCATCACAAA
AATCGACGCTCAAGTCAGAGGTGGCGAAACCCGACAGGACTATAAAGATACCAGGCGTT
TCCCCCTGGAAGCTCCCTCGTGCGCTCTCCTGTTCCGACCCTGCCGCTTACCGGATACCTG
TCCGCCTTTCTCCCTTCGGGAAGCGTGGCGCTTTCTCATAGCTCACGCTGTAGGTATCTCA
GTTCGGTGTAGGTCGTTCGCTCCAAGCTGGGCTGTGTGCACGAACCCCCCGTTCAGCCCG
ACCGCTGCGCCTTATCCGGTAACTATCGTCTTGAGTCCAACCCGGTAAGACACGACTTAT
CGCCACTGGCAGCAGCCACTGGTAACAGGATTAGCAGAGCGAGGTATGTAGGCGGTGCT
ACAGAGTTCTTGAAGTGGTGGCCTAACTACGGCTACACTAGAAGAACAGTATTTGGTATC
TGCGCTCTGCTGAAGCCAGTTACCTTCGGAAAAAGAGTTGGTAGCTCTTGATCCGGCAAA
CAAACCACCGCTGGTAGCGGTGGTTTTTTTGTTTGCAAGCAGCAGATTACGCGCAGAAAA
AAAGGATCTCAAGAAGATCCTTTGATCTTTTCTACGGGGTCTGACGCTCAGTGGAACGAA
AACTCACGTTAAGGGATTTTGGTCATGAGATTATCAAAAAGGATCTTCACCTAGATCCTT
TTAAATTAAAAATGAAGTTTTAAATCAATCTAAAGTATATATGAGTAAACTTGGTCTGAC
AGTTACCAATGCTTAATCAGTGAGGCACCTATCTCAGCGATCTGTCTATTTCGTTCATCCA
TAGTTGCCTGACTCCCCGTCGTGTAGATAACTACGATACGGGAGGGCTTACCATCTGGCC
CCAGTGCTGCAATGATACCGCGAGACCCACGCTCACCGGCTCCAGATTTATCAGCAATAA
ACCAGCCAGCCGGAAGGGCCGAGCGCAGAAGTGGTCCTGCAACTTTATCCGCCTCCATC
CAGTCTATTAATTGTTGCCGGGAAGCTAGAGTAAGTAGTTCGCCAGTTAATAGTTTGCGC
AACGTTGTTGCCATTGCTACAGGCATCGTGGTGTCACGCTCGTCGTTTGGTATGGCTTCAT
TCAGCTCCGGTTCCCAACGATCAAGGCGAGTTACATGATCCCCCATGTTGTGCAAAAAAG
CGGTTAGCTCCTTCGGTCCTCCGATCGTTGTCAGAAGTAAGTTGGCCGCAGTGTTATCAC
TCATGGTTATGGCAGCACTGCATAATTCTCTTACTGTCATGCCATCCGTAAGATGCTTTTC
TGTGACTGGTGAGTACTCAACCAAGTCATTCTGAGAATAGTGTATGCGGCGACCGAGTTG
CTCTTGCCCGGCGTCAATACGGGATAATACCGCGCCACATAGCAGAACTTTAAAAGTGCT
CATCATTGGAAAACGTTCTTCGGGGCGAAAACTCTCAAGGATCTTACCGCTGTTGAGATC
CAGTTCGATGTAACCCACTCGTGCACCCAACTGATCTTCAGCATCTTTTACTTTCACCAGC
GTTTCTGGGTGAGCAAAAACAGGAAGGCAAAATGCCGCAAAAAAGGGAATAAGGGCGA
CACGGAAATGTTGAATACTCATACTCTTCCTTTTTCAATATTATTGAAGCATTTATCAGGG
TTATTGTCTCATGAGCGGATACATATTTGAATGTATTTAGAAAAATAAACAAATAGGGGT
TCCGCGCACATTTCCCCGAAAAGTGCCACCTGACGTCTAAGAAACCATTATTATCATGAC
ATTAACCTATAAAAATAGGCGTATCACGAGGCCCTGATGGCTCTTTGCGGCACCCATCGT
TCGTAATGTTCCGTGGCACCGAGGACAACCCTCAAGAGAAAATGTAATCACACTGGCTC
ACCTTCGGGTGGGCCTTTCTGCGTTTATAAGGAGACACTTTATGTTTAAGAAGGTTGGTA
AATTCCTTGCGGCTTTGGCAGCCAAGCTAGATCCGGCTGTGGAATGTGTGTCAGTTAGGG
TGTGGAAAGTCCCCAGGCTCCCCAGCAGGCAGAAGTATGCAAAGCATGCATCTCAATTA
GTCAGCAACCAGGTGTGGAAAGTCCCCAGGCTCCCCAGCAGGCAGAAGTATGCAAAGCA
TGCATCTCAATTAGTCAGCAACCATAGTCCCGCCCCTAACTCCGCCCATCCCGCCCCTAA
CTCCGCCCAGTTCCGCCCATTCTCCGCCCCATGGCTGACTAATTTTTTTTATTTATGCAGA
GGCCGAGGCCGCCTCGGCCTCTGAGCTATTCCAGAAGTAGTGAGGAGGCTTTTTTGGAGG
CCTAGGCTTTTGCAAAAAGCTAGCTTGGGGCCACCGCTCAGAGCACCTTCCACCATGGCC
ACCTCAGCAAGTTCCCACTTGAACAAAAACATCAAGCAAATGTACTTGTGCCTGCCCCAG
GGTGAGAAAGTCCAAGCCATGTATATCTGGGTTGATGGTACTGGAGAAGGACTGCGCTG
CAAAACCCGCACCCTGGACTGTGAGCCCAAGTGTGTAGAAGAGTTACCTGAGTGGAATT
TTGATGGCTCTAGTACCTTTCAGTCTGAGGGCTCCAACAGTGACATGTATCTCAGCCCTG
TTGCCATGTTTCGGGACCCCTTCCGCAGAGATCCCAACAAGCTGGTGTTCTGTGAAGTTT
TCAAGTACAACCGGAAGCCTGCAGAGACCAATTTAAGGCACTCGTGTAAACGGATAATG
GACATGGTGAGCAACCAGCACCCCTGGTTTGGAATGGAACAGGAGTATACTCTGATGGG
AACAGATGGGCACCCTTTTGGTTGGCCTTCCAATGGCTTTCCTGGGCCCCAAGGTCCGTA
TTACTGTGGTGTGGGCGCAGACAAAGCCTATGGCAGGGATATCGTGGAGGCTCACTACC
GCGCCTGCTTGTATGCTGGGGTCAAGATTACAGGAACAAATGCTGAGGTCATGCCTGCCC
AGTGGGAGTTCCAAATAGGACCCTGTGAAGGAATCCGCATGGGAGATCATCTCTGGGTG
GCCCGTTTCATCTTGCATCGAGTATGTGAAGACTTTGGGGTAATAGCAACCTTTGACCCC
AAGCCCATTCCTGGGAACTGGAATGGTGCAGGCTGCCATACCAACTTTAGCACCAAGGC
CATGCGGGAGGAGAATGGTCTGAAGCACATCGAGGAGGCCATCGAGAAACTAAGCAAG
CGGCACCGGTACCACATTCGAGCCTACGATCCCAAGGGGGGCCTGGACAATGCCCGTCG
TCTGACTGGGTTCCACGAAACGTCCAACATCAACGACTTTTCTGCTGGTGTCGCCAATCG
CAGTGCCAGCATCCGCATTCCCCGGACTGTCGGCCAGGAGAAGAAAGGTTACTTTGAAG
ACCGCCGCCCCTCTGCCAATTGTGACCCCTTTGCAGTGACAGAAGCCATCGTCCGCACAT
GCCTTCTCAATGAGACTGGCGACGAGCCCTTCCAATACAAAAACTAATTAGACTTTGAGT
GATCTTGAGCCTTTCCTAGTTCATCCCACCCCGCCCCAGAGAGATCTTTGTGAAGGAACC
TTACTTCTGTGGTGTGACATAATTGGACAAACTACCTACAGAGATTTAAAGCTCTAAGGT
AAATATAAAATTTTTAAGTGTATAATGTGTTAAACTACTGATTCTAATTGTTTGTGTATTT
TAGATTCCAACCTATGGAACTGATGAATGGGAGCAGTGGTGGAATGCCTTTAATGAGGA
AAACCTGTTTTGCTCAGAAGAAATGCCATCTAGTGATGATGAGGCTACTGCTGACTCTCA
ACATTCTACTCCTCCAAAAAAGAAGAGAAAGGTAGAAGACCCCAAGGACTTTCCTTCAG
AATTGCTAAGTTTTTTGAGTCATGCTGTGTTTAGTAATAGAACTCTTGCTTGCTTTGCTAT
TTACACCACAAAGGAAAAAGCTGCACTGCTATACAAGAAAATTATGGAAAAATATTCTG
TAACCTTTATAAGTAGGCATAACAGTTATAATCATAACATACTGTTTTTTCTTACTCCACA
CAGGCATAGAGTGTCTGCTATTAATAACTATGCTCAAAAATTGTGTACCTTTAGCTTTTTA
ATTTGTAAAGGGGTTAATAAGGAATATTTGATGTATAGTGCCTTGACTAGAGATCATAAT
CAGCCATACCACATTTGTAGAGGTTTTACTTGCTTTAAAAAACCTCCCACACCTCCCCCTG
AACCTGAAACATAAAATGAATGCAATTGTTGTTGTTAACTTGTTTATTGCAGCTTATAAT
GGTTACAAATAAAGCAATAGCATCACAAATTTCACAAATAAAGCATTTTTTTCACTGCAT
TCTAGTTGTGGTTTGTCCAAACTCATCAATGTATCTTATCATGTCTGGATCTCTAGCTTCG
TGTCAAGGACGGTGAGG
pMA-RQ luciferase vector - Synp-HYP-001
(SEQ ID NO: 196)
AATAAAATATCTTTATTTTCATTACATCTGTGTGTTGGTTTTTTGTGTGCTGCACGTACTG
CACGTACTGCACGTACTGCACGTATGGGTACCGTCGACGATATCGGATCCAGGTCTATAT
AAGCAGAGCTCGTTTAGTGAACCGTCAGATCGCCTAGATACGCCATCCACGCTGTTTTGA
CCTCCATAGAAGATCGCCACCATGGAAGATGCCAAAAACATTAAGAAGGGCCCAGCGCC
ATTCTACCCACTCGAAGACGGGACCGCCGGCGAGCAGCTGCACAAAGCCATGAAGCGCT
ACGCCCTGGTGCCCGGCACCATCGCCTTTACCGACGCACATATCGAGGTGGACATTACCT
ACGCCGAGTACTTCGAGATGAGCGTTCGGCTGGCAGAAGCTATGAAGCGCTATGGGCTG
AATACAAACCATCGGATCGTGGTGTGCAGCGAGAATAGCTTGCAGTTCTTCATGCCCGTG
TTGGGTGCCCTGTTCATCGGTGTGGCTGTGGCCCCAGCTAACGACATCTACAACGAGCGC
GAGCTGCTGAACAGCATGGGCATCAGCCAGCCCACCGTCGTATTCGTGAGCAAGAAAGG
GCTGCAAAAGATCCTCAACGTGCAAAAGAAGCTACCGATCATACAAAAGATCATCATCA
TGGATAGCAAGACCGACTACCAGGGCTTCCAAAGCATGTACACCTTCGTGACTTCCCATT
TGCCACCCGGCTTCAACGAGTACGACTTCGTGCCCGAGAGCTTCGACCGGGACAAAACC
ATCGCCCTGATCATGAACAGTAGTGGCAGTACCGGATTGCCCAAGGGCGTAGCCCTACC
GCACCGCACCGCTTGTGTCCGATTCAGTCATGCCCGCGACCCCATCTTCGGCAACCAGAT
CATCCCCGACACCGCTATCCTCAGCGTGGTGCCATTTCACCACGGCTTCGGCATGTTCAC
CACGCTGGGCTACTTGATCTGCGGCTTTCGGGTCGTGCTCATGTACCGCTTCGAGGAGGA
GCTATTCTTGCGCAGCTTGCAAGACTATAAGATTCAATCTGCCCTGCTGGTGCCCACACT
ATTTAGCTTCTTCGCTAAGAGCACTCTCATCGACAAGTACGACCTAAGCAACTTGCACGA
GATCGCCAGCGGCGGGGCGCCGCTCAGCAAGGAGGTAGGTGAGGCCGTGGCCAAACGCT
TCCACCTACCAGGCATCCGCCAGGGCTACGGCCTGACAGAAACAACCAGCGCCATTCTG
ATCACCCCCGAAGGGGACGACAAGCCTGGCGCAGTAGGCAAGGTGGTGCCCTTCTTCGA
GGCTAAGGTGGTGGACTTGGACACCGGTAAGACACTGGGTGTGAACCAGCGCGGCGAGC
TGTGCGTCCGTGGCCCCATGATCATGAGCGGCTACGTTAACAACCCCGAGGCTACAAAC
GCTCTCATCGACAAGGACGGCTGGCTGCACAGCGGCGACATCGCCTACTGGGACGAGGA
CGAGCACTTCTTCATCGTGGACCGGCTGAAGAGCCTGATCAAATACAAGGGCTACCAGG
TAGCCCCAGCCGAACTGGAGAGCATCCTGCTGCAACACCCCAACATCTTCGACGCCGGG
GTCGCCGGCCTGCCCGACGACGATGCCGGCGAGCTGCCCGCCGCAGTCGTCGTGCTGGA
ACACGGTAAAACCATGACCGAGAAGGAGATCGTGGACTATGTGGCCAGCCAGGTTACAA
CCGCCAAGAAGCTGCGCGGTGGTGTTGTGTTCGTGGACGAGGTGCCTAAAGGACTGACC
GGCAAGTTGGACGCCCGCAAGATCCGCGAGATTCTCATTAAGGCCAAGAAGGGCGGCAA
GATCGCCGTGTAATGAAAGCTTGGTCTCTACGAGTAATAGACGCCCAGTTGAATTCCTTC
GAGCAGACATGATAAGATACATTGATGAGTTTGGACAAACCACAACTAGAATGCAGTGA
AAAAAATGCTTTATTTGTGAAATTTGTGATGCTATTGCTTTATTTGTAACCATTATAAGCT
GCAATAAACAAGTTAACAACAACAATTGCATTCATTTTATGTTTCAGGTTCAGGGGGAGG
TGTGGGAGGTTTTTTAAAGCAAGTAAAACCTCTACAAATGTGGTAAAATCGATAAGGAT
CCGTCTGGGCCTCATGGGCCTTCCGCTCACTGCCCGCTTTCCAGTCGGGAAACCTGTCGT
GCCAGCTGCATTAACATGGTCATAGCTGTTTCCTTGCGTATTGGGCGCTCTCCGCTTCCTC
GCTCACTGACTCGCTGCGCTCGGTCGTTCGGGTAAAGCCTGGGGTGCCTAATGAGCAAAA
GGCCAGCAAAAGGCCAGGAACCGTAAAAAGGCCGCGTTGCTGGCGTTTTTCCATAGGCT
CCGCCCCCCTGACGAGCATCACAAAAATCGACGCTCAAGTCAGAGGTGGCGAAACCCGA
CAGGACTATAAAGATACCAGGCGTTTCCCCCTGGAAGCTCCCTCGTGCGCTCTCCTGTTC
CGACCCTGCCGCTTACCGGATACCTGTCCGCCTTTCTCCCTTCGGGAAGCGTGGCGCTTTC
TCATAGCTCACGCTGTAGGTATCTCAGTTCGGTGTAGGTCGTTCGCTCCAAGCTGGGCTG
TGTGCACGAACCCCCCGTTCAGCCCGACCGCTGCGCCTTATCCGGTAACTATCGTCTTGA
GTCCAACCCGGTAAGACACGACTTATCGCCACTGGCAGCAGCCACTGGTAACAGGATTA
GCAGAGCGAGGTATGTAGGCGGTGCTACAGAGTTCTTGAAGTGGTGGCCTAACTACGGC
TACACTAGAAGAACAGTATTTGGTATCTGCGCTCTGCTGAAGCCAGTTACCTTCGGAAAA
AGAGTTGGTAGCTCTTGATCCGGCAAACAAACCACCGCTGGTAGCGGTGGTTTTTTTGTT
TGCAAGCAGCAGATTACGCGCAGAAAAAAAGGATCTCAAGAAGATCCTTTGATCTTTTCT
ACGGGGTCTGACGCTCAGTGGAACGAAAACTCACGTTAAGGGATTTTGGTCATGAGATT
ATCAAAAAGGATCTTCACCTAGATCCTTTTAAATTAAAAATGAAGTTTTAAATCAATCTA
AAGTATATATGAGTAAACTTGGTCTGACAGTTACCAATGCTTAATCAGTGAGGCACCTAT
CTCAGCGATCTGTCTATTTCGTTCATCCATAGTTGCCTGACTCCCCGTCGTGTAGATAACT
ACGATACGGGAGGGCTTACCATCTGGCCCCAGTGCTGCAATGATACCGCGAGAACCACG
CTCACCGGCTCCAGATTTATCAGCAATAAACCAGCCAGCCGGAAGGGCCGAGCGCAGAA
GTGGTCCTGCAACTTTATCCGCCTCCATCCAGTCTATTAATTGTTGCCGGGAAGCTAGAG
TAAGTAGTTCGCCAGTTAATAGTTTGCGCAACGTTGTTGCCATTGCTACAGGCATCGTGG
TGTCACGCTCGTCGTTTGGTATGGCTTCATTCAGCTCCGGTTCCCAACGATCAAGGCGAG
TTACATGATCCCCCATGTTGTGCAAAAAAGCGGTTAGCTCCTTCGGTCCTCCGATCGTTGT
CAGAAGTAAGTTGGCCGCAGTGTTATCACTCATGGTTATGGCAGCACTGCATAATTCTCT
TACTGTCATGCCATCCGTAAGATGCTTTTCTGTGACTGGTGAGTACTCAACCAAGTCATTC
TGAGAATAGTGTATGCGGCGACCGAGTTGCTCTTGCCCGGCGTCAATACGGGATAATACC
GCGCCACATAGCAGAACTTTAAAAGTGCTCATCATTGGAAAACGTTCTTCGGGGCGAAA
ACTCTCAAGGATCTTACCGCTGTTGAGATCCAGTTCGATGTAACCCACTCGTGCACCCAA
CTGATCTTCAGCATCTTTTACTTTCACCAGCGTTTCTGGGTGAGCAAAAACAGGAAGGCA
AAATGCCGCAAAAAAGGGAATAAGGGCGACACGGAAATGTTGAATACTCATACTCTTCC
TTTTTCAATATTATTGAAGCATTTATCAGGGTTATTGTCTCATGAGCGGATACATATTTGA
ATGTATTTAGAAAAATAAACAAATAGGGGTTCCGCGCACATTTCCCCGAAAAGTGCCAC
CTAAATTGTAAGCGTTAATATTTTGTTAAAATTCGCGTTAAATTTTTGTTAAATCAGCTCA
TTTTTTAACCAATAGGCCGAA

Sequences—Forskolin-Inducible Promoters:

Synp-FORCSV-10
(SEQ ID NO: 205)
CCATTGACGTCACGATTACCATTGACGTCACGATTACCATTGACGTCACGATTACCATTG
ACGTCACGATTACCATTGACGTCAGCGATTAAGATGACTCAGCGATTAAGATGACTCAGC
GATTAAGATGACTCAGCGATTAAGATGACTCACTAGCCCGGGCTCGAGATCTGCGATCT
GCATCTCAATTAGTCAGCAACCATAGTCCCGCCCCTAACTCCGCCCATCCCGCCCCT
AACTCCGCCCAGTTCCGCCCATTCTCCGCCCCATCGCTGACTAATTTTTTTTATTTA
TGCAGAGGCCGAGGCCGCCTCGGCCTCTGAGCTATTCCAGAAGTAGTGAGGAGGCT
TTTTTGGAGGCCTAGGCTTTTGCAAAAAGCTTGGCATTCCGGTACTGTTGGTAAAGCC
ACC
CAMPRE and AP1 underlined, minimal promoter bold
Synp-FORCMV-09
(SEQ ID NO: 206)
CCATTGACGTCACGATTACCATTGACGTCACGATTACCATTGACGTCACGATTACCATTG
ACGTCACGATTACCATTGACGTCAGCGATTAAGATGACTCAGCGATTAAGATGACTCAGC
GATTAAGATGACTCAGCGATTAAGATGACTCAGCGATTAATCCATATGCAGGTCTATAT
AAGCAGAGCTCGTTTAGTGAACCGTCAGATCGCCTAGATACGCCATCCACGCTGTT
TTGACCTCCATAGAAGATCGCCACC
CAMPRE and API underlined, minimal promoter bold
Synp-FMP-02
(SEQ ID NO: 8)
TGAGTCAGATGATGCGTAGCTAGTAGTTGAGTCAGATGATGCGTAGCTAGTAGTTGAGTC
AGATGATGCGTAGCTAGTAGTTGAGTCAGATGATGCGTAGCTAGTAGTTGAGTCAGATG
ATGCGTAGCTAGTAGTTGAGTCAGATGATGCGTAGCTAGTAGTTGAGTCAGATGATGCGT
AGCTAGTAGTTGAGTCAGTAGTCGTATGCTGATGCGCAGTTAGCGTAGCTGAGGTACCGT
CGACGATATCGGATCCTTCGCATATTAAGGTGACGCGTGTGGCCTCGAACACCGAGC
GACCCTGCAGCGACCCGCTTAA
AP1 underlined, minimal promoter bold
Synp-FLP-01
(SEQ ID NO: 9)
TGAGTCAGATGATGCGTAGCTAGTAGTTGAGTCAGATGATGCGTAGCTAGTAGTTGAGTC
AGATGATGCGTAGCTAGTAGTTGAGTCAGATGATGCGTAGCTAGTAGTTGAGTCAGATG
ATGCGTAGCTAGTAGTTGAGTCAGATGATGCGTAGCTAGTAGTTGAGTCAGATGATGCGT
AGCTAGTAGTTGAGTCAGTAGTCGTATGCTGATGCGCAGTTAGCGTAGCTGAGGTACCGT
CGACGATATCGGATCCGGGCATATAAAACAGGGGCAAGGCACAGACTCATAGCAGA
GCAATCACCACCAAGCCTGGAATAACTGCAGCCACC
AP1 underlined, minimal promoter bold
FORNYB-REP (forskolin responsive enhancer element in combination with YB-
Tata minimal promoter)
(SEQ ID NO: 197)
AATAAAATATCTTTATTTTCATTACATCTGTGTGTTGGTTTTTTGTGTGCCATTGACGTCA
CGATTTGACGTCAACCATTGACGTCACGATTTGACGTCAACCATTGACGTCACGATTTGA
CGTCACGATTTGACGTCAACCATTGACTCACGATTTGACTCAACCATTGACTCACGATTT
GACTCAACCATTGACTCACGATTTGACTCACGATTGCGATTAATCCATATGCTCTAGAGG
GTATATAATGGGGGCCACTAGTCTACTACCAGAAAGCTTGGTACCGAGCTCGGATCCAG
CCACCCTCGAGATGCCGGGGTTTTACGAGATTGTGATTAAGGTCCCCAGCGACCTTGACG
AGCATCTGCCCGGCATTTCTGACAGCTTTGTGAACTGGGTGGCCGAGAAGGAGTGGGAG
TTGCCGCCAGATTCTGACTTGGATCTGAATCTGATTGAGCAGGCACCCCTGACCGTGGCC
GAGAAGCTGCAGCGCGACTTTCTGACGGAGTGGCGCCGTGTGAGTAAGGCCCCGGAGGC
CCTTTTCTTTGTGCAATTTGAGAAGGGAGAGAGCTACTTCCACTTACACGTGCTCGTGGA
AACCACCGGGGTGAAATCCTTAGTTTTGGGACGTTTCCTGAGTCAGATTCGCGAAAAACT
GATTCAGAGAATTTACCGCGGGATCGAGCCGACTTTGCCAAACTGGTTCGCGGTCACAA
AGACCAGAAACGGCGCCGGAGGCGGGAACAAGGTGGTGGACGAGTGCTACATCCCCAA
TTACTTGCTCCCCAAAACCCAGCCTGAGCTCCAGTGGGCGTGGACTAATATAGAACAGTA
TTTAAGCGCCTGTTTGAATCTCACGGAGCGTAAACGGTTGGTGGCGCAGCATCTGACGCA
CGTGTCGCAGACGCAGGAGCAGAACAAAGAGAATCAGAATCCCAATTCTGACGCGCCGG
TGATCAGATCAAAAACTTCAGCCAGGTACGGAGAGCTGGTCGGGTGGCTCGTGGACAAG
GGGATTACCTCGGAGAAGCAGTGGATCCAGGAGGACCAGGCCTCATACATCTCCTTCAA
TGCGGCCTCCAACTCGCGGTCCCAAATCAAGGCTGCCTTGGACAATGCGGGAAAGATTA
TGAGCCTGACTAAAACCGCCCCCGACTACCTGGTGGGCCAGCAGCCCGTGGAGGACATT
TCCAGCAATCGGATTTATAAAATTTTGGAACTAAACGGGTACGATCCCCAATATGCGGCT
TCCGTCTTTCTGGGATGGGCCACGAAAAAGTTCGGCAAGAGGAACACCATCTGGCTGTTT
GGGCCTGCAACTACCGGGAAGACCAACATCGCGGAGGCCATAGCCCACACTGTGCCCTT
CTACGGGTGCGTAAACTGGACCAATGAGAACTTTCCCTTCAACGACTGTGTCGACAAGAT
GGTGATCTGGTGGGAGGAGGGGAAGATGACCGCCAAGGTCGTGGAGTCGGCCAAAGCC
ATTCTCGGAGGAAGCAAGGTGCGCGTGGACCAGAAATGCAAGTCCTCGGCCCAGATAGA
CCCGACTCCCGTGATCGTCACCTCCAACACCAACATGTGCGCCGTGATTGACGGGAACTC
AACGACCTTCGAACACCAGCAGCCGTTGCAAGACCGGATGTTCAAATTTGAACTCACCC
GCCGTCTGGATCATGACTTTGGGAAGGTCACCAAGCAGGAAGTCAAAGACTTTTTCCGGT
GGGCAAAGGATCACGTGGTTGAGGTGGAGCATGAATTCTACGTCAAAAAGGGTGGAGCC
AAGAAAAGACCCGCCCCCAGTGACGCAGATATAAGTGAGCCCAAACGGGTGCGCGAGTC
AGTTGCGCAGCCATCGACGTCAGACGCGGAAGCTTCGATCAACTACGCAGACAGGTACC
AAAACAAATGTTCTCGTCACGTGGGCATGAATCTGATGCTGTTTCCCTGCAGACAATGCG
AGAGAATGAATCAGAATTCAAATATCTGCTTCACTCACGGACAGAAAGACTGTTTAGAG
TGCTTTCCCGTGTCAGAATCTCAACCCGTTTCTGTCGTCAAAAAGGCGTATCAGAAACTG
TGCTACATTCATCATATCATGGGAAAGGTGCCAGACGCTTGCACTGCCTGCGATCTGGTC
AATGTGGATTTGGATGACTGCATCTTTGAACAATAATGAAAGCTTGGTCTCTACGAGTAA
TAGACGCCCAGTTGAATTCCTTCGAGCAGACATGATAAGATACATTGATGAGTTTGGACA
AACCACAACTAGAATGCAGTGAAAAAAATGCTTTATTTGTGAAATTTGTGATGCTATTGC
TTTATTTGTAACCATTATAAGCTGCAATAAACAAGTT
FORNMLP (forskolin responsive enhancer element in combination with MLP
minimal promoter)
(SEQ ID NO: 198)
AATAAAATATCTTTATTTTCATTACATCTGTGTGTTGGTTTTTTGTGTGCCATTGACGTCA
CGATTTGACGTCAACCATTGACGTCACGATTTGACGTCAACCATTGACGTCACGATTTGA
CGTCACGATTTGACGTCAACCATTGACTCACGATTTGACTCAACCATTGACTCACGATTT
GACTCAACCATTGACTCACGATTTGACTCACGATTGGGGGGCTATAAAAGGGGGTGGGG
GCGTTCGTCCTCACTCTGCCACC
FORNSV40 (forskolin responsive enhancer element in combination with SV40
minimal promoter)
(SEQ ID NO: 199)
AATAAAATATCTTTATTTTCATTACATCTGTGTGTTGGTTTTTTGTGTGCCATTGACGTCA
CGATTTGACGTCAACCATTGACGTCACGATTTGACGTCAACCATTGACGTCACGATTTGA
CGTCACGATTTGACGTCAACCATTGACTCACGATTTGACTCAACCATTGACTCACGATTT
GACTCAACCATTGACTCACGATTTGACTCACGATTTGCATCTCAATTAGTCAGCAACCAT
AGTCCCGCCCCTAACTCCGCCCATCCCGCCCCTAACTCCGCCCAGTTCCGCCCATTCTCCG
CCCCATCGCTGACTAATTTTTTTTATTTATGCAGAGGCCGAGGCCGCCTCGGCCTCTGAGC
TATTCCAGAAGTAGTGAGGAGGCTTTTTTGGAGGCCTAGGCTTTTGCAAAAAGCTTGCCA
CC
FORNpJB42 (forskolin responsive enhancer element in combination with pJB42
minimal promoter)
(SEQ ID NO: 211)
AATAAAATATCTTTATTTTCATTACATCTGTGTGTTGGTTTTTTGTGTGCCATTGACGTCA
CGATTTGACGTCAACCATTGACGTCACGATTTGACGTCAACCATTGACGTCACGATTTGA
CGTCACGATTTGACGTCAACCATTGACTCACGATTTGACTCAACCATTGACTCACGATTT
GACTCAACCATTGACTCACGATTTGACTCACGATTCTGACAAATTCAGTATAAAAGCTTG
GGGCTGGGGCCGAGCACTGGGGACTTTGAGGGTGGCCAGGCCAGCGTAGGAGGCCAGC
GTAGGATCCTGCTGGGAGCGGGGAACTGAGGGAAGCGACGCCGAGAAAGCAGGCGTAC
CACGGAGGGAGAGAAAAGCTCCGGAAGCCCAGCAGCGGCCACC
FORNTATAm6a (forskolin responsive enhancer element in combination with
TATA-m6A minimal promoter)
(SEQ ID NO: 209)
AATAAAATATCTTTATTTTCATTACATCTGTGTGTTGGTTTTTTGTGTGCCATTGACGTCA
CGATTTGACGTCAACCATTGACGTCACGATTTGACGTCAACCATTGACGTCACGATTTGA
CGTCACGATTTGACGTCAACCATTGACTCACGATTTGACTCAACCATTGACTCACGATTT
GACTCAACCATTGACTCACGATTTGACTCACGATTTATAAAAGGCAGAGCTCGTTTAGTG
AACCGaagcttggactaaagcggacttgtctcgag
FORNMinTK (forskolin responsive enhancer element in combination with MinTK
minimal promoter)
(SEQ ID NO: 200)
AATAAAATATCTTTATTTTCATTACATCTGTGTGTTGGTTTTTTGTGTGCCATTGACGTCA
CGATTTGACGTCAACCATTGACGTCACGATTTGACGTCAACCATTGACGTCACGATTTGA
CGTCACGATTTGACGTCAACCATTGACTCACGATTTGACTCAACCATTGACTCACGATTT
GACTCAACCATTGACTCACGATTTGACTCACGATTTTCGCATATTAAGGTGACGCGTGTG
GCCTCGAACACCGAGCGACCCTGCAGCGACCCGCTTAAGCCACC
FORNCMV (forskolin responsive enhancer element in combination with CMV
minimal promoter
(SEQ ID NO: 201)
AATAAAATATCTTTATTTTCATTACATCTGTGTGTTGGTTTTTTGTGTGCCATTGACGTCA
CGATTTGACGTCAACCATTGACGTCACGATTTGACGTCAACCATTGACGTCACGATTTGA
CGTCACGATTTGACGTCAACCATTGACTCACGATTTGACTCAACCATTGACTCACGATTT
GACTCAACCATTGACTCACGATTTGACTCACGATTGTAGGCGTGTACGGTGGGAGGTCTA
TATAAGCAGAGCTCGTTTAGTGAACCGTCAGATCGCCACC
FORNCMV53 (forskolin responsive enhancer element in combination with CMV53
minimal promoter)
(SEQ ID NO: 202)
AATAAAATATCTTTATTTTCATTACATCTGTGTGTTGGTTTTTTGTGTGCCA
TTGACGTCACGATTTGACGTCAACCATTGACGTCACGATTTGACGTCAACCATTGACGTC
ACGATTTGACGTCACGATTTGACGTCAACCATTGACTCACGATTTGACTCAACCATTGAC
TCACGATTTGACTCAACCATTGACTCACGATTTGACTCACGATTCAACAAAATGTCGTAA
CAAGGGCGGTAGGCGTGTACGGTGGGAGGTCTATATAAGCAGAGCTCGTTTAGTGAACC
GGCCACC
PM-RQ vector
(SEQ ID NO: 203)
ATGGAAGATGCCAAAAACATTAAGAAGGGCCCAGCGCCATTCTACCCACTCGAAGACGG
GACCGCCGGCGAGCAGCTGCACAAAGCCATGAAGCGCTACGCCCTGGTGCCCGGCACCA
TCGCCTTTACCGACGCACATATCGAGGTGGACATTACCTACGCCGAGTACTTCGAGATGA
GCGTTCGGCTGGCAGAAGCTATGAAGCGCTATGGGCTGAATACAAACCATCGGATCGTG
GTGTGCAGCGAGAATAGCTTGCAGTTCTTCATGCCCGTGTTGGGTGCCCTGTTCATCGGT
GTGGCTGTGGCCCCAGCTAACGACATCTACAACGAGCGCGAGCTGCTGAACAGCATGGG
CATCAGCCAGCCCACCGTCGTATTCGTGAGCAAGAAAGGGCTGCAAAAGATCCTCAACG
TGCAAAAGAAGCTACCGATCATACAAAAGATCATCATCATGGATAGCAAGACCGACTAC
CAGGGCTTCCAAAGCATGTACACCTTCGTGACTTCCCATTTGCCACCCGGCTTCAACGAG
TACGACTTCGTGCCCGAGAGCTTCGACCGGGACAAAACCATCGCCCTGATCATGAACAG
TAGTGGCAGTACCGGATTGCCCAAGGGCGTAGCCCTACCGCACCGCACCGCTTGTGTCCG
ATTCAGTCATGCCCGCGACCCCATCTTCGGCAACCAGATCATCCCCGACACCGCTATCCT
CAGCGTGGTGCCATTTCACCACGGCTTCGGCATGTTCACCACGCTGGGCTACTTGATCTG
CGGCTTTCGGGTCGTGCTCATGTACCGCTTCGAGGAGGAGCTATTCTTGCGCAGCTTGCA
AGACTATAAGATTCAATCTGCCCTGCTGGTGCCCACACTATTTAGCTTCTTCGCTAAGAG
CACTCTCATCGACAAGTACGACCTAAGCAACTTGCACGAGATCGCCAGCGGCGGGGCGC
CGCTCAGCAAGGAGGTAGGTGAGGCCGTGGCCAAACGCTTCCACCTACCAGGCATCCGC
CAGGGCTACGGCCTGACAGAAACAACCAGCGCCATTCTGATCACCCCCGAAGGGGACGA
CAAGCCTGGCGCAGTAGGCAAGGTGGTGCCCTTCTTCGAGGCTAAGGTGGTGGACTTGG
ACACCGGTAAGACACTGGGTGTGAACCAGCGCGGCGAGCTGTGCGTCCGTGGCCCCATG
ATCATGAGCGGCTACGTTAACAACCCCGAGGCTACAAACGCTCTCATCGACAAGGACGG
CTGGCTGCACAGCGGCGACATCGCCTACTGGGACGAGGACGAGCACTTCTTCATCGTGG
ACCGGCTGAAGAGCCTGATCAAATACAAGGGCTACCAGGTAGCCCCAGCCGAACTGGAG
AGCATCCTGCTGCAACACCCCAACATCTTCGACGCCGGGGTCGCCGGCCTGCCCGACGAC
GATGCCGGCGAGCTGCCCGCCGCAGTCGTCGTGCTGGAACACGGTAAAACCATGACCGA
GAAGGAGATCGTGGACTATGTGGCCAGCCAGGTTACAACCGCCAAGAAGCTGCGCGGTG
GTGTTGTGTTCGTGGACGAGGTGCCTAAAGGACTGACCGGCAAGTTGGACGCCCGCAAG
ATCCGCGAGATTCTCATTAAGGCCAAGAAGGGCGGCAAGATCGCCGTGTAAGTGTAATG
AAAGCTTGGTCTCTACGAGTAATAGACGCCCAGTTGAATTCCTTCGAGCAGACATGATAA
GATACATTGATGAGTTTGGACAAACCACAACTAGAATGCAGTGAAAAAAATGCTTTATTT
GTGAAATTTGTGATGCTATTGCTTTATTTGTAACCATTATAAGCTGCAATAAACAAGTTA
ACAACAACAATTGCATTCATTTTATGTTTCAGGTTCAGGGGGAGGTGTGGGAGGTTTTTT
AAAGCAAGTAAAACCTCTACAAATGTGGTAAAATCGATAAGGATCCGTAACAACAACAA
TTGCATTCATTTTATGTTTCAGGTTCAGGGGGAGGTGTGGGAGGTTTTTTAAAGCAAGTA
AAACCTCTACAAATGTGGTAAAATCGATAAGGATCCGTCTGGGCCTCATGGGCCTTCCGC
TCACTGCCCGCTTTCCAGTCGGGAAACCTGTCGTGCCAGCTGCATTAACATGGTCATAGC
TGTTTCCTTGCGTATTGGGCGCTCTCCGCTTCCTCGCTCACTGACTCGCTGCGCTCGGTCG
TTCGGGTAAAGCCTGGGGTGCCTAATGAGCAAAAGGCCAGCAAAAGGCCAGGAACCGTA
AAAAGGCCGCGTTGCTGGCGTTTTTCCATAGGCTCCGCCCCCCTGACGAGCATCACAAAA
ATCGACGCTCAAGTCAGAGGTGGCGAAACCCGACAGGACTATAAAGATACCAGGCGTTT
CCCCCTGGAAGCTCCCTCGTGCGCTCTCCTGTTCCGACCCTGCCGCTTACCGGATACCTGT
CCGCCTTTCTCCCTTCGGGAAGCGTGGCGCTTTCTCATAGCTCACGCTGTAGGTATCTCAG
TTCGGTGTAGGTCGTTCGCTCCAAGCTGGGCTGTGTGCACGAACCCCCCGTTCAGCCCGA
CCGCTGCGCCTTATCCGGTAACTATCGTCTTGAGTCCAACCCGGTAAGACACGACTTATC
GCCACTGGCAGCAGCCACTGGTAACAGGATTAGCAGAGCGAGGTATGTAGGCGGTGCTA
CAGAGTTCTTGAAGTGGTGGCCTAACTACGGCTACACTAGAAGAACAGTATTTGGTATCT
GCGCTCTGCTGAAGCCAGTTACCTTCGGAAAAAGAGTTGGTAGCTCTTGATCCGGCAAAC
AAACCACCGCTGGTAGCGGTGGTTTTTTTGTTTGCAAGCAGCAGATTACGCGCAGAAAAA
AAGGATCTCAAGAAGATCCTTTGATCTTTTCTACGGGGTCTGACGCTCAGTGGAACGAAA
ACTCACGTTAAGGGATTTTGGTCATGAGATTATCAAAAAGGATCTTCACCTAGATCCTTT
TAAATTAAAAATGAAGTTTTAAATCAATCTAAAGTATATATGAGTAAACTTGGTCTGACA
GTTACCAATGCTTAATCAGTGAGGCACCTATCTCAGCGATCTGTCTATTTCGTTCATCCAT
AGTTGCCTGACTCCCCGTCGTGTAGATAACTACGATACGGGAGGGCTTACCATCTGGCCC
CAGTGCTGCAATGATACCGCGAGAACCACGCTCACCGGCTCCAGATTTATCAGCAATAA
ACCAGCCAGCCGGAAGGGCCGAGCGCAGAAGTGGTCCTGCAACTTTATCCGCCTCCATC
CAGTCTATTAATTGTTGCCGGGAAGCTAGAGTAAGTAGTTCGCCAGTTAATAGTTTGCGC
AACGTTGTTGCCATTGCTACAGGCATCGTGGTGTCACGCTCGTCGTTTGGTATGGCTTCAT
TCAGCTCCGGTTCCCAACGATCAAGGCGAGTTACATGATCCCCCATGTTGTGCAAAAAAG
CGGTTAGCTCCTTCGGTCCTCCGATCGTTGTCAGAAGTAAGTTGGCCGCAGTGTTATCAC
TCATGGTTATGGCAGCACTGCATAATTCTCTTACTGTCATGCCATCCGTAAGATGCTTTTC
TGTGACTGGTGAGTACTCAACCAAGTCATTCTGAGAATAGTGTATGCGGCGACCGAGTTG
CTCTTGCCCGGCGTCAATACGGGATAATACCGCGCCACATAGCAGAACTTTAAAAGTGCT
CATCATTGGAAAACGTTCTTCGGGGCGAAAACTCTCAAGGATCTTACCGCTGTTGAGATC
CAGTTCGATGTAACCCACTCGTGCACCCAACTGATCTTCAGCATCTTTTACTTTCACCAGC
GTTTCTGGGTGAGCAAAAACAGGAAGGCAAAATGCCGCAAAAAAGGGAATAAGGGCGA
CACGGAAATGTTGAATACTCATACTCTTCCTTTTTCAATATTATTGAAGCATTTATCAGGG
TTATTGTCTCATGAGCGGATACATATTTGAATGTATTTAGAAAAATAAACAAATAGGGGT
TCCGCGCACATTTCCCCGAAAAGTGCCAC
pAAV vector:
(SEQ ID NO: 204)
CGATAGATCTAGGAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCGGGCGGCCTC
AGTGAGCGAGCGAGCGCGCAGCTGCCTGCAGGCAGCTTGGCACTGGCCGTCGTTTTACA
ACGTCGTGACTGGGAAAACCCTGGCGTTACCCAACTTAATCGCCTTGCAGCACATCCCCC
TTTCGCCAGCTGGCGTAATAGCGAAGAGGCCCGCACCGATCGCCCTTCCCAACAGTTGCG
CAGCCTGAATGGCGAATGGCGCCTGATGCGGTATTTTCTCCTTACGCATCTGTGCGGTAT
TTCACACCGCATACGTCAAAGCAACCATAGTACGCGCCCTGTAGCGGCGCATTAAGCGC
GGCGGGTGTGGTGGTTACGCGCAGCGTGACCGCTACACTTGCCAGCGCCCTAGCGCCCG
CTCCTTTCGCTTTCTTCCCTTCCTTTCTCGCCACGTTCGCCGGCTTTCCCCGTCAAGCTCTA
AATCGGGGGCTCCCTTTAGGGTTCCGATTTAGTGCTTTACGGCACCTCGACCCCAAAAAA
CTTGATTTGGGTGATGGTTCACGTAGTGGGCCATCGCCCTGATAGACGGTTTTTCGCCCTT
TGACGTTGGAGTCCACGTTCTTTAATAGTGGACTCTTGTTCCAAACTGGAACAACACTCA
ACCCTATCTCGGGCTATTCTTTTGATTTATAAGGGATTTTGCCGATTTCGGCCTATTGGTT
AAAAAATGAGCTGATTTAACAAAAATTTAACGCGAATTTTAACAAAATATTAACGTTTAC
AATTTTATGGTGCACTCTCAGTACAATCTGCTCTGATGCCGCATAGTTAAGCCAGCCCCG
ACACCCGCCAACACCCGCTGACGCGCCCTGACGGGCTTGTCTGCTCCCGGCATCCGCTTA
CAGACAAGCTGTGACCGTCTCCGGGAGCTGCATGTGTCAGAGGTTTTCACCGTCATCACC
GAAACGCGCGAGACGAAAGGGCCTCGTGATACGCCTATTTTTATAGGTTAATGTCATGAT
AATAATGGTTTCTTAGACGTCAGGTGGCACTTTTCGGGGAAATGTGCGCGGAACCCCTAT
TTGTTTATTTTTCTAAATACATTCAAATATGTATCCGCTCATGAGACAATAACCCTGATAA
ATGCTTCAATAATATTGAAAAAGGAAGAGTATGAGTATTCAACATTTCCGTGTCGCCCTT
ATTCCCTTTTTTGCGGCATTTTGCCTTCCTGTTTTTGCTCACCCAGAAACGCTGGTGAAAG
TAAAAGATGCTGAAGATCAGTTGGGTGCACGAGTGGGTTACATCGAACTGGATCTCAAC
AGCGGTAAGATCCTTGAGAGTTTTCGCCCCGAAGAACGTTTTCCAATGATGAGCACTTTT
AAAGTTCTGCTATGTGGCGCGGTATTATCCCGTATTGACGCCGGGCAAGAGCAACTCGGT
CGCCGCATACACTATTCTCAGAATGACTTGGTTGAGTACTCACCAGTCACAGAAAAGCAT
CTTACGGATGGCATGACAGTAAGAGAATTATGCAGTGCTGCCATAACCATGAGTGATAA
CACTGCGGCCAACTTACTTCTGACAACGATCGGAGGACCGAAGGAGCTAACCGCTTTTTT
GCACAACATGGGGGATCATGTAACTCGCCTTGATCGTTGGGAACCGGAGCTGAATGAAG
CCATACCAAACGACGAGCGTGACACCACGATGCCTGTAGCAATGGCAACAACGTTGCGC
AAACTATTAACTGGCGAACTACTTACTCTAGCTTCCCGGCAACAATTAATAGACTGGATG
GAGGCGGATAAAGTTGCAGGACCACTTCTGCGCTCGGCCCTTCCGGCTGGCTGGTTTATT
GCTGATAAATCTGGAGCCGGTGAGCGTGGGTCTCGCGGTATCATTGCAGCACTGGGGCC
AGATGGTAAGCCCTCCCGTATCGTAGTTATCTACACGACGGGGAGTCAGGCAACTATGG
ATGAACGAAATAGACAGATCGCTGAGATAGGTGCCTCACTGATTAAGCATTGGTAACTG
TCAGACCAAGTTTACTCATATATACTTTAGATTGATTTAAAACTTCATTTTTAATTTAAAA
GGATCTAGGTGAAGATCCTTTTTGATAATCTCATGACCAAAATCCCTTAACGTGAGTTTT
CGTTCCACTGAGCGTCAGACCCCGTAGAAAAGATCAAAGGATCTTCTTGAGATCCTTTTT
TTCTGCGCGTAATCTGCTGCTTGCAAACAAAAAAACCACCGCTACCAGCGGTGGTTTGTT
TGCCGGATCAAGAGCTACCAACTCTTTTTCCGAAGGTAACTGGCTTCAGCAGAGCGCAGA
TACCAAATACTGTTCTTCTAGTGTAGCCGTAGTTAGGCCACCACTTCAAGAACTCTGTAG
CACCGCCTACATACCTCGCTCTGCTAATCCTGTTACCAGTGGCTGCTGCCAGTGGCGATA
AGTCGTGTCTTACCGGGTTGGACTCAAGACGATAGTTACCGGATAAGGCGCAGCGGTCG
GGCTGAACGGGGGGTTCGTGCACACAGCCCAGCTTGGAGCGAACGACCTACACCGAACT
GAGATACCTACAGCGTGAGCTATGAGAAAGCGCCACGCTTCCCGAAGGGAGAAAGGCG
GACAGGTATCCGGTAAGCGGCAGGGTCGGAACAGGAGAGCGCACGAGGGAGCTTCCAG
GGGGAAACGCCTGGTATCTTTATAGTCCTGTCGGGTTTCGCCACCTCTGACTTGAGCGTC
GATTTTTGTGATGCTCGTCAGGGGGGCGGAGCCTATGGAAAAACGCCAGCAACGCGGCC
TTTTTACGGTTCCTGGCCTTTTGCTGGCCTTTTGCTCACATGTTCTTTCCTGCGTTATCCCC
TGATTCTGTGGATAACCGTATTACCGCCTTTGAGTGAGCTGATACCGCTCGCCGCAGCCG
AACGACCGAGCGCAGCGAGTCAGTGAGCGAGGAAGCGGAAGAGCGCCCAATACGCAAA
CCGCCTCTCCCCGCGCGTTGGCCGATTCATTAATGCAGCTGGCACGACAGGTTTCCCGAC
TGGAAAGCGGGCAGTGAGCGCAACGCAATTAATGTGAGTTAGCTCACTCATTAGGCACC
CCAGGCTTTACACTTTATGCTTCCGGCTCGTATGTTGTGTGGAATTGTGAGCGGATAACA
ATTTCACACAGGAAACAGCTATGACCATGATTACGAATTGCCTGCAGGCAGCTGCGCGCT
CGCTCGCTCACTGAGGCCGCCCGGGCAAAGCCCGGGCGTCGGGCGACCTTTGGTCGCCC
GGCCTCAGTGAGCGAGCGAGCGCGCAGAGAGGGAGTGGCCAACTCCATCACTAGGGGTT
CCTATC

The foregoing is illustrative of the present invention, and is not to be construed as limiting thereof. The invention is defined by the following claims, with equivalents of the claims to be included therein.

### REFERENCES

Craig, J. C. et al., 2001. Consensus and Variant cAMP-regulated Enhancers Have Distinct CREB-binding Properties. THE JOURNAL OF BIOLOGICAL CHEMISTRY, 276(15), pp. 11719-11728.
Ede, C., Chen, X., Lin, M.-Y. & Chen, Y. Y., 2016. Quantitative Analyses of Core Promoters Enable Precise Engineering of Regulated Gene Expression in Mammalian Cells. ACS Synth Biol., 5(5), p. 395-404.
Hess, H., Angel, P. & Schorpp-Kistner, M., 2004. AP-1 subunits: quarrel and harmony among siblings. Journal of Cell Science, Volume 117, pp. 5965-5973.
Javan, B. & Shanbazi, M., 2017. Hypoxia-inducible tumour-specific promoters as a dual-targeting transcriptional regulation system for cancer gene therapy. Ecancer, 11(751), pp. 1-10.
Kaluz, S., Kaluzová, M. & Stanbridge, E. J., 2008. Rational design of minimal hypoxia-inducible enhancers. Biochem Biophys Res Commun., 370(4), p. 613-618.
Schödel, J. et al., 2011. High-resolution genome-wide mapping of HIF-binding sites by ChIP-seq. Blood, 117(23), pp. e207-e217.
Samali, A., FitzGerald, U., Deegan, S. & Gupta, S., 2010. Methods for Monitoring Endoplasmic Reticulum Stress and the Unfolded Protein Response. International Journal of Cell Biology, pp. 1-11.
Sharma, C. S. & Richards, J. S., 2000. Regulation ofAPI (Jun/Fos) Factor Expression and Activation in. THE JOURNAL OF BIOLOGICAL CHEMISTRY, 275(43), p. 33718-33728.
Yan, K. et al., 2016. The cyclic AMP signaling pathway: Exploring targets for successful drug discovery (Review). MOLECULAR MEDICINE REPORTS, Volume 13, pp. 3715-3723.

1. A nucleic acid construct comprising:
a nucleic acid sequence comprising at least one of: a nucleic acid sequence encoding a E4 protein, a nucleic acid sequence encoding a E2A protein, and a nucleic acid sequence encoding a VA RNA, wherein each nucleic acid sequences encoding any one of E4 protein, E2A protein, and VA RNA is operatively linked to a first regulatable promoter; and
a nucleic acid sequence encoding a Rep protein, wherein the nucleic acid encoding a Rep protein is under the control of a second regulatable promoter or heterologous transcriptional activator,
wherein the first and the second regulatable promoters are different.

2. The nucleic acid construct of claim 1, wherein the Rep protein is a modified Rep protein.

3. The nucleic acid construct of claim 2, wherein the modified Rep protein has a lysine to arginine mutation at amino acid 84.

4. The nucleic acid construct of claim 2, wherein the nucleic acid encoding the Rep protein further comprises the nucleic acid encoding a ribozyme at its 3′ end.

5. The nucleic acid construct of claim 1, wherein the regulatable promoter operatively linked to the nucleic acid encoding the Rep protein is an inducible promoter or comprises a binding site for the heterologous transcriptional activator.

6. The nucleic acid construct of claim 5, wherein the inducible promoter is selected from the group consisting of a forskolin inducible promoter, a hypoxia inducible promoter, a tetracycline inducible promoter, an alcohol inducible promoter, a steroid inducible promoter, an RU486 inducible promoter, an ecdysone inducible promoter, a rapamycin inducible promoter, a metallothionein inducible promoter, a hormone inducible promoter and a metal inducible promoter

7. The nucleic acid construct of claim 5, wherein the inducible promoter comprises at least one of: lacks a minimal promoter, comprises a TATA box sequence, or a p5 replication sequence, or both a TATA box sequence and p5 replication sequence.

8.-14. (canceled)

15. A cell comprising the nucleic acid construct of claim 1.

16.-22. (canceled)

23. The cell of claim 15, further comprising at least one of
a nucleic acid construct comprising a nucleic acid sequence encoding a tetracycline-responsive transactivator protein operatively linked to a constitutive promoter,
a nucleic acid construct comprising a nucleic acid sequence encoding a marker protein, or
a nucleic acid construct comprising a nucleic acid sequence encoding a marker protein, wherein the nucleic sequence encoding a marker protein is flanked by recombinase recognition sequences (RRSs) in the same orientation with respect to each other.

24. (canceled)

25. (canceled)

26. The cell of claim 15, wherein expression of the nucleic acid construct is a stable expression or a transient expression.

27. (canceled)

28. The cell of claim 15, wherein the cell comprises at least two nucleic acid constructs, and the expression of the at least two nucleic acid constructs is a stable expression,
wherein the cell comprises at least two nucleic acid constructs, and the expression of the at least two nucleic acid constructs is a transient expression, or
wherein the cell comprises at least two nucleic acid constructs, and the expression of at least one nucleic acid construct is a stable expression.

29.-40. (canceled)

41. A nucleic acid construct comprising
a first nucleic acid construct comprising in a 5′ to 3′ direction: a promoter, a stop nucleic acid sequence flanked by a first pair of recombinase recognition sequences (RRS), and nucleic acid sequence encoding a Rep protein, wherein the promoter is operatively linked to the nucleic acid encoding the Rep protein, and
a second nucleic acid construct comprising, in a 5′ to 3′ direction: a promoter, a stop nucleic acid sequence flanked by a second pair of recombinase recognition sequences (RRSs), and nucleic acid sequence encoding one or more of E2A, E4, and VA RNA, wherein the promoter is operatively linked to the nucleic acid encoding the one or more of E2A, E4, and VA RNA.

42. The nucleic acid construct of claim 41, wherein the nucleic acid construct further comprises a nucleic acid encoding one or more selection markers flanked between a third pair of recombinase recognition sequences (RRSs), wherein the pair of RRS are in the same orientation with respect to each other, and wherein the nucleic acid encoding the one or more selection markers is operatively linked to one or more promoters from claim 41.

43. The nucleic acid construct of claim 41, wherein the first pair of RRS and the second pair of RRS are in the same orientation with respect to each other,
wherein the first pair of RRS and the second pair of RRS are in the inverse orientation with respect to each other,
wherein the first pair of RRS, second pair of RRS, and third pair of RRS are each responsive to different tyrosine recombinase or serine integrase enzymes,
wherein the first pair of RRS and second pair of RRS, are responsive to the same tyrosine recombinase or serine integrase enzyme,
wherein the first pair of RRS, or second pair of RRS, or both are Cre-responsive RRS, and/or
wherein the third pair of RRSs are Flipase-responsive RRS.

44.-48. (canceled)

49. A cell comprising the nucleic acid construct of claim 41.

50. (canceled)

51. A method of producing viral particles, comprising;
providing any cell line claim 15 in a viral expression system;
culturing the cells for a time sufficient and under conditions in which the at least one nucleic acid under the control of an regulatable promoter is expressed;
culturing the cells under conditions in which viral particles are produced; and
optionally isolating the viral particles.

52.-64. (canceled)

65. A method of producing viral particles, comprising;
a. providing the cell line of claim 49;
b. culturing the cells for a time sufficient and under conditions in which at least the nucleic acid sequence encoding the E4 protein, the nucleic acid sequence encoding the E2A protein, or the nucleic acid sequence encoding the VA RNA is expressed first a viral vector production protocol;
c. culturing the cells for a time sufficient and under conditions in which the nucleic acid sequence encoding a toxic protein or Rep protein is expressed second in the viral vector production protocol;
d. culturing the cells under conditions in which viral particles are produced; and
e. optionally isolating the viral particles.

66. The method of claim 65, wherein culturing in step (b) is culturing with a recombinase specific for the first pair of recombinase recognition sequences (RRSs).

67. The method of claim 65, wherein culturing in step (c) is culturing with a recombinase specific for the second pair of recombinase recognition sequences (RRSs).

68. The method of claim 1, wherein the transcriptional activator is a zinc finger transcriptional activator (ZF-TA).
